(19) United States
(12) Patent Application Publication
(10) Pub. No.: US 2026/0274842 A1
(43) Pub. Date: Sep. 17, 2026

(54) FUSED BICYCLIC COMPOUNDS AS VRK1 INHIBITORS

(71) Applicant: Incyte Corporation, Wilmington, DE (US)

(72) Inventors: Le Zhao, Wilmington, DE (US); Yanran Ai, West Chester, PA (US); Huan Chen, Glen Mills, PA (US); Dipendu Das, Wilmington, DE (US); Balu Dherange, Wilmington, DE (US); Kelly Federowicz, Garnet Valley, PA (US); Gia Hoang, Wilmington, DE (US); Bin Hu, Garnet Valley, PA (US); Javier A. Feliciano Leal, Bryn Mawr, PA (US); Yong Li, Newark, DE (US); Leslie Brooke Epling McQuirter, Wilmington, DE (US); Xiaozhao Wang, Moorestown, NJ (US); Eddy W. Yue, Landenberg, PA (US); Guofeng Zhang, Kennett Square, PA (US)

(21) Appl. No.: 19/566,771

(22) Filed: Mar. 13, 2026

Related U.S. Application Data
(60) Provisional application No. 63/771,181, filed on Mar. 13, 2025.
(60) Provisional application No. 63/771,166, filed on Mar. 13, 2025.
(60) Provisional application No. 63/771,190, filed on Mar. 13, 2025.
(60) Provisional application No. 63/818,492, filed on Jun. 5, 2025.
(60) Provisional application No. 63/938,639, filed on Dec. 11, 2025.
(60) Provisional application No. 63/938,611, filed on Dec. 11, 2025.
(60) Provisional application No. 63/938,661, filed on Dec. 11, 2025.

(51) Int. Cl. C07D 471/04; A61K 31/437; A61K 31/439; A61K 31/444; A61K 31/4545; A61K 31/4985; A61K 31/519; A61P 35/00; C07B 59/00; C07D 519/00

(52) U.S. Cl. CPC C07D 471/04; A61K 31/437; A61K 31/439; A61K 31/444; A61K 31/4545; A61K 31/4985; A61K 31/519; A61P 35/00; C07B 59/002; C07D 519/00

(57) ABSTRACT

Disclosed are compounds of Formula (I):

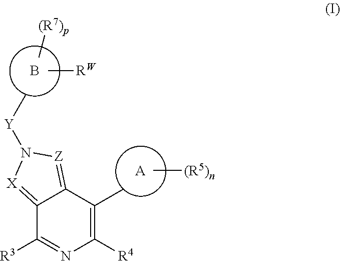

and pharmaceutically acceptable salts thereof, and methods of using the compounds for treatment of cancer and inhibiting VRK1, as well as pharmaceutical compositions comprising such compounds.

### CLAIM OF PRIORITY

This application claims the benefit of priority of U.S. Prov. Appl. Nos. 63/771,166, filed Mar. 13, 2025; 63/771,181, filed Mar. 13, 2025; 63/771,190, filed Mar. 13, 2025; 63/818,492, filed Jun. 5, 2025; 63/938,611, filed Dec. 11, 2025; 63/938,639, filed Dec. 11, 2025; and 63/938,661, filed Dec. 11, 2025, each of which are incorporated herein by reference in their entirety.

### FIELD OF THE DISCLOSURE

The present application is concerned with pharmaceutically active compounds. The disclosure provides compounds as well as their compositions and methods of use. The compounds are inhibitors of VRK1 and are useful in the treatment of various diseases including cancer.

### BACKGROUND

Vaccinia related kinase (VRK) family are characterized by notable sequence homology to the virus-encoded B1 kinase (vvB1). VRK1 is a member of a subgroup of the casein kinase-1 (CK1) family that also contains VRK2 and the pseudokinase VRK3 (J. Biol. Chem. 279:7934-7946 (2004)). VRK1 and VRK2 have been associated with various cellular processes, including cell replication, chromatin remodeling, and DNA damage response (ACS Med. Chem. Lett. 2019, 10, 9, 1266-1271).

VRK1 is a serine/threonine-protein kinase that plays a pivotal role in regulating cell cycle, DNA damage response, nuclear condensation, and transcriptional control. VRK1 has been found to be overexpressed in multiple cancer types, including breast cancer, lung cancer, colorectal cancer, head and neck carcinomas, liver cancer, multiple myeloma, esophageal carcinoma, and all hematopoietic tumors, suggesting its involvement in tumorigenesis. Functionally, VRK1 phosphorylates multiple substrates involved in both cell cycle progression and cell-cycle arrest. VRK1 has been reported to phosphorylate histone H3 and H2AX to regulate chromatin remodeling, transcription factors including p53, c-Jun, ATF2, CREB and Sox2 to activate of transcription, regulate assembly and disassembly of the nuclear envelop by phosphorylating BANF1 in cell cycle progression (Cancer Letters, Volume 503, 10 Apr. 2021, Pages 117-128). Due to its role in cell proliferation and survival of tumor cells, VRK1 has emerged as a potential therapeutic target for cancer treatment.

Genetic knockdown evidence suggests that VRK1 depletion sensitizes cells to cancer treatments based on DNA damage such as ionizing radiation or doxorubicin by impairing the DNA damage response (Cellular and Molecular Life Sciences (2018) 75:2375-2388). Depletion of VRK1 also slows tumor cell proliferation and tumor growth and metastasis in vivo (Oncogenesis volume 2, page 48 (2013)). Genome-scale CRISPR/Cas9 loss of functions screens have identified a paralog-based synthetic lethality role of VRK1 in adult and pediatric gliomas and neuroblastoma, where VRK2 is silenced by promoter methylation (JCI Insight. 2022; 7(19):e158755; Cancer Res. 2022 Nov. 2; 82(21):4044-4057). The VRK1-VRK2 synthetic-lethal interaction was dependent on VKR1 kinase activity and was rescued by ectopic expression of VRK2 (Cancer Res. 2022 Nov. 2; 82(21):4044-4057). These results indicated that inhibiting VRK1 kinase activity with small molecule inhibitors could be a viable therapeutic strategy in VRK2-methylated GBM and neuroblastoma. Other then glioblastoma and neuroblastoma, preclinical data indicated other cancer types where VRK1 ply as a critical role in cancer cell proliferation, including but not limited to Ewing Sarcoma, Astrocytoma, Diffuse Glioma, Bone Sarcoma, Solid tumors e.g. esophageal squamous cell carcinoma, colorectal adenocarcinoma (DepMap; Oncotarget. 2017; 8:65642-65658; Cancer Cell 28, 668-681, Nov. 9, 2014; Pathology—Research and Practice 214 (2018) 112-11). These observations suggests that inhibitors of VRK1 can be of potential use in cancer treatments, by themselves or in combinations, by facilitating inhibition of proliferation and at the same time sensitizing cells to treatments based on DNA damage.

Currently, there are no potent and specific small molecule inhibitors of VRK1. Some studies that have made progresses in developing VRK1 inhibitors including: (1) one study discovered an aminopyridine scaffold as a basis for VRK1 inhibitors and the most potent compound in this study had an IC50 value of 150 nM (ACS Med. Chem. Lett. 2019, 10, 9, 1266-1271); (2) Another study developed VRK1 inhibitors based on BI-D1870, a pteridinone inhibitor of RSK kinases. The optimized VRK1 inhibitor #36 was more selective and mimicked the cellular outcomes of VRK1 depletion (J. Med. Chem. 2024, 67, 11, 8609-8629).

Accordingly, there is a need for new compounds that inhibit VRK1 and which are useful in treating cancers. This application address this need and others.

### SUMMARY

The present disclosure provides, inter alia, compounds of Formula (I):

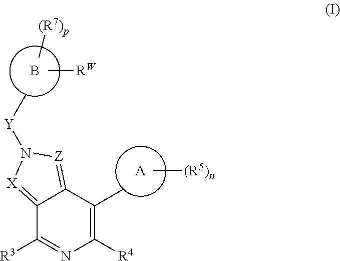

or a pharmaceutically acceptable salt thereof, wherein constituent variables are defined herein.

The present disclosure further provides a VRK1 inhibitor, or a pharmaceutically acceptable salt thereof.

The present disclosure further provides a pharmaceutical composition comprising a compound disclosed herein, or a pharmaceutically acceptable salt thereof, and one or more pharmaceutically acceptable excipient or carrier.

The present disclosure further provides methods of inhibiting VRK1, comprising contacting the VRK1 with a VRK1 inhibitor, or a pharmaceutically acceptable salt thereof.

The present disclosure further provides methods of inhibiting VRK1, comprising administering to a patient a compound disclosed herein, or a pharmaceutically acceptable salt thereof. In some embodiments, the compounds, and pharmaceutically acceptable salts thereof, are selective for VRK1 over VRK2.

The present disclosure further provides methods of treating a disease or disorder associated with VRK1 activity, comprising administering to a patient in need thereof a therapeutically effective amount of a compound of disclosed herein, or a pharmaceutically acceptable salt thereof. In some embodiments, the method is a method of treating sarcoma, colorectal adenocarcinoma, acute myeloid leukemia, glioblastoma, or neuroblastoma. In some embodiments, the method is a method of treating bone cancer, cancer of the neck and head, rectal cancer, esophageal cancer, sarcoma of soft tissue, neoplasm of the central nervous system (CNS), brain stem, glioma, colon cancer, Ewing's sarcoma, brain cancer, astrocytoma, neuroblastoma, intestinal cancer, nervous system cancers, osteosarcoma, cancer of the small bowel (adenocarcinoma, lymphoma, carcinoid tumors, Kaposi's sarcoma, leiomyoma, hemangioma, lipoma, neurofibroma, fibroma), or cancer of the large bowel (adenocarcinoma, tubular adenoma, villous adenoma, hamartoma, leiomyoma).

The present disclosure also provides use of a compound of disclosed herein, or a pharmaceutically acceptable salt thereof, for preparation of a medicament for use in any of the methods described herein.

The present disclosure also provides a compound of disclosed herein, or a pharmaceutically acceptable salt thereof, for use in any of the methods described herein.

### DETAILED DESCRIPTION

The present disclosure provides a VRK1 inhibitor, or a pharmaceutically acceptable salt thereof.

In some embodiments, the VRK1 inhibitor, or a pharmaceutically acceptable salt thereof, covalently binds with a cysteine in VRK1. In some embodiments, the VRK1 inhibitor, or a pharmaceutically acceptable salt thereof, binds with a water in the binding pocket of VRK1. In some embodiments, the VRK1 inhibitor, or a pharmaceutically acceptable salt thereof, binds with Phe134 of VRK1. In some embodiments, the VRK1 inhibitor, or a pharmaceutically acceptable salt thereof, binds with Lys71 of VRK1. In some embodiments, the VRK1 inhibitor, or a pharmaceutically acceptable salt thereof, binds with Asp132 of VRK1. In some embodiments, the VRK1 inhibitor, or a pharmaceutically acceptable salt thereof, binds with the phosphate-binding loop of VRK1.

In some embodiments, the VRK1 inhibitor, or a pharmaceutically acceptable salt thereof, covalently binds with a cysteine in VRK1, a water in the binding pocket of VRK1, and Phe134 of VRK1.

In some embodiments, the VRK1 inhibitor, or a pharmaceutically acceptable salt thereof, covalently binds with a cysteine in VRK1, binds with a water in the binding pocket of VRK1, binds with Phe134 of VRK1, binds with Lys71 of VRK1, binds with Asp132 of VRK1, and binds with the phosphate-binding loop of VRK1.

The VRK1 inhibitor can include a compound of the present disclosure, or a pharmaceutically acceptable salt thereof.

The present disclosure provides a compound of Formula (I) is:

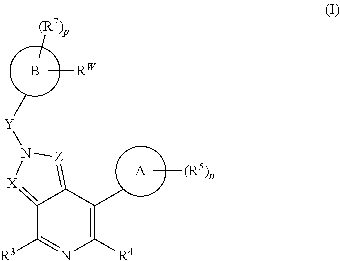

or a pharmaceutically acceptable salt thereof, wherein:
n is 0, 1, 2, 3, 4, 5, or 6;
p is 0, 1, 2, 3, 4, 5, or 6;
r is 1 or 2;
X is N or CR¹;
Z is N or CR²;
Y is —(Cy¹)ₘ-(Y′)ₛ—, wherein Y¹ is attached to Ring moiety B;
m is 1; s is 1; and Y¹ is a bond; or
m is 1; s is 1; and Y¹ is —(CR⁶R⁶⁰)ᵣ—, —CR⁶R⁶⁰—C(O)—, —C(O)—CR⁶R⁶⁰—, or C(O); or
m is 0; s is 1; and Y¹ is —(CR⁶R⁶⁰)ᵣ—, —CR⁶R⁶⁰—C(O)—, —C(O)—CR⁶R⁶⁰—, or C(O);
Cy¹ is C₃₋₁₀ cycloalkyl, 6-10 membered aryl, 4-10 membered heterocycloalkyl, or 5-10 membered heteroaryl, which is optionally substituted by 1, 2, 3, 4, 5, or 6 independently selected R¹⁰ substituents;
Ring moiety A is C₃₋₁₂ cycloalkyl, 6-10 membered aryl, 4-12 membered heterocycloalkyl, or 5-10 membered heteroaryl;
Ring moiety B is C₃₋₁₂ cycloalkyl, 6-10 membered aryl, 4-12 membered heterocycloalkyl, or 5-10 membered heteroaryl;
R¹ is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{b1}, SR^{b1}, NHOR^{a11}, C(O)R^{b1}, C(O)NR^{c1}R^{d1}, C(O)NR^{c1}(OR^{a1}), C(O)OR^{a11}, OC(O)R^{b1}, OC(O)NR^{c1}R^{d1}, NR^{c1}R^{d1}, NR^{c1}NR^{c1}R^{d1}, NR^{c1}C(O)R^{b1}, NR^{c1}C(O)OR^{a11}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}C(═NR^{e1})R^{b1}, NR^{c1}S(O)NR^{c1}R^{d1}, NR^{c1}S(O)R^{b1}, NR^{c1}S(O)₂R^{b1}, NR^{c1}S(O)(═NR^{e1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, OS(O)₂R^{b1}, S(O)(═NR^{e1})R^{b1}, SF₅, P(O)R^{f1}R^{g1}, OP(O)(OR^{h1})(ORⁱ¹), P(O)(OR^{h1})(ORⁱ¹), and BR^{j1}R^{k1}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R¹ is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h1} and Rⁱ¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j1} and R^{k1} is independently selected from OH, C₁₋₆ alkoxy, and C₁₋₆ haloalkoxy;
or any R^{j1} and R^{k1} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1′} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f11} and R^{k11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h11} and Rⁱ¹¹ is independently selected from H, C₁₋₆ alkyl, C₁₋₆haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j11} and R^{k11} is independently selected from OH, C₁₋₆ alkoxy, and C₁₋₆ haloalkoxy;
or any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, R^{c12}C(O)NR^{c12}R^{d12}, C(═NR^{e12})R^{b12}, C(═NR^{e12})NR^{c12}R^{d12}, NR^{c12}C(═NR^{e12})NR^{c12}R^{d12}, NR^{c12}C(═NR^{e2})R^{b12}, NR^{c12}S(O)NR^{c12}R^{d12}, NR^{c12}S(O)R^{b12}, NR^{c12}S(O)₂R^{b12}, NR^{c12}S(O)(═NR^{e12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{ji2}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a12}, R^{e12}, and R^{d12} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents; or, any R^{c12} and R^{d12} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl; R² is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{b2}, SR^{b2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, S(O)(═NR^{e2})R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h2} and Rⁱ² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})NR^{c21}R^{d21}, NR^{c21}C(═NR^{e21})R^{b21}, NR^{c21}S(O)NR^{c21}R^{d21}, NR^{c21}S(O)R^{b21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
or, any R^{c21} and R^{d21} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2B} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f21} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a22}, SR^{a22}, NHOR^{a22}C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR^{c22}NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22})R^{b22}, NR^{c22}S(O)NR^{c22}R^{d22}, NR^{c22}S(O)R^{b22}, NR^{c22}S(O)₂R^{b22}, NR^{c22}S(O)(═NR^{e22})R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, OS(O)(═NR^{e22})R^{b22}, OS(O)₂R^{b22}, S(O)(═NR^{e22})R^{b22}, SF₅, P(O)R^{f22}R^{g22}, OP(O)(OR^{h22})(ORⁱ²²), P(O)(OR^{h22})(ORⁱ²²), and BR^{j22}R^{k22}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c22} and R^{d22} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b22} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j22} and R^{g22} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h22} and Rⁱ²² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j22} and R^{k22} is independently selected from OH, C₁₋₆ alkoxy, and C₁₋₆ haloalkoxy;
or any R^{j22} and R^{k22} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b3} is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy; R⁴ is selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4}, NR^{c4}C(═NR^{e4})R^{b4}, NR^{c4}S(O)NR^{c4}R^{d4}, NR^{c4}S(O)R^{b4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
each R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b4} is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e4} is independently selected from H, OH, CN, C₁₋₆ alkyl, C₁₋₆ alkoxy, C₁₋₆ haloalkyl, and C₁₋₆ haloalkoxy;
each R⁵ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
or, any R^{c5} and R^{d5} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a51}, SR^{a51}, NHOR^{a51}, C(O)R^{b51}, C(O)NR^{c51}R^{d51}, C(O)NR^{c5}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)NR^{c51}R^{d51}, NR^{c51}S(O)R^{b51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, OS(O)₂R^{b51}, S(O)(═NR^{e51})R^{b51}, SF₅, P(O)R^{f51}R^{g51}, OP(O)(OR^{h51})(ORⁱ⁵¹), P(O)(OR^{h51})(ORⁱ⁵¹), and BR^{j51}R^{k51}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c51} and R^{d51} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b51} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f51} and R^{g51} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h51} and Rⁱ⁵¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j51} and R^{k51} is independently selected from OH, C₁₋₆ alkoxy, and C₁₋₆ haloalkoxy;
or any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶ is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c6}R^{d6}, C(═NR^{e6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)NR^{c6}R^{d6}, NR^{c6}S(O)R^{b6}, NR^{c6}S(O)₂R^{b6}, NR^{c6}S(O)(═NR^{e6})R^{b6}, NR^{c6}S(O)₂NR^{c6}R^{d6}, S(O)R^{b6}, S(O)NR^{c6}R^{d6}, S(O)₂R^{b6}, S(O)₂NR^{c6}R^{d6}, OS(O)(═NR^{e6})R^{b6}, OS(O)₂R^{b6}, S(O)(═NR^{e6})R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
or, any R^{c6} and R^{d6} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected RA substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f6} and R^{g6} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h6} and Rⁱ⁶ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j6} and R^{k6} is independently selected from OH, C₁₋₆ alkoxy, and C₁₋₆ haloalkoxy;
or any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, NHOR^{a61}, C(O)R^{b61}, C(O)NR^{c61}R^{d61}, C(O)NR^{c61}(OR^{a61}), C(O)OR^{a61}, OC(O)R^{b61}, OC(O)NR^{c6}R^{d61}, NR^{c61}R^{d61}, NR^{c61}NR^{c61}R^{d61}, NR^{c61}C(O)R^{b61}, NR^{c61}C(O)OR^{a61}, NR^{c61}C(O)NR^{c61}R^{d61}, C(═NR^{e61})R^{b61}, C(═NR^{e61})NR^{c61}R^{d61}, NR^{c61}C(═NR^{e61})NR^{c61}R^{d61}, NR^{c61}C(═NR^{e61})R^{b61}, NR^{c61}S(O)NR^{c61}R^{d61}, NR^{c6}, S(O)R^{b61}, NR^{c61}S(O)₂R^{b61}, NR^{c61}S(O)(═NR^{e61})R^{b61}, NR^{c61}S(O)₂NR^{c61}R^{d61}, S(O)R^{b61}, S(O)NR^{c61}R^{d61}, S(O)₂R^{b61}, S(O)₂NR^{c6}R^{d61}, OS(O)(═NR^{e61})R^{b61}, OS(O)₂R^{b61}, S(O)(═NR^{e61})R^{b61}, SF₅, P(O)R^{f61}R^{g61}, OP(O)(OR^{h61})(ORⁱ⁶¹), P(O)(OR^{h61})(ORⁱ⁶¹), and BRⁱ⁶¹R^{k61}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{6B} substituents;
each R⁶¹ is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{e61} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f61} and R^{g61} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h61} and Rⁱ⁶¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j61} and R^{k61} is independently selected from OH, C₁₋₆ alkoxy, and C₁₋₆ haloalkoxy;
or any R^{j61} and R^{k61} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, NHOR^{a62}C(O)R^{b62}, C(O)NR^{c62}R^{d62}, C(O)NR^{c62}(OR^{a62}), C(O)OR^{a62}, OC(O)R^{b62}, OC(O)NR^{c62}R^{d62}, NR^{c62}R^{d62}, NR^{c62}NR^{c62}R^{d62}, NR^{c62}C(O)R^{b62}, NR^{c62}C(O)OR^{a62}, NR^{c62}C(O)NR^{c62}R^{d62}, C(═NR^{e62})R^{b62}, C(═NR^{e62})NR^{c62}R^{d62}, NR^{c62}C(═NR^{e62})NR^{c62}R^{d62}, NR^{c62}C(═NR^{e62})R^{b62}, NR^{c62}S(O)NR^{c62}R^{d62}, NR^{c62}S(O)R^{b62}, NR^{c62}S(O)₂R^{b62}, NR^{c62}S(O)(═NR^{e62})R^{b62}, NR^{c62}S(O)₂NR^{c62}R^{d62}, S(O)R^{b62}, S(O)NR^{c62}R^{d62}, S(O)₂R^{b62}, S(O)₂NR^{c62}R^{d62}, OS(O)(═NR^{e62})R^{b62}, OS(O)₂R^{b62}, S(O)(═NR^{e62})R^{b62}, SF₅, P(O)R^{f62}R^{g62}, OP(O)(OR^{h62})(ORⁱ⁶²), P(O)(OR^{h62})(ORⁱ⁶²), and BR^{j62}R^{k62}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a62}, R^{c62}, and R^{d62} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c62} and R^{d62} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b62} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e62} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f62} and R^{g62} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h62} and Rⁱ⁶² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j62} and R^{k62} is independently selected from OH, C₁₋₆ alkoxy, and C₁₋₆ haloalkoxy;
or any R^{j62} and R^{k62} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶⁰ is independently selected from H, D, C₁₋₆ alkyl, and C₁₋₆ haloalkyl;
or R⁶ and R⁶⁰, together with the carbon atom to which they are attached, form a C₃₋₁₀ cycloalkyl or a 4-10 membered heterocycloalkyl moiety, each of which is optionally substituted with 1, 2, 3, or 4 independently selected R^{7A} substituents;
each R⁷ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)NR^{c7}R^{d7}, NR^{c7}S(O)R^{b7}, NR^{c7}S(O)₂R^{b7}, NR^{c7}S(O)(═NR^{c7})R^{b7}, NR^{c7}S(O)₂NR^{c7}R^{d7} S(O)R^{b7}, S(O)NR^{c7}R^{d7}, S(O)₂R^{b7}, S(O)₂NR^{c7}R^{d7}, OS(O)(═NR^{e7})R^{b7}, OS(O)₂R^{b7}, S(O)(═NR^{e7})R^{b7}, SF₅, P(O)R^{f7}R^{g7}, OP(O)(OR^{h7})(ORⁱ⁷), P(O)(OR^{h7})(ORⁱ⁷), and BR^{j7}R^{k7}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
or, any R^{c7} and R^{d7} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h7} and Rⁱ⁷ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j7} and R^{k7} is independently selected from OH, C₁₋₆ alkoxy, and C₁₋₆ haloalkoxy;
or any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7A} is independently selected from D, halo, CN, NO₂, C₁₋₆ alkyl, C₂₋₆ alkenyl, C₂₋₆ alkynyl, C₁₋₆ haloalkyl, C₃₋₇cycloalkyl, phenyl, 4-7 membered heterocycloalkyl, 5-6 membered heteroaryl, C₃₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, NHOR^{a71}, C(O)R^{b71}, C(O)NR^{c71}R^{d71}, C(O)NR^{c71}(OR^{a71}), C(O)OR^{a71}, OC(O)R^{b71}, OC(O)NR^{c71}R^{d71}, NR^{c71}R^{d71}, NR^{c71}NR^{c71}R^{d71}, NR^{c71}C(O)R^{b71}, NR^{c71}C(O)OR^{a71}, NR^{c71}C(O)NR^{c71}R^{d71}, C(═NR^{e71})R^{b71}, C(═NR^{e71})NR^{c71}R^{d71}, NR^{c71}C(═NR^{e71})NR^{c71}R^{d71}, NR^{c71}C(═NR^{e71})R^{b71}, NR^{c71}S(O)NR^{c71}R^{d71}, N^{c71}S(O)R^{b71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, OS(O)₂R^{b71}, S(O)(═NR^{e71})R^{b71}, SF₅, P(O)R^{f71}R^{g71}, OP(O)(OR^{h71})(ORⁱ⁷¹), P(O)(OR^{h71})(ORⁱ⁷¹), and BR^{j71}R^{k71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B}
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R⁷¹ is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f71} and R^{g71} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h71} and Rⁱ⁷¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j71} and R^{k71} is independently selected from OH, C₁₋₆ alkoxy, and C₁₋₆ haloalkoxy;
or any R^{j71} and R^{k71} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a72}, SR^{a72}, NHOR^{a72}C(O)R^{b72}, C(O)NR^{c72}R^{d72}, C(O)NR^{c72}(OR^{a72}), C(O)OR^{a72}, OC(O)R^{b72}, OC(O)NR^{c72}R^{d72}, NR^{c72}R^{d72}, NR^{c72}NR^{c72}R^{d72}, NR^{c72}C(O)R^{b72}, NR^{c72}C(O)OR^{a72}, NR^{c72}C(O)NR^{c72}R^{d72}, C(═NR^{e72})R^{b72}, C(═NR^{e72})NR^{c72}R^{d72}, NR^{c72}C(═NR^{e72})NR^{c72}R^{d72}, NR^{c72}C(═NR^{e72})R^{b72}, NR^{c72}S(O)NR^{c72}R^{d72}, NR^{c72}S(O)R^{b72}, NR^{c72}S(O)₂R^{b72}, NR^{c72}S(O)(═NR^{e72})R^{b72}, NR^{c72}S(O)₂NR^{c72}R^{d72}, S(O)R^{b72}, S(O)NR^{c72}R^{d72}, S(O)₂R^{b72}, S(O)₂NR^{c72}R^{d72} OS(O)(═NR^{e72})R^{b72}, OS(O)₂R^{b72}, S(O)(═NR^{e72})R^{b72}, SF₅, P(O)R^{f72}R^{g72}, OP(O)(OR^{h72})(ORⁱ⁷²), P(O)(OR^{h72})(ORⁱ⁷²), and BR^{j72}R^{k72}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a72}, R^{c72}, and R^{d72} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c72} and R^{d72} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b72} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e72} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h72} and R^{g72} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h72} and Rⁱ⁷² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j72} and R^{k72} is independently selected from OH, C₁₋₆ alkoxy, and C₁₋₆ haloalkoxy;
or any R^{j72} and R^{k72} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{W} is a covalent warhead moiety having a reactive functional group capable of covalently binding to one or more cysteine residues in a protein;
each R¹⁰ is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a10}, SR^{a10}, NHOR^{a10}, C(O)R^{b10}, C(O)NR^{c10}R^{d10}, C(O)NR^{c10}(OR^{a10}), C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)NR^{c10}R^{d10}, NR^{c10}S(O)R^{b10}, NR^{c10}S(O)₂R^{b10}, NR^{c10}S(O)₂NR^{c10}R^{d10}, S(O)R^{b10}, S(O)NR^{c10}R^{d10}, S(O)₂R^{b10}, and S(O)₂NR^{c10}R^{d10}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a10}, R^{c10}, and R^{d10} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c10} and R^{d10} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{3A} substituents;
each R^{b10} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{3A} substituents; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, N(CD₃)₂, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, the compound is of Formula (I):

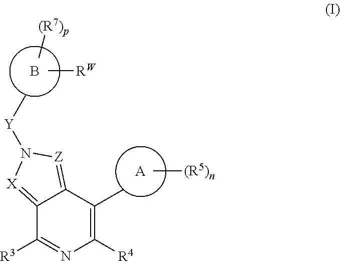

or a pharmaceutically acceptable salt thereof, wherein:
n is 0, 1, 2, 3, 4, 5, or 6;
p is 0, 1, 2, 3, 4, 5, or 6;
r is 1 or 2;
X is N or CR¹;
Z is N or CR²;
Y is —(Cy¹)ₘ-(Y¹)ₛ—, wherein Y¹ is attached to Ring moiety B;
m is 1; s is 1; and Y¹ is a bond; or
m is 1; s is 1; and Y¹ is —(CR⁶R⁶⁰)ᵣ—, —CR⁶R⁶⁰—C(O)—, —C(O)—CR⁶R⁶⁰—, or C(O);
m is 0; s is 1; and Y¹ is —(CR⁶R⁶⁰)ᵣ—, —CR⁶R⁶⁰—C(O)—, —C(O)—CR⁶R⁶⁰—, or C(O);
Cy¹ is C₃₋₁₀ cycloalkyl, 6-10 membered aryl, 4-10 membered heterocycloalkyl, or 5-10 membered heteroaryl, which is optionally substituted by 1, 2, 3, 4, 5, or 6 independently selected R¹⁰ substituents;
Ring moiety A is C₃₋₁₂ cycloalkyl, 6-10 membered aryl, 4-12 membered heterocycloalkyl, or 5-10 membered heteroaryl;
Ring moiety B is C₃₋₁₂ cycloalkyl, 6-10 membered aryl, 4-12 membered heterocycloalkyl, or 5-10 membered heteroaryl;
R¹ is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{b1}, SR^{b1}, NHOR^{a11}, C(O)R^{b1}, C(O)NR^{c1}R^{d1}, C(O)NR^{c1}(OR^{a1}), C(O)OR^{a11}, OC(O)R^{b1}, OC(O)NR^{c1}R^{d1}, NR^{c1}R^{d1}, NR^{c1}NR^{c1}R^{d1}, NR^{c1}C(O)R^{b1}, NR^{c1}C(O)OR^{a11}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}C(═NR^{e1})R^{b1}, NR^{c1}S(O)NR^{c1}R^{d1}, NR^{c1}S(O)R^{b1}, NR^{c1}S(O)₂R^{b1}, NR^{c1}S(O)(═NR^{e1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, OS(O)₂R^{b1}, S(O)(═NR^{e1})R^{b1}, SF₅, P(O)R^{f1}R^{g1}, OP(O)(OR^{h1})(ORⁱ¹), P(O)(OR^{h1})(ORⁱ¹), and BR^{j1}R^{k1}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h1} and Rⁱ¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j1} and R^{k1} is independently selected from OH, C₁₋₆ alkoxy, and C₁₋₆ haloalkoxy;
or any R^{j1} and R^{k1} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h11} and Rⁱ¹¹ is independently selected from H, C₁₋₆ alkyl, C₁₋₆haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j11} and R^{k11} is independently selected from OH, C₁₋₆ alkoxy, and C₁₋₆ haloalkoxy;
or any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{c12}C(═NR^{e12})R^{c12}R^{d12}, NR^{c12}C(═NR^{e12})R^{b12}, NR^{c12}S(O)NR^{c12}R^{d12}, NR^{c12}S(O)R^{b12}, NR^{c12}S(O)₂R^{b12}, NR^{c12}S(O)(═NR^{e12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c12} and R^{d12} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{b2}, SR^{b2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})R^{c2}R^{d2}, NR^{c2}C(═NR^{e2})R^{b2}, NR^{c2}S(O)NR^{c2}R^{d2}, NR^{c2}S(O)R^{b2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, S(O)(═NR^{e2})R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A}
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h2} and Rⁱ² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21}NR^{c21}R^{d21}, NR^{c2}C(═NR^{e21})R^{b21}, NR^{c21}S(O)NR^{c21}R^{d21}, NR^{c21}S(O)R^{b21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
or, any R^{c21} and R^{d21} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2B} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f21} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from D, halo, CN, NO₂, C₁₋₆ alkyl, C₂₋₆ alkenyl, C₂₋₆ alkynyl, C₁₋₆ haloalkyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a22}, SR^{a22}, NHOR^{a22}, C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, N^{c22}NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22})R^{b22}, NR^{c22}S(O)NR^{c22}R^{d22}, NR^{c22}S(O)R^{b22}, NR^{c22}S(O)₂R^{b22}, NR^{c22}S(O)(═NR^{e22})R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22} OS(O)(═NR^{e22})R^{b22}, OS(O)₂R^{b22}, S(O)(═NR^{e22})R^{b22}, SF₅, P(O)R^{f22}R^{g22}, OP(O)(OR^{h22})(ORⁱ²²), P(O)(OR^{h22})(ORⁱ²²), and BR^{j22}R^{k22}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c22} and R^{d22} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b22} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f22} and R^{g22} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h22} and Rⁱ²² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j22} and R^{k22} is independently selected from OH, C₁₋₆ alkoxy, and C₁₋₆ haloalkoxy;
or any R^{j22} and R^{k22} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b3} is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4}, NR^{c4}C(═NR^{e4})R^{b4}, NR^{c4}S(O)NR^{c4}R^{d4}, NR^{c4}S(O)R^{b4}, NR^{c4}S(O)₂R^{b4}, NR^{c4}S(O)(═NR^{e4})R^{b4}, NR^{c4}S(O)₂NR^{C4}R^{d4}, S(O)R^{b4}, S(O)NR^{c4}R^{d4}, S(O)₂R^{b4}, and S(O)₂NR^{c4}R^{d4}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
each R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b4} is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e4} is independently selected from H, OH, CN, C₁₋₆ alkyl, C₁₋₆ alkoxy, C₁₋₆ haloalkyl, and C₁₋₆ haloalkoxy;
each R⁵ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
or, any R^{c5} and R^{d5} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}, NR^{c51}R^{d51}NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)NR^{c51}R^{d51}, NR^{c51}S(O)R^{b51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, OS(O)₂R^{b51}, S(O)(═NR^{e51})R^{b51}, SF₅, P(O)R^{f51}R^{g51}, OP(O)(OR^{h51})(ORⁱ⁵¹), P(O)(OR^{h51})(ORⁱ⁵¹), and BRⁱ⁵¹R^{k51}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c51} and R^{d51} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b51} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f51} and R^{g51} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h51} and Rⁱ⁵¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j51} and R^{k51} is independently selected from OH, C₁₋₆ alkoxy, and C₁₋₆ haloalkoxy;
or any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶ is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c6}R^{d6}, C(═NR^{e6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)NR^{c6}R^{d6}, NR^{c6}S(O)R^{b6}, NR^{c6}S(O)₂R^{b6}, NR^{c6}S(O)(═NR^{e6})R^{b6}, NR^{c6}S(O)₂NR^{c6}R^{d6}, S(O)R^{b6}, S(O)NR^{c6}R^{d6}, S(O)₂R^{b6}, S(O)₂NR^{c6}R^{d6}, OS(O)(═NR^{e6})R^{b6}, OS(O)₂R^{b6}, S(O)(═NR^{e6})R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
or, any R^{c6} and R^{d6} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6A} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f6} and R^{g6} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h6} and Rⁱ⁶ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j6} and R^{k6} is independently selected from OH, C₁₋₆ alkoxy, and C₁₋₆ haloalkoxy;
or any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, NHOR^{a61}, C(O)R^{b61}, C(O)NR^{c61}R^{d61}, C(O)NR^{c61}(OR^{a61}), C(O)OR^{a61}, OC(O)R^{b61}, OC(O)NR^{c61}R^{d61}, NR^{c61}R^{d61}, NR^{c61}NR^{c61}R^{d61}, NR^{c61}C(O)R^{b61}, NR^{c61}C(O)OR^{a61}, NR^{c61}C(O)NR^{c61}R^{d61}, C(═NR^{e61})R^{b61}, C(═NR^{e61})R^{c61}R^{d61}, NR^{c61}C(═NR^{e61})NR^{c61}R^{d61}, NR^{c61}C(═NR^{e61})R^{b61}, NR^{c61}S(O)NR^{c6}R^{d61}, NR^{c6}, S(O)R^{b61}, NR^{c61}S(O)₂R^{b61}, NR^{c61}S(O)(═NR^{e61})R^{b61}, NR^{c61}S(O)₂NR^{c61}R^{d61}, S(O)R^{b61}, S(O)NR^{c61}R^{d61}, S(O)₂R^{b61}, S(O)₂NR^{c6}R^{d61}, OS(O)(═NR^{e61})R^{b61}, OS(O)₂R^{b61}, S(O)(═NR^{e61})R^{b61}, SF₅, p(O)R^{f6}1R^{g61}, OP(O)(OR^{h61})(ORⁱ⁶¹), P(O)(OR^{h61})(ORⁱ⁶¹), and BRⁱ⁶¹R^{k61}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{6B} substituents;
each R⁶¹ is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{e61} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f61} and R^{g61} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h61} and Rⁱ⁶¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j61} and R^{k61} is independently selected from OH, C₁₋₆ alkoxy, and C₁₋₆ haloalkoxy;
or any R^{j61} and R^{k61} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, NHOR^{a62}C(O)R^{b62}, C(O)NR^{c62}R^{d62}, C(O)NR^{c62}(OR^{a62}), C(O)OR^{a62}, OC(O)R^{b62}, OC(O)NR^{c62}R^{d62}, NR^{c62}R^{d62}, NR^{c62}NR^{c62}R^{d62}, R^{c62}C(O)R^{b62}, NR^{c62}C(O)OR^{a62}, NR^{c62}C(O)NR^{c62}R^{d62}, C(═NR^{e62})R^{b62}, C(═NR^{e62})NR^{c62}R^{d62}, NR^{c62}C(═NR^{e62})NR^{c62}R^{d62}, NR^{c62}C(═NR^{e62})R^{b62}, NR^{c62}S(O)NR^{c62}R^{d62}, NR^{c62}S(O)R^{b62}, NR^{c62}S(O)₂R^{b62}, NR^{c62}S(O)(═NR^{e62})R^{b62}, NR^{c62}S(O)₂NR^{c62}R^{d62}, S(O)R^{b62}, S(O)NR^{c62}R^{d62}, S(O)₂R^{b62}, S(O)₂NR^{c62}R^{d62}, OS(O)(═NR^{e62})R^{b62}, OS(O)₂R^{b62}, S(O)(═NR^{e62})R^{b62}, SF₅, p(O)R^{f62}R^{g62} OP(O)(OR^{h62})(ORⁱ⁶²), P(O)(OR^{h62})(ORⁱ⁶²), and BR^{j62}R^{k62}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a62}, R^{c62}, and R^{d62} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c62} and R^{d62} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b62} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e62} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f62} and R^{g62} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h62} and Rⁱ⁶² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j62} and R^{k62} is independently selected from OH, C₁₋₆ alkoxy, and C₁₋₆ haloalkoxy;
or any R^{j62} and R^{k62} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶⁰ is independently selected from H, D, C₁₋₆ alkyl, and C₁₋₆ haloalkyl;
or R⁶ and R⁶⁰, together with the carbon atom to which they are attached, form a 4-10 membered heterocycloalkyl moiety, which is optionally substituted with 1, 2, 3, or 4 independently selected R^{7A} substituents;
each R⁷ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{c7})R^{b7}, NR^{c7}S(O)NR^{c7}R^{d7}, NR^{c7}S(O)R^{b7}, NR^{c7}S(O)₂R^{b7}, NR^{c7}S(O)(═NR^{e7})R^{b7}, NR^{c7}S(O)₂NR^{c7}R^{d7} S(O)R^{b7}, S(O)NR^{c7}R^{d7}, S(O)₂R^{b7}, S(O)₂NR^{c7}R^{d7}, OS(O)(═NR^{e7})R^{b7}, OS(O)₂R^{b7}, S(O)(═NR^{e7})R^{b7}, SF₅, P(O)R^{f7}R^{g7}, OP(O)(OR^{h7})(ORⁱ⁷), P(O)(OR^{h7})(ORⁱ⁷), and BR^{j7}R^{k7}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
or, any R^{c7} and R^{d7} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h7} and Rⁱ⁷ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j7} and R^{k7} is independently selected from OH, C₁₋₆ alkoxy, and C₁₋₆ haloalkoxy;
or any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, NHOR^{a71}, C(O)R^{b71}, C(O)NR^{c71}R^{d71}, C(O)NR^{c71}(OR^{a71}), C(O)OR^{a71}, OC(O)R^{b71}, OC(O)NR^{c71}R^{d71}, NR^{c71}R^{d71}, NR^{c71}NR^{c71}R^{d71}, NR^{c71}C(O)R^{b71}, NR^{c71}C(O)OR^{a71}, NR^{c71}C(O)NR^{c71}R^{d71}, C(═NR^{e71})R^{b71}, C(═NR^{e71})NR^{c7}R^{d71}, NR^{c71}C(═NR^{e71})NR^{c71}R^{d71}, NR^{c71}C(═NR^{e71})R^{b71}, NR^{c71}S(O)NR^{c71}R^{d71}, NR^{c71}S(O)R^{b71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, OS(O)₂R^{b71}, S(O)(═NR^{e71})R^{b71}, SF₅, P(O)R^{f71}R^{g71}, OP(O)(OR^{h71})(ORⁱ⁷¹), P(O)(OR^{h71})(ORⁱ⁷¹), and BR^{j71}R^{k71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁷¹ substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R⁷¹ is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f71} and R^{g71} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h71} and Rⁱ⁷¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j71} and R^{k71} is independently selected from OH, C₁₋₆ alkoxy, and C₁₋₆ haloalkoxy;
or any R^{j71} and R^{k71} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a72}, SR^{a72}, NHOR^{a72}, C(O)R^{b72}, C(O)NR^{c72}R^{d72}, C(O)NR^{c72}(OR^{a72}), C(O)OR^{a72}, OC(O)R^{b72}, OC(O)NR^{c72}R^{d72}, NR^{c72}R^{d72}, NR^{c72}NR^{c72}R^{d72}, NR^{c72}C(O)R^{b72}, NR^{c72}C(O)OR^{a72}, NR^{c72}C(O)NR^{c72}R^{d72}, C(═NR^{e72})R^{b72}, C(═NR^{e72})R^{c72}R^{d72}, NR^{c72}C(═NR^{e72})NR^{c72}R^{d72}, NR^{c72}C(═NR^{e72})R^{b72}, NR^{c72}S(O)NR^{c72}R^{d72}, NR^{c72}S(O)R^{b72}, NR^{c72}S(O)₂R^{b72}, NR^{c72}S(O)(═NR^{e72})R^{b72}, NR^{c72}S(O)₂NR^{c72}R^{d72}, S(O)R^{b72}, S(O)NR^{c72}R^{d72}, S(O)₂R^{b72}, S(O)₂NR^{c72}R^{d72} OS(O)(═NR^{e72})R^{b72}, OS(O)₂R^{b72}, S(O)(═NR^{e72})R^{b72}, SF₅, P(O)R^{f72}R^{g72}, OP(O)(OR^{h72})(ORⁱ⁷²), P(O)(OR^{h72})(ORⁱ⁷²), and BR^{j72}R^{k72}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a72}, R^{c72}, and R^{d72} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c72} and R^{d72} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b72} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e72} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f72} and R^{g72} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h72} and Rⁱ⁷² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j72} and R^{k72} is independently selected from OH, C₁₋₆ alkoxy, and C₁₋₆ haloalkoxy;
or any R^{j72} and R^{k72} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{W} is a covalent warhead moiety having a reactive functional group capable of covalently binding to one or more cysteine residues in a protein;
each R¹⁰ is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a10}, SR^{a10}, NHOR^{a10}, C(O)R^{b10}, C(O)NR^{c10}R^{d10}, C(O)NR^{c10}(OR^{a10}), C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)NR^{c10}R^{d10}, NR^{c10}S(O)R^{b10}, NR^{c10}S(O)₂R^{b10}, NR^{c10}S(O)₂NR^{c10}R^{d10}, S(O)R^{b10}, S(O)NR^{c10}R^{d10}, S(O)₂R^{b10}, and S(O)₂NR^{c10}R^{d10}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a10}, R^{c10}, and R^{d10} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c10} and R^{d10} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{3A} substituents;
each R¹⁰ is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{3A} substituents; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Alternatively, in the preceding embodiment, R^{W} is a means for covalently binding to cysteine residue in a protein (e.g., VRK1). In some embodiments, R^{W} is a means for covalently binding to cysteine residue in the VRK1 protein.

Chemical moieties capable of binding to cysteine are known in the art and can include, but are not limited to those described in Huang, et al., “CovalentWarheads. Targeting Cysteine Residue: The Promising Approach in Drug Development”, Molecules 2022, 27, 7728; and the sections discussing cysteine warheads in the following references: Mehta, et al., “The expanding repertoire of covalent warheads for drug discovery”, Drug Discov Today 2023, 28(12):103799; and Martin, et al., “Characterising covalent warhead reactivity”, Bioorg Med Chem 2019, 15; 27(10):2066-2074, each of which is incorporated herein in its entirety.

In some embodiments, R^{W} is:

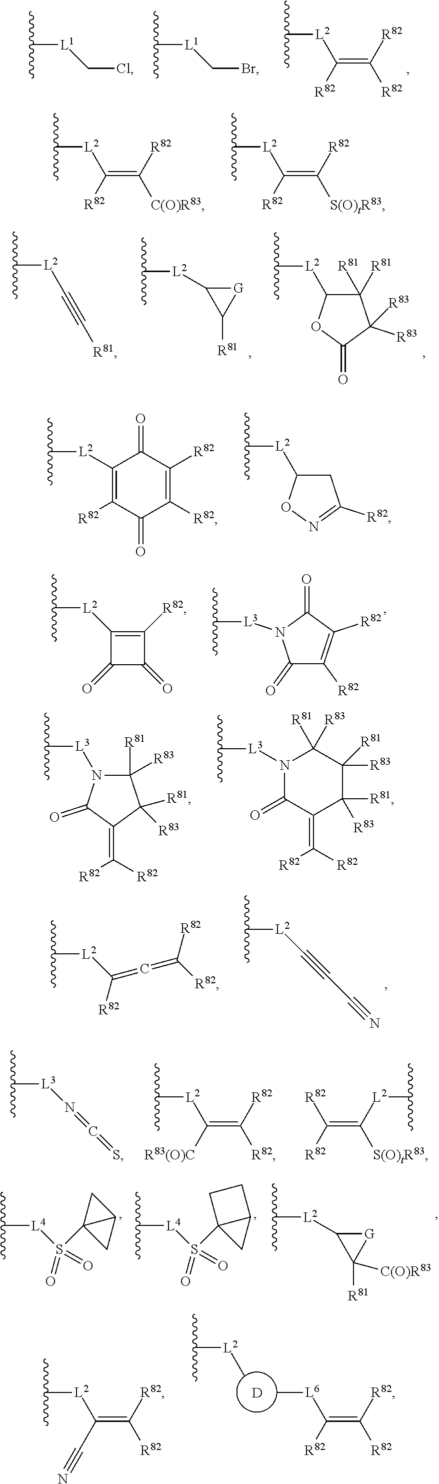

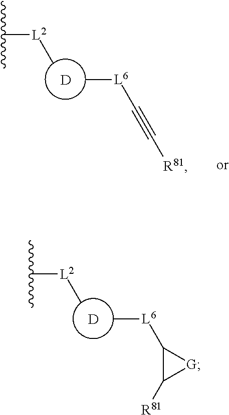

L¹ is -L-C(O)—, -L-NR⁹C(O)—, -L-OC(O)—, -L-S(O)—, -L-S(O)₂—, -L-NR⁹S(O)—, -L-OS(O)—, -L-NR⁹S(O)₂—, or -L-OS(O)₂—, wherein L¹ is attached to Ring moiety B through the L linking group;
L² is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NR⁹S(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)— or -L-OS(O)₂NR⁹—, wherein L² is attached to Ring moiety B through the L linking group;
L³ is -L-, -L-C(O)—, -L-NR⁹C(O)—, -L-OC(O)—, -L-S(O)—, -L-S(O)₂—, -L-NR⁹(O)—, -L-OS(O)—, -L-NR⁹S(O)₂—, or -L-OS(O)₂—, wherein L³ is attached to Ring moiety B through the L linking group;
L⁴ is -L-, -L-O—, L-S—, or -L-NR⁹—, wherein L⁴ is attached to Ring moiety B through the L linking group;
L⁶ is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NR⁹S(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—, wherein L⁶ is attached to Ring moiety D through the L linking group;
L is a bond or C₁₋₆ alkylene, wherein said C₁₋₆ alkylene is optionally substituted by 1, 2, 3, or 4 independently selected R^{G} substituents; or
L is —O—C₁₋₆ alkyl or —N(R^{N})—C₁₋₆ alkyl, wherein L is attached to Ring moiety A via the oxygen of the —O—C₁₋₆ alkyl or the nitrogen atom of the —N(R^{N})—C₁₋₆ alkyl group;
each R^{N} is independently H or C₁₋₆ alkyl;
Ring D is a 4-12 membered heterocycloalkyl, C₃₋₁₂ cycloalkyl, C₆₋₁₀ aryl, or a 5-10 membered heteroaryl, each of which is optionally substituted with 1, 2, 3, or 4 independently selected C₁₋₆ alkyl groups;
G is O or NR⁹;
each t is independently 0, 1, 2, or 3;
each R⁸¹ and R⁸³ are independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{c8}R^{d8}, NR^{c8}R^{d8}, NR^{c8}NR^{c8}R^{d8}, NR^{c8}C(O)R^{b8}, NR^{c8}C(O)OR^{as}, NR^{c8}C(O)NR^{c8}R^{d8}, C(═NR^{e8})R^{b8}, C(═NR^{e8})NR^{c8}R^{d8}, NR^{c8}C(═NR^{e8})R^{c8}R^{d8}, NR^{c8}C(═NR^{e8})R^{b8}, NR^{c8}S(O)NR^{c8}R^{d8}, NR^{c1}S(O)R^{b8}, NR^{c8}S(O)₂R^{b8}, NR^{c1}S(O)(═NR^{e8})R^{b8}, NR^{c8}S(O)₂NR^{c8}R^{d8}, S(O)R^{b8}, S(O)NR^{c8}R^{d8}, S(O)₂R^{b8}, S(O)₂NR^{c8}R^{d8}, 0S(O)(═NR^{e8})R^{b8}, OS(O)₂R^{b8}, S(O)(═NR^{e8})R^{b8}, SF₅, P(O)R^{f8}R^{f8}, OP(O)(OR^{h8})(ORⁱ⁸), P(O)(OR^{h8})(ORⁱ⁸), and BR^{j8}R^{k8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R⁸² is independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, C(O)R^{b8}, C(O)NR^{c8}R^{d8}, C(O)OR^{a8}, C(═NR^{e8})R^{h8}, C(═NR^{e8})NR^{c8}R^{d8}, S(O)R^{b8}, S(O)NR^{c8}R^{d8}, S(O)₂R^{b8}, and S(O)₂NR^{c8}R^{d8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a8}, R^{e8}, and R^{d8} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{h8} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h8} and R^{g8} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j8} and R^{k8} is independently selected from OH, C₁₋₆ alkoxy, and C₁₋₆ haloalkoxy;
or any R^{j8} and R^{k8} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
or any two R⁸¹ and R⁸² together with the atoms to which they are attached, form C₃₋₇ cycloalkyl, 4-7 membered heterocycloalkyl, phenyl, or 5-6-membered heteroaryl ring, each of which is optionally substituted with 1, 2, 3, or 4 independently selected R^{G} substituents;
each R⁹ is independently selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, OR^{a9}, SR^{a9}, NHOR^{a9}, C(O)R^{b9}, C(O)NR^{c9}R^{d9}, C(O)NR^{c9}(OR^{a9}), C(O)OR^{a9}, OC(O)R^{b9}, OC(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, C(═NR^{e9})R^{b9}, C(═NR^{e9})NR^{c9}R^{d9}, NR^{c9}C(═NR^{e9})NR^{c9}R^{d9}, NR^{c9}C(═NR^{e9})R^{b9}, NR^{c9}S(O)NR^{c9}R^{d9}, NR^{c9}S(O)R^{b9}, NR^{c9}S(O)₂R^{b9}, NR^{c9}S(O)(═NR^{e9})R^{b9}, NR^{c9}S(O)₂NR^{c9}R^{d9}, S(O)R^{b9}, S(O)NR^{b9}R^{d9}, S(O)₂R^{b9}, S(O)₂NR^{b9}R^{d9} OS(O)(═NR^{e9})R^{b9}, OS(O)₂R^{b9}, and S(O)(═NR^{e9})R^{b9}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a9}, R^{c9}, and R^{d9} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c9} and R^{d9} attached to the same N atom, together with the N atom to which they are attached, form a 4-7 membered heterocycloalkyl group, which is optionally substituted with 1, 2, 3, or 4 independently selected R^{G} substituents;
each R⁹ is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents; and
each R^{e9} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl.

In some embodiments, R^{W} is selected from:

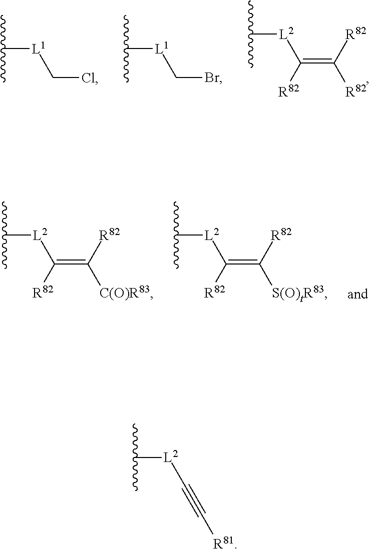

In some embodiments, R^{W} is selected from:

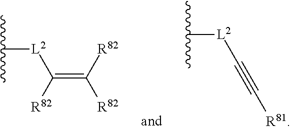

In some embodiments, R^{W} is

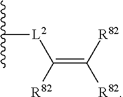

In some embodiments, R^{W} is

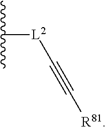

In some embodiments, L¹ is -L-C(O)—, -L-NR⁹C(O)—, -L-S(O)₂—, -L-NR⁹S(O)—, or -L-NR⁹S(O)₂—, or wherein L¹ is attached to Ring moiety B through the L linking group; L is a bond; and R⁹ is selected from H and C₁₋₆ alkyl.

In some embodiments, L² is -L-C(O)—, -L-NR⁹C(O)—, -L-S(O)₂—, or -L-NR⁹S(O)₂—, wherein L² is attached to Ring moiety B through the L linking group; L is a bond; and R⁹ is selected from H and C₁₋₆ alkyl.

In some embodiments, L² is —C(O)—, —NR⁹C(O)—, —S(O)₂—, or —NR⁹S(O)₂—; and R⁹ is selected from H and C₁₋₃ alkyl.

In some embodiments, when L² is —C(O)— or —S(O)₂—, then L² is attached to a nitrogen ring member of Ring moiety B.

In some embodiments, when L² is —C(O)— or —S(O)₂— and Ring moiety B is

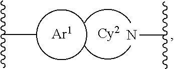

then L² is attached to the nitrogen ring member of Cy².

In some embodiments, R⁸¹, R⁸², and R⁸³ are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, OR^{a8}, C(O)R^{b8}, C(O)NR^{c8}R^{d8}, and C(O)OR^{a8}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, di(C₁₋₃ alkyl)amino, and N(CD₃)₂.

In some embodiments, R⁸¹, R⁸², and R⁸³ are each independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{e8}R^{d8}, NR^{e8}R^{d8}, NR^{c8}NR^{c8}R^{d8}, NR^{c8}C(O)R^{b8}, NR^{c8}C(O)OR^{a8}, NR^{c8}C(O)NR^{e8}R^{d8}, C(═NR^{e8})R^{b8}, C(═NR^{e8})NR^{c8}R^{d8}, NR^{c8}C(═NR^{e8})R^{c8}R^{d8}, NR^{c8}C(═NR^{e8})R^{b8}, NR^{c8}S(O)NR^{c8}R^{d8}, NR^{c8}(O)R^{b5}, NR^{c8}S(O)₂R^{b8}, NR^{c1}S(O)(═NR^{e8})R^{b8}, NR^{c8}S(O)₂NR^{c8}R^{d8}, S(O)R^{b8}, S(O)NR^{c8}R^{d8}, S(O)₂R^{b8}, S(O)₂NR^{c8}R^{d8}, OS(O)(═NR^{e8})R^{b8}, OS(O)₂R^{b8}, and S(O)(═NR^{e8})R^{b8}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents.

In some embodiments, R⁸¹, R⁸², and R⁸³ are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, OR^{a8}, C(O)R^{b8}, C(O)NR^{c8}R^{d8}, and C(O)OR^{a8}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

In some embodiments, n is 0, 1, 2, 3, or 4.

In some embodiments, n is 0, 1, 2, or 3.

In some embodiments, p is 0, 1, 2, 3, or 4.

In some embodiments, p is 0, 1, or 2.

In some embodiments, X is N.

In some embodiments, X is CR¹.

In some embodiments, R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b1}, SR^{b1}, C(O)R^{b1}, C(O)NR^{c1}R^{d1}, C(O)OR^{a11}, OC(O)R^{b1}, OC(O)NR^{c1}R^{d1}, NR^{c1}R^{d1}, NR^{c1}C(O)R, NR^{c1}C(O)OR^{a11}, NR^{c1}C(O)NR^{c1}R^{d1}, NR^{c1}S(O)₂R, NR^{c8}(O)₂NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents.

In some embodiments, R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents.

In some embodiments, R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1A} substituents.

In some embodiments:
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
or, any R^{c} and R^{d1} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R¹ substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R¹ substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R¹ substituents;
each R^{1B} is independently selected from D, halo, CN, C₁₋₆ alkyl, C₂₋₆ alkenyl, C₂₋₆ alkynyl, C₁₋₆ haloalkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c2}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12}, and R^{d12} is independently selected from H, C₁₋₆ alkyl, and C₁₋₆ haloalkyl; and
each Rⁱ² is independently selected from C₁₋₆ alkyl, and C₁₋₆ haloalkyl.

In some embodiments:
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11} is independently selected from H, C₁₋₆ alkyl, and C₁₋₆ haloalkyl; and
each R^{b11} is independently selected from C₁₋₆ alkyl and C₁₋₆ haloalkyl.

In some embodiments:
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}1R^{d11}, NR^{c11}C(O)R^{b11}, NR^{c11}C(O)OR^{a11}, NR^{c11}C(O)NR^{c11}R^{d11}, NR^{c11}S(O)₂R^{b1}, NR^{c11}S(O)₂NR^{c11}R^{d11}, S(O)₂R^{b11}, and S(O)₂NR^{c11}R^{d11};
each R^{a11}, R^{c11}, and R^{d11} is independently selected from H, C₁₋₆ alkyl, and C₁₋₆ haloalkyl; and
each R^{b11} is independently selected from C₁₋₆ alkyl and C₁₋₆ haloalkyl.

In some embodiments, Z is N.

In some embodiments, Z is CR².

In some embodiments, R² is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b2}, SR^{b2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents.

In some embodiments, R² is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents.

In some embodiments, R² is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{2A} substituents.

In some embodiments, R² is selected from H, D, halo, CN, C₁₋₆ alkyl, C₂₋₆ alkenyl, C₂₋₆ alkynyl, C₁₋₆ haloalkyl, and C₃₋₅ cycloalkyl, wherein said C₁₋₆ alkyl, C₂₋₆ alkenyl, C₂₋₆ alkynyl, C₁₋₆ haloalkyl, and C₃₋₅ cycloalkyl are each optionally substituted with 1, 2, 3, or 4 independently selected R^{2A} substituents.

In some embodiments, R² is selected from H, D, halo, CN, C₁₋₆ alkyl, C₂₋₆ alkenyl, C₂₋₆ alkynyl, and C₁₋₆ haloalkyl.

In some embodiments, R² is H.

In some embodiments:
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2A} substituents;
each R^{2A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R²¹ substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R¹ substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R¹ substituents;
each R^{1B} is independently selected from D, halo, CN, C₁₋₆ alkyl, C₂₋₆ alkenyl, C₂₋₆ alkynyl, C₁₋₆ haloalkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12}; and
each R^{a12}, R^{c12}, and R^{d12} is independently selected from H, C₁₋₆ alkyl, and C₁₋₆ haloalkyl.

In some embodiments:
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2A} substituents;
each R^{2A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21}.
each R^{a21}, R^{c21}, and R^{d21} is independently selected from H, C₁₋₆ alkyl, and C₁₋₆ haloalkyl; and
each R^{b21} is independently selected from C₁₋₆ alkyl and C₁₋₆ haloalkyl.

In some embodiments:
each R^{2A} is independently selected from D, halo, CN, C₁₋₆ alkyl, C₂₋₆ alkenyl, C₂₋₆ alkynyl, C₁₋₆ haloalkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21};
each R^{a21}, R^{c21}, and R^{d21} is independently selected from H, C₁₋₆ alkyl, and C₁₋₆ haloalkyl; and
each R^{b21} is independently selected from C₁₋₆ alkyl and C₁₋₆ haloalkyl.

In some embodiments, m is 1; s is 1; and Y¹ is a bond.

In some embodiments, m is 1; s is 1; and Y¹ is —(CR⁶R⁶⁰)ᵣ—, —CR⁶R⁶⁰—C(O)—, —C(O)—CR⁶R⁶⁰—, or C(O).

In some embodiments, m is 1; s is 1; and Y¹ is —CR⁶R⁶⁰—, —CR⁶R⁶⁰—C(O)—, —C(O)—CR⁶R⁶⁰—, or C(O).

In some embodiments, m is 1; s is 1; and Y¹ is —CR⁶R⁶⁰— or C(O).

In some embodiments, Y¹ is bound to a nitrogen ring member of Cy¹.

In some embodiments, Cy¹ is 4-10 membered heterocycloalkyl, which is optionally substituted by 1, 2, 3, or 4 independently selected R¹⁰ substituents.

In some embodiments, Cy¹ is 4-10 membered heterocycloalkyl having 1, 2, or 3 nitrogen ring members, which is optionally substituted by 1, 2, 3, or 4 independently selected R¹⁰ substituents.

In some embodiments, Cy¹ is 4-7 membered heterocycloalkyl, which is optionally substituted by 1, 2, 3, or 4 independently selected R¹⁰ substituents.

In some embodiments, Cy¹ is 4-7 membered heterocycloalkyl having 1 or 2 nitrogen ring members, which is optionally substituted by 1, 2, 3, or 4 independently selected R¹⁰ substituents.

In some embodiments, Cy¹ is an azetidine ring, a pyrrolidine ring, a piperidine ring, a piperazine ring, or an azepine ring, which are each optionally substituted by 1, 2, 3, or 4 independently selected R¹⁰ substituents.

In some embodiments, Cy¹ is an azetidine ring, a pyrrolidine ring, or a piperidine ring, which are each optionally substituted by 1 or 2 independently selected R¹⁰ substituents.

In some embodiments, m is 0; s is 1; and Y¹ is —(CR⁶R⁶⁰)ᵣ—, —CR⁶R⁶⁰—C(O)—, —C(O)—CR⁶R⁶⁰—, or C(O).

In some embodiments, m is 0; s is 1; and Y¹ is —CR⁶R⁶⁰—, —CR⁶R⁶⁰—C(O)—, —C(O)—CR⁶R⁶⁰—, or C(O).

In some embodiments, m is 0; s is 1; and Y¹ is —CR⁶R⁶⁰— or C(O).

In some embodiments:
each R¹⁰ is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂N^{c10}R^{d10}, S(O)₂R¹⁰, and S(O)₂NR^{c10}R^{d10}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a10}, R^{c10}, and R^{d10} is independently selected from H, C₁₋₆ alkyl, and C₁₋₆ haloalkyl; and
each R¹⁰ is independently selected from C₁₋₆ alkyl and C₁₋₆ haloalkyl.

In some embodiments:
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl; and
each R¹⁰ is independently selected from C₁₋₆ alkyl and C₁₋₆ haloalkyl.

In some embodiments:
each R⁶ is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR a, SR a, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}, S(O)₂R^{b6}, and S(O)₂NR^{c6}R^{d6}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents; and
each R⁶⁰ is independently selected from H, C₁₋₆ alkyl, and C₁₋₆ haloalkyl;
or R⁶ and R⁶⁰, together with the carbon atom to which they are attached, form a 4-10 membered heterocycloalkyl moiety, which is optionally substituted with 1, 2, 3, or 4 independently selected R^{G} substituents.

In some embodiments:
each R⁶ is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}, S(O)₂R^{b6}, and S(O)₂NR^{c6}R^{d6}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents; and
each R⁶⁰ is independently selected from H, C₁₋₆ alkyl, and C₁₋₆ haloalkyl;
or R⁶ and R⁶⁰, together with the carbon atom to which they are attached, form a C₃₋₁₀ cycloalkyl or a 4-10 membered heterocycloalkyl moiety, each of which is optionally substituted with 1, 2, 3, or 4 independently selected R^{G} substituents.

In some embodiments:
each R⁶ is independently selected from H, D, halo, CN, C₁₋₆ alkyl, C₂₋₆ alkenyl, C₂₋₆ alkynyl, C₁₋₆ haloalkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}, S(O)₂R^{b6}, and S(O)₂NR^{c6}R^{d6}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents; and
each R⁶⁰ is independently selected from H, C₁₋₆ alkyl, and C₁₋₆ haloalkyl;
or R⁶ and R⁶⁰, together with the carbon atom to which they are attached, form a 4-10 membered heterocycloalkyl moiety, which is optionally substituted with 1, 2, 3, or 4 independently selected R^{7A} substituents.

In some embodiments:
each R⁶ is independently selected from H, D, halo, CN, C₁₋₆ alkyl, C₂₋₆ alkenyl, C₂₋₆ alkynyl, C₁₋₆ haloalkyl, C₃₋₇ cycloalkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}, S(O)₂R^{b6}, and S(O)₂NR^{c6}R^{d6}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents; and
each R⁶⁰ is independently selected from H, C₁₋₆ alkyl, and C₁₋₆ haloalkyl;
or R⁶ and R⁶⁰, together with the carbon atom to which they are attached, form a C₃₋₁₀ cycloalkyl or a 4-10 membered heterocycloalkyl moiety, each of which is optionally substituted with 1, 2, 3, or 4 independently selected R^{7A} substituents.

In some embodiments, each R⁶ is independently selected from H, D, halo, CN, C₁₋₆ alkyl, C₁₋₆ haloalkyl, and C₃₋₇ cycloalkyl.

In some embodiments, each R⁶ is independently selected from H, D, halo, CN, C₁₋₆ alkyl, and C₁₋₆ haloalkyl.

In some embodiments, R⁶ is independently selected from H, D, C₁₋₃ alkyl, and C₃₋₇ cycloalkyl.

In some embodiments, each R⁶ is independently selected from H, D, and C₁₋₃ alkyl.

In some embodiments, each R⁶ is independently selected from H, C₁₋₃ alkyl, and C₃₋₇ cycloalkyl.

In some embodiments, each R⁶ is independently selected from H and C₁₋₃ alkyl.

In some embodiments, each R⁶⁰ is independently selected from H, D and C₁₋₃ alkyl.

In some embodiments, each R⁶⁰ is independently selected from H and C₁₋₃ alkyl.

In some embodiments, R⁶ and R⁶⁰, together with the carbon atom to which they are attached, form a C₃₋₁₀ cycloalkyl or a 4-10 membered heterocycloalkyl moiety, each of which is optionally substituted with 1, 2, 3, or 4 independently selected R^{7A} substituents.

In some embodiments, R⁶ and R⁶⁰, together with the carbon atom to which they are attached, form a C₃₋₁₀ cycloalkyl moiety, which is optionally substituted with 1, 2, 3, or 4 independently selected R^{7A} substituents.

In some embodiments:
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b6}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61} NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁶¹ substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R⁶¹ is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, NR^{c62}C(O)R^{b62}, NR^{c62}C(O)OR^{a62}, NR^{c62}C(O)NR^{c62}R^{d62}, NR^{c62}S(O)₂R^{b62}, NR^{c62}S(O)₂NR^{c62}R^{d62}, S(O)₂R^{b62}, and S(O)₂NR^{c62}R^{d62};
each R^{a62}, R^{c62}, and R^{d62} is independently selected from H, C₁₋₆ alkyl, C₁₋₆ haloalkyl, C₂₋₆ alkenyl, and C₂₋₆ alkynyl; and
each R^{b62} is independently selected from C₁₋₆ alkyl, C₁₋₆ haloalkyl, C₂₋₆ alkenyl, and C₂₋₆ alkynyl.

In some embodiments:
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c6}C(O)R^{b61}, NR^{c61}C(O)OR^{a61} NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61};
each R^{a61}, R^{c61}, and R^{d61} is independently selected from H, C₁₋₆ alkyl, and C₁₋₆ haloalkyl; and
each R^{b61} is independently selected from C₁₋₆ alkyl and C₁₋₆ haloalkyl.

In some embodiments:
each R^{a6}, R^{c6}, and R^{d6} is independently selected from H, C₁₋₆ alkyl, C₁₋₆ haloalkyl, C₂₋₆ alkenyl, and C₂₋₆ alkynyl, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, and C₂₋₆ alkynyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61} NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61};
each R^{a61}, R^{c61}, and R^{d61} is independently selected from H, C₁₋₆ alkyl, and C₁₋₆ haloalkyl; and
each R^{b61} is independently selected from C₁₋₆ alkyl and C₁₋₆ haloalkyl.

In some embodiments, Ring moiety Bis phenyl, naphthyl, 5-10 membered heteroaryl, or 9-12 membered heterocycloalkyl.

In some embodiments, Ring moiety Bis phenyl, naphthyl, 5-6 membered heteroaryl, 9-10 membered heteroaryl, or 9-12 membered heterocycloalkyl.

In some embodiments, Ring moiety Bis phenyl.

In some embodiments, Ring moiety Bis naphthyl.

In some embodiments, Ring moiety Bis 5-6 membered heteroaryl.

In some embodiments, Ring moiety Bis 9-10 membered heteroaryl.

In some embodiments, Ring moiety Bis 9-12 membered heterocycloalkyl.

In some embodiments, Ring moiety B is

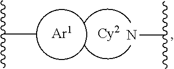

wherein Ar¹ is fused to Cy², Ar¹ is bonded to Y, Ar¹ is phenyl or 5-6 membered heteroaryl, and Cy² is 4-8 membered heterocycloalkyl.

In some embodiments, Ring moiety B is phenyl, naphthyl, 5-6 membered heteroaryl, 9-10 membered heteroaryl, or

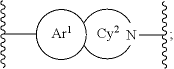

wherein:
Ar¹ is fused to Cy² and Ar¹ is bonded to Y;
Ar¹ is phenyl or 5-6 membered heteroaryl; and
Cy² is 4-8 membered heterocycloalkyl.

In some embodiments, R³ is selected from H, D, halo, CN, C₁₋₆ alkyl, C₂₋₆ alkenyl, C₂₋₆ alkynyl, C₁₋₆ haloalkyl, C₃₋₅ cycloalkyl, OR^{a3}, SR^{a3}, NHOR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}.

In some embodiments, each R^{a3}, R^{c3}, and R^{d3} is independently selected from H, C₁₋₆ alkyl, C₂₋₆ alkenyl, C₂₋₆ alkynyl, and C₁₋₆ haloalkyl; and each R^{b3} is independently selected from C₁₋₆ alkyl, C₂₋₆ alkenyl, C₂₋₆ alkynyl, and C₁₋₆ haloalkyl.

In some embodiments, R³ is selected from H, D, halo, CN, C₁₋₆ alkyl, C₂₋₆ alkenyl, C₂₋₆ alkynyl, C₁₋₆ haloalkyl, OR^{a3}, and NR^{c3}R^{d3}.

In some embodiments, R³ is selected from H, D, halo, CN, C₁₋₆ alkyl, C₂₋₆ alkenyl, C₂₋₆ alkynyl, and C₁₋₆ haloalkyl.

In some embodiments, R³ is H.

In some embodiments, each R^{a3}, R^{c3}, and R^{d3} is independently selected from H and C₁₋₆ alkyl.

In some embodiments, R⁴ is selected from H, D, halo, CN, C₁₋₆ alkyl, C₂₋₆ alkenyl, C₂₋₆ alkynyl, C₁₋₆ haloalkyl, C₃₋₅ cycloalkyl, OR^{a4}, SR^{a4}, NHOR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)₂R^{b4}, NR^{c4}S(O)₂NR^{c4}R^{d4}, S(O)₂R^{b4}, and S(O)₂NR^{c4}R^{d4}.

In some embodiments, each R^{a4}, R^{c4}, and R^{d4} is independently selected from H, C₁₋₆ alkyl, C₂₋₆ alkenyl, C₂₋₆ alkynyl, and C₁₋₆ haloalkyl; and each R^{b4} is independently selected from C₁₋₆ alkyl, C₂₋₆ alkenyl, C₂₋₆ alkynyl, and C₁₋₆ haloalkyl.

In some embodiments, R⁴ is selected from H, D, halo, CN, C₁₋₆ alkyl, C₂₋₆ alkenyl, C₂₋₆ alkynyl, and C₁₋₆ haloalkyl.

In some embodiments, R⁴ is selected from H.

In some embodiments, Ring moiety A is 6-10 membered aryl or 5-10 membered heteroaryl.

In some embodiments, Ring moiety A is phenyl or 5-10 membered heteroaryl.

In some embodiments, Ring moiety A is phenyl, 5-6 membered heteroaryl, and 9-10 membered heteroaryl.

In some embodiments, Ring moiety A is 6-10 membered aryl.

In some embodiments, Ring moiety A is phenyl.

In some embodiments, Ring moiety A is 5-10 membered heteroaryl.

In some embodiments, Ring moiety A is 5-6 membered heteroaryl or 9-10 membered heteroaryl.

In some embodiments, Ring moiety A is 5-6 membered heteroaryl.

In some embodiments, Ring moiety A is 9-10 membered heteroaryl.

In some embodiments, each R⁵ is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a8}, NHOR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5 20}, S(O)₂R^{b5}, and S(O)₂NR^{c5}R^{d5}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents.

In some embodiments, each R⁵ is independently selected from D, halo, CN, C₁₋₆ alkyl, C₂₋₆ alkenyl, C₂₋₆ alkynyl, C₁₋₆ haloalkyl, OR^{as} and NR^{c5}R^{d5}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{5A} substituents.

In some embodiments:
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl which are each optionally substituted with 1, 2, 3, or 4 independently selected R^{5A} substituents; and
each R^{5A} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, wherein said C₁₋₆ alkyl, C₂₋₆ alkenyl, C₂₋₆ alkynyl, C₁₋₆ haloalkyl, C₃₋₄ cycloalkyl, and 4-5 membered heterocycloalkyl are each optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents; and
each R^{5A} is independently selected from OH, CN, halo, C₁₋₃ alkyl, C₁₋₃ haloalkyl, amino, C₁₋₃ alkylamino, and di(C₁₋₃ alkyl)amino.

In some embodiments, each R⁵ is independently selected from halo, CN, C₁₋₃ alkyl, C₁₋₃ haloalkyl, OH, C₁₋₃ alkoxy, and C₁₋₃ haloalkoxy.

In some embodiments, each R⁵ is independently selected from Cl, F, CN, CH₃, OH, and OCH₃.

In some embodiments, each R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7} S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents.

In some embodiments, each R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{b7}R^{d7}, S(O)₂R^{b7}, and S(O)₂NR^{b7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents.

In some embodiments, each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents.

In some embodiments:
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c7}C(O)OR^{a71}, NR^{c71}C(O)NR^{c71}R^{d71}, NR^{c71}S(O)₂R^{b71}, NR^{c71}S(O)₂NR^{c71}R^{d71}, S(O)₂R^{b71}, and S(O)₂NR^{c71}R^{d71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl; and
each R^{b71} is independently selected from C₁₋₆ alkyl and C₁₋₆ haloalkyl.

In some embodiments:
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents; and
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino.

In some embodiments:
n is 0, 1, 2, 3, or 4;
p is 0, 1, 2, 3, or 4;
r is 1 or 2;
m is 1; s is 1; and Y¹ is a bond; or
m is 1; s is 1; and Y¹ is —(CR⁶R⁶⁰)ᵣ—, —CR⁶R⁶⁰—C(O)—, —C(O)—CR⁶R⁶⁰—, or C(O); or
m is 0; s is 1; and Y¹ is —(CR⁶R⁶⁰)ᵣ—, —CR⁶R⁶⁰—C(O)—, —C(O)—CR⁶R⁶⁰—, or C(O);
Cy¹ is 4-10 membered heterocycloalkyl having 1, 2, or 3 nitrogen ring members, which is optionally substituted by 1, 2, 3, or 4 independently selected R¹⁰ substituents;
Ring moiety A is phenyl or 5-10 membered heteroaryl;
Ring moiety B is phenyl, naphthyl, 5-6 membered heteroaryl, 9-10 membered heteroaryl, or

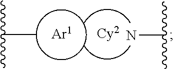

Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b1}, SR^{b1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
or, any R^{c} and R^{d1} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{b11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from D, halo, CN, C₁₋₆ alkyl, C₂₋₆ alkenyl, C₂₋₆ alkynyl, C₁₋₆ haloalkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12}, and R^{d12} is independently selected from H, C₁₋₆ alkyl, and C₁₋₆ haloalkyl;
each R^{b12} is independently selected from C₁₋₆ alkyl, and C₁₋₆ haloalkyl;
R² is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{2A} substituents.
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2A} substituents;
each R^{2A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21};
each R^{a21}, R^{c21}, and R^{d21} is independently selected from H, C₁₋₆ alkyl, and C₁₋₆ haloalkyl;
each R^{b21} is independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, C₁₋₆ haloalkyl, C₃₋₅ cycloalkyl, OR^{a3}, SR^{a3}, NHOR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3};
each R^{a3}, R^{c3}, and R^{d3} is independently selected from H, C₁₋₆ alkyl, C₂₋₆ alkenyl, C₂₋₆ alkynyl, and C₁₋₆ haloalkyl;
each R^{b3} is independently selected from C₁₋₆ alkyl, C₂₋₆ alkenyl, C₂₋₆ alkynyl, and C₁₋₆ haloalkyl;
R⁴ is selected from H, D, halo, CN, C₁₋₆ alkyl, C₂₋₆ alkenyl, C₂₋₆ alkynyl, C₁₋₆ haloalkyl, C₃₋₅ cycloalkyl, OR^{a4}, SR^{a4}, NHOR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)₂R^{b4}, NR^{c4}S(O)₂NR^{c4}R^{d4}, S(O)₂R^{b4}, and S(O)₂NR^{c4}R^{d4};
each R^{a4}, R^{c4}, and R^{d4} is independently selected from H, C₁₋₆ alkyl, C₂₋₆ alkenyl, C₂₋₆ alkynyl, and C₁₋₆ haloalkyl;
each R^{b4} is independently selected from C₁₋₆ alkyl, C₂₋₆ alkenyl, C₂₋₆ alkynyl, and C₁₋₆ haloalkyl;
each R⁵ is independently selected from D, halo, CN, C₁₋₆ alkyl, C₂₋₆ alkenyl, C₂₋₆ alkynyl, C₁₋₆ haloalkyl, OR^{as} and NR^{c5}R^{d5}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{5A} substituents;
each R^{a5}, R^{c5}, and R^{d5} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{5A} substituents;
each R^{5A} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁. 3 alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁-3 alkyl)aminosulfonylamino, aminocarbonylamino, C₁₋₃ alkylaminocarbonylamino, and di(C₁₋₃ alkyl)aminocarbonylamino;
each R⁶ is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}, S(O)₂R^{b6}, and S(O)₂NR^{c6}R^{d6}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents;
each R⁶⁰ is independently selected from H, C₁₋₆ alkyl, and C₁₋₆ haloalkyl;
or R⁶ and R⁶⁰, together with the carbon atom to which they are attached, form a C₃₋₁₀ cycloalkyl or a 4-10 membered heterocycloalkyl moiety, each of which are optionally substituted with 1, 2, 3, or 4 independently selected R^{7A} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61}.
each R^{a61}, R^{c61}, and R^{d61} is independently selected from H, C₁₋₆ alkyl, and C₁₋₆ haloalkyl;
each R^{b61} is independently selected from C₁₋₆ alkyl and C₁₋₆ haloalkyl.
each R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{d7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c71}C(O)OR^{a71}, NR^{c71}C(O)NR^{c71}R^{d71}, NR^{c71}S(O)₂R^{b71}, NR^{c71}S(O)₂NR^{c71}R^{d71}, S(O)₂R^{b71}, and S(O)₂NR^{c71}R^{d71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁷¹ substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{7B} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R^{W} is selected from:

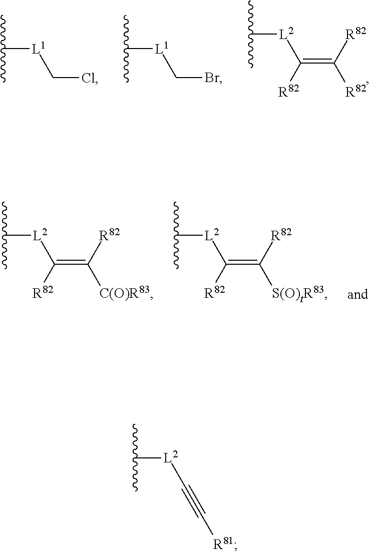

L¹ is -L-C(O)—, -L-NR⁹C(O)—, -L-S(O)₂—, -L-NR⁹S(O)—, or -L-NR⁹S(O)₂—, or wherein L¹ is attached to Ring moiety B through the L linking group;
L² is -L-C(O)—, -L-NR⁹C(O)—, -L-S(O)₂—, or -L-NR⁹S(O)₂—, wherein L² is attached to Ring moiety B through the L linking group;
L is a bond;
R⁹ is selected from H and C₁₋₆ alkyl;
R⁸¹, R⁸², and R⁸³ are each independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{c8}R^{d8}, NR^{c8}R^{d8}, NR^{c8}NR^{c8}R^{d8}, NR^{c8}C(O)R^{b8}, NR^{c8}C(O)OR^{a8}, NR^{c8}C(O)NR^{c8}R^{d8}, C(═NR^{e8})R^{b8}, C(═NR^{e8})R^{c8}R^{d8}, NR^{c8}C(═NR^{e8})NR^{c8}R^{d8}, NR^{c8}C(═NR^{e8})R^{b8}, NR^{c8}S(O)NR^{c8}R^{d8}, NR^{c8}S(O)R^{b8}, NR^{c8}S(O)₂R^{b8}, NR^{c8}S(O)(═NR^{e8})R^{b8}, NR^{c8}S(O)₂NR^{c8}R^{d8}, S(O)R^{b8}, S(O)NR^{c8}R^{d8}, S(O)₂R^{b8}, S(O)₂NR^{c8}R^{d8}, OS(O)(═NR^{e8})R^{b8}, OS(O)₂R^{b8}, and S(O)(═NR^{e8})R^{b8}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a8}, R^{c8}, and R^{d8} is independently selected from H and C₁₋₆ alkyl;
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl;
each R^{b10} is independently selected from C₁₋₆ alkyl and C₁₋₆ haloalkyl; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, N(CD₃)₂, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
n is 0, 1, 2, 3, or 4;
p is 0, 1, 2, 3, or 4;
r is 1 or 2;
m is 1; s is 1; and Y¹ is a bond; or
m is 1; s is 1; and Y¹ is —(CR⁶R⁶⁰)ᵣ—, —CR⁶R⁶⁰—C(O)—, —C(O)—CR⁶R⁶⁰—, or C(O); or
m is 0; s is 1; and Y¹ is —(CR⁶R⁶⁰)ᵣ—, —CR⁶R⁶⁰—C(O)—, —C(O)—CR⁶R⁶⁰—, or C(O); Cy¹ is 4-10 membered heterocycloalkyl having 1, 2, or 3 nitrogen ring members, which is optionally substituted by 1, 2, 3, or 4 independently selected R¹⁰ substituents;
Ring moiety A is phenyl or 5-10 membered heteroaryl;
Ring moiety B is phenyl, naphthyl, 5-6 membered heteroaryl, 9-10 membered heteroaryl, or

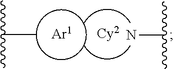

Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b1}, SR^{b1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a1}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from D, halo, CN, C₁₋₆ alkyl, C₂₋₆ alkenyl, C₂₋₆ alkynyl, C₁₋₆ haloalkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c2}C(O)OR^{a12}, NR^{c12}C(O)NR^{c12}R^{d12}, NR^{c12}S(O)₂R¹², NR^{c12}S(O)₂NR^{c12}R^{d12}, S(O)₂R^{b12}, and S(O)₂NR^{c12}R^{d12};
each R^{a12}, R^{c12}, and R^{d12} is independently selected from H, C₁₋₆ alkyl, and C₁₋₆ haloalkyl;
each R¹² is independently selected from C₁₋₆ alkyl, and C₁₋₆ haloalkyl;
R² is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{2A} substituents.
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2A} substituents;
each R^{2A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21}.
each R^{a21}, R^{c21}, and R^{d21} is independently selected from H, C₁₋₆ alkyl, and C₁₋₆ haloalkyl;
each R^{b21} is independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, C₁₋₆ haloalkyl, C₃₋₅ cycloalkyl, OR^{a3}, SR^{a3}, NHOR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3};
each R^{a3}, R^{c3}, and R^{d3} is independently selected from H, C₁₋₆ alkyl, C₂₋₆ alkenyl, C₂₋₆ alkynyl, and C₁₋₆ haloalkyl;
each R^{b3} is independently selected from C₁₋₆ alkyl, C₂₋₆ alkenyl, C₂₋₆ alkynyl, and C₁₋₆ haloalkyl;
R⁴ is selected from H, D, halo, CN, C₁₋₆ alkyl, C₂₋₆ alkenyl, C₂₋₆ alkynyl, C₁₋₆ haloalkyl, C₃₋₅ cycloalkyl, OR^{a4}, SR^{a4}, NHOR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)₂R^{b4}, NR^{c4}S(O)₂NR^{c4}R^{d4}, S(O)₂R^{b4}, and S(O)₂NR^{c4}R^{d4};
each R^{a4}, R^{c4}, and R^{d4} is independently selected from H, C₁₋₆ alkyl, C₂₋₆ alkenyl, C₂₋₆ alkynyl, and C₁₋₆ haloalkyl;
each R^{b4} is independently selected from C₁₋₆ alkyl, C₂₋₆ alkenyl, C₂₋₆ alkynyl, and C₁₋₆ haloalkyl;
each R⁵ is independently selected from D, halo, CN, C₁₋₆ alkyl, C₂₋₆ alkenyl, C₂₋₆ alkynyl, C₁₋₆ haloalkyl, OR^{as} and NR^{c5}R^{d5}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{5A} substituents;
each R^{a5}, R^{c5}, and R^{d5} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{5A} substituents;
each R^{5A} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁-3 alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
each R⁶ is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}, S(O)₂R^{b6}, and S(O)₂NR^{c6}R^{d6}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents;
each R⁶⁰ is independently selected from H, C₁₋₆ alkyl, and C₁₋₆ haloalkyl;
or R⁶ and R⁶⁰, together with the carbon atom to which they are attached, form a 4-10 membered heterocycloalkyl moiety, which is optionally substituted with 1, 2, 3, or 4 independently selected R^{7A} substituents.
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b6}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61} NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61};
each R^{a61}, R^{c61}, and R^{d61} is independently selected from H, C₁₋₆ alkyl, and C₁₋₆ haloalkyl;
each R^{b61} is independently selected from C₁₋₆ alkyl and C₁₋₆ haloalkyl.
each R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c7}C(O)OR^{a71}, NR^{c71}C(O)NR^{c71}R^{d71}, NR^{c71}S(O)₂R^{b71}, NR^{c71}S(O)₂NR^{c71}R^{d71}, S(O)₂R^{b71}, and S(O)₂NR^{c71}R^{d71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{7B} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R^{W} is selected from:

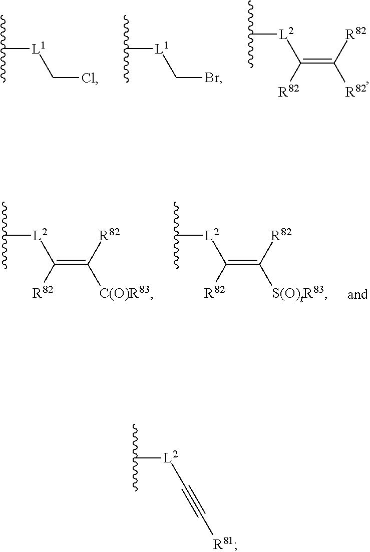

L¹ is -L-C(O)—, -L-NR⁹C(O)—, -L-S(O)₂—, -L-NR⁹S(O)—, or -L-NR⁹S(O)₂—, or wherein L¹ is attached to Ring moiety B through the L linking group;
L² is -L-C(O)—, -L-NR⁹C(O)—, -L-S(O)₂—, or -L-NR⁹S(O)₂—, wherein L² is attached to Ring moiety B through the L linking group;
L is a bond;
R⁹ is selected from H and C₁₋₆ alkyl;
R⁸¹, R⁸², and R⁸³ are each independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{e8}R^{d8}, NR^{c8}R^{d8}, NR^{c8}NR^{c8}R^{d8}, NR^{c8}C(O)R^{b8}, NR^{c8}C(O)OR^{a8}, NR^{c8}C(O)NR^{e8}R^{d8}, C(═NR^{e8})R^{b8}, C(═NR^{e8})R^{c8}R^{d8}, NR^{c8}C(═NR^{e8})NR^{e8}R^{d8}, NR^{c8}C(═NR^{e8})R^{b8}, NR^{c8}S(O)NR^{e8}R^{d8}, NR^{c8}S(O)R^{b8}, NR^{c8}S(O)₂R^{b8}, NR^{c8}S(O)(═NR^{e8})R^{b8}, NR^{c8}S(O)₂NR^{c8}R^{d8}, S(O)R^{b8}, S(O)NR^{e8}R^{d8}, S(O)₂R^{b8}, S(O)₂NR^{e8}R^{d8}, OS(O)(═NR^{e8})R^{b8}, OS(O)₂R^{b8}, and S(O)(═NR^{e8})R^{b8}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a8}, R^{c8}, and R^{d8} is independently selected from H and C₁₋₆ alkyl;
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl;
each R^{b10} is independently selected from C₁₋₆ alkyl and C₁₋₆ haloalkyl; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
n is 0, 1, 2, 3, or 4;
p is 0, 1, 2, 3, or 4;
m is 1; s is 1; and Y¹ is a bond; or
m is 1; s is 1; and Y¹ is —CR⁶R⁶⁰—, —(CR⁶R⁶⁰)₂—, —CR⁶R⁶⁰—C(O)—, —C(O)—CR⁶R⁶⁰—, or C(O); or
m is 0; s is 1; and Y¹ is —CR⁶R⁶⁰—, —(CR⁶R⁶⁰)₂—, —CR⁶R⁶⁰—C(O)—, —C(O)—CR⁶R⁶⁰—, or C(O);
Cy¹ is 4-7 membered heterocycloalkyl having 1 or 2 nitrogen ring members, which is optionally substituted by 1, 2, 3, or 4 independently selected R¹⁰ substituents;
Ring moiety A is phenyl, 5-6 membered heteroaryl, or 9-10 membered heteroaryl;
Ring moiety B is phenyl, naphthyl, 5-6 membered heteroaryl, 9-10 membered heteroaryl, or

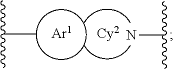

Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, N^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a1}, R^{c11}, and R^{d11} is independently selected from H, C₁₋₆ alkyl, and C₁₋₆ haloalkyl;
each R^{b11} is independently selected from C₁₋₆ alkyl and C₁₋₆ haloalkyl;
R² is selected from H, D, halo, CN, C₁₋₆ alkyl, C₂₋₆ alkenyl, C₂₋₆ alkynyl, C₁₋₆ haloalkyl, and C₃₋₅ cycloalkyl, wherein said C₁₋₆ alkyl, C₂₋₆ alkenyl, C₂₋₆ alkynyl, C₁₋₆ haloalkyl, and C₃₋₅ cycloalkyl are each optionally substituted with 1, 2, 3, or 4 independently selected R^{2A} substituents;
each R^{2A} is independently selected from D, halo, CN, C₁₋₆ alkyl, C₂₋₆ alkenyl, C₂₋₆ alkynyl, C₁₋₆ haloalkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21};
each R^{a21}, R^{c21}, and R^{d21} is independently selected from H, C₁₋₆ alkyl, and C₁₋₆ haloalkyl;
each R^{b21} is independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, C₁₋₆ haloalkyl, OR^{a3}, and NR^{c3}R^{d3};
each R^{a3}, R^{c3}, and R^{d3} is independently selected from H and C₁₋₆ alkyl;
R⁴ is selected from H, D, halo, CN, C₁₋₆ alkyl, C₂₋₆ alkenyl, C₂₋₆ alkynyl, and C₁₋₆ haloalkyl;
R⁵ is independently selected from D, halo, CN, C₁₋₆ alkyl, C₂₋₆ alkenyl, C₂₋₆ alkynyl, C₁₋₆ haloalkyl, C₃₋₄ cycloalkyl, OR^{a5} and NR^{c5}R^{d5}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{5A} substituents;
each R^{a5}, R^{c5}, and R^{d5} is independently selected from C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents;
each R^{5A} is independently selected from OH, CN, halo, C₁₋₃ alkyl, C₁₋₃ haloalkyl, amino, C₁₋₃ alkylamino, and di(C₁₋₃ alkyl)amino;
each R⁶ is independently selected from H, D, halo, CN, C₁₋₆ alkyl, C₂₋₆ alkenyl, C₂₋₆ alkynyl, C₁₋₆ haloalkyl, C₃₋₇ cycloalkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}, S(O)₂R^{b6}, and S(O)₂NR^{c6}R^{d6}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents;
each R⁶⁰ is independently selected from H, C₁₋₆ alkyl, and C₁₋₆ haloalkyl;
or R⁶ and R⁶⁰, together with the carbon atom to which they are attached, form a C₃₋₇ cycloalkyl or a 4-7 membered heterocycloalkyl moiety, each of which are optionally substituted with 1, 2, 3, or 4 independently selected R^{7A} substituents;
each R^{a6}, R^{c6}, and R^{d6} is independently selected from H, C₁₋₆ alkyl, C₁₋₆ haloalkyl, C₂₋₆ alkenyl, and C₂₋₆ alkynyl, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, and C₂₋₆ alkynyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, OR^{a61}, SR^{a61}, C(O)R^{b61}, C(O)NR^{c61}R^{d61}, C(O)OR^{a11}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61};
each R^{a61}, R^{c61}, and R^{d61} is independently selected from H, C₁₋₆ alkyl, and C₁₋₆ haloalkyl;
each R⁶¹ is independently selected from C₁₋₆ alkyl and C₁₋₆ haloalkyl;
each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl;
each R^{b7} is independently selected from C₁₋₆ alkyl and C₁₋₆ haloalkyl;
R^{W} is selected from:

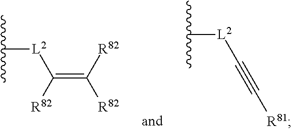

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, OR^{a8}, C(O)R^{b8}, C(O)NR^{c8}R^{d8}, and C(O)OR^{a8}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each R^{a8}, R^{c8}, and R^{d8} is independently selected from H and C₁₋₆ alkyl;
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl;
each R^{b10} is independently selected from C₁₋₆ alkyl and C₁₋₆ haloalkyl; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, N(CD₃)₂, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
n is 0, 1, 2, 3, or 4;
p is 0, 1, 2, 3, or 4;
m is 1; s is 1; and Y¹ is a bond; or
m is 1; s is 1; and Y¹ is —CR⁶R⁶⁰—, —CR⁶R⁶⁰—C(O)—, —C(O)—CR⁶R⁶⁰—, or C(O); or
m is 0; s is 1; and Y¹ is —CR⁶R⁶⁰—, —CR⁶R⁶⁰—C(O)—, —C(O)—CR⁶R⁶⁰—, or C(O); Cy¹ is 4-7 membered heterocycloalkyl having 1 or 2 nitrogen ring members, which is optionally substituted by 1, 2, 3, or 4 independently selected R¹⁰ substituents;
Ring moiety A is phenyl, 5-6 membered heteroaryl, or 9-10 membered heteroaryl;
Ring moiety B is phenyl, naphthyl, 5-6 membered heteroaryl, 9-10 membered heteroaryl, or

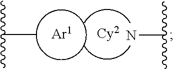

Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11} is independently selected from H, C₁₋₆ alkyl, and C₁₋₆ haloalkyl;
each R^{b11} is independently selected from C₁₋₆ alkyl and C₁₋₆ haloalkyl;
R² is selected from H, D, halo, CN, C₁₋₆ alkyl, C₂₋₆ alkenyl, C₂₋₆ alkynyl, C₁₋₆ haloalkyl, and C₃₋₅ cycloalkyl, wherein said C₁₋₆ alkyl, C₂₋₆ alkenyl, C₂₋₆ alkynyl, C₁₋₆ haloalkyl, and C₃₋₅ cycloalkyl are each optionally substituted with 1, 2, 3, or 4 independently selected R^{2A} substituents;
each R^{2A} is independently selected from D, halo, CN, C₁₋₆ alkyl, C₂₋₆ alkenyl, C₂₋₆ alkynyl, C₁₋₆ haloalkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21};
each R^{a21}, R^{c21}, and R^{d21} is independently selected from H, C₁₋₆ alkyl, and C₁₋₆ haloalkyl;
each R^{b21} is independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, and C₁₋₆ haloalkyl;
R⁴ is selected from H, D, halo, CN, C₁₋₆ alkyl, C₂₋₆ alkenyl, C₂₋₆ alkynyl, and C₁₋₆ haloalkyl;
R⁵ is independently selected from D, halo, CN, C₁₋₆ alkyl, C₂₋₆ alkenyl, C₂₋₆ alkynyl, C₁₋₆ haloalkyl, C₃₋₄ cycloalkyl, OR^{a5} and NR^{c5}R^{d5}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{5A} substituents;
each R^{a5}, R^{c5}, and R^{d5} is independently selected from C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents;
each R^{5A} is independently selected from OH, CN, halo, C₁₋₃ alkyl, C₁₋₃ haloalkyl, amino, C₁₋₃ alkylamino, and di(C₁₋₃ alkyl)amino;
each R⁶ is independently selected from H, D, halo, CN, C₁₋₆ alkyl, C₂₋₆ alkenyl, C₂₋₆ alkynyl, C₁₋₆ haloalkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}, S(O)₂R^{b6}, and S(O)₂NR^{c6}R^{d6}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents;
each R⁶⁰ is independently selected from H, C₁₋₆ alkyl, and C₁₋₆ haloalkyl;
or R⁶ and R⁶⁰, together with the carbon atom to which they are attached, form a 4-10 membered heterocycloalkyl moiety, which is optionally substituted with 1, 2, 3, or 4 independently selected R^{G} substituents;
each R^{a6}, R^{c6}, and R^{d6} is independently selected from H, C₁₋₆ alkyl, C₁₋₆ haloalkyl, C₂₋₆ alkenyl, and C₂₋₆ alkynyl, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A}
each R^{b6} is independently selected from C₁₋₆ alkyl, C₁₋₆ haloalkyl, C₂₋₆ alkenyl, and C₂₋₆ alkynyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61} NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61};
each R^{a61}, R^{c61}, and R^{d61} is independently selected from H, C₁₋₆ alkyl, and C₁₋₆ haloalkyl;
each R⁶¹ is independently selected from C₁₋₆ alkyl and C₁₋₆ haloalkyl;
each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl;
each R^{b7} is independently selected from C₁₋₆ alkyl and C₁₋₆ haloalkyl;
R^{W} is selected from:

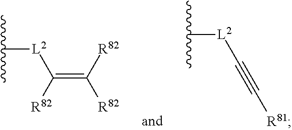

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl; R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, OR^{a8}, C(O)R^{b8}, C(O)NR^{c8}R^{d8}, and C(O)OR^{a8}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each R^{a8}, R^{c8}, and R^{d8} is independently selected from H and C₁₋₆ alkyl;
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl;
each R^{b10} is independently selected from C₁₋₆ alkyl and C₁₋₆ haloalkyl; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments:
n is 0, 1, 2, 3, or 4;
p is 0, 1, 2, 3, or 4;
m is 1; s is 1; and Y¹ is a bond; or
m is 1; s is 1; and Y¹ is —CR⁶R⁶⁰—, —(CR⁶R⁶⁰)₂—, or C(O); or
m is 0; s is 1; and Y¹ is —CR⁶R⁶⁰—, —(CR⁶R⁶⁰)₂—, or C(O); Cy¹ is an azetidine ring, a pyrrolidine ring, or a piperidine ring, which are each optionally substituted by 1 or 2 independently selected R¹⁰ substituents;
Ring moiety A is phenyl, 5-6 membered heteroaryl, or 9-10 membered heteroaryl;
Ring moiety Bis phenyl, naphthyl, 5-6 membered heteroaryl, 9-10 membered heteroaryl, or

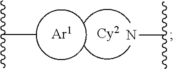

Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, OR^{a11}, SR^{a11}, C(O)R^{b11}, C(O)NR^{c11}R^{d11}, C(O)OR^{a11}, OC(O)R^{b11}, NR^{c11}S(O)₂R^{b11}, NR^{c11}S(O)₂NR^{c11}R^{d11}, S(O)₂R^{b11}, and S(O)₂NR^{c11}R^{d11};
each R^{a1}, R^{c11}, and R^{d11} is independently selected from H, C₁₋₆ alkyl, and C₁₋₆ haloalkyl;
each R^{b11} is independently selected from C₁₋₆ alkyl and C₁₋₆ haloalkyl;
R² is selected from H, D, halo, CN, C₁₋₆ alkyl, C₂₋₆ alkenyl, C₂₋₆ alkynyl, and C₁₋₆ haloalkyl;
R³ is selected from H, D, halo, CN, C₁₋₆ alkyl, C₂₋₆ alkenyl, C₂₋₆ alkynyl, and C₁₋₆ haloalkyl;
R⁴ is selected from H, D, halo, CN, C₁₋₆ alkyl, C₂₋₆ alkenyl, C₂₋₆ alkynyl, and C₁₋₆ haloalkyl;
each R⁵ is independently selected from halo, CN, C₁₋₃ alkyl, C₁₋₃ haloalkyl, OH, C₁₋₃ alkoxy, and C₁₋₃ haloalkoxy;
each R⁶ is independently selected from H, D, halo, CN, C₁₋₆ alkyl, and C₁₋₆ haloalkyl;
each R⁶⁰ is independently selected from H and C₁₋₃ alkyl;
or R⁶ and R⁶⁰, together with the carbon atom to which they are attached, form a C₃₋₇ cycloalkyl moiety optionally substituted with 1 or 2 independently selected R^{7A} substituents;
each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c7}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from:

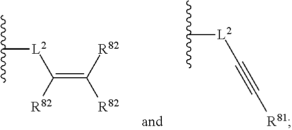

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, and C(O)OR^{a8}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each R^{a8} is independently selected from H and C₁₋₆ alkyl; and
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl; and
each R^{b10} is independently selected from C₁₋₆ alkyl and C₁₋₆ haloalkyl.

In some embodiments:
n is 0, 1, 2, 3, or 4;
p is 0, 1, 2, 3, or 4;
m is 1; s is 1; and Y¹ is a bond; or
m is 1; s is 1; and Y¹ is —CR⁶R⁶⁰— or C(O); or
m is 0; s is 1; and Y¹ is —CR⁶R⁶⁰— or C(O); Cy¹ is an azetidine ring, a pyrrolidine ring, or a piperidine ring, which are each optionally substituted by 1 or 2 independently selected R¹⁰
Ring moiety A is phenyl, 5-6 membered heteroaryl, or 9-10 membered heteroaryl;
Ring moiety Bis phenyl, naphthyl, 5-6 membered heteroaryl, 9-10 membered heteroaryl, or

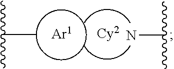

Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, OR^{a11}, SR^{a11}, C(O)R^{b11}, C(O)NR^{c11}R^{d1}, C(O)OR^{a11}, OC(O)R^{b11}, OC(O)NR^{c11}R^{d11}, NR^{c11}R^{d11}, NR^{c11}C(O)R^{b11}, NR^{c11}(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11} is independently selected from H, C₁₋₆ alkyl, and C₁₋₆ haloalkyl;
each R^{b11} is independently selected from C₁₋₆ alkyl and C₁₋₆ haloalkyl;
R² is selected from H, D, halo, CN, C₁₋₆ alkyl, C₂₋₆ alkenyl, C₂₋₆ alkynyl, and C₁₋₆ haloalkyl;
R³ is selected from H, D, halo, CN, C₁₋₆ alkyl, C₂₋₆ alkenyl, C₂₋₆ alkynyl, and C₁₋₆ haloalkyl;
R⁴ is selected from H, D, halo, CN, C₁₋₆ alkyl, C₂₋₆ alkenyl, C₂₋₆ alkynyl, and C₁₋₆ haloalkyl;
each R⁵ is independently selected from halo, CN, C₁₋₃ alkyl, C₁₋₃ haloalkyl, OH, C₁₋₃ alkoxy, and C₁₋₃ haloalkoxy;
each R⁶ is independently selected from H, D, halo, CN, C₁₋₆ alkyl, and C₁₋₆ haloalkyl;
each R⁶⁰ is independently selected from H and C₁₋₃ alkyl;
each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from:

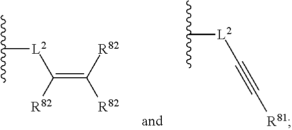

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, and C(O)OR^{a8}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each R^{a8} is independently selected from H and C₁₋₆ alkyl; and
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl; and
each R¹⁰ is independently selected from C₁₋₆ alkyl and C₁₋₆ haloalkyl.

In some embodiments:

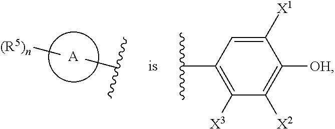

wherein X¹ and X² are each independently Cl or F, and X³ is H, Cl or F; or

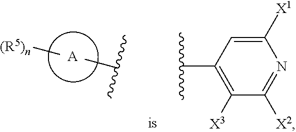

wherein X¹ and X² are each independently Cl, F, OH, and OCH₃; and X³ is H, Cl, or F.

In some embodiments,

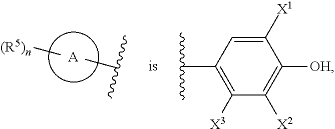

wherein X¹ and X² are each independently Cl or F, and X³ is H, Cl or F.

In some embodiments,

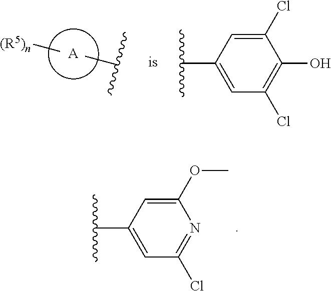

In some embodiments,

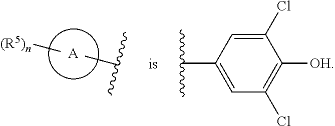

In some embodiments,

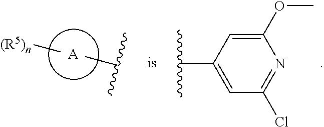

In some embodiments,

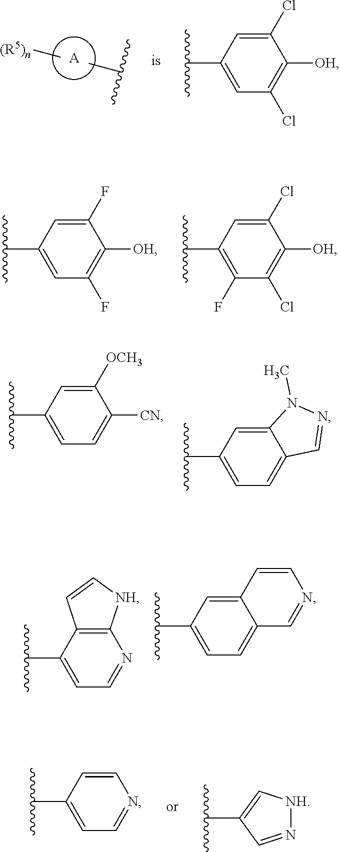

In some embodiments,

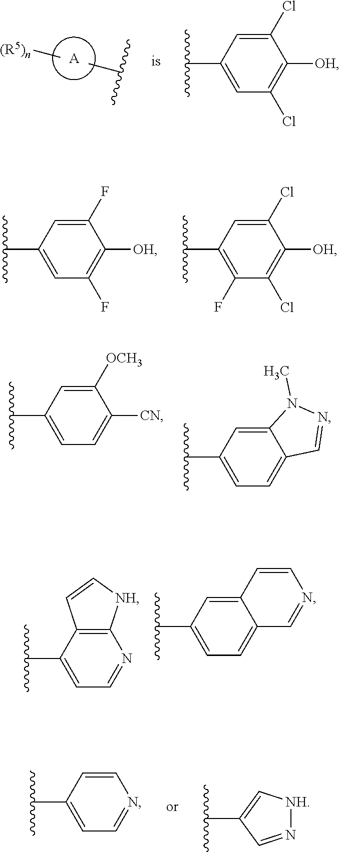

In some embodiments,

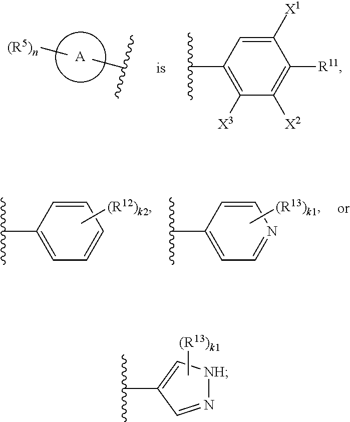

X¹ is Cl, F, or OCH₃;
X² is H, Cl, or F;
X³ is H, Cl or F;
R¹¹ is OH or CN;
k1 is 0, 1, or 2;
k2 is 2;
two R¹² substituents, together with the carbon atoms to which they are attached, form a pyrazole, pyrrole, or pyrimidine ring, which is optionally substituted by 1 or 2 independently selected R⁵ substituents selected from halo, CN, and C₁₋₃ alkyl; and
each R¹³ is independently selected from halo, CN, C₁₋₃ alkyl, and C₁₋₃ alkoxy.

In some embodiments,

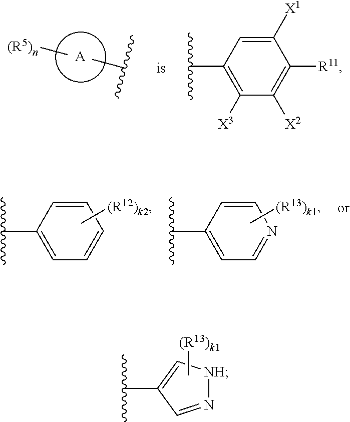

X¹ is Cl, F, or OCH₃;
X² is H, Cl, or F;
X³ is H, Cl or F;
R¹¹ is OH or CN;
k1 is 0, 1, or 2;
k2 is 2;
two R¹² substituents, together with the carbon atoms to which they are attached, form a pyrazole, pyrrole, or pyrimidine ring, which is optionally substituted by 1 or 2 independently selected R⁵ substituents selected from halo, CN, and C₁₋₃ alkyl; and
each R¹³ is independently selected from halo, CN, and C₁₋₃ alkyl.

In some embodiments, the compound is a compound of Formula (II):

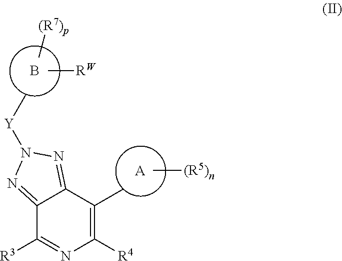

or a pharmaceutically acceptable salt thereof, and wherein Ring moiety A, ring moiety B, Y, R³, R⁴, R⁵, R⁷, R^{W}, n, and p are as defined according to any other embodiment described herein.

In some embodiments, the compound is a compound of Formula (III):

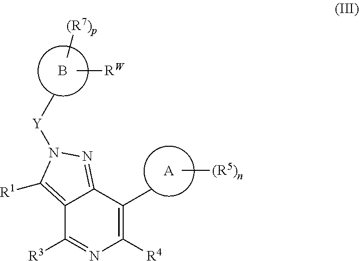

or a pharmaceutically acceptable salt thereof, and wherein Ring moiety A, ring moiety B, Y, R¹, R³, R⁴, R⁵, R⁷, R^{W}, n, and p are as defined according to any other embodiment described herein.

In some embodiments, the compound is a compound of Formula (IV):

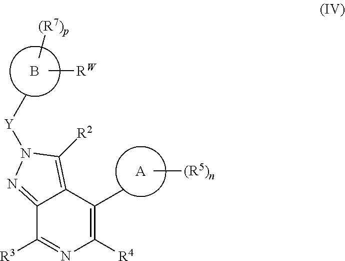

or a pharmaceutically acceptable salt thereof, and wherein Ring moiety A, ring moiety B, Y, R², R³, R⁴, R⁵, R⁷, R^{W}, n, and p are as defined according to any other embodiment described herein.

In some embodiments, the compound is a compound of Formula (V):

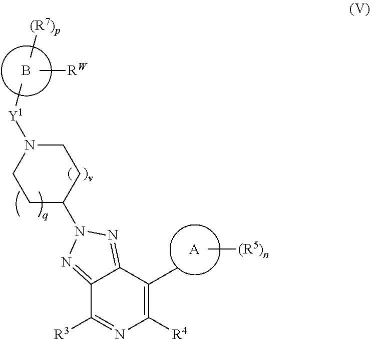

or a pharmaceutically acceptable salt thereof, wherein q is 0 or 1; v is 0 or 1; and Ring moiety A, ring moiety B, Y¹, R³, R⁴, R⁵, R⁷, R^{W}, n, and p are as defined according to any other embodiment described herein.

In some embodiments, the compound is a compound of Formula (VI):

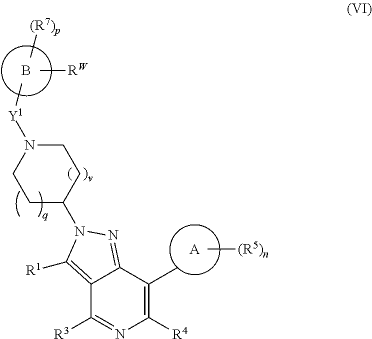

or a pharmaceutically acceptable salt thereof, wherein q is 0 or 1; v is 0 or 1; and Ring moiety A, ring moiety B, Y¹, R¹, R³, R⁴, R⁵, R⁷, R^{W}, n, and p are as defined according to any other embodiment described herein.

In some embodiments, the compound is a compound of Formula (VII):

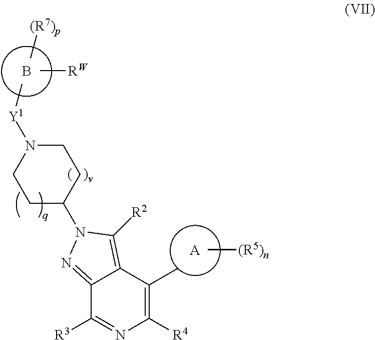

or a pharmaceutically acceptable salt thereof, wherein q is 0 or 1; v is 0 or 1; and Ring moiety A, ring moiety B, Y¹, R², R³, R⁴, R⁵, R⁷, R^{W}, n, and p are as defined according to any other embodiment described herein.

In some embodiments, the compound is selected from:
N-(4-(3-(7-(3,5-dichloro-4-hydroxyphenyl)-2H-[1,2,3]triazolo[4,5-c]pyridin-2-yl)azetidine-1-carbonyl)phenyl)acrylamide;
(S)—N-(4-(3-(7-(3,5-dichloro-4-hydroxyphenyl)-2H-[1,2,3]triazolo[4,5-c]pyridin-2-yl)pyrrolidine-1-carbonyl)phenyl)acrylamide;
rac-N-(4-(3-(7-(3,5-dichloro-4-hydroxyphenyl)-2H-[1,2,3]triazolo[4,5-c]pyridin-2-yl)piperidine-1-carbonyl)phenyl)acrylamide;
rac-N-(4-((2R,4S)-4-(7-(3,5-dichloro-4-hydroxyphenyl)-2H-[1,2,3]triazolo[4,5-c]pyridin-2-yl)-2-methylpyrrolidine-1-carbonyl)phenyl)acrylamide;
(S)—N-(4-(3-(7-(3,5-dichloro-4-hydroxyphenyl)-2H-[1,2,3]triazolo[4,5-c]pyridin-2-yl)pyrrolidine-1-carbonyl)-2-methoxyphenyl)acrylamide;
rac-N-(4-(3-(7-(3,5-dichloro-4-hydroxyphenyl)-3-(1-methyl-2-oxo-1,2-dihydropyridin-4-yl)-2H-pyrazolo[4,3-c]pyridin-2-yl)pyrrolidine-1-carbonyl)phenyl)acrylamide;
N-(4-((7-(3,5-dichloro-4-hydroxyphenyl)-2H-[1,2,3]triazolo[4,5-c]pyridin-2-yl)methyl)phenyl)acrylamide;
1-(5-((7-(3,5-dichloro-4-hydroxyphenyl)-2H-[1,2,3]triazolo[4,5-c]pyridin-2-yl)methyl)isoindolin-2-yl)prop-2-en-1-one;
rac-1-(5-(1-(7-(3,5-dichloro-4-hydroxyphenyl)-2H-[1,2,3]triazolo[4,5-c]pyridin-2-yl)ethyl)isoindolin-2-yl)prop-2-en-1-one;
1-(5-((4-(3,5-dichloro-4-hydroxyphenyl)-2H-pyrazolo[3,4-c]pyridin-2-yl)methyl)isoindolin-2-yl)prop-2-en-1-one;
1-(5-((7-(3,5-dichloro-4-hydroxyphenyl)-2H-pyrazolo[4,3-c]pyridin-2-yl)methyl)isoindolin-2-yl)prop-2-en-1-one;
(E)-2-(5-((7-(3,5-dichloro-4-hydroxyphenyl)-2H-[1,2,3]triazolo[4,5-c]pyridin-2-yl)methyl)isoindoline-2-carbonyl)-4-methylpent-2-enenitrile;
1-(5-((7-(3,5-dichloro-4-hydroxyphenyl)-2H-[1,2,3]triazolo[4,5-c]pyridin-2-yl)methyl)isoindolin-2-yl)but-2-yn-1-one;
1-(5-((7-(3,5-dichloro-4-hydroxyphenyl)-2H-[1,2,3]triazolo[4,5-c]pyridin-2-yl)methyl)isoindolin-2-yl)-2-fluoroprop-2-en-1-one;
(E)-1-(5-((7-(3,5-dichloro-4-hydroxyphenyl)-2H-[1,2,3]triazolo[4,5-c]pyridin-2-yl)methyl)isoindolin-2-yl)-4-(1H-1,2,4-triazol-1-yl)but-2-en-1-one;
(E)-1-(5-((7-(3,5-dichloro-4-hydroxyphenyl)-2H-[1,2,3]triazolo[4,5-c]pyridin-2-yl)methyl)isoindolin-2-yl)-3-(pyridin-4-yl)prop-2-en-1-one;
(E)-1-(5-((7-(3,5-dichloro-4-hydroxyphenyl)-2H-[1,2,3]triazolo[4,5-c]pyridin-2-yl)methyl)isoindolin-2-yl)-4,4,4-trifluorobut-2-en-1-one;
(E)-1-(5-((7-(3,5-dichloro-4-hydroxyphenyl)-2H-[1,2,3]triazolo[4,5-c]pyridin-2-yl)methyl)isoindolin-2-yl)-4-hydroxybut-2-en-1-one;
methyl (E)-4-(5-((7-(3,5-dichloro-4-hydroxyphenyl)-2H-[1,2,3]triazolo[4,5-c]pyridin-2-yl)methyl)isoindolin-2-yl)-4-oxobut-2-enoate;
(E)-1-(5-((7-(3,5-dichloro-4-hydroxyphenyl)-2H-pyrazolo[4,3-c]pyridin-2-yl)methyl)isoindolin-2-yl)-4,4-difluorobut-2-en-1-one;
(E)-4-(azetidin-1-yl)-1-(5-((7-(3,5-dichloro-4-hydroxyphenyl)-2H-[1,2,3]triazolo[4,5-c]pyridin-2-yl)methyl)isoindolin-2-yl)but-2-en-1-one;
1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-b]pyridin-6-yl)prop-2-en-1-one;
(E)-1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-b]pyridin-6-yl)-4-(diethylamino)but-2-en-1-one;
(E)-1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-b]pyridin-6-yl)-4-(piperidin-1-yl)but-2-en-1-one;
1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-d]pyrimidin-6-yl)prop-2-en-1-one; and
(E)-1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-d]pyrimidin-6-yl)-4-(piperidin-1-yl)but-2-en-1-one;
   or a pharmaceutically acceptable salt of any of the aforementioned.

In some embodiments, the compound is selected from:
rac-(E)-1-(2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridin-6-yl)-4-(diethylamino)but-2-en-1-one;
(R,E)-1-(2-(1-(7-(3,5-dichloro-4-hydroxyphenyl)-4-methyl-2H-[1,2,3]triazolo[4,5-c]118yridine-2-yl)ethyl)-5,7-dihydro-6H-pyrrolo[3,4-b]118yridine-6-yl)-4-(diethylamino)but-2-en-1-one;
(S,E)-1-(2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridin-6-yl)-4-(diethylamino)but-2-en-1-one;
(E)-1-(2-(2-(7-(3,5-dichloro-4-hydroxyphenyl)-4-methyl-2H-[1,2,3]triazolo[4,5-c]pyridin-2-yl)propan-2-yl)-5,7-dihydro-6H-pyrrolo[3,4-b]pyridin-6-yl)-4-(dimethylamino)but-2-en-1-one;
(S,E)-1-(2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridin-6-yl)-4-(2,2-dimethylpyrrolidin-1-yl)but-2-en-1-one;
rac-1-(2-(1-(7-(3,5-dichloro-4-hydroxyphenyl)-4-methyl-2H-[1,2,3]triazolo[4,5-c]pyridin-2-yl)ethyl)-3-fluoro-5,7-dihydro-6H-pyrrolo[3,4-b]pyridin-6-yl)prop-2-en-1-one;
(E)-1-(2-(1-(7-(3,5-dichloro-4-hydroxyphenyl)-2H-[1,2,3]triazolo[4,5-c]pyridin-2-yl)cyclobutyl)-5,7-dihydro-6H-pyrrolo[3,4-b]pyridin-6-yl)-4-(diethylamino)but-2-en-1-one;
rac-(E)-1-(2-(cyclopropyl(7-(3,5-dichloro-4-hydroxyphenyl)-4-methyl-2H-[1,2,3]triazolo[4,5-c]pyridin-2-yl)methyl)-5,7-dihydro-6H-pyrrolo[3,4-b]pyridin-6-yl)-4-(dimethylamino)but-2-en-1-one;
rac-(E)-1-(2-(1-(7-(3,5-dichloro-4-hydroxyphenyl)-2H-[1,2,3]triazolo[4,5-c]pyridin-2-yl)propan-2-yl)-5,7-dihydro-6H-pyrrolo[3,4-b]pyridin-6-yl)-4-(diethylamino)but-2-en-1-one;
rac-(E)-1-(2-(1-(4-amino-7-(3,5-dichloro-4-hydroxyphenyl)-2H-[1,2,3]triazolo[4,5-c]pyridin-2-yl)ethyl)-5,7-dihydro-6H-pyrrolo[3,4-b]pyridin-6-yl)-4-(2,2-dimethylpyrrolidin-1-yl)but-2-en-1-one;
(E)-7-(3,5-dichloro-4-hydroxyphenyl)-2-((6-(4-(diethylamino)but-2-enoyl)-3-methoxy-6,7-dihydro-5H-pyrrolo[3,4-b]pyridin-2-yl)methyl)-2H-[1,2,3]triazolo[4,5-c]pyridine-4-carbonitrile;
rac-(E)-4-(3-azabicyclo[3.1.1]heptan-3-yl)-1-(3-(1-(7-(3,5-dichloro-4-hydroxyphenyl)-4-ethyl-2H-[1,2,3]triazolo[4,5-c]pyridin-2-yl)ethyl)-2-methoxy-5,7-dihydro-6H-pyrrolo[3,4-b]pyridin-6-yl)but-2-en-1-one;
rac-1-(2-(1-(7-(3,5-dichloro-4-hydroxyphenyl)-4-methoxy-2H-[1,2,3]triazolo[4,5-c]pyridin-2-yl)ethyl)-5,7-dihydro-6H-pyrrolo[3,4-b]pyridin-6-yl)prop-2-en-1-one;
rac-(E)-1-(2-(1-(6-chloro-7-(3,5-dichloro-4-hydroxyphenyl)-2H-[1,2,3]triazolo[4,5-c]pyridin-2-yl)ethyl)-5,7-dihydro-6H-pyrrolo[3,4-b]pyridin-6-yl)-4-(dimethylamino)but-2-en-1-one;
(E)-1-(5-(2-(7-(2-chloro-6-methoxypyridin-4-yl)-4-methyl-2H-[1,2,3]triazolo[4,5-c]pyridin-2-yl)propan-2-yl)indolin-1-yl)-4-(dimethylamino)but-2-en-1-one;
(E)-4-(bis(methyl-d3)amino)-1-(5-((7-(3,5-dichloro-4-hydroxyphenyl)-4-methyl-2H-[1,2,3]triazolo[4,5-c]pyridin-2-yl)methyl)-4-fluoroindolin-1-yl)but-2-en-1-one;
(E)-1-(2-((7-(3,5-dichloro-4-hydroxyphenyl)-4-methyl-2H-[1,2,3]triazolo[4,5-c]pyridin-2-yl)methyl)-3-ethyl-5,7-dihydro-6H-pyrrolo[3,4-b]pyridin-6-yl)-4-(piperidin-1-yl)but-2-en-1-one;
(E)-4-(3-azabicyclo[3.1.1]heptan-3-yl)-1-(2-(2-(7-(3,5-dichloro-4-hydroxyphenyl)-4-methyl-2H-[1,2,3]triazolo[4,5-c]pyridin-2-yl)propan-2-yl)-4-methoxy-5,7-dihydro-6H-pyrrolo[3,4-b]pyridin-6-yl)but-2-en-1-one;
(E)-4-(3-azabicyclo[3.1.1]heptan-3-yl)-1-(2-((7-(3,5-dichloro-4-hydroxyphenyl)-4-methyl-2H-[1,2,3]triazolo[4,5-c]pyridin-2-yl)methyl)-3-methoxy-5,7-dihydro-6H-pyrrolo[3,4-b]pyrazin-6-yl)but-2-en-1-one;
rac-(E)-1-(2-(1-(7-(3,5-dichloro-4-hydroxyphenyl)-4-methyl-2H-[1,2,3]triazolo[4,5-c]pyridin-2-yl)ethyl)-4-methoxy-5,7-dihydro-6H-pyrrolo[3,4-d]pyrimidin-6-yl)-4-(piperidin-1-yl)but-2-en-1-one;
rac-(E)-1-(5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[2,3-c]pyridin-1-yl)-4-(dimethylamino)but-2-en-1-one;
(S,E)-1-(5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[3,2-b]pyridin-1-yl)-4-(dimethylamino)but-2-en-1-one; and
(E)-1-(5-(2-(7-(2-chloro-6-methoxypyridin-4-yl)-4-methyl-2H-[1,2,3]triazolo[4,5-c]pyridin-2-yl)propan-2-yl)-2,3-dihydro-1H-pyrrolo[2,3-c]pyridin-1-yl)-4-(diethylamino)but-2-en-1-one;
   or a pharmaceutically acceptable salt of any of the aforementioned.

In some embodiments, compounds of Formula (I), or a pharmaceutically acceptable salt thereof, can include one or more hydrogen atoms replaced by deuterium. In some embodiments, 1, 2, 3, 4, 5, 6, 7, or 8 hydrogen atoms, attached to carbon atoms of “alkyl”, “alkenyl”, “alkynyl”, “aryl”, “phenyl”, “cycloalkyl”, “heterocycloalkyl”, or “heteroaryl” substituents or “—C₁₋₄ alkyl-” and “alkylene” linking groups, as described herein, are optionally replaced by deuterium atoms.

It is further appreciated that certain features of the present disclosure, which are, for clarity, described in the context of separate embodiments, can also be provided in combination in a single embodiment (while the embodiments are intended to be combined as if written in multiply dependent form). Conversely, various features of the present disclosure which are, for brevity, described in the context of a single embodiment, can also be provided separately or in any suitable subcombination. Thus, it is contemplated as features described as embodiments of the compounds of Formula (I) can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ— includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency, that the designated atom's normal valency is not exceeded, and that the substitution results in a stable compound.

As used herein, the term “independently selected from” means that each occurrence of a variable or substituent are independently selected at each occurrence from the applicable list.

As used herein, the phrase “each ‘variable’ is independently selected from” means substantially the same as wherein “at each occurrence ‘variable’ is selected from.”

When any variable (e.g., R^{G}) occurs more than one time in any constituent or formula for a compound, its definition at each occurrence is independent of its definition at every other occurrence. Thus, for example, if a group is shown to be substituted with 1, 2, 3, or 4 independently selected R^{G} substituents, then said group may optionally be substituted with up to four R^{G} groups and R^{G} at each occurrence is selected independently from the definition of R^{G}.

In some embodiments, when an optionally multiple substituent is designated in the form:

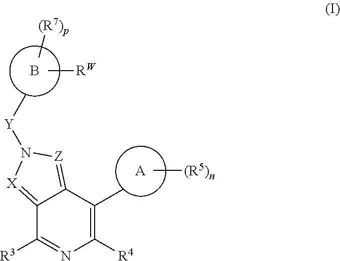

then it is to be understood that substituent R⁵ can occur n number of times on the ring, and R⁵ can be a different moiety at each occurrence. It is to be understood that each R⁵ group may replace any hydrogen atom attached to a ring atom. It is to be understood that substituent R⁷ can occur p number of times on the ring, and R⁷ can be a different moiety at each occurrence. It is to be understood that each R⁷ group may replace any hydrogen atom attached to a ring atom.

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (i-Pr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms. As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “C₁₋₄ alkylidene-C₃₋₇ cycloalkyl” refers to a group of formula

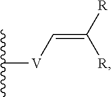

wherein the V is a bond or C₁₋₃ alkylene, and R and R, taken together with the carbon atom to which they are attached, form a C₃₋₇ cycloalkyl ring.

As used herein, “C₁₋₄ alkylidene-4-7-membered heterocycloalkyl” refers to a group of formula

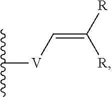

wherein the V is a bond or C₁₋₃ alkylene, and R and R, taken together with the carbon atom to which they are attached, form a 4-7 membered heterocycloalkyl ring.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. In some embodiments, the aryl group has 6 to 10 carbon atoms. In some embodiments, the aryl group is phenyl or naphthyl. In some embodiments, the aryl is phenyl.

As used herein, “halo” refers to F, Cl, Br, or I. In some embodiments, halo is F, Cl, or Br. In some embodiments, halo is F or Cl. In some embodiments, halo is F. In some embodiments, halo is Cl.

As used herein, “Cₙ₋ₘ haloalkoxy” refers to a group of formula —O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group of the haloalkyl has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, the term “Cₙ₋ₘ fluoroalkyl” refers to an alkyl group having from one fluoro atom to 2s+1 fluoro atoms, where “s” is the number of carbon atoms in the alkyl group, wherein the alkyl group has n to m carbon atoms. In some embodiments, the alkyl group of the fluoroalkyl has 1 to 6, 1 to 4, or 1 to 3 carbon atoms. Example fluoroalkyl groups include CF₃, C₂F₅, CHF₂, CH₂F, and the like.

As used herein, the term “thio” refers to a group of formula —SH.

As used herein, the term “Cₙ₋ₘ alkylamino” refers to a group of formula —NH(alkyl), wherein the alkyl group has n to m carbon atoms. In some embodiments, the alkyl group of the alkylamino has 1 to 6, 1 to 4, or 1 to 3 carbon atoms.

As used herein, the term “Cₙ₋ₘ alkoxycarbonyl” refers to a group of formula —C(O)O— alkyl, wherein the alkyl group has n to m carbon atoms. In some embodiments, the alkyl group of the alkoxycarbonyl has 1 to 6, 1 to 4, or 1 to 3 carbon atoms.

As used herein, the term “Cₙ₋ₘ alkylcarbonyl” refers to a group of formula —C(O)— alkyl, wherein the alkyl group has n to m carbon atoms. In some embodiments, the alkyl group of the alkylcarbonyl has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group of the alkylcarbonylamino has 1 to 6, 1 to 4, or 1 to 3 carbon atoms.

As used herein, the term “Cₙ₋ₘ alkoxycarbonylamino” refers to a group of formula —NHC(O)O(Cₙ₋ₘ alkyl), wherein the alkyl group has n to m carbon atoms. In some embodiments, the alkyl group of the alkoxycarbonylamino has 1 to 6, 1 to 4, or 1 to 3 carbon atoms.

As used herein, the term “Cn-m alkylsulfonylamino” refers to a group of formula —NHS(O)₂-alkyl, wherein the alkyl group has n to m carbon atoms. In some embodiments, the alkyl group of the alkylsulfonylamino has 1 to 6, 1 to 4, or 1 to 3 carbon atoms.

As used herein, the term “aminosulfonyl” refers to a group of formula —S(O)₂NH₂.

As used herein, the term “Cn-m alkylaminosulfonyl” refers to a group of formula —S(O)₂NH(alkyl), wherein the alkyl group has n to m carbon atoms. In some embodiments, the alkyl group of the alkylaminosulfonyl has 1 to 6, 1 to 4, or 1 to 3 carbon atoms.

As used herein, the term “di(Cn-m alkyl)aminosulfonyl” refers to a group of formula —S(O)₂N(alkyl)₂, wherein each alkyl group independently has n to m carbon atoms.

In some embodiments, each alkyl group of the dialkylaminosulfonyl has, independently, 1 to 6, 1 to 4, or 1 to 3 carbon atoms.

As used herein, the term “aminosulfonylamino” refers to a group of formula —NHS(O)₂NH₂.

As used herein, the term “Cn-m alkylaminosulfonylamino” refers to a group of formula —NHS(O)₂NH(alkyl), wherein the alkyl group has n to m carbon atoms. In some embodiments, the alkyl group of the alkylaminosulfonylamino has 1 to 6, 1 to 4, or 1 to 3 carbon atoms.

As used herein, the term “di(Cn-m alkyl)aminosulfonylamino” refers to a group of formula —NHS(O)₂N(alkyl)₂, wherein each alkyl group independently has n to m carbon atoms. In some embodiments, each alkyl group of the dialkylaminosulfonylamino has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group of the alkylaminocarbonylamino has 1 to 6, 1 to 4, or 1 to 3 carbon atoms.

As used herein, the term “di(Cn-m alkyl)aminocarbonylamino” refers to a group of formula —NHC(O)N(alkyl)₂, wherein each alkyl group independently has n to m carbon atoms. In some embodiments, each alkyl group of the dialkylaminocarbonylamino has, independently, 1 to 6, 1 to 4, or 1 to 3 carbon atoms.

As used herein, the term “Cn-m alkylcarbamyl” refers to a group of formula —C(O)—NH(alkyl), wherein the alkyl group has n to m carbon atoms. In some embodiments, the alkyl group of the alkylcarbamyl has 1 to 6, 1 to 4, or 1 to 3 carbon atoms.

As used herein, the term “Cn-m alkylthio” refers to a group of formula —S-alkyl, wherein the alkyl group has n to m carbon atoms. In some embodiments, the alkyl group of the alkylthio has 1 to 6, 1 to 4, or 1 to 3 carbon atoms.

As used herein, the term “Cn-m alkylsulfinyl” refers to a group of formula —S(O)-alkyl, wherein the alkyl group has n to m carbon atoms. In some embodiments, the alkyl group of the alkylsulfinyl has 1 to 6, 1 to 4, or 1 to 3 carbon atoms.

As used herein, the term “Cn-m alkylsulfonyl” refers to a group of formula —S(O)₂-alkyl, wherein the alkyl group has n to m carbon atoms. In some embodiments, the alkyl group of the alkylsulfonyl has 1 to 6, 1 to 4, or 1 to 3 carbon atoms.

As used herein, the term “cyano-Cn-m alkyl” refers to a group of formula —(Cₙ₋ₘ alkylene)-CN, wherein the alkylene group has n to m carbon atoms. As used herein, the term “cyano-C₁₋₆ alkyl” refers to a group of formula —(C₁₋₆ alkylene)-CN. As used herein, the term “cyano-C₁₋₃ alkyl” refers to a group of formula —(C₁₋₃ alkylene)-CN.

As used herein, the term “HO—Cₙ₋ₘ alkyl” refers to a group of formula —(Cₙ₋ₘ alkylene)-OH, wherein the alkylene group has n to m carbon atoms. As used herein, the term “HO—C₁₋₃ alkyl” refers to a group of formula —(C₁₋₃ alkylene)-OH.

As used herein, the term “Cₙ₋ₘ alkoxy-Cₒ₋ₚ alkyl” refers to a group of formula —(Cₙ₋ₘ alkylene)-O(Cₒ₋ₚ alkyl), wherein the alkylene group has n to m carbon atoms and the alkyl group has o to p carbon atoms. As used herein, the term “C₁₋₆ alkoxy-C₁₋₆ alkyl” refers to a group of formula —(C₁₋₆ alkylene)-O(C₁₋₆ alkyl). As used herein, the term “C₁₋₃ alkoxy-C₁₋₃ alkyl” refers to a group of formula —(C₁₋₃ alkylene)-O(C₁₋₃ alkyl).

As used herein, the term “carboxy” refers to a group of formula —C(O)OH.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group of the dialkylamino independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group of the dialkylcarbamyl independently has 1 to 6, 1 to 4, or 1 to 3 carbon atoms.

As used herein, the term “Cₙ₋ₘ alkylcarbonyloxy” is a group of formula —OC(O)-alkyl, wherein the alkyl group has n to m carbon atoms. In some embodiments, the alkyl group of the alkylcarbonyloxy has 1 to 6, 1 to 4, or 1 to 3 carbon atoms.

As used herein, “aminocarbonyloxy” is a group of formula —OC(O)—NH₂.

As used herein, “Cₙ₋ₘ alkylaminocarbonyloxy” is a group of formula —OC(O)—NH— alkyl, wherein the alkyl group has n to m carbon atoms. In some embodiments, the alkyl group of the alkylaminocarbonyloxy has 1 to 6, 1 to 4, or 1 to 3 carbon atoms.

As used herein, “di(Cₙ₋ₘalkyl)aminocarbonyloxy” is a group of formula —OC(O)—N(alkyl)₂, wherein each alkyl group has, independently, n to m carbon atoms. In some embodiments, each alkyl group of the dialkylaminocarbonyloxy independently has 1 to 6, 1 to 4, or 1 to 3 carbon atoms.

As used herein “Cₙ₋ₘ alkoxycarbonylamino” refers to a group of formula —NHC(O)—O— alkyl, wherein the alkyl group has n to m carbon atoms.

As used herein, the term “carbamyl” to a group of formula —C(O)NH₂.

As used herein, the term “carbonyl”, employed alone or in combination with other terms, refers to a —C(O)— group.

As used herein, “cycloalkyl” refers to non-aromatic cyclic hydrocarbons including cyclized alkyl and alkenyl groups. Cycloalkyl groups can include mono- or polycyclic (e.g., having 2, 3 or 4 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃-10). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2, 3, or 4 fused rings) aromatic heterocycle having at least one heteroatom ring member selected from N, O, or S. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, and S. In some embodiments, the heteroaryl group contains 5 to 10 or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, pyridyl, pyrimidinyl, pyrazinyl, pyridazinyl, pyrrolyl, pyrazolyl, azolyl, oxazolyl, isoxazolyl, thiazolyl, isothiazolyl, imidazolyl, furyl, thienyl, triazolyl (e.g., 1,2,3-triazolyl, 1,2,4-triazolyl, 1,3,4-triazolyl), tetrazolyl, thiadiazolyl (e.g., 1,2,3-thiadiazolyl, 1,2,4-thiadiazolyl, 1,3,4-thiadiazolyl), quinolinyl, isoquinolinyl, indolyl, benzothienyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, oxadiazolyl (e.g., 1,2,3-oxadiazolyl, 1,2,4-oxadiazolyl, 1,3,4-oxadiazolyl), 1,2-dihydro-1,2-azoborinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, or S, and wherein the ring-forming carbon atoms and heteroatoms of the heterocycloalkyl group can be optionally substituted by one or more oxo or sulfido (e.g., C(O), S(O), C(S), or S(O)₂, etc.). Heterocycloalkyl groups include monocyclic and polycyclic (e.g., having 2 fused rings) systems. Included in heterocycloalkyl are monocyclic and polycyclic 4-10-, 4-7-, and 5-6-membered heterocycloalkyl groups. Heterocycloalkyl groups can also include spirocycles and bridged rings. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 4 to 10 ring-forming atoms, 4 to 7 ring-forming atoms, 4 to 6 ring-forming atoms or 5 to 6 ring-forming atoms. In some embodiments, the heterocycloalkyl group has 1 to 4 heteroatoms, 1 to 3 heteroatoms, 1 to 2 heteroatoms or 1 heteroatom.

In some embodiments, the heterocycloalkyl is a 4-10 membered monocyclic, bicyclic, or tricyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-10 membered bicyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido.

In some embodiments, the heterocycloalkyl is a 4-7 membered monocyclic heterocycloalkyl having 1 or 2 ring-forming heteroatoms independently selected from N, O, and S, and wherein 1, 2 or 3 ring-forming carbon or heteroatoms can be optionally substituted by one or more oxo or sulfido. In some embodiments, the heterocycloalkyl is a monocyclic 4-6 membered heterocycloalkyl having 1 or 2 heteroatoms independently selected from N, O, S, and B and having one or more oxidized ring members.

Examples of heterocycloalkyl groups include pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, 1,2,3,4-tetrahydroisoquinoline, azabicyclo[3.1.0]hexanyl, diazabicyclo[3.1.0]hexanyl, oxabicyclo[2.1.1]hexanyl, azabicyclo[2.2.1]heptanyl, azabicyclo[2.2.1]heptan-7-yl, azabicyclo[2.2.1]heptan-2-yl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon ring members and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, the term “alkylene” refers a divalent straight chain or branched alkyl linking group. Examples of “alkylene groups” include methylene, ethan-1,1-diyl, ethan-1,2-diyl, propan-1,3-dilyl, propan-1,2-diyl, propan-1,1-diyl and the like.

As used herein, the term “alkenylene” refers a divalent straight chain or branched alkenyl linking group. Examples of “alkenylene groups” include ethen-1,1-diyl, ethen-1,2-diyl, propen-1,3-diyl, 2-buten-1,4-diyl, 3-penten-1,5-diyl, 3-hexen-1,6-diyl, 3-hexen-1,5-diyl, and the like.

As used herein, the term “alkynylene” refers a divalent straight chain or branched alkynyl linking group. Examples of “alkynylene groups” include propyn-1,3-diyl, 2-butyn-1,4-diyl, 3-pentyn-1,5-diyl, 3-hexyn-1,6-diyl, 3-hexyn-1,5-diyl, and the like.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula (I), (II), etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g., hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected.

Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes, such as those provided in the Schemes below.

Compounds of Formula (I) can be prepared as shown in Scheme 1A. Suitable starting material triazolopyridine/pyrazolopyridine/pyrrolopyridine 1A-1 can be converted to coupling product 1A-3, under alkylation or Mitsunobu conditions, wherein Ring moiety B is a cyclic structure bearing a protected amino group that can be a substituent in (1A-2a) or off (1A-2b) of the ring; X⁴ is a halide; X⁵ is a halide or a hydroxyl group. Cross-coupling reaction between halopyridine 1A-3 and compound 1A-4, wherein X⁶ is a metal (e.g., X⁶ is B(OR)₂, Sn(Alkyl)₃, Zn-Hal, etc.), H or an appropriate group can behave like a leaving group under cross-coupling conditions, gives compound 1A-5. Protecting group in 1A-5 can be removed to provide free amine 1A-6. 1A-7 of Formula (I) is made from 1A-6 under standard amide/sulfonamide formation conditions (e.g., in the presence of a carboxylic acid, carboxylic chloride or sulfonyl chloride) and alkylation of amine/reductive amination conditions (e.g., in the presence of an alkyl halide or aldehyde).

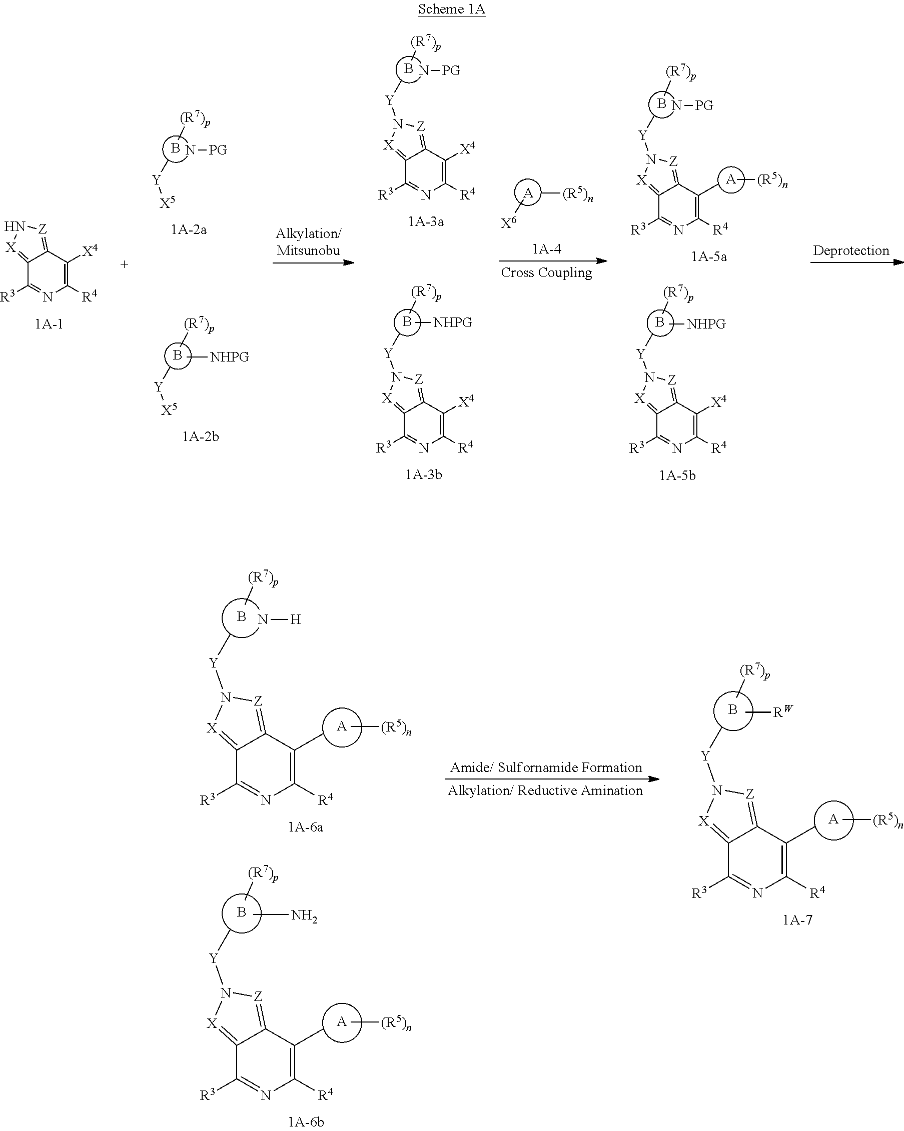

To introduce a broader scope of warhead piece R^{W}, free amine 1A-6b could be converted to 2A-1 under Sandmeyer and Miyaura/Negishi/Stille conditions, wherein X⁷ is a halide, metal (e.g., X⁶ is B(OR)₂, Sn(Alkyl)₃, Zn-Hal, etc.) or alcohol. 1A-7 of Formula (I) is made from 2A-1 under standard cross coupling conditions with 2A-2 (e.g., X⁸ is a halide, metal, such as B(OR)₂, Sn(Alkyl)₃, Zn-Hal, etc.) or alkylation of alcohol conditions (e.g., X⁸ is a halide).

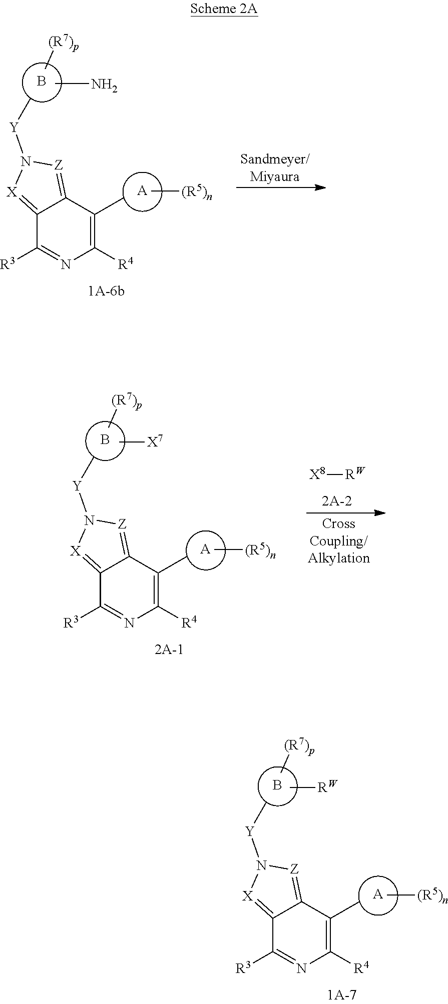

Compounds of Formula (I) can be prepared as shown in Scheme 1B. Suitable starting material triazolopyridine/pyrazolopyridine/pyrrolopyridine 1B-1 can be converted to coupling product 1B-3, under alkylation or Mitsunobu conditions, wherein Ring moiety B is a cyclic structure bearing a protected amino group that can be a substituent in (1B-2a) or off (1B-2b) of the ring; X⁴ is a halide; X⁵ is a halide or a hydroxyl group; X⁶ is a halide or a hydrogen. Cross-coupling reaction between halopyridine 1B-3 and compound 1B-4, wherein X⁷ is a metal (e.g., X⁷ is B(OR)₂, Sn(Alkyl)₃, Zn-Hal, etc.), H or an appropriate group can behave like a leaving group under cross-coupling conditions, gives compound 1B-5. A second cross-coupling reaction between halopyridine 1B-5 and compound 1B-6, wherein X⁸ is a metal (e.g., X⁸ is B(OR)₂, Sn(Alkyl)₃, Zn-Hal, etc.), H or an appropriate group can behave like a leaving group under cross-coupling conditions, gives compound 1B-7.

Protecting groups in 1B-7 can be removed to provide free amine 1B-8. 1B-9 of Formula (I) is made from 1B-8 under standard amide/sulfonamide formation conditions (e.g., in the presence of a carboxylic acid, carboxylic chloride or sulfonyl chloride) and alkylation of amine/reductive amination conditions (e.g., in the presence of an alkyl halide or aldehyde).

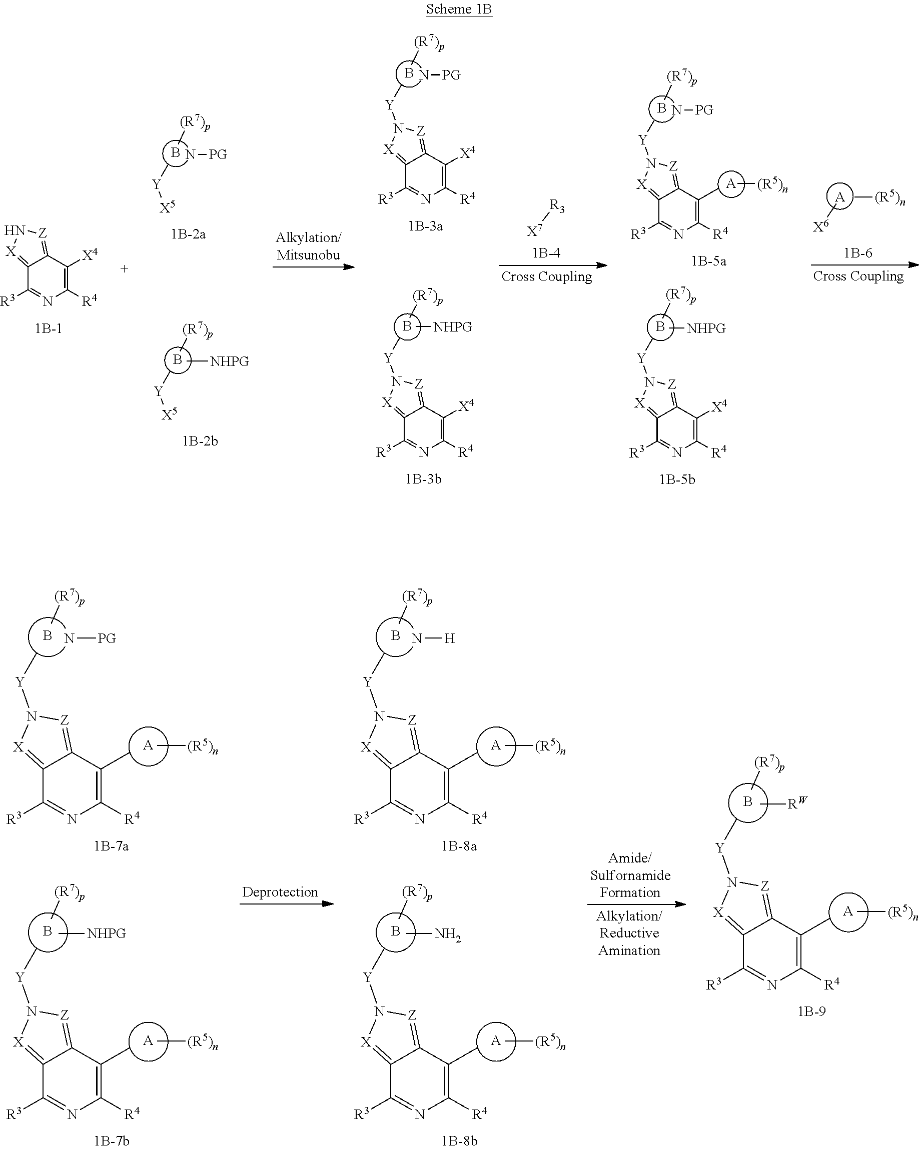

To introduce a broader scope of warhead piece R^{W}, free amine 1B-8b could be converted to 2B-1 under Sandmeyer and Miyaura/Negishi/Stille conditions, wherein X⁹ is a halide, metal (e.g., X¹⁰ is B(OR)₂, Sn(Alkyl)₃, Zn-Hal, etc.) or alcohol. 1B-9 of Formula (I) is made from 2B-1 under standard cross coupling conditions with 2B-2 (e.g., X¹⁰ is a halide, metal, such as B(OR)₂, Sn(Alkyl)₃, Zn-Hal, etc.) or alkylation of alcohol conditions (e.g., X¹⁰ is a halide).

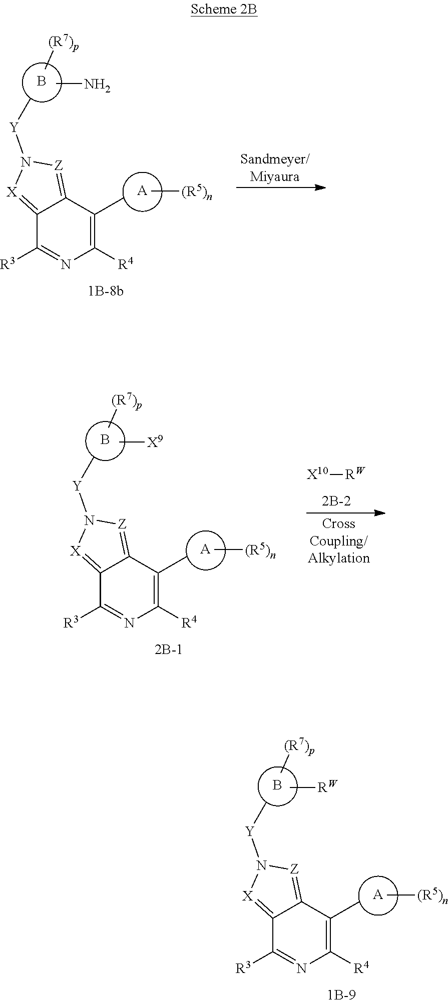

The reactions for preparing compounds described herein can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “r.t.” as used herein, are understood in the art, and refer generally to a temperature, e.g., a reaction temperature, that is about the temperature of the room in which the reaction is carried out, for example, a temperature from about 20° C. to about 30° C.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Methods of Use

The present disclosure further provides methods of inhibiting VRK1, comprising contacting the VRK1 with a VRK1 inhibitor, or a pharmaceutically acceptable salt thereof. The present disclosure further provides methods of selectively inhibiting VRK1 over VRK2, comprising contacting VRK1 and VRK2 with a VRK1 inhibitor, or a pharmaceutically acceptable salt thereof.

In some embodiments, the VRK1 inhibitor, or a pharmaceutically acceptable salt thereof, covalently binds with a cysteine in VRK1. In some embodiments, the VRK1 inhibitor, or a pharmaceutically acceptable salt thereof, covalently binds with Cys50 in VRK1.

In some embodiments, the VRK1 inhibitor, or a pharmaceutically acceptable salt thereof, binds with a water in the binding pocket of VRK1. In some embodiments, the water in the binding pocket of VRK1 interacts with one or more of Asp197, Tyr87, and Glu83 of VRK1. In some embodiments, the water in the binding pocket of VRK1 interacts with Asp197, Tyr87, and Glu83 of VRK1. In some embodiments, the water in the binding pocket of VRK1 interacts with Asp197 of VRK1. In some embodiments, the water in the binding pocket of VRK1 interacts with Tyr87 of VRK1. In some embodiments, the water in the binding pocket of VRK1 interacts with Glu83 of VRK1.

In some embodiments, the VRK1 inhibitor, or a pharmaceutically acceptable salt thereof, binds with Phe134 of VRK1.

In some embodiments, the VRK1 inhibitor, or a pharmaceutically acceptable salt thereof, binds with Lys71 of VRK1.

In some embodiments, the VRK1 inhibitor, or a pharmaceutically acceptable salt thereof, binds with Asp132 of VRK1.

In some embodiments, the VRK1 inhibitor, or a pharmaceutically acceptable salt thereof, binds with the phosphate-binding loop of VRK1.

In some embodiments, the VRK1 inhibitor, or a pharmaceutically acceptable salt thereof, covalently binds with a cysteine in VRK1, binds with a water in the binding pocket of VRK1, and binds with Phe134 of VRK1.

In some embodiments, the VRK1 inhibitor, or a pharmaceutically acceptable salt thereof, covalently binds with a cysteine in VRK1, binds with a water in the binding pocket of VRK1, binds with Phe134 of VRK1, binds with Lys71 of VRK1, binds with Asp132 of VRK1, and binds with the phosphate-binding loop of VRK1.

In some embodiments, the VRK1 inhibitor, or a pharmaceutically acceptable salt thereof, covalently bonds with VRK1.

In some embodiments, the VRK1 inhibitor, or a pharmaceutically acceptable salt thereof, binds with VRK1 through one or more strong interactions and one or more weaker interactions.

In some embodiments, the VRK1 inhibitor, or a pharmaceutically acceptable salt thereof, binds with VRK1 through one or more strong interactions. In some embodiments, the strong interactions are selected from hydrogen bond interactions and salt bridge interactions.

In some embodiments, the strong interactions are one or more hydrogen bond interactions and one or more salt bridge interactions. In some embodiments, the strong interactions are one or more hydrogen bond interactions. In some embodiments, the strong interactions are one or more salt bridge interactions.

In some embodiments, the VRK1 inhibitor, or a pharmaceutically acceptable salt thereof, binds with VRK1 through one or more weaker interactions. In some embodiments, the weaker interactions are selected from CH-7c interactions, C—H hydrogen bonding interactions, and van der Waals interactions. In some embodiments, the weaker interactions are one or more CH-7c interactions, one or more C—H hydrogen bonding interactions, and one or more van der Waals interactions. In some embodiments, the weaker interactions are one or more CH-7c interactions. In some embodiments, the weaker interactions are one or more C—H hydrogen bonding interactions. In some embodiments, the weaker interactions are one or more van der Waals interactions.

The VRK1 inhibitor can include a compound of the present disclosure, or a pharmaceutically acceptable salt thereof.

Compounds of the present disclosure (and pharmaceutically acceptable salts thereof) can inhibit VRK1 and therefore are useful for treating diseases wherein the underlying pathology is, wholly or partially, mediated by VRK1. In some embodiments, the compounds, and pharmaceutically acceptable salts thereof, inhibit VRK1. In some embodiments, the compounds, and pharmaceutically acceptable salts thereof, are selective for VRK1 over VRK2. In some embodiments, the selectivity referenced in each of the embodiments of this paragraph is at least 10-fold, at least 100-fold selective, at least 500-fold selective, or at least 1000 fold selective.

In some embodiments, R^{W} of Formula (I) is a means for covalently binding with a cysteine (Cys).

In some embodiments, R^{W} of Formula (I) is a means for covalently binding with a Cys in VRK1.

In some embodiments, R^{W} of Formula (I) is a means for covalently binding with Cys50 in VRK1.

In some embodiments, Ring moiety A of Formula (I) is a means for binding with a lyseine (Lys).

In some embodiments, Ring moiety A of Formula (I) is a means for binding with a Lys in VRK1.

In some embodiments, Ring moiety A of Formula (I) is a means for binding with Lys71 in VRK1.

In some embodiments, Ring moiety A of Formula (I) is a means for binding with a water in the binding pocket of VRK1.

In some embodiments, the 2H-pyrrolo[3,4-c]pyridinyl (e.g., when X is CR¹ and Z is CR²), 2H-pyrazolo[4,3-c]pyridinyl (e.g., when X is CR¹ and Z is N), 2H-pyrazolo[3,4-c]pyridinyl (e.g., when X is N and Z is CR²), or 2H-[1,2,3]triazolo[4,5-c]pyridinyl (e.g., when X is N and Z is N) of Formula (I) is a means for binding with a phenylalanine (Phe).

In some embodiments, the 2H-pyrrolo[3,4-c]pyridinyl (e.g., when X is CR¹ and Z is CR²), 2H-pyrazolo[4,3-c]pyridinyl (e.g., when X is CR¹ and Z is N), 2H-pyrazolo[3,4-c]pyridinyl (e.g., when X is N and Z is CR²), or 2H-[1,2,3]triazolo[4,5-c]pyridinyl (e.g., when X is N and Z is N) of Formula (I) is a means for binding with a Phe in VRK1.

In some embodiments, the 2H-pyrrolo[3,4-c]pyridinyl (e.g., when X is CR¹ and Z is CR²), 2H-pyrazolo[4,3-c]pyridinyl (e.g., when X is CR¹ and Z is N), 2H-pyrazolo[3,4-c]pyridinyl (e.g., when X is N and Z is CR²), or 2H-[1,2,3]triazolo[4,5-c]pyridinyl (e.g., when X is N and Z is N) of Formula (I) is a means for binding with Phe134 in VRK1.

In one embodiment, the present disclosure provides a method for inhibiting growth of tumor cells in vitro. The method includes contacting the tumor cells in vitro with a compound of Formula (I) or of any of the formulas as described herein, or of a compound as recited in any of the claims and described herein, or of a salt thereof. The method includes administering to the individual or patient in need thereof a therapeutically effective amount of a compound of Formula (I) or of any of the formulas as described herein, or of a compound as recited in any of the claims and described herein, or a salt or a stereoisomer thereof.

In some embodiments, provided herein is a method of inhibiting VRK1, comprising contacting the VRK1 with a compound of Formula (I) or any of the formulas as described herein, a compound as recited in any of the claims and described herein, or a salt thereof. In some embodiments, provided herein is a method of inhibiting VRK1 in a patient, comprising administering to the patient a compound of Formula (I) or any of the formulas as described herein, a compound as recited in any of the claims and described herein, or a salt thereof.

In some embodiments, the disclosure includes a method of treating a disease or disorder associated with VRK1 in a patient, comprising administering to the patient a therapeutically effective amount of the compound described herein, or pharmaceutically acceptable salt thereof. Such diseases include cancer and other diseases with proliferation disorder. In some embodiments, the present disclosure provides treatment of an individual or a patient in vivo using a compound of Formula (I) or a salt thereof such that growth of cancerous tumors is inhibited. A compound of Formula (I) or of any of the formulas as described herein, or a compound as recited in any of the claims and described herein, or a salt thereof, can be used to inhibit the growth of cancerous tumors with aberrations that activate the VRK1 kinase and/or VRK1 activity.

In some embodiments, provided herein is a method of treating a disease or disorder associated with VRK1 in a patient, comprising administering to the patient a therapeutically effective amount of a compound of Formula (I) or any of the formulas as described herein, a compound as recited in any of the claims and described herein, or a salt thereof.

In some embodiments, provided herein is a method for treating cancer. The method includes administering to a patient (in need thereof), a therapeutically effective amount of a compound of Formula (I) or any of the formulas as described herein, a compound as recited in any of the claims and described herein, or a salt thereof. In some embodiments, the cancer is sarcoma, colorectal adenocarcinoma, acute myeloid leukemia, glioblastoma, or neuroblastoma. In some embodiments, the glioblastoma or neuroblastoma has overexpression of VRK1. In some embodiments, the glioblastoma or neuroblastoma has underexpression of VRK2. In some embodiments, the cancer is bone cancer, cancer of the neck and head, rectal cancer, esophageal cancer, sarcoma of soft tissue, neoplasm of the central nervous system (CNS), brain stem, glioma, colon cancer, Ewing's sarcoma, brain cancer, astrocytoma, neuroblastoma, intestinal cancer, nervous system cancers, osteosarcoma, cancer of the small bowel (adenocarcinoma, lymphoma, carcinoid tumors, Kaposi's sarcoma, leiomyoma, hemangioma, lipoma, neurofibroma, fibroma), or cancer of the large bowel (adenocarcinoma, tubular adenoma, villous adenoma, hamartoma, leiomyoma).

Examples of cancers that are treatable using the compounds of the present disclosure include, but are not limited to, glioblastoma and neuroblastoma having underexpression of VRK2, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include melanoma (e.g., metastatic malignant melanoma, BRAF and HSP90 inhibition-resistant melanoma), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer, urothelial cancer (e.g., bladder) and cancers with high microsatellite instability (MSI^{high}). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including follicular lymphoma, including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma, and giant cell tumors.

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre -tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

It is believed that compounds of Formula (I), or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

The terms “individual”, “patient,” and “subject” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

I. Cancer therapies

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, and CDK4/6 kinase inhibitors such as, for example, those described in WO 2006/056399 can be used in combination with the compounds of the present disclosure for treatment of VRK1-associated diseases, disorders or conditions. Other agents such as therapeutic antibodies can be used in combination with the compounds of the present disclosure for treatment of VRK1-associated diseases, disorders or conditions. The one or more additional pharmaceutical agents can be administered to a patient simultaneously or sequentially.

In some embodiments, the compound or salt is administered or used in combination with a BCL2 inhibitor.

The compounds as disclosed herein can be used in combination with one or more other enzyme/protein/receptor inhibitors therapies for the treatment of diseases, such as cancer and other diseases or disorders described herein. Examples of diseases and indications treatable with combination therapies include those as described herein. Examples of cancers include solid tumors and non-solid tumors, such as liquid tumors, and blood cancers.

Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK4/6, TGF-□R, PKA, PKG, PKC, CaM-kinase, phosphorylase kinase, MEKK, ERK, MAPK, mTOR, EGFR, HER2, HER3, HER4, INS—R, IDH2, IGF-1R, IR—R, PDGF□R, PDGF□R, PI3K (alpha, beta, gamma, delta, and multiple or selective), CSF1R, KIT, FLK-II, KDR/FLK-1, FLK-4, fit-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2, e.g., ruxolitinib or baricitinib; JAKI, e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872 and INCB60003), a TDO inhibitor, a PI3K-delta inhibitor (e.g., parsaclisib (INCB50465) or INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., INCB00928); or combinations thereof.

In some embodiments, the compound or salt described herein is administered with a PI3K6 inhibitor. In some embodiments, the compound or salt described herein is administered with a JAK inhibitor. In some embodiments, the compound or salt described herein is administered with a JAK1 or JAK2 inhibitor (e.g., baricitinib or ruxolitinib). In some embodiments, the compound or salt described herein is administered with a JAK1 inhibitor. In some embodiments, the compound or salt described herein is administered with a JAK1 inhibitor, which is selective over JAK2.

Example antibodies for use in combination therapy include, but are not limited to, trastuzumab (e.g., anti-HER2), ranibizumab (e.g., anti-VEGF-A), bevacizumab (AVASTIN™, e.g., anti-VEGF), panitumumab (e.g., anti-EGFR), cetuximab (e.g., anti-EGFR), rituxan (e.g., anti-CD20), and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptosar, topotecan, paclitaxel, docetaxel, epothilones, tamoxifen, 5-fluorouracil, methotrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide, 17-alpha-ethinylestradiol, diethylstilbestrol, testosterone, prednisone, fluoxymesterone, dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesterone acetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-I agonists, oncolytic virotherapy and immunomodulating small molecules, including thalidomide or JAK1/2 inhibitor, PI3K6 inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a compound or salt of the present disclosure with an additional agent.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self -antigens. Examples of pathogens for which this therapeutic approach may be particularly useful include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas Aeruginosa.

Viruses causing infections treatable by methods of the present disclosure include, but are not limited to human papillomavirus, influenza, hepatitis A, B, C or D viruses, adenovirus, poxvirus, herpes simplex viruses, human cytomegalovirus, severe acute respiratory syndrome virus, Ebola virus, measles virus, herpes virus (e.g., VZV, HSV-1, HAV-6, HSV-II, and CMV, Epstein Barr virus), flaviviruses, echovirus, rhinovirus, coxsackie virus, cornavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

### II. Immune-Checkpoint Therapies

Compounds of the present disclosure can be used in combination with one or more immune checkpoint inhibitors for the treatment of diseases, such as cancer or infections.

Exemplary immune checkpoint inhibitors include inhibitors against immune checkpoint molecules such as CBL-B, CD20, CD28, CD40, CD70, CD122, CD96, CD73, CD47, CDK2, CDK4, CDK12, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, WO 03042402, WO 2008156712, WO 2010089411, WO 2010036959, WO 2011066342, WO 2011159877, WO 2011082400, or WO 2011161699, which are each incorporated herein by reference in its entirety.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012. In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736.

In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333.

In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F₉-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1B).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, RO7009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

As provided throughout, the additional compounds, inhibitors, agents, etc. can be combined with the present compound in a single or continuous dosage form, or they can be administered simultaneously or sequentially as separate dosage forms.

### Labeled Compounds and Assay Methods

The compounds of the present disclosure can further be useful in investigations of biological processes in normal and abnormal tissues. Thus, another aspect of the present disclosure relates to labeled compounds of the present disclosure (radio-labeled, fluorescent-labeled, etc.) that would be useful not only in imaging techniques but also in assays, both in vitro and in vivo, for localizing and quantitating VRK1 protein in tissue samples, including human, and for identifying VRK1 ligands by inhibition binding of a labeled compound. Accordingly, the present disclosure includes VRK1 binding assays that contain such labeled compounds.

The present disclosure further includes isotopically-labeled compounds of the disclosure. An “isotopically” or “radio-labeled” compound is a compound of the present disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as -CD₃ being substituted for —CH₃). In some embodiments, alkyl groups in Formula (I) can be perdeuterated.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (See e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro VRK1 protein labeling and competition assays, compounds that incorporate ³H, ¹⁴C, ⁸²Br, ¹²⁵I, ¹³¹I, ³⁵S or will generally be most useful. For radio-imaging applications ¹¹C, ⁸F, ¹²⁵J, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the present disclosure can be used in a screening assay to identify and/or evaluate compounds. For example, a newly synthesized or identified compound (i.e., test compound) which is labeled can be evaluated for its ability to bind a VRK1 protein by monitoring its concentration variation when contacting with the VRK1 protein, through tracking of the labeling. For example, a test compound (labeled) can be evaluated for its ability to reduce binding of another compound which is known to bind to a VRK1 protein (i.e., standard compound). Accordingly, the ability of a test compound to compete with the standard compound for binding to the VRK1 protein directly correlates to its binding affinity. Conversely, in some other screening assays, the standard compound is labeled and test compounds are unlabeled.

Accordingly, the concentration of the labeled standard compound is monitored in order to evaluate the competition between the standard compound and the test compound, and the relative binding affinity of the test compound is thus ascertained.

### ENUMERATED EMBODIMENTS

Embodiment 1. A compound of Formula (I):

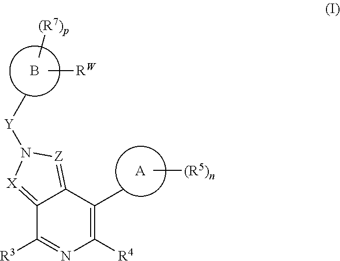

or a pharmaceutically acceptable salt thereof, wherein:
n is 0, 1, 2, 3, 4, 5, or 6;
p is 0, 1, 2, 3, 4, 5, or 6;
r is 1 or 2;
X is N or CR¹;
Z is N or CR²;
Y is -(Cy¹)ₘ-(Y′)ₛ—, wherein Y¹ is attached to Ring moiety B;
m is 1; s is 1; and Y¹ is a bond; or
m is 1; s is 1; and Y¹ is —(CR⁶R⁶⁰)ᵣ—, —CR⁶R⁶⁰—C(O)—, —C(O)—CR⁶R⁶⁰—, or C(O); or
m is 0; s is 1; and Y¹ is —(CR⁶R⁶⁰)ᵣ—, —CR⁶R⁶⁰—C(O)—, —C(O)—CR⁶R⁶⁰—, or C(O);
Cy¹ is C₃₋₁₀ cycloalkyl, 6-10 membered aryl, 4-10 membered heterocycloalkyl, or 5-10 membered heteroaryl, which is optionally substituted by 1, 2, 3, 4, 5, or 6 independently selected R¹⁰ substituents;
Ring moiety A is C₃₋₁₂ cycloalkyl, 6-10 membered aryl, 4-12 membered heterocycloalkyl, or 5-10 membered heteroaryl;
Ring moiety B is C₃₋₁₂ cycloalkyl, 6-10 membered aryl, 4-12 membered heterocycloalkyl, or 5-10 membered heteroaryl;
R¹ is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{b1}, SR^{b1}, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}R^{d1}, NR^{c1}NR^{c1}R^{d1}, NR^{c}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}C(═NR^{e1})R^{b1}, NR^{c1}S(O)NR^{c1}R^{d1}, NR^{c1}S(O)R^{b1}, NR^{c1}S(O)₂R^{b1}, NR^{c1}(O)(═NR^{e1})R^{b1}, NR^{c1}(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, OS(O)₂R^{b1}, S(O)(═NR^{e1})R^{b1}, SF₅, P(O)R^{f1}R^{g1}, OP(O)(OR^{h1})(ORⁱ¹), P(O)(OR^{h1})(ORⁱ¹), and BR^{j1}R^{k1}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h1} and Rⁱ¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j1} and R^{k1} is independently selected from OH, C₁₋₆ alkoxy, and C₁₋₆ haloalkoxy;
or any R^{j1} and R^{k1} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}, NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h11} and Rⁱ¹¹ is independently selected from H, C₁₋₆ alkyl, C₁₋₆haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j11} and R^{k11} is independently selected from OH, C₁₋₆ alkoxy, and C₁₋₆ haloalkoxy;
or any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, R^{c12}C(O)NR^{c12}R^{d12}, C(═NR^{e12})R^{b12}, C(═NR^{e12})NR^{c12}R^{d12}, NR^{c12}C(═NR^{e12}NR^{c12}R^{d12}, NR^{c12}C(═NR^{e12})R^{b12}, NR^{c12}S(O)NR^{c12}R^{d12}, NR^{c12}S(O)R^{b12}, NR^{c12}S(O)₂NR^{b12}, NR^{c12}S(O)(═NR^{e12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c12} and R^{d12} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{b2}, SR^{b2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, S(O)(═NR^{e2})R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h2} and Rⁱ² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j2} and R^{k2} is independently selected from OH, C₁₋₆ alkoxy, and C₁₋₆ haloalkoxy;
or any R^{j2} and Rⁱ² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})R^{c21}R^{d21}, NR^{c21}C(═NR^{e21})NR^{c21}R^{d21}, NR^{c21}C(═NR^{e21})R^{b21}, NR^{c21}S(O)NR^{c21}R^{d21}, NR^{c21}S(O)R^{b21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
or, any R^{c21} and R^{d21} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2B} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f21} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a22}, SR^{a22}, NHOR^{a22}C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR^{c22}NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22})R^{b22}, NR^{c22}S(O)NR^{c22}R^{d22}, NR^{c22}S(O)R^{b22}, NR^{c22}S(O)₂R^{b22}, NR^{c22}S(O)(═NR^{e22})R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22} OS(O)(═NR^{e22})R^{b22}, OS(O)₂R^{b22}, S(O)(═NR^{e22})R^{b22}, SF₅, P(O)R^{f22}R^{g22}, OP(O)(OR^{h22})(ORⁱ²²), P(O)(OR^{h22})(ORⁱ²²), and BR^{j22}R^{k22}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c22} and R^{d22} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b22} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f22} and R^{g22} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h22} and Rⁱ²² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j22} and R^{k22} is independently selected from OH, C₁₋₆ alkoxy, and C₁₋₆ haloalkoxy;
or any R^{j22} and R^{k22} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3} NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b3} is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4}, NR^{c4}C(═NR^{e4})R^{b4}, NR^{c4}S(O)NR^{c4}R^{d4}, NR^{c4}S(O)R^{b4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
each R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b4} is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e4} is independently selected from H, OH, CN, C₁₋₆ alkyl, C₁₋₆ alkoxy, C₁₋₆ haloalkyl, and C₁₋₆ haloalkoxy;
each R⁵ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
or, any R^{c5} and R^{d5} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{b51}R^{d51}, NR^{c51}NR^{c51}R^{d51} NR^{c51}C(O)R^{b51}, NR^{c51}, C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)NR^{c51}R^{d51}, NR^{c51}S(O)R^{b51}, NR^{c51}S(O)₂R^{b51}, NR^{c51}S(O)(═NR^{e5})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, OS(O)₂R^{b51}, S(O)(═NR^{e51})R^{b51}, SF₅, P(O)R^{f51}R⁵¹, OP(O)(OR^{h51})(ORⁱ⁵¹), P(O)(OR^{h51})(ORⁱ⁵¹), and BR^{j51}R^{k51}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R⁵¹, R^{c51}, and R^{d51} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c51} and R^{d51} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b51} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R⁵¹ is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f51} and R^{g51} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h51} and Rⁱ⁵¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j51} and R^{k51} is independently selected from OH, C₁₋₆ alkoxy, and C₁₋₆ haloalkoxy;
or any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶ is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a6}, SR a, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c6}R^{d6}, C(═NR^{e6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)NR^{c6}R^{d6}, NR^{c6}S(O)R^{b6}, NR^{c6}S(O)₂R^{b6}, NR^{c6}S(O)(═NR^{e6})R^{b6}, NR^{c6}S(O)₂NR^{e6}R^{d6}, S(O)R^{b6}, S(O)NR^{e6}R^{d6}, S(O)₂R^{b6}, S(O)₂NR^{c6}R^{d6}, OS(O)(═NR^{e6})R^{b6}, OS(O)₂R^{b6}, S(O)(═NR^{e6})R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
or, any R^{c6} and R^{d6} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6A} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f6} and R^{g6} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h6} and Rⁱ⁶ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j6} and R^{k6} is independently selected from OH, C₁₋₆ alkoxy, and C₁₋₆ haloalkoxy;
or any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, NHOR^{a61}, C(O)R^{b61}, C(O)NR^{c61}R^{d61}, C(O)NR^{c61}(OR^{a61}), C(O)OR^{a61}, OC(O)R^{b61}, OC(O)NR^{c61}R^{d61}, NR^{c61}R^{d61}, NR^{c61}NR^{c61}R^{d61}, NR^{c61}C(O)R^{b61}, NR^{c61}C(O)OR^{a61}, NR^{c61}C(O)NR^{c61}R^{d61}, C(═NR^{e61})R^{b61}, C(═NR^{e61})NR^{c61}R^{d61}, NR^{c61}C(═NR^{e61}NR^{c61}R^{d61}, NR^{c61}C(═NR^{e61})R^{b61}, NR^{c61}S(O)NR^{c61}R^{d61}, NR^{c61}S(O)R^{b61}, NR^{c61}S(O)₂R^{b61}, NR^{c61}S(O)(═NR^{e61})R^{b61}, NR^{c61}S(O)₂NR^{c61}R^{d61}, S(O)R^{b61}, S(O)NR^{c61}R^{d61}, S(O)₂R^{b61}, S(O)₂NR^{c61}R^{d61}, OS(O)(═NR^{e61})R^{b61}, OS(O)₂R^{b61}, S(O)(═NR^{e61})R^{b61}, SF₅, P(O)R^{f6}1R^{g61}, OP(O)(OR^{h61})(ORⁱ⁶¹), P(O)(OR^{h61})(ORⁱ⁶¹), and BR^{j61}R^{k61}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{e61} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f61} and R^{g61} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h61} and Rⁱ⁶¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j61} and R^{k61} is independently selected from OH, C₁₋₆ alkoxy, and C₁₋₆ haloalkoxy;
or any R^{j61} and R^{k61} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, NHOR^{a62}C(O)R^{b62}, C(O)NR^{c62}R^{d62}, C(O)NR^{c62}(OR^{a62}), C(O)OR^{a62}, OC(O)R^{b62}, OC(O)NR^{c62}R^{d62}, NR^{c62}R^{d62}, NR^{c62}NR^{c62}R^{d62}, NR^{c62}C(O)R^{b62}, NR^{c62}C(O)OR^{a62}, NR^{c62}C(O)NR^{c62}R^{d62}, C(═NR^{e62})R^{b62}, C(═NR^{e62})R^{c62}R^{d62}, NR^{c62}C(═NR^{e62})NR^{c62}R^{d62}, NR^{c62}C(═NR^{e62})R^{b62}, NR^{c62}S(O)NR^{c62}R^{d62}, NR^{c62}S(O)R^{b62}, NR^{c62}S(O)₂R^{b62}, NR^{c62}S(O)(═NR^{e62})R^{b62}, NR^{c62}S(O)₂NR^{c62}R^{d62}, S(O)R^{b62}, S(O)NR^{c62}R^{d62}, S(O)₂R^{b62}, S(O)₂NR^{c62}R^{d62}, OS(O)(═NR^{e62})R^{b62}, OS(O)₂R^{b62}, S(O)(═NR^{e62})R^{b62}, SF₅, P(O)R^{f62}R^{g62}, OP(O)(OR^{h62})(ORⁱ⁶²), P(O)(OR^{h62})(ORⁱ⁶²), and BR^{j62}R^{k62}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a62}, R^{c62}, and R^{d62} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c62} and R^{d62} attached to the same N atom, together with the N atom to which they are attached, form a 4-7 membered heterocycloalkyl group, wherein the 4-7 membered heterocycloalkyl group is optionally substituted with 1, 2, 3, or 4 independently selected RG substituents;
each R^{b62} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e62} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f62} and R^{g62} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h62} and Rⁱ⁶² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j62} and R^{k62} is independently selected from OH, C₁₋₆ alkoxy, and C₁₋₆ haloalkoxy;
or any R^{j62} and R^{k62} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶⁰ is independently selected from H, D, C₁₋₆ alkyl, and C₁₋₆ haloalkyl;
or R⁶ and R⁶⁰, together with the carbon atom to which they are attached, form a C₃₋₁₀ cycloalkyl or a 4-10 membered heterocycloalkyl moiety, each of which is optionally substituted with 1, 2, 3, or 4 independently selected R^{7A} substituents;
each R⁷ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7}, NR^{c7}C(O)OR^{a7}, NR^{c7}C(O)NR^{c7}R^{d7} C(═NR^{e7})R^{b7}, C(═NR^{e7})NR^{c7}R^{d7}, NR^{c7}R^{d7}C(═NR^{e7})NR^{c7}R^{d7}, NR^{c7}C(═NR^{c7})R^{b7}, NR^{c7}S(O)NR^{c7}R^{d7}, NR^{c7}S(O)R^{b7}, NR^{c7}S(O)₂R^{b7}, NR^{c7}S(O)(═NR^{b7})R^{b7}, NR^{c7}S(O)₂NR^{c7}R^{d7} S(O)R^{b7}, S(O)NR^{c7}R^{d7}, S(O)₂R^{b7}, S(O)₂NR^{c7}R^{d7}, OS(O)(═NR^{e7})R^{b7}, OS(O)₂R^{b7}, S(O)(═NR^{e7})R^{b7}, SF₅, P(O)R^{f7}R^{g7}, OP(O)(OR^{h7})(ORⁱ⁷), P(O)(OR^{h7})(ORⁱ⁷), and BR^{j7}R^{k7}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
or, any R^{c7} and R^{d7} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h7} and Rⁱ⁷ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j7} and R^{k7} is independently selected from OH, C₁₋₆ alkoxy, and C₁₋₆ haloalkoxy;
or any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, NHOR^{a71}, C(O)R^{b71}, C(O)NR^{c71}R^{d71}, C(O)NR^{c71}(OR^{a71}), C(O)OR^{a71}, OC(O)R^{b71}, OC(O)NR^{c71}R^{d71}, NR^{c71}R^{d71}, NR^{c71}NR^{c71}R^{d71}, NR^{c71}C(O)R^{b7}, NR^{c71}C(O)OR^{a71}, NR^{c71}C(O)NR^{c71}R^{d71}, C(═NR^{e71})R^{b71}, C(═NR^{e71})NR^{c71}R^{d71}, NR^{c71}C(═NR^{e71})NR^{c71}R^{d71}, NR^{c71}C(═NR^{e71})R^{b71}, NR^{c71}S(O)NR^{c71}R^{d71}, N^{c71} S(O)R^{b71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71} OS(O)(═NR^{e71})R^{b71}, OS(O)₂R^{b71}, S(O)(═NR^{e71})R^{b71}, SF₅, P(O)R^{f71}R^{g71}, OP(O)(OR^{h71})(ORⁱ⁷¹), P(O)(OR^{h71})(ORⁱ⁷¹), and BR^{j71}R^{k71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{e71} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f71} and R^{g71} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h71} and Rⁱ⁷¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j71} and R^{k71} is independently selected from OH, C₁₋₆ alkoxy, and C₁₋₆ haloalkoxy;
or any R^{j71} and R^{k71} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a72}, SR^{a72}, NHOR^{a72}C(O)R^{b72}, C(O)NR^{c72}R^{d72}, C(O)NR^{c72}(OR^{a72}), C(O)OR^{a72}, OC(O)R^{b72}, OC(O)NR^{c72}R^{d72}, NR^{c72}R^{d72}, NR^{c72}NR^{c72}R^{d72}, NR^{c72}C(O)R^{b72}, NR^{c72}C(O)OR^{a72}, NR^{c72}C(O)NR^{c72}R^{d72}, C(═NR^{e72})R^{b72}, C(═NR^{e72})R^{c72}R^{d72}, NR^{c72}C(═NR^{e72})R^{c72}R^{d72}, NR^{c72}C(═NR^{e72})R^{b72}, NR^{c72}S(O)NR^{c72}R^{d72}, NR^{c72}S(O)R^{b72}, NR^{c72}S(O)₂R^{b72}, NR^{c72}S(O)(═NR^{e72})R^{b72}, NR^{c72}S(O)₂NR^{c72}R^{d72}, S(O)R^{b72}, S(O)NR^{c72}R^{d72}, S(O)₂R^{b72}, S(O)₂NR^{c72}R^{d72} OS(O)(═NR^{e72})R^{b72}, OS(O)₂R^{b72}, S(O)(═NR^{e72})R^{b72}, SF₅, P(O)R^{f72}R^{g72}, OP(O)(OR^{h72})(ORⁱ⁷²), P(O)(OR^{h72})(ORⁱ⁷²), and BR^{j72}R^{k72}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a72}, R^{c72}, and R^{d72} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c72} and R^{d72} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b72} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e72} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f72} and R^{g72} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h72} and Rⁱ⁷² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j72} and R^{k72} is independently selected from OH, C₁₋₆ alkoxy, and C₁₋₆ haloalkoxy;
or any R^{j72} and R^{k72} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{W} is a covalent warhead moiety having a reactive functional group capable of covalently binding to one or more cysteine residues in a protein;
each R¹⁰ is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a10}, SR^{a10}, NHOR^{a10}C(O)R^{b10}, C(O)NR^{c10}R^{d10}, C(O)NR^{c10}(OR^{a10}), C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)NR^{c10}R^{d10}, NR^{c10}S(O)R^{b10}, NR^{c10}S(O)₂R^{b10}, NR^{c10}S(O)₂NR^{c10}R^{d10}, S(O)R^{b10}, S(O)NR^{c10}R^{d10}, S(O)₂R^{b10}, and S(O)₂NR^{c10}R^{d10}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a10}, R^{c10}, and R^{d10} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c10} and R^{d10} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{3A}
each R^{b10} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{3A} substituents; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, N(CD₃)₂, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 2. A compound of Formula (I):

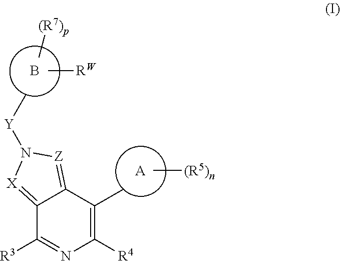

or a pharmaceutically acceptable salt thereof, wherein:
n is 0, 1, 2, 3, 4, 5, or 6;
p is 0, 1, 2, 3, 4, 5, or 6;
r is 1 or 2;
X is N or CR¹;
Z is N or CR²;
Y is -(Cy¹)ₘ-(Y¹)ₛ—, wherein Y¹ is attached to Ring moiety B;
m is 1; s is 1; and Y¹ is a bond; or
m is 1; s is 1; and Y¹ is —(CR⁶R⁶⁰)ᵣ—, —CR⁶R⁶⁰—C(O)—, —C(O)—CR⁶R⁶⁰—, or C(O);
m is 0; s is 1; and Y¹ is —(CR⁶R⁶⁰)ᵣ—, —CR⁶R⁶⁰—C(O)—, —C(O)—CR⁶R⁶⁰—, or C(O);
Cy¹ is C₃₋₁₀ cycloalkyl, 6-10 membered aryl, 4-10 membered heterocycloalkyl, or 5-10 membered heteroaryl, which is optionally substituted by 1, 2, 3, 4, 5, or 6 independently selected R¹⁰ substituents;
Ring moiety A is C₃₋₁₂ cycloalkyl, 6-10 membered aryl, 4-12 membered heterocycloalkyl, or 5-10 membered heteroaryl;
Ring moiety B is C₃₋₁₂ cycloalkyl, 6-10 membered aryl, 4-12 membered heterocycloalkyl, or 5-10 membered heteroaryl;
R¹ is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{b1}, SR^{b1}, NHOR^{a11}, C(O)R^{b1}, C(O)NR^{c1}R^{d1}, C(O)NR^{c1}(OR^{a1}), C(O)OR^{a11}, OC(O)R^{h1}, OC(O)NR^{c1}R^{d1}, NR^{c1}R^{d1}, NR^{e1}NR^{c1}R^{d1}, NR^{c1}C(O)R^{h1}, NR^{c1}C(O)OR^{a11}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}C(═NR^{e1})R^{b1}, NR^{c1}S(O)NR^{c1}R^{d1}, NR^{c1}S(O)R^{b1}, NR^{c1}S(O)₂R^{b1}, NR^{c1}S(O)(═NR^{e1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, OS(O)₂R^{b1}, S(O)(═NR^{e1})R^{b1}, SF₅, P(O)R^{f1}R^{g1}, OP(O)(OR^{h1})(ORⁱ¹), P(O)(OR^{h1})(ORⁱ¹), and BR^{j1}R^{k1}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h1} and Rⁱ¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j1} and R^{k1} is independently selected from OH, C₁₋₆ alkoxy, and C₁₋₆ haloalkoxy;
or any R^{j1} and R^{k1} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b1}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h11} and Rⁱ¹¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j11} and R^{k11} is independently selected from OH, C₁₋₆ alkoxy, and C₁₋₆ haloalkoxy;
or any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, OC(O)R^{b12}, OC(O)NR^{c12}R^{d12}, C(═NR^{e12})R^{b12}, C(═NR^{e12})NR^{c12}R^{d12}, NR^{c12}C(═NR^{e12})NR^{c12}R^{d12}, NR^{c12}C(═NR^{e12})R^{b12}, NR^{c12}S(O)NR^{c12}R^{d12}, NR^{c12}S(O)R^{b12}, NR^{c12}S(O)₂R^{b12}, NR^{c12}S(O)(═NR^{e12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R¹², S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c12} and R^{d12} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G}
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{b2}, SR^{b2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, S(O)(═NR^{e2})R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h2} and Rⁱ² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j2} and R^{k2} is independently selected from OH, C₁₋₆ alkoxy, and C₁₋₆ haloalkoxy;
or any R^{j2} and Rⁱ² attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from D, halo, CN, NO₂, C₁₋₆ alkyl, C₂₋₆ alkenyl, C₂₋₆ alkynyl, C₁₋₆ haloalkyl, C₃₋₇cycloalkyl, phenyl, 4-7 membered heterocycloalkyl, 5-6 membered heteroaryl, C₃₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})NR^{c21}R^{d21}, NR^{c21}C(═NR^{e21})R^{b21}, NR^{c21}S(O)NR^{c21}R^{d21}, NR^{c21}S(O)R^{b21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
or, any R^{c21} and R^{d21} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2B}
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f21} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a22}, SR^{a22}, NHOR^{a22}C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR^{c22}NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22})R^{b22}, NR^{c22}S(O)NR^{c22}R^{d22}, NR^{c22}S(O)R^{b22}, NR^{c22}S(O)₂R^{b22}, NR^{c22}S(O)(═NR^{e22})R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22} OS(O)(═NR^{e22})R^{b22}, OS(O)₂R^{b22}, S(O)(═NR^{e22})R^{b22}, SF₅, P(O)R^{f22}R^{g22}, OP(O)(OR^{h22})(ORⁱ²²), P(O)(OR^{h22})(ORⁱ²²), and BR^{j22}R^{k22}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c22} and R^{d22} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b22} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f22} and R^{g22} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h22} and Rⁱ²² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j22} and R^{k22} is independently selected from OH, C₁₋₆ alkoxy, and C₁₋₆ haloalkoxy;
or any R^{j22} and R^{k22} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b3} is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4} NR^{c4}C(═NR^{e4})R^{b4}, NR^{c4}S(O)NR^{c4}R^{d4}, NR^{c4}S(O)R^{b4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, and S(O)₂NR^{c4}R^{d4}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
each R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b4} is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e4} is independently selected from H, OH, CN, C₁₋₆ alkyl, C₁₋₆ alkoxy, C₁₋₆ haloalkyl, and C₁₋₆ haloalkoxy;
each R⁵ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
or, any R^{c5} and R^{d5} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}, C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)NR^{c51}R^{d51}, NR^{c51}S(O)R^{b51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, OS(O)₂R^{b51}, S(O)(═NR^{e51})R^{b51}, SF₅, P(O)R^{f51}R^{g51}, OP(O)(OR^{h51})(ORⁱ⁵¹), P(O)(OR^{h51})(ORⁱ⁵¹), and BR^{j51}R^{k51}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c51} and R^{d51} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b51} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f51} and R^{g51} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h51} and R^{g51} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j51} and R^{k51} is independently selected from OH, C₁₋₆ alkoxy, and C₁₋₆ haloalkoxy;
or any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶ is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c6}R^{d6}, C(═NR^{e6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)NR^{c6}R^{d6}, NR^{c6}S(O)R^{b6}, NR^{c6}S(O)₂R^{b6}, NR^{c6}S(O)(═NR^{e6})R^{b6}, NR^{c6}S(O)₂NR^{c6}R^{d6}, S(O)R^{b6}, S(O)NR^{c6}R^{d6}, S(O)₂R^{b6}, S(O)₂NR^{c6}R^{d6}, OS(O)(═NR^{e6})R^{b6}, OS(O)₂R^{b6}, S(O)(═NR^{e6})R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
or, any R^{c6} and R^{d6} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{6A} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f6} and R^{g6} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h6} and Rⁱ⁶ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j6} and R^{k6} is independently selected from OH, C₁₋₆ alkoxy, and C₁₋₆ haloalkoxy;
or any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, NHOR^{a61}, C(O)R^{b61}, C(O)NR^{c61}R^{d61}, C(O)NR^{c61}(OR^{a61}), C(O)OR^{a61}, OC(O)R^{b61}, OC(O)NR^{c6}R^{d61}, NR^{c61}R^{d61}, NR^{c61}NR^{c61}R^{d61}, NR^{c61}C(O)R^{b61}, NR^{c61}C(O)OR^{a61} NR^{c61}C(O)NR^{c61}R^{d61}, C(═NR^{e61})R^{b61}, C(═NR^{e61})NR^{c61}R^{d61}, NR^{c61}C(═NR^{e61}NR^{c61}R^{d61}, NR^{c61}C(═NR^{e61})R^{b61}, NR^{c61}S(O)NR^{c61}R^{d61}, NR^{c61}S(O)R^{b61}, NR^{c61}S(O)₂R^{b61}, NR^{c61}S(O)(═NR^{e61})R^{b61}, NR^{c61}S(O)₂NR^{c61}R^{d61}, S(O)R^{b61}, S(O)NR^{c61}R^{d61}, S(O)₂R^{b61}, S(O)₂NR^{c61}R^{d61}, OS(O)(═NR^{e61})R^{b61}, OS(O)₂R^{b61}, S(O)(═NR^{e61})R^{b61}, SF₅, P(O)R^{f6}1R^{g61}, OP(O)(OR^{h61})(ORⁱ⁶¹), P(O)(OR^{h61})(ORⁱ⁶¹), and BRⁱ⁶¹R^{k61}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{e61} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f61} and R^{g61} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h61} and Rⁱ⁶¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j61} and R^{k61} is independently selected from OH, C₁₋₆ alkoxy, and C₁₋₆ haloalkoxy;
or any R^{j61} and R^{k61} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6B} is independently selected from D, halo, CN, NO₂, C₁₋₆ alkyl, C₂₋₆ alkenyl, C₂₋₆ alkynyl, C₁₋₆ haloalkyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, NHOR^{a62}C(O)R^{b62}, C(O)NR^{c62}R^{d62}, C(O)NR^{c62}(OR^{a62}), C(O)OR^{a62}, OC(O)R^{b62}, OC(O)NR^{c62}R^{d62}, NR^{c62}R^{d62}, N^{c62}NR^{c62}R^{d62}, NR^{c62}C(O)R^{b62}, NR^{c62}C(O)OR^{a62}, NR^{c62}C(O)NR^{c62}R^{d62}, C(═NR^{e62})R^{b62}, C(═NR^{e62})NR^{c62}R^{d62}, NR^{c62}C(═NR^{e62}NR^{c62}R^{d62}, NR^{c62}C(═NR^{e62})R^{b62}, NR^{c62}S(O)NR^{c62}R^{d62}, NR^{c62}S(O)R^{b62}, NR^{c62}S(O)₂R^{b62}, NR^{c62}S(O)(═NR^{e62})R^{b62}, NR^{c62}S(O)₂NR^{c62}R^{d62}, S(O)R^{b62}, S(O)NR^{c62}R^{d62}, S(O)₂R^{b62}, S(O)₂NR^{c62}R^{d62}, OS(O)(═NR^{e62})R^{b62}, OS(O)₂R^{b62}, S(O)(═NR^{e62})R^{b62}, SF₅, P(O)R^{f62}R^{g62}, OP(O)(OR^{h62})(ORⁱ⁶²), P(O)(OR^{h62})(ORⁱ⁶²), and BR^{j62}R^{k62}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a62}, R^{c62}, and R^{d62} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c62} and R^{d62} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b62} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e62} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f62} and R^{g62} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h62} and Rⁱ⁶² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j62} and R^{k62} is independently selected from OH, C₁₋₆ alkoxy, and C₁₋₆ haloalkoxy;
or any R^{j62} and R^{k62} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶⁰ is independently selected from H, D, C₁₋₆ alkyl, and C₁₋₆ haloalkyl;
or R⁶ and R⁶⁰, together with the carbon atom to which they are attached, form a 4-10 membered heterocycloalkyl moiety, which is optionally substituted with 1, 2, 3, or 4 independently selected R^{7A} substituents;
each R⁷ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)NR^{c7}R^{d7}, NR^{c7}S(O)R^{b7}, NR^{c7}S(O)₂R^{b7}, NR^{c7}S(O)(═NR^{e7})R^{b7}, NR^{c7}S(O)₂NR^{c7}R^{d7} S(O)R^{b7}, S(O)NR^{c7}R^{d7}, S(O)₂R^{b7}, S(O)₂NR^{c7}R^{d7}, OS(O)(═NR^{e7})R^{b7}, OS(O)₂R^{b7}, S(O)(═NR^{e7})R^{b7}, SF₅, P(O)R^{f7}R^{g7}, OP(O)(OR^{h7})(ORⁱ⁷), P(O)(OR^{h7})(ORⁱ⁷), and BR^{j7}R^{k7}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
or, any R^{c7} and R^{d7} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h7} and Rⁱ⁷ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j7} and R^{k7} is independently selected from OH, C₁₋₆ alkoxy, and C₁₋₆ haloalkoxy;
or any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, NHOR^{a71}, C(O)R^{b71}, C(O)NR^{c71}R^{d71}, C(O)NR^{c71}(OR^{a71}), C(O)OR^{a71}, OC(O)R^{b71}, OC(O)NR^{c71}R^{d71}, NR^{c71}R^{d7171}, NR^{c71}NR^{c7}R^{d71}, NR^{c71}C(O)R^{b71}, NR^{c71}C(O)OR^{a71}, NR^{c71}C(O)NR^{c71}R^{d71}, C(═NR^{e71})R^{b71}, C(═NR^{e71})NR^{c71}R^{d71}, NR^{c71}C(═NR^{e71})NR^{c71}R^{d71}, NR^{c71}C(═NR^{e71})R^{b71}, NR^{c71}S(O)NR^{c71}R^{d71}, NR^{c71}, S(O)R^{b71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71} OS(O)(═NR^{e71})R^{b71}, OS(O)₂R^{b71}, S(O)(═NR^{e71})R^{b71}, SF₅, P(O)R^{f71}R^{g71}, OP(O)(OR^{h71})(ORⁱ⁷¹), P(O)(OR^{h71})(ORⁱ⁷¹), and BR^{j71}R^{k71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{e71} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f71} and R^{g71} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h71} and Rⁱ⁷¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j71} and R^{k71} is independently selected from OH, C₁₋₆ alkoxy, and C₁₋₆ haloalkoxy;
or any R^{j71} and R^{k71} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a72}, SR^{a72}, NHOR^{a72}C(O)R^{b72}, C(O)NR^{c72}R^{d72}, C(O)NR^{c72}(OR^{a72}), C(O)OR^{a72}, OC(O)R^{b72}, OC(O)NR^{c72}R^{d72}, NR^{c72}R^{d72}, NR^{c72}NR^{c72}R^{d72}, NR^{c72}C(O)R^{b72}, NR^{c72}C(O)OR^{a72}, NR^{c72}C(O)NR^{c72}R^{d72}, C(═NR^{e72})R^{b72}, C(═NR^{e72})NR^{c72}R^{d72}, NR^{c72}C(═NR^{e72})NR^{c72}R^{d72}, NR^{c72}C(═NR^{e72})R^{b72}, NR^{c72}S(O)NR^{c72}R^{d72}, NR^{c72}S(O)R^{b72}, NR^{c72}S(O)₂R^{b72}, NR^{c72}S(O)(═NR^{e72})R^{b72}, NR^{c72}S(O)₂NR^{c72}R^{d72}, S(O)R^{b72}, S(O)NR^{c72}R^{d72}, S(O)₂R^{b72}, S(O)₂NR^{c72}R^{d72} OS(O)(═NR^{e72})R^{b72}, OS(O)₂R^{b72}, S(O)(═NR^{e72})R^{b72}, SF₅, P(O)R^{f72}R^{g72}, OP(O)(OR^{h72})(ORⁱ⁷²), P(O)(OR^{h72})(ORⁱ⁷²), and BR^{j72}R^{k72}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a72}, R^{c72}, and R^{d72} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c72} and R^{d72} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b72} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e72} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f72} and R^{g72} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h72} and Rⁱ⁷² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j72} and R^{k72} is independently selected from OH, C₁₋₆ alkoxy, and C₁₋₆ haloalkoxy;
or any R^{j72} and R^{k72} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{W} is a covalent warhead moiety having a reactive functional group capable of covalently binding to one or more cysteine residues in a protein;
each R¹⁰ is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a10}, SR^{a10}, NHOR^{a10}, C(O)R^{b10}, C(O)NR^{c10}R^{d10}, C(O)NR^{c10}(OR^{a10}), C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)NR^{c10}R^{d10}, NR^{c10}S(O)R^{b10}, NR^{c10}S(O)₂R^{b10}, NR^{c10}S(O)₂NR^{c10}R^{d10}, S(O)R^{b10}, S(O)NR^{c10}R^{d10}, S(O)₂R^{b10}, and S(O)₂NR^{c10}R^{d10}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a10}, R^{c10}, and R^{d10} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c10} and R^{d10} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{3A} substituents;
each R^{b10} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{3A} substituents; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 3. The compound of embodiment 1 or 2, or a pharmaceutically acceptable salt thereof, wherein R^{W} is a means for covalently binding to cysteine residue in a protein.

Embodiment 4. The compound of embodiment 1 or 2, or a pharmaceutically acceptable salt thereof, wherein R^{W} is:

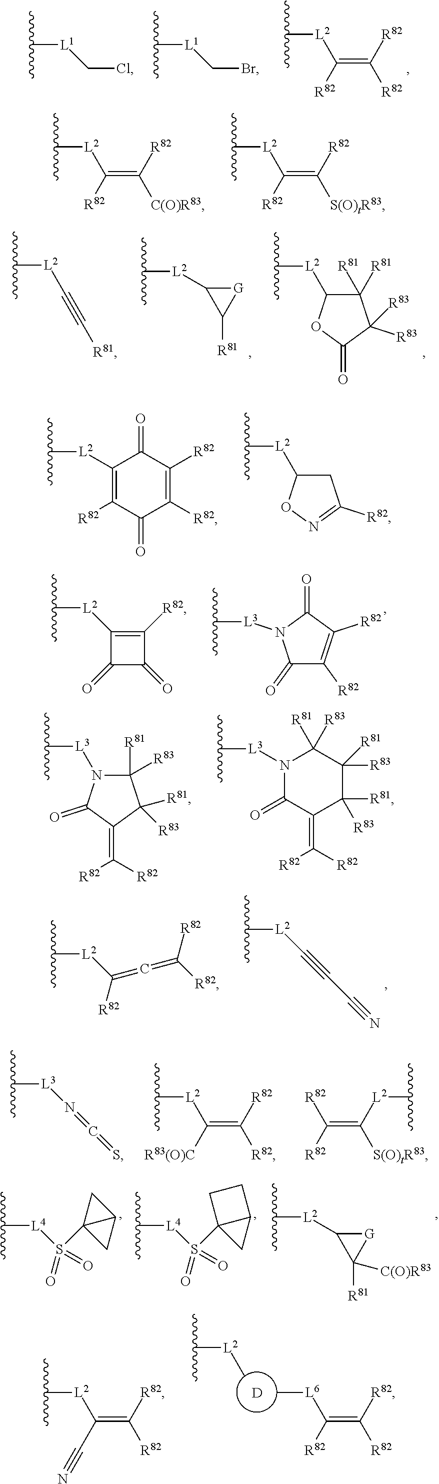

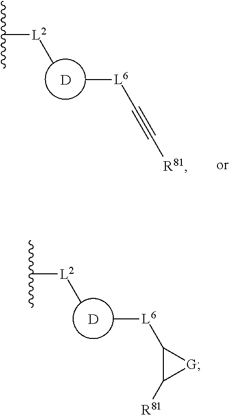

L¹ is -L-C(O)—, -L-NR⁹C(O)—, -L-OC(O)—, -L-S(O)—, -L-S(O)₂—, -L-NR⁹S(O)—, -L-OS(O)—, -L-NR⁹S(O)₂—, or -L-OS(O)₂—, wherein L¹ is attached to Ring moiety B through the L linking group;
L² is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NR⁹(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—, wherein L² is attached to Ring moiety B through the L linking group;
L³ is -L-, -L-C(O)—, -L-NR⁹C(O)—, -L-OC(O)—, -L-S(O)—, -L-S(O)₂—, -L-NR⁹(O)—, -L-OS(O)—, -L-NR⁹S(O)₂—, or -L-OS(O)₂—, wherein L³ is attached to Ring moiety B through the L linking group;
L⁴ is -L-, -L-O—, L-S—, or -L-NR⁹—, wherein L⁴ is attached to Ring moiety B through the L linking group;
L⁶ is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NR⁹S(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—, wherein L⁶ is attached to Ring moiety D through the L linking group;
L is a bond or C₁₋₆ alkylene, wherein said C₁₋₆ alkylene is optionally substituted by 1, 2, 3, or 4 independently selected R^{G} substituents; or
L is —O—C₁₋₆ alkyl or —N(R^{N})—C₁₋₆ alkyl, wherein L is attached to Ring moiety A via the oxygen of the —O—C₁₋₆ alkyl or the nitrogen atom of the —N(R^{N})—C₁₋₆ alkyl group;
each R^{N} is independently H or C₁₋₆ alkyl;
Ring D is a 4-12 membered heterocycloalkyl, C₃₋₁₂ cycloalkyl, C₆₋₁₀ aryl, or a 5-10 membered heteroaryl, each of which is optionally substituted with 1, 2, 3, or 4 independently selected C₁₋₆ alkyl groups;
G is O or NR⁹;
each t is independently 0, 1, 2, or 3;
each R⁸¹ and R⁸³ are independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{c8}R^{d8}, NR^{c8}R^{d8}, NR^{c8}NR^{c8}R^{d8}, NR^{c8}C(O)R^{b8}, NR^{c8}C(O)OR^{as}, NR^{c8}C(O)NR^{c8}R^{d8}, C(═NR^{e8})R^{b8}, C(═NR^{e8})NR^{c8}R^{d8}, NR^{c8}C(═NR^{e8})R^{c8}R^{d8}, NR^{c8}C(═NR^{e8})R^{b8}, NR^{c8}S(O)NR^{c8}R^{d8}, NR^{c1}S(O)R^{b8}, NR^{c8}S(O)₂R^{b8}, NR^{c1}S(O)(═NR^{e8})R^{b8}, NR^{c8}S(O)₂NR^{c8}R^{d8}, S(O)R^{b8}, S(O)NR^{c8}R^{d8}, S(O)₂R^{b8}, S(O)₂NR^{c8}R^{d8}, 0S(O)(═NR^{e8})R^{b8}, OS(O)₂R^{b8}, S(O)(═NR^{e8})R^{b8}, SF₅, P(O)R^{f8}R^{g8}, OP(O)(OR^{h8})(ORⁱ⁸), P(O)(OR^{h8})(ORⁱ⁸), and BR^{j8}R^{k8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R⁸² is independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, C(O)R^{b8}, C(O)NR^{c8}R^{d8}, C(O)OR^{a8}, C(═NR^{e8})R^{h8}, C(═NR^{e8})NR^{c8}R^{d8}, S(O)R^{b8}, S(O)NR^{c8}R^{d8}, S(O)₂R^{b8}, and S(O)₂NR^{c8}R^{d8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a8}, R^{e8}, and R^{d8} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b8} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h8} and Rⁱ⁸ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j8} and R^{k8} is independently selected from OH, C₁₋₆ alkoxy, and C₁₋₆ haloalkoxy;
or any R^{j8} and R^{k8} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
or any two R⁸¹ and R⁸² together with the atoms to which they are attached, form C₃₋₇ cycloalkyl, 4-7 membered heterocycloalkyl, phenyl, or 5-6-membered heteroaryl ring, each of which is optionally substituted with 1, 2, 3, or 4 independently selected R^{G} substituents;
each R⁹ is independently selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, OR^{a9}, SR^{a9}, NHOR^{a9}, C(O)R^{b9}, C(O)NR^{c9}R^{d9}, C(O)NR^{c9}(OR^{a9}), C(O)OR^{a9}, OC(O)R^{b9}, OC(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, C(═NR^{e9})R^{b9}, C(═NR^{e9})NR^{c9}R^{d9}, NR^{c9}C(═NR^{e9})NR^{c9}R^{d9}, NR^{c9}C(═NR^{e9})R^{b9}, NR^{c9}S(O)NR^{c9}R^{d9}, NR^{c9}S(O)R^{b9}, NR^{c9}S(O)₂R^{b9}, NR^{c9}S(O)(═NR^{e9})R^{b9}, NR^{c9}S(O)₂NR^{c9}R^{d9}, S(O)R^{b9}, S(O)NR^{b9}R^{d9}, S(O)₂R^{b9}, S(O)₂NR^{b9}R^{d9} OS(O)(═NR^{e9})R^{b9}, OS(O)₂R^{b9}, and S(O)(═NR^{e9})R^{b9}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a9}, R^{c9}, and R^{d9} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c9} and R^{d9} attached to the same N atom, together with the N atom to which they are attached, form a 4-7 membered heterocycloalkyl group, which is optionally substituted with 1, 2, 3, or 4 independently selected R^{G} substituents;
each R⁹ is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents; and
each R⁹ is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl.

Embodiment 5. The compound of embodiment 1 or 2, or a pharmaceutically acceptable salt thereof, wherein R^{W} is selected from:

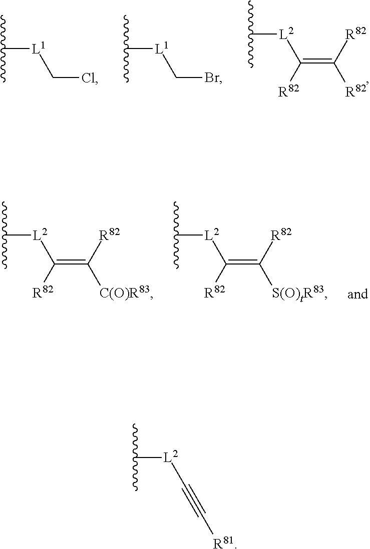

Embodiment 6. The compound of embodiment 1 or 2, or a pharmaceutically acceptable salt thereof, wherein R^{W} is selected from:

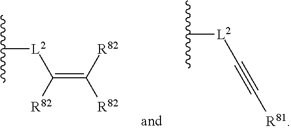

Embodiment 7. The compound of embodiment 1 or 2, or a pharmaceutically acceptable salt thereof, wherein R^{W} is

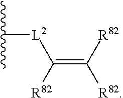

Embodiment 8. The compound of embodiment 1 or 2, or a pharmaceutically acceptable salt thereof, wherein R^{W} is

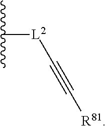

Embodiment 9. The compound of any one of embodiments 4-8, or a pharmaceutically acceptable salt thereof, wherein L¹ is -L-C(O)—, -L-NR⁹C(O)—, -L-S(O)₂—, -L-NR⁹S(O)—, or -L-NR⁹S(O)₂—, or wherein L¹ is attached to Ring moiety B through the L linking group; L is a bond; and R⁹ is selected from H and C₁₋₆ alkyl.

Embodiment 10. The compound of any one of embodiments 4-8, or a pharmaceutically acceptable salt thereof, wherein L² is -L-C(O)—, -L-NR⁹C(O)—, -L-S(O)₂—, or -L-NR⁹S(O)₂—, wherein L² is attached to Ring moiety B through the L linking group; L is a bond; and R⁹ is selected from H and C₁₋₆ alkyl.

Embodiment 11. The compound of any one of embodiments 4-8, or a pharmaceutically acceptable salt thereof, wherein L² is —C(O)—, —NR⁹C(O)—, —S(O)₂—, or —NR⁹S(O)₂—; and R⁹ is selected from H and C₁₋₃ alkyl.

Embodiment 12. The compound of any one of embodiments 4-8, or a pharmaceutically acceptable salt thereof, wherein when L² is —C(O)— or —S(O)₂—, then L² is attached to a nitrogen ring member of Ring moiety B.

Embodiment 13. The compound of any one of embodiments 4-8, or a pharmaceutically acceptable salt thereof, wherein when L² is —C(O)— or —S(O)₂— and Ring moiety B is

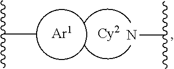

then L² is attached to the nitrogen ring member of Cy².

Embodiment 14. The compound of any one of embodiments 4-13, or a pharmaceutically acceptable salt thereof, wherein R⁸¹, R⁸², and R⁸³ are each independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{c8}R^{d8}, NR^{c8}R^{d8}, NR^{c8}NR^{c8}R^{d8}, NR^{c8}C(O)R^{b8}, NR^{c8}C(O)OR^{a8}, NR^{c8}C(O)NR^{c8}R^{d8}, C(═NR^{e8})R^{b8}, C(═NR^{e8})NR^{c8}R^{d8}, NR^{c8}C(═NR^{e8})NR^{e8}R^{d8}, NR^{c8}C(═NR^{e8})R⁸, NR^{c8}S(O)NR^{c8}R^{d8}, NR^{c8}S(O)R^{b8}, NR^{c8}S(O)₂R^{b8}, NR^{c8}S(O)(═NR^{e8})R^{b8}, NR^{c8}(O)₂NR^{c8}R^{d8}, S(O)R^{b8}, S(O)NR^{c8}R^{d8}, S(O)₂R^{b8}, S(O)₂NR^{c8}R^{d8}, OS(O)(═NR^{e8})R^{b8}, OS(O)₂R^{b8}, and S(O)(═NR^{e8})R^{b8}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents.

Embodiment 15. The compound of embodiment any one of embodiments 4-13, or a pharmaceutically acceptable salt thereof, wherein R⁸¹, R⁸², and R⁸³ are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, OR^{a8}, C(O)R^{b8}, C(O)NR^{c8}R^{d8}, and C(O)OR^{a8}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, di(C₁₋₃ alkyl)amino, and N(CD₃)₂.

Embodiment 16. The compound of embodiment any one of embodiments 4-13, or a pharmaceutically acceptable salt thereof, wherein R⁸¹, R⁸², and R⁸³ are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, OR^{a8}, C(O)R^{b8}, C(O)NR^{c8}R^{d8}, and C(O)OR^{a8}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

Embodiment 17. The compound of any one of embodiments 1-16, or a pharmaceutically acceptable salt thereof, wherein n is 0, 1, 2, 3, or 4.

Embodiment 18. The compound of any one of embodiments 1-16, or a pharmaceutically acceptable salt thereof, wherein n is 0, 1, 2, or 3.

Embodiment 19. The compound of any one of embodiments 1-18, or a pharmaceutically acceptable salt thereof, wherein p is 0, 1, 2, 3, or 4.

Embodiment 20. The compound of any one of embodiments 1-18, or a pharmaceutically acceptable salt thereof, wherein p is 0, 1, or 2.

Embodiment 21. The compound of any one of embodiments 1-20, or a pharmaceutically acceptable salt thereof, wherein X is N.

Embodiment 22. The compound of any one of embodiments 1-20, or a pharmaceutically acceptable salt thereof, wherein X is CR¹.

Embodiment 23. The compound of any one of embodiments 1-20 and 22, or a pharmaceutically acceptable salt thereof, wherein R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b1}, SR^{b1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}CC(O)OR^{a1}, NR^{c1}CC(O)NR^{c1}R^{d1}, NR^{c1}S(O)₂R^{b1}, NR^{c1}(O)₂NR^{c1}R^{d1}, S(O)₂R^{b1}, and S(O)₂NR^{c1}R^{d1}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents.

Embodiment 24. The compound of any one of embodiments 1-20 and 22, or a pharmaceutically acceptable salt thereof, wherein R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents.

Embodiment 25. The compound of any one of embodiments 1-20 and 22, or a pharmaceutically acceptable salt thereof, wherein R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1A} substituents.

Embodiment 26. The compound of any one of embodiments 1-20 and 22-25, or a pharmaceutically acceptable salt thereof, wherein:
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c1}C(O)R^{b1}, NR^{c11}C(O)OR^{a11}, NR^{c11}C(O)NR^{c11}R^{d11}NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R¹ substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R¹ substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R¹ substituents;
each R^{1B} is independently selected from D, halo, CN, C₁₋₆ alkyl, C₂₋₆ alkenyl, C₂₋₆ alkynyl, C₁₋₆ haloalkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12}, and R^{d12} is independently selected from H, C₁₋₆ alkyl, and C₁₋₆ haloalkyl; and
each R¹² is independently selected from C₁₋₆ alkyl, and C₁₋₆ haloalkyl.

Embodiment 27. The compound of any one of embodiments 1-20 and 20-25, or a pharmaceutically acceptable salt thereof, wherein:
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11} is independently selected from H, C₁₋₆ alkyl, and C₁₋₆ haloalkyl; and
each R^{b11} is independently selected from C₁₋₆ alkyl and C₁₋₆ haloalkyl.

Embodiment 28. The compound of any one of embodiments 1-20 and 20-25, or a pharmaceutically acceptable salt thereof, wherein:
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11} is independently selected from H, C₁₋₆ alkyl, and C₁₋₆ haloalkyl; and
each R^{b11} is independently selected from C₁₋₆ alkyl and C₁₋₆ haloalkyl.

Embodiment 29. The compound of any one of embodiments 1-28, or a pharmaceutically acceptable salt thereof, wherein Z is N.

Embodiment 30. The compound of any one of embodiments 1-28, or a pharmaceutically acceptable salt thereof, wherein Z is CR².

Embodiment 31. The compound of any one of embodiments 1-27 and 30, or a pharmaceutically acceptable salt thereof, wherein R² is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b2}, SR^{b2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents.

Embodiment 32. The compound of any one of embodiments 1-27 and 30, or a pharmaceutically acceptable salt thereof, wherein R² is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents.

Embodiment 33. The compound of any one of embodiments 1-27 and 30, or a pharmaceutically acceptable salt thereof, wherein R² is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{2A} substituents.

Embodiment 34. The compound of any one of embodiments 1-27 and 30, or a pharmaceutically acceptable salt thereof, wherein R² is selected from H, D, halo, CN, C₁₋₆ alkyl, C₂₋₆ alkenyl, C₂₋₆ alkynyl, C₁₋₆ haloalkyl, and C₃₋₅ cycloalkyl, wherein said C₁₋₆ alkyl, C₂₋₆ alkenyl, C₂₋₆ alkynyl, C₁₋₆ haloalkyl, and C₃₋₅ cycloalkyl are each optionally substituted with 1, 2, 3, or 4 independently selected R^{2A} substituents.

Embodiment 35. The compound of any one of embodiments 1-27 and 30, or a pharmaceutically acceptable salt thereof, wherein R² is selected from H, D, halo, CN, C₁₋₆ alkyl, C₂₋₆ alkenyl, C₂₋₆ alkynyl, and C₁₋₆ haloalkyl.

Embodiment 36. The compound of any one of embodiments 1-27 and 30, or a pharmaceutically acceptable salt thereof, wherein R² is H.

Embodiment 37. The compound of any one of embodiments 1-27 and 30-36, or a pharmaceutically acceptable salt thereof, wherein:
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2A} substituents;
each R^{2A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from D, halo, CN, C₁₋₆ alkyl, C₂₋₆ alkenyl, C₂₋₆ alkynyl, C₁₋₆ haloalkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12}; and
each R^{a12}, R^{c12}, and R^{d12} is independently selected from H, C₁₋₆ alkyl, and C₁₋₆ haloalkyl.

Embodiment 38. The compound of any one of embodiments 1-27 and 30-36, or a pharmaceutically acceptable salt thereof, wherein:
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2A} substituents;
each R^{2A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21};
each R^{a21}, R^{c21}, and R^{d21} is independently selected from H, C₁₋₆ alkyl, and C₁₋₆ haloalkyl; and
each R^{b21} is independently selected from C₁₋₆ alkyl and C₁₋₆ haloalkyl.

Embodiment 39. The compound of any one of embodiments 1-27 and 30-36, or a pharmaceutically acceptable salt thereof, wherein:
each R^{2A} is independently selected from D, halo, CN, C₁₋₆ alkyl, C₂₋₆ alkenyl, C₂₋₆ alkynyl, C₁₋₆ haloalkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21};
each R^{a21}, R^{c21}, and R^{d21} is independently selected from H, C₁₋₆ alkyl, and C₁₋₆ haloalkyl; and
each R^{b21} is independently selected from C₁₋₆ alkyl and C₁₋₆ haloalkyl.

Embodiment 40. The compound of any one of embodiments 1-39, or a pharmaceutically acceptable salt thereof, wherein m is 1; s is 1; and Y¹ is a bond.

Embodiment 41. The compound of any one of embodiments 1-39, or a pharmaceutically acceptable salt thereof, wherein m is 1; s is 1; and Y¹ is —(CR⁶R⁶⁰)ᵣ—, —CR⁶R⁶⁰—C(O)—. —C(O)—CR⁶R⁶⁰—, or C(O).

Embodiment 42. The compound of any one of embodiments 1-39, or a pharmaceutically acceptable salt thereof, wherein m is 1; s is 1; and Y¹ is —CR⁶R⁶⁰—, —CR⁶R⁶⁰—C(O)—. —C(O)—CR⁶R⁶⁰—, or C(O).

Embodiment 43. The compound of any one of embodiments 1-39, or a pharmaceutically acceptable salt thereof, wherein m is 1; s is 1; and Y¹ is —CR⁶R⁶⁰— or C(O).

Embodiment 44. The compound of any one of embodiments 1-43, or a pharmaceutically acceptable salt thereof, wherein Y¹ is bound to a nitrogen ring member of Cy¹.

Embodiment 45. The compound of any one of embodiments 1-44, or a pharmaceutically acceptable salt thereof, wherein Cy¹ is 4-10 membered heterocycloalkyl, which is optionally substituted by 1, 2, 3, or 4 independently selected R¹⁰ substituents.

46. The compound of any one of embodiments 1-44, or a pharmaceutically acceptable salt thereof, wherein Cy¹ is 4-10 membered heterocycloalkyl having 1, 2, or 3 nitrogen ring members, which is optionally substituted by 1, 2, 3, or 4 independently selected R¹⁰ substituents.

Embodiment 47. The compound of any one of embodiments 1-44, or a pharmaceutically acceptable salt thereof, wherein Cy¹ is 4-7 membered heterocycloalkyl, which is optionally substituted by 1, 2, 3, or 4 independently selected R¹⁰ substituents.

Embodiment 48. The compound of any one of embodiments 1-44, or a pharmaceutically acceptable salt thereof, wherein Cy¹ is 4-7 membered heterocycloalkyl having 1 or 2 nitrogen ring members, which is optionally substituted by 1, 2, 3, or 4 independently selected R¹⁰ substituents.

Embodiment 49. The compound of any one of embodiments 1-44, or a pharmaceutically acceptable salt thereof, wherein Cy¹ is an azetidine ring, a pyrrolidine ring, a piperidine ring, a piperazine ring, or an azepine ring, which are each optionally substituted by 1, 2, 3, or 4 independently selected R¹⁰ substituents.

Embodiment 50. The compound of any one of embodiments 1-44, or a pharmaceutically acceptable salt thereof, wherein Cy¹ is an azetidine ring, a pyrrolidine ring, or a piperidine ring, which are each optionally substituted by 1 or 2 independently selected R¹⁰ substituents.

Embodiment 51. The compound of any one of embodiments 1-39, or a pharmaceutically acceptable salt thereof, wherein m is 0; s is 1; and Y¹ is —(CR⁶R⁶⁰)ᵣ—, —CR⁶R⁶⁰—C(O)—. —C(O)—CR⁶R⁶⁰—, or C(O).

Embodiment 52. The compound of any one of embodiments 1-39, or a pharmaceutically acceptable salt thereof, wherein m is 0; s is 1; and Y¹ is —CR⁶R⁶⁰—, —CR⁶R⁶⁰—C(O)—. —C(O)—CR⁶R⁶⁰—, or C(O).

Embodiment 53. The compound of any one of embodiments 1-39, or a pharmaceutically acceptable salt thereof, wherein m is 0; s is 1; and Y¹ is —CR⁶R⁶⁰— or C(O).

Embodiment 54. The compound of any one of embodiments 1-50, or a pharmaceutically acceptable salt thereof, wherein each R¹⁰ is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a10}, R^{c10}, and R^{d10} is independently selected from H, C₁₋₆ alkyl, and C₁₋₆ haloalkyl; and
each R^{b10} is independently selected from C₁₋₆ alkyl and C₁₋₆ haloalkyl.

Embodiment 55. The compound of any one of embodiments 1-50, or a pharmaceutically acceptable salt thereof, wherein each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}OC(O)OR^{a10}, NR^{c10}C(O)NR^{c10}R^{d10}, NR^{c10}S(O)₂R^{b10}, NR^{c10}S(O)₂NR^{c10}R^{d10}S(O)₂R^{b10}, and S(O)₂NR^{c10}OR^{d10};
each R^{a10}, R^{c10}, and R^{d10} is independently selected from H, C₁₋₆ alkyl, and C₁₋₆ haloalkyl; and
each R^{b10} is independently selected from C₁₋₆ alkyl and C₁₋₆ haloalkyl.

Embodiment 56. The compound of any one of embodiments 1-39 and 41-55, or a pharmaceutically acceptable salt thereof, wherein each R⁶ is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}S(O)₂R^{b6}, and S(O)₂NR^{c6}R^{d6}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents; and
each R⁶⁰ is independently selected from H, C₁₋₆ alkyl, and C₁₋₆ haloalkyl;
or R⁶ and R⁶⁰, together with the carbon atom to which they are attached, form a C₃₋₁₀ cycloalkyl or a 4-10 membered heterocycloalkyl moiety, each of which is optionally substituted with 1, 2, 3, or 4 independently selected R^{G} substituents.

Embodiment 57. The compound of any one of embodiments 1-39 and 41-55, or a pharmaceutically acceptable salt thereof, wherein each R⁶ is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}S(O)₂R^{b6}, and S(O)₂NR^{c6}R^{d6}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents; and
each R⁶⁰ is independently selected from H, C₁₋₆ alkyl, and C₁₋₆ haloalkyl;
or R⁶ and R⁶⁰, together with the carbon atom to which they are attached, form a 4-10 membered heterocycloalkyl moiety, each of which is optionally substituted with 1, 2, 3, or 4 independently selected R^{G} substituents.

Embodiment 58. The compound of any one of embodiments 1-39 and 41-55, or a pharmaceutically acceptable salt thereof, wherein each R⁶ is independently selected from H, D, halo, CN, C₁₋₆ alkyl, C₂₋₆ alkenyl, C₂₋₆ alkynyl, C₁₋₆ haloalkyl, C₃₋₇ cycloalkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6} NR^{c6}C(O)OR^{a6}, NR^{c6}C(O)NR^{c6}R^{d6}, NR^{c6}S(O)₂NR^{c6}R^{d6}S(O)₂R^{b6}, and S(O)₂NR^{c6}R^{d6}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents; and
each R⁶⁰ is independently selected from H, C₁₋₆ alkyl, and C₁₋₆ haloalkyl;
or R⁶ and R⁶⁰, together with the carbon atom to which they are attached, form a C₃₋₁₀ cycloalkyl or a 4-10 membered heterocycloalkyl moiety, each of which is optionally substituted with 1, 2, 3, or 4 independently selected R^{7A} substituents.

Embodiment 59. The compound of any one of embodiments 1-39 and 41-55, or a pharmaceutically acceptable salt thereof, wherein each R⁶ is independently selected from H, D, halo, CN, C₁₋₆ alkyl, C₂₋₆ alkenyl, C₂₋₆ alkynyl, C₁₋₆ haloalkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R, NR^{c6}C(O)OR^{a6}, NR^{c6}C(O)NR^{c6}R^{d6}, NR^{c6}S(O)₂NR^{c6}R^{d6}S(O)₂R^{b6}, and S(O)₂NR^{c6}R^{d6}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents; and
each R⁶⁰ is independently selected from H, C₁₋₆ alkyl, and C₁₋₆ haloalkyl;
or R⁶ and R⁶⁰, together with the carbon atom to which they are attached, form a 4-10 membered heterocycloalkyl moiety, which is optionally substituted with 1, 2, 3, or 4 independently selected R^{7A} substituents.

Embodiment 60. The compound of any one of embodiments 1-39 and 41-55, or a pharmaceutically acceptable salt thereof, wherein each R⁶ is independently selected from H, D, halo, CN, C₁₋₆ alkyl, C₁₋₆ haloalkyl, and C₃₋₇ cycloalkyl.

Embodiment 61. The compound of any one of embodiments 1-39 and 41-55, or a pharmaceutically acceptable salt thereof, wherein each R⁶ is independently selected from H, D, halo, CN, C₁₋₆ alkyl, and C₁₋₆ haloalkyl.

Embodiment 62. The compound of any one of embodiments 1-39 and 41-55, or a pharmaceutically acceptable salt thereof, wherein each R⁶ is independently selected from H, D, C₁₋₃ alkyl, and C₃₋₇ cycloalkyl.

Embodiment 63. The compound of any one of embodiments 1-39 and 41-55, or a pharmaceutically acceptable salt thereof, wherein each R⁶ is independently selected from H, D, and C₁₋₃ alkyl.

Embodiment 64. The compound of any one of embodiments 1-39 and 41-55, or a pharmaceutically acceptable salt thereof, wherein each R⁶ is independently selected from H, C₁₋₃ alkyl, and C₃₋₇ cycloalkyl.

Embodiment 65. The compound of any one of embodiments 1-39 and 41-55, or a pharmaceutically acceptable salt thereof, wherein each R⁶ is independently selected from H and C₁₋₃ alkyl.

Embodiment 66. The compound of any one of embodiments 1-39 and 41-65, or a pharmaceutically acceptable salt thereof, wherein each R⁶⁰ is independently selected from H, D and C₁₋₃ alkyl.

Embodiment 67. The compound of any one of embodiments 1-39 and 41-65, or a pharmaceutically acceptable salt thereof, wherein each R⁶⁰ is independently selected from H and C₁₋₃ alkyl.

Embodiment 68. The compound of any one of embodiments 1-39 and 41-65, or a pharmaceutically acceptable salt thereof, wherein R⁶ and R⁶⁰, together with the carbon atom to which they are attached, form a C₃₋₁₀ cycloalkyl or a 4-10 membered heterocycloalkyl moiety, each of which is optionally substituted with 1, 2, 3, or 4 independently selected R^{7A} substituents.

Embodiment 69. The compound of any one of embodiments 1-39 and 41-65, or a pharmaceutically acceptable salt thereof, wherein R⁶ and R⁶⁰, together with the carbon atom to which they are attached, form a C₃₋₁₀ cycloalkyl moiety, which is optionally substituted with 1, 2, 3, or 4 independently selected R^{7A} substituents.

Embodiment 70. The compound of any one of embodiments 1-39 and 41-65, or a pharmaceutically acceptable salt thereof, wherein:
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b6}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{6B} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, C(O)R^{b62}, C(O)NR^{c62}R^{d62}, C(O)OR^{a62}, OC(O)R^{b62}, OC(O)NR^{c62}R^{d62}, NR^{c62}R^{d62}, R^{c62}C(O)R^{b62}, NR^{c62}C(O)OR^{a62}, NR^{c62}C(O)NR^{c62}R^{d62}, NR^{c62}S(O)₂R^{b62}, NR^{c62}S(O)₂NR^{c62}R^{d62}, S(O)₂R^{b62}, and S(O)₂NR^{c62}R^{d62};
each R^{a62}, R^{c62}, and R^{d62} is independently selected from H, C₁₋₆ alkyl, C₁₋₆ haloalkyl, C₂₋₆ alkenyl, and C₂₋₆ alkynyl; and
each R^{b62} is independently selected from C₁₋₆ alkyl, C₁₋₆ haloalkyl, C₂₋₆ alkenyl, and C₂₋₆ alkynyl.

Embodiment 71. The compound of any one of embodiments 1-39 and 41-65, or a pharmaceutically acceptable salt thereof, wherein:
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c6}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61};
each R^{a61}, R^{c61}, and R^{d61} is independently selected from H, C₁₋₆ alkyl, and C₁₋₆ haloalkyl; and
each R^{b61} is independently selected from C₁₋₆ alkyl and C₁₋₆ haloalkyl.

Embodiment 72. The compound of any one of embodiments 1-39 and 41-65, or a pharmaceutically acceptable salt thereof, wherein:
each R^{a6}, R^{c6}, and R^{d6} is independently selected from H, C₁₋₆ alkyl, C₁₋₆ haloalkyl, C₂₋₆ alkenyl, and C₂₋₆ alkynyl, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, and C₂₋₆ alkynyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, OR^{a61}, SR^{a61}, C(O)R^{b61}, C(O)NR^{c61}R^{d61}, C(O)OR^{a11}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61};
each R^{a61}, R^{c61}, and R^{d61} is independently selected from H, C₁₋₆ alkyl, and C₁₋₆ haloalkyl; and
each R^{b61} is independently selected from C₁₋₆ alkyl and C₁₋₆ haloalkyl.

Embodiment 73. The compound of any one of embodiments 1-72, or a pharmaceutically acceptable salt thereof, wherein Ring moiety B is phenyl, naphthyl, 5-10 membered heteroaryl, or 9-12 membered heterocycloalkyl.

Embodiment 74. The compound of any one of embodiments 1-72, or a pharmaceutically acceptable salt thereof, wherein Ring moiety B is phenyl, naphthyl, 5-6 membered heteroaryl, 9-10 membered heteroaryl, or 9-12 membered heterocycloalkyl.

Embodiment 75. The compound of any one of embodiments 1-72, or a pharmaceutically acceptable salt thereof, wherein Ring moiety B is phenyl.

Embodiment 76. The compound of any one of embodiments 1-72, or a pharmaceutically acceptable salt thereof, wherein Ring moiety B is naphthyl.

Embodiment 77. The compound of any one of embodiments 1-72, or a pharmaceutically acceptable salt thereof, wherein Ring moiety B is 5-6 membered heteroaryl.

Embodiment 78. The compound of any one of embodiments 1-72, or a pharmaceutically acceptable salt thereof, wherein Ring moiety B is 9-10 membered heteroaryl.

Embodiment 79. The compound of any one of embodiments 1-72, or a pharmaceutically acceptable salt thereof, wherein Ring moiety B is 9-12 membered heterocycloalkyl.

Embodiment 80. The compound of any one of embodiments 1-72, or a pharmaceutically acceptable salt thereof, wherein Ring moiety B is

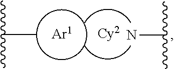

wherein Ar¹ is fused to Cy², Ar¹ is bonded to Y, Ar¹ is phenyl or 5-6 membered heteroaryl, and Cy² is 4-8 membered heterocycloalkyl.

Embodiment 81. The compound of any one of embodiments 1-72, or a pharmaceutically acceptable salt thereof, wherein Ring moiety B is phenyl, naphthyl, 5-6 membered heteroaryl, 9-10 membered heteroaryl, or

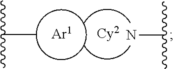

wherein:
Ar¹ is fused to Cy² and Ar¹ is bonded to Y;
Ar¹ is phenyl or 5-6 membered heteroaryl; and
Cy² is 4-8 membered heterocycloalkyl.

Embodiment 82. The compound of any one of embodiments 1-81, or a pharmaceutically acceptable salt thereof, wherein R³ is selected from H, D, halo, CN, C₁₋₆ alkyl, C₂₋₆ alkenyl, C₂₋₆ alkynyl, C₁₋₆ haloalkyl, C₃₋₅ cycloalkyl, OR^{a3}, SR^{a3}, NHOR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}.

Embodiment 83. The compound of any one of embodiments 1-82, or a pharmaceutically acceptable salt thereof, wherein each R^{a3}, R^{c3}, and R^{d3} is independently selected from H, C₁₋₆ alkyl, C₂₋₆ alkenyl, C₂₋₆ alkynyl, and C₁₋₆ haloalkyl; and each R^{b3} is independently selected from C₁₋₆ alkyl, C₂₋₆ alkenyl, C₂₋₆ alkynyl, and C₁₋₆ haloalkyl.

Embodiment 84. The compound of any one of embodiments 1-81, or a pharmaceutically acceptable salt thereof, wherein R³ is selected from H, D, halo, CN, C₁₋₆ alkyl, C₂₋₆ alkenyl, C₂₋₆ alkynyl, C₁₋₆ haloalkyl, OR^{a3}, and NR^{c3}R^{d3}.

Embodiment 85. The compound of any one of embodiments 1-81, or a pharmaceutically acceptable salt thereof, wherein R³ is selected from H, D, halo, CN, C₁₋₆ alkyl, C₂₋₆ alkenyl, C₂₋₆ alkynyl, and C₁₋₆ haloalkyl.

Embodiment 86. The compound of any one of embodiments 1-81, or a pharmaceutically acceptable salt thereof, wherein R³ is H.

Embodiment 87. The compound of any one of embodiments 1-82, or a pharmaceutically acceptable salt thereof, wherein each R^{a3}, R^{c3}, and R^{d3} is independently selected from H and C₁₋₆ alkyl.

Embodiment 88. The compound of any one of embodiments 1-87, or a pharmaceutically acceptable salt thereof, wherein R⁴ is selected from H, D, halo, CN, C₁₋₆ alkyl, C₂₋₆ alkenyl, C₂₋₆ alkynyl, C₁₋₆ haloalkyl, C₃₋₅ cycloalkyl, OR^{a4}, SR^{a4}, NHOR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)₂R^{b4}, NR^{c4}S(O)₂NR^{c4}R^{d4}, S(O)₂R^{b4}, and S(O)₂NR^{c4}R^{d4}.

Embodiment 89. The compound of any one of embodiments 1-88, or a pharmaceutically acceptable salt thereof, wherein each R^{a4}, R^{c4}, and R^{d4} is independently selected from H, C₁₋₆ alkyl, C₂₋₆ alkenyl, C₂₋₆ alkynyl, and C₁₋₆ haloalkyl; and each R⁴ is independently selected from C₁₋₆ alkyl, C₂₋₆ alkenyl, C₂₋₆ alkynyl, and C₁₋₆ haloalkyl.

Embodiment 90. The compound of any one of embodiments 1-87, or a pharmaceutically acceptable salt thereof, wherein R⁴ is selected from H, D, halo, CN, C₁₋₆ alkyl, C₂₋₆ alkenyl, C₂₋₆ alkynyl, and C₁₋₆ haloalkyl.

Embodiment 91. The compound of any one of embodiments 1-87, or a pharmaceutically acceptable salt thereof, wherein R⁴ is selected from H.

Embodiment 92. The compound of any one of embodiments 1-91, or a pharmaceutically acceptable salt thereof, wherein Ring moiety A is 6-10 membered aryl or 5-10 membered heteroaryl.

Embodiment 93. The compound of any one of embodiments 1-91, or a pharmaceutically acceptable salt thereof, wherein Ring moiety A is phenyl or 5-10 membered heteroaryl.

Embodiment 94. The compound of any one of embodiments 1-91, or a pharmaceutically acceptable salt thereof, wherein Ring moiety A is phenyl, 5-6 membered heteroaryl, and 9-10 membered heteroaryl.

Embodiment 95. The compound of any one of embodiments 1-91, or a pharmaceutically acceptable salt thereof, wherein Ring moiety A is 6-10 membered aryl.

Embodiment 96. The compound of any one of embodiments 1-91, or a pharmaceutically acceptable salt thereof, wherein Ring moiety A is phenyl.

Embodiment 97. The compound of any one of embodiments 1-91, or a pharmaceutically acceptable salt thereof, wherein Ring moiety A is 5-10 membered heteroaryl.

Embodiment 98. The compound of any one of embodiments 1-91, or a pharmaceutically acceptable salt thereof, wherein Ring moiety A is 5-6 membered heteroaryl or 9-10 membered heteroaryl.

Embodiment 99. The compound of any one of embodiments 1-91, or a pharmaceutically acceptable salt thereof, wherein Ring moiety A is 5-6 membered heteroaryl.

Embodiment 100. The compound of any one of embodiments 1-91, or a pharmaceutically acceptable salt thereof, wherein Ring moiety A is 9-10 membered heteroaryl.

Embodiment 101. The compound of any one of embodiments 1-100, or a pharmaceutically acceptable salt thereof, wherein each R⁵ is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, NHOR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents.

Embodiment 102. The compound of any one of embodiments 1-100, or a pharmaceutically acceptable salt thereof, wherein each R⁵ is independently selected from D, halo, CN, C₁₋₆ alkyl, C₂₋₆ alkenyl, C₂₋₆ alkynyl, C₁₋₆ haloalkyl, OR^{a5} and NR^{c5}R^{d5}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{5A} substituents.

Embodiment 103. The compound of any one of embodiments 1-102, or a pharmaceutically acceptable salt thereof, wherein:
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl which are each optionally substituted with 1, 2, 3, or 4 independently selected R^{5A} substituents; and
each R^{5A} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 104. The compound of any one of embodiments 1-102, or a pharmaceutically acceptable salt thereof, wherein:
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, wherein said C₁₋₆ alkyl, C₂₋₆ alkenyl, C₂₋₆ alkynyl, C₁₋₆ haloalkyl, C₃₋₄ cycloalkyl, and 4-5 membered heterocycloalkyl are each optionally substituted with 1, 2, 3, or 4 independently selected R^{SA} substituents;
each R^{b5} is independently selected from C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents; and
each R^{5A} is independently selected from OH, CN, halo, C₁₋₃ alkyl, C₁₋₃ haloalkyl, amino, C₁₋₃ alkylamino, and di(C₁₋₃ alkyl)amino.

Embodiment 105. The compound of any one of embodiments 1-100, or a pharmaceutically acceptable salt thereof, wherein each R⁵ is independently selected from halo, CN, C₁₋₃ alkyl, C₁₋₃ haloalkyl, OH, C₁₋₃ alkoxy, and C₁₋₃ haloalkoxy.

Embodiment 106. The compound of any one of embodiments 1-100, or a pharmaceutically acceptable salt thereof, wherein each R⁵ is independently selected from Cl, F, CN, CH₃, OH, and OCH₃.

Embodiment 107. The compound of any one of embodiments 1-91, or a pharmaceutically acceptable salt thereof, wherein:

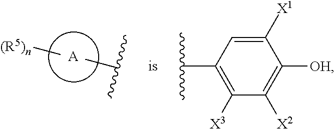

wherein X¹ and X² are each independently Cl or F, and X³ is H, Cl or F.

Embodiment 108. The compound of any one of embodiments 1-91, or a pharmaceutically acceptable salt thereof, wherein:

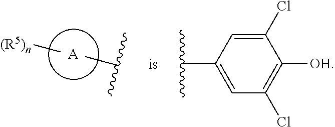

Embodiment 109. The compound of any one of embodiments 1-91, or a pharmaceutically acceptable salt thereof, wherein:

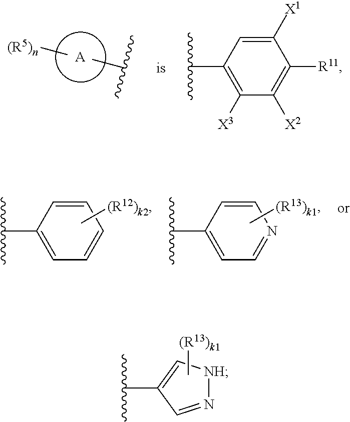

X¹ is Cl, F, or OCH₃;
X² is H, Cl, or F;
X³ is H, Cl or F;
R¹¹ is OH or CN;
k1 is 0, 1, or 2;
k2 is 2;
two R¹² substituents, together with the carbon atoms to which they are attached, form a pyrazole, pyrrole, or pyrimidine ring, which is optionally substituted by 1 or 2 substituents independently selected from halo, CN, and C₁₋₃ alkyl; and
each R¹³ is independently selected from halo, CN, C₁₋₃ alkyl, and methoxy.

Embodiment 110. The compound of any one of embodiments 1-91, or a pharmaceutically acceptable salt thereof, wherein:

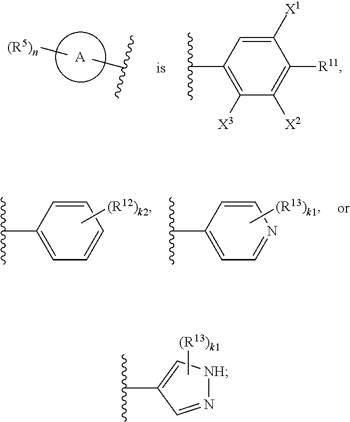

X¹ is Cl, F, or OCH₃;
X² is H, Cl, or F;
X³ is H, Cl or F;
R¹¹ is OH or CN;
k1 is 0, 1, or 2;
k2 is 2;
two R¹² substituents, together with the carbon atoms to which they are attached, form a pyrazole, pyrrole, or pyrimidine ring, which is optionally substituted by 1 or 2 substituents independently selected from halo, CN, and C₁₋₃ alkyl; and
each R¹³ is independently selected from halo, CN, and C₁₋₃ alkyl.

Embodiment 111. The compound of any one of embodiments 1-91, or a pharmaceutically acceptable salt thereof, wherein:

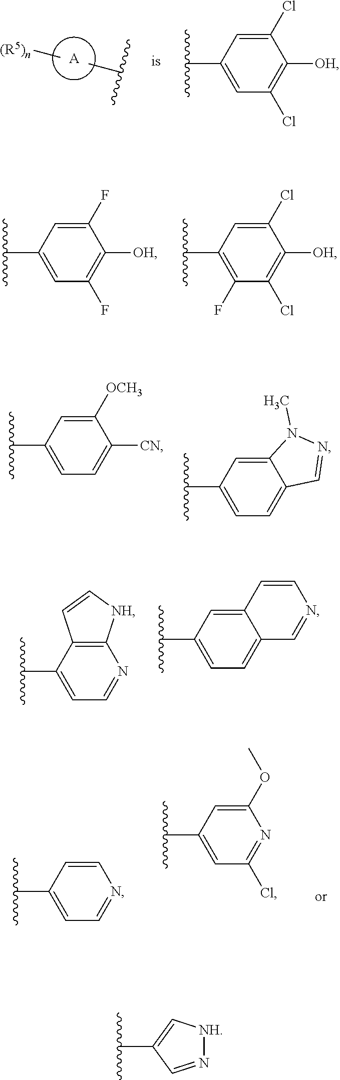

Embodiment 112. The compound of any one of embodiments 1-91, or a pharmaceutically acceptable salt thereof, wherein:

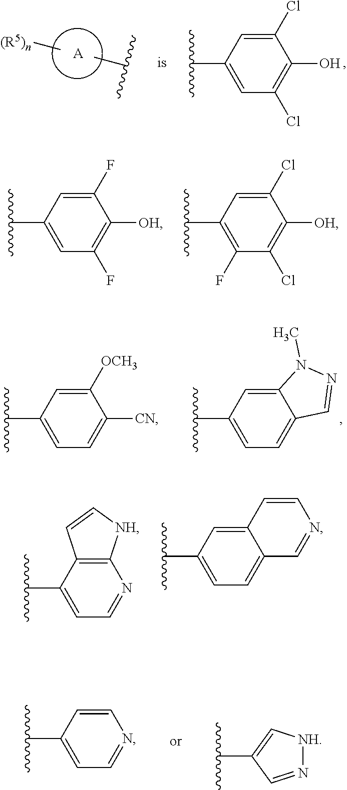

Embodiment 113. The compound of any one of embodiments 1-112, or a pharmaceutically acceptable salt thereof, wherein each R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₁₀ cycloalkyl, 6-10 membered aryl, 4-10 membered heterocycloalkyl, 5-membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents.

Embodiment 114. The compound of any one of embodiments 1-112, or a pharmaceutically acceptable salt thereof, wherein each R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents.

Embodiment 115. The compound of any one of embodiments 1-112, or a pharmaceutically acceptable salt thereof, wherein each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃-7 cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents.

Embodiment 116. The compound of any one of embodiments 1-115, or a pharmaceutically acceptable salt thereof, wherein:
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c71}C(O)OR^{a71}, NR^{c71}C(O)NR^{c71}R^{d71}, NR^{c71}S(O)₂R^{b71}, NR^{c71}S(O)₂NR^{c71}R^{d71}, S(O)₂R^{b71}, and S(O)₂NR^{c71}R^{d71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 117. The compound of any one of embodiments 1-115, or a pharmaceutically acceptable salt thereof, wherein:
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl; and
each R^{b71} is independently selected from C₁₋₆ alkyl and C₁₋₆ haloalkyl.

Embodiment 118. The compound of any one of embodiments 1-115, or a pharmaceutically acceptable salt thereof, wherein:
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents; and
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino.

Embodiment 119. The compound of embodiment 1, or a pharmaceutically acceptable salt thereof, wherein:
n is 0, 1, 2, 3, or 4;
p is 0, 1, 2, 3, or 4;
r is 1 or 2;
m is 1; s is 1; and Y¹ is a bond; or
m is 1; s is 1; and Y¹ is —(CR⁶R⁶⁰)ᵣ—, —CR⁶R⁶⁰—C(O)—. —C(O)—CR⁶R⁶⁰—, or C(O); or
m is 0; s is 1; and Y¹ is —(CR⁶R⁶⁰)ᵣ—, —CR⁶R⁶⁰—C(O)—. —C(O)—CR⁶R⁶⁰—, or C(O);
Cy¹ is 4-10 membered heterocycloalkyl having 1, 2, or 3 nitrogen ring members, which is optionally substituted by 1, 2, 3, or 4 independently selected R¹⁰ substituents;
Ring moiety A is phenyl or 5-10 membered heteroaryl;
Ring moiety B is phenyl, naphthyl, 5-6 membered heteroaryl, 9-10 membered heteroaryl, or

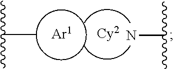

Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b1}, SR^{b1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}S(O)₂R^{b11}, and S(O)₂NR^{c1}R^{d1}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{a1}, R^{c}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
or, any R^{c} and R^{d1} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R¹¹ is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from D, halo, CN, C₁₋₆ alkyl, C₂₋₆ alkenyl, C₂₋₆ alkynyl, C₁₋₆ haloalkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12}, and R^{d12} is independently selected from H, C₁₋₆ alkyl, and C₁₋₆ haloalkyl;
each R¹² is independently selected from C₁₋₆ alkyl, and C₁₋₆ haloalkyl; R² is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{2A} substituents.
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2A}
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2A} substituents;
each R^{2A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21}.
each R^{a21}, R^{c21}, and R^{d21} is independently selected from H, C₁₋₆ alkyl, and C₁₋₆ haloalkyl;
each R^{b21} is independently selected from C₁₋₆ alkyl and C₁₋₆ haloalkyl; R³ is selected from H, D, halo, CN, C₁₋₆ alkyl, C₂₋₆ alkenyl, C₂₋₆ alkynyl, C₁₋₆ haloalkyl, C₃₋₅ cycloalkyl, OR^{a3}, SR^{a3}, NHOR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}S(O)₂R^{b3}, and S(O)₂NR^{c3}R^{d3};
each R^{a3}, R^{c3}, and R^{d3} is independently selected from H, C₁₋₆ alkyl, C₂₋₆ alkenyl, C₂₋₆ alkynyl, and C₁₋₆ haloalkyl;
each R^{b3} is independently selected from C₁₋₆ alkyl, C₂₋₆ alkenyl, C₂₋₆ alkynyl, and C₁₋₆ haloalkyl;
R⁴ is selected from H, D, halo, CN, C₁₋₆ alkyl, C₂₋₆ alkenyl, C₂₋₆ alkynyl, C₁₋₆ haloalkyl, C₃₋₅ cycloalkyl, OR^{a4}, SR^{a4}, NHOR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)₂R^{b4}, NR^{c4}S(O)₂NR^{c4}R^{d4}, S(O)₂R^{b4}, and S(O)₂NR^{c4}R^{d4};
each R^{a4}, R^{c4}, and R^{d4} is independently selected from H, C₁₋₆ alkyl, C₂₋₆ alkenyl, C₂₋₆ alkynyl, and C₁₋₆ haloalkyl;
each R^{b4} is independently selected from C₁₋₆ alkyl, C₂₋₆ alkenyl, C₂₋₆ alkynyl, and C₁₋₆ haloalkyl;
each R⁵ is independently selected from D, halo, CN, C₁₋₆ alkyl, C₂₋₆ alkenyl, C₂₋₆ alkynyl, C₁₋₆ haloalkyl, OR^{as} and NR^{c5}R^{d5}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{5A} substituents;
each R^{a5}, R^{c}, and R^{d5} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{5A} substituents;
each R^{5A} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
each R⁶ is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}S(O)₂R^{b6}, and S(O)₂NR^{c6}R^{d6}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents;
each R⁶⁰ is independently selected from H, C₁₋₆ alkyl, and C₁₋₆ haloalkyl;
or R⁶ and R⁶⁰, together with the carbon atom to which they are attached, form a C₃₋₁₀ cycloalkyl or a 4-10 membered heterocycloalkyl moiety, each of which are optionally substituted with 1, 2, 3, or 4 independently selected R^{7A} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61};
each R^{a61}, R^{c61}, and R^{d61} is independently selected from H, C₁₋₆ alkyl, and C₁₋₆ haloalkyl;
each R^{b61} is independently selected from C₁₋₆ alkyl and C₁₋₆ haloalkyl.
each R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c71}C(O)OR^{a71}, NR^{c71}C(O)NR^{c71}R^{d71}, NR^{c71}S(O)₂R^{b71}, NR^{c71}S(O)₂NR^{c71}R^{d71}, S(O)₂R^{b71}, and S(O)₂NR^{c71}R^{d71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{7B} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R^{W} is selected from.

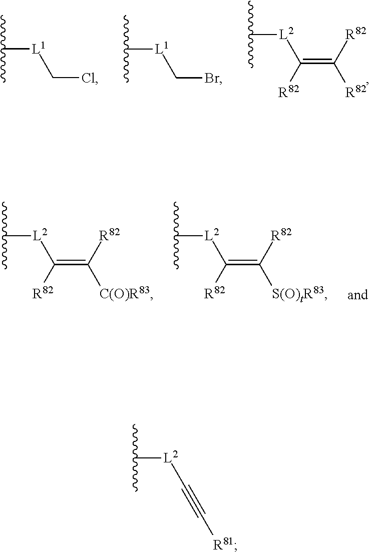

L¹ is -L-C(O)—, -L-NR⁹C(O)—, -L-S(O)₂—, -L-NR⁹S(O)—, or -L-NR⁹S(O)₂—, or wherein L¹ is attached to Ring moiety B through the L linking group;
L² is -L-C(O)—, -L-NR⁹C(O)—, -L-S(O)₂—, or -L-NR⁹S(O)₂—, wherein L² is attached to Ring moiety B through the L linking group;
L is a bond;
R⁹ is selected from H and C₁₋₆ alkyl;
R⁸¹, R⁸², and R⁸³ are each independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{c8}R^{d8}, NR^{c8}R^{d8}, NR^{c8}NR^{c8}R^{d8}, NR^{c8}C(O)R^{b8}, NR^{c8}C(O)OR^{a8}, NR^{c8}C(O)NR^{c8}R^{d8}, C(═NR^{e8})R^{b8}, C(═NR^{e8})NR^{c8}R^{d8}, NR^{c8}C(═NR^{e8})NR^{c8}R^{d8}, NR^{c8}C(═NR^{e8})R^{b8}, NR^{c8}S(O)NR^{c8}R^{d8}, NR^{c8}S(O)R^{b8}, NR^{c8}S(O)₂R^{b8}, NR^{c8}S(O)(═NR^{e8})R^{b8}, NR^{c8}S(O)₂NR^{c8}R^{d8}S(O)R^{b8}, S(O)NR^{c8}R^{d8}, S(O)₂R^{b8}, S(O)₂NR^{c8}R^{d8}, OS(O)(═NR^{e8})R^{b8}, OS(O)₂R^{b8}, and S(O)(═NR^{e8})R^{b8}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a8}, R^{c8}, and R^{d8} is independently selected from H and C₁₋₆ alkyl;
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b11}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl;
each R^{b10} is independently selected from C₁₋₆ alkyl and C₁₋₆ haloalkyl; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, N(CD₃)₂, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 120. The compound of embodiment 1, or a pharmaceutically acceptable salt thereof, wherein:
n is 0, 1, 2, 3, or 4;
p is 0, 1, 2, 3, or 4;
r is 1 or 2;
m is 1; s is 1; and Y¹ is a bond; or
m is 1; s is 1; and Y¹ is —(CR⁶R⁶⁰)ᵣ—, —CR⁶R⁶⁰—C(O)—. —C(O)—CR⁶R⁶⁰—, or C(O); or
m is 0; s is 1; and Y¹ is —(CR⁶R⁶⁰)ᵣ—, —CR⁶R⁶⁰—C(O)—. —C(O)—CR⁶R⁶⁰—, or C(O);
Cy¹ is 4-10 membered heterocycloalkyl having 1, 2, or 3 nitrogen ring members, which is optionally substituted by 1, 2, 3, or 4 independently selected R¹⁰ substituents;
Ring moiety A is phenyl or 5-10 membered heteroaryl;
Ring moiety B is phenyl, naphthyl, 5-6 membered heteroaryl, 9-10 membered heteroaryl, or

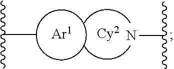

Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b1}, SR^{b1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b11}, and S(O)₂NR^{c1}R^{d1}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{a1}, R^{c}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
or, any R^{c} and R^{d1} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR¹¹S(O)₂NR^{b11}R^{d11}, S(O)₂R^{b11}, and S(O)₂NR^{c11}R^{d11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from D, halo, CN, C₁₋₆ alkyl, C₂₋₆ alkenyl, C₂₋₆ alkynyl, C₁₋₆ haloalkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12};
each R^{a12}, R^{c12}, and R^{d12} is independently selected from H, C₁₋₆ alkyl, and C₁₋₆ haloalkyl;
each R¹² is independently selected from C₁₋₆ alkyl, and C₁₋₆ haloalkyl;
R² is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{2A} substituents.
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2A} substituents;
each R^{2A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21}.
each R^{a21}, R^{c21}, and R^{d21} is independently selected from H, C₁₋₆ alkyl, and C₁₋₆ haloalkyl;
each R^{b21} is independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, C₁₋₆ haloalkyl, C₃₋₅ cycloalkyl, OR^{a3}, SR^{a3}, NHOR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}S(O)₂R^{b3}, and S(O)₂NR^{c3}R^{d3};
each R^{a3}, R^{c3}, and R^{d3} is independently selected from H, C₁₋₆ alkyl, C₂₋₆ alkenyl, C₂₋₆ alkynyl, and C₁₋₆ haloalkyl;
each R^{b3} is independently selected from C₁₋₆ alkyl, C₂₋₆ alkenyl, C₂₋₆ alkynyl, and C₁₋₆ haloalkyl;
R⁴ is selected from H, D, halo, CN, C₁₋₆ alkyl, C₂₋₆ alkenyl, C₂₋₆ alkynyl, C₁₋₆ haloalkyl, C₃₋₅ cycloalkyl, OR^{a4}, SR^{a4}, NHOR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)₂R^{b4}, NR^{c4}S(O)₂NR^{c4}R^{d4}S(O)₂R^{b4}, and S(O)₂NR^{c4}R^{d4};
each R^{a4}, R^{c4}, and R^{d4} is independently selected from H, C₁₋₆ alkyl, C₂₋₆ alkenyl, C₂₋₆ alkynyl, and C₁₋₆ haloalkyl;
each R^{b4} is independently selected from C₁₋₆ alkyl, C₂₋₆ alkenyl, C₂₋₆ alkynyl, and C₁₋₆ haloalkyl;
each R⁵ is independently selected from D, halo, CN, C₁₋₆ alkyl, C₂₋₆ alkenyl, C₂₋₆ alkynyl, C₁₋₆ haloalkyl, OR^{as} and NR^{c5}R^{d5}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{5A} substituents;
each R^{a5}, R^{c5}, and R^{d5} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{5A} substituents;
each R^{5A} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
each R⁶ is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}S(O)₂R^{b6}, and S(O)₂NR^{c6}R^{d6}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents;
each R⁶⁰ is independently selected from H, C₁₋₆ alkyl, and C₁₋₆ haloalkyl;
or R⁶ and R⁶⁰, together with the carbon atom to which they are attached, form a 4-10 membered heterocycloalkyl moiety, which is optionally substituted with 1, 2, 3, or 4 independently selected R^{7A} substituents.
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61};
each R^{a61}, R^{c61}, and R^{d61} is independently selected from H, C₁₋₆ alkyl, and C₁₋₆ haloalkyl;
each R^{b61} is independently selected from C₁₋₆ alkyl and C₁₋₆ haloalkyl.
each R⁷ is independently selected from D, halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)₂R^{b7}, NR^{c7}S(O)₂NR^{c7}R^{d7}, S(O)₂R^{b7}, and S(O)₂NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, C(O)R^{b71}, C(O)NR^{c71}R^{d71}, C(O)OR^{a71}, OC(O)R^{b71}, OC(O)NR^{c71}R^{d71}, NR^{c71}R^{d71}, NR^{c71}C(O)R^{b71}, NR^{c71}C(O)OR^{a71}, NR^{c71}C(O)NR^{c71}R^{d71}, NR^{c71}S(O)₂R^{b71}, NR^{c71}S(O)₂NR^{c71}R^{d71}, S(O)₂R^{b71}, and S(O)₂NR^{c71}R^{d71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c7}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{7B} is independently selected from D, OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R^{W} is selected from:

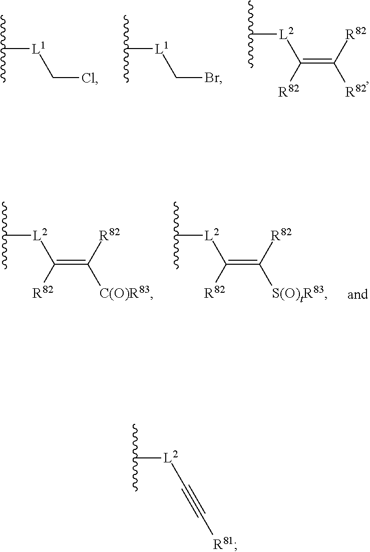

L¹ is -L-C(O)—, -L-NR⁹C(O)—, -L-S(O)₂—, -L-NR⁹S(O)—, or -L-NR⁹S(O)₂—, or wherein L¹ is attached to Ring moiety B through the L linking group;
L² is -L-C(O)—, -L-NR⁹C(O)—, -L-S(O)₂—, or -L-NR⁹S(O)₂—, wherein L² is attached to Ring moiety B through the L linking group;
L is a bond;
R⁹ is selected from H and C₁₋₆ alkyl;
R⁸¹, R⁸², and R⁸³ are each independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8}, SR^{a8}, NHOR^{a8}, C(O)R^{b8}, C(O)NR^{c8}R^{d8}, C(O)NR^{c8}(OR^{a8}), C(O)OR^{a8}, OC(O)R^{b8}, OC(O)NR^{c8}R^{d8}, NR^{c8}R^{d8}, NR^{c8}NR^{c8}R^{d8}, NR^{c8}C(O)R^{b8}, NR^{c8}C(O)OR^{a8}, NR^{c8}C(O)NR^{c8}R^{d8}, C(═NR^{e8})R^{b8}, C(═NR^{e8})R^{c8}R^{d8}, NR^{c8}C(═NR^{e8})NR^{c8}R^{d8}, NR^{c8}C(═NR^{e8})R^{b8}, NR^{c8}S(O)NR^{c8}R^{d8}, NR^{c8}S(O)R^{b8}, NR^{c8}S(O)₂R^{b8}, NR^{c8}S(O)(═NR^{e8})R^{b8}, NR^{c8}S(O)₂NR^{c8}R^{d8}S(O)R^{b8}, S(O)₂R^{c8}R^{d8}S(O)₂R^{b8}, S(O)₂NR^{c8}R^{d8}, OS(O)(═NR^{e8})R^{b8}, OS(O)₂R^{b8}, and S(O)(═NR^{e8})R^{b8}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a5}, R^{c8}, and R^{d8} is independently selected from H and C₁₋₆ alkyl;
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl;
each R^{b10} is independently selected from C₁₋₆ alkyl and C₁₋₆ haloalkyl; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 121. The compound of embodiment 1, or a pharmaceutically acceptable salt thereof, wherein:
n is 0, 1, 2, 3, or 4;
p is 0, 1, 2, 3, or 4;
m is 1; s is 1; and Y¹ is a bond; or
m is 1; s is 1; and Y¹ is —CR⁶R⁶⁰—, —(CR⁶R⁶⁰)₂—, —CR⁶R⁶⁰—C(O)—. —C(O)—CR⁶R⁶⁰—, or C(O); or
m is 0; s is 1; and Y¹ is —CR⁶R⁶⁰—, —(CR⁶R⁶⁰)₂—, —CR⁶R⁶⁰—C(O)—. —C(O)—CR⁶R⁶⁰—, or C(O);
Cy¹ is 4-7 membered heterocycloalkyl having 1 or 2 nitrogen ring members, which is optionally substituted by 1, 2, 3, or 4 independently selected R¹⁰ substituents; Ring moiety A is phenyl, 5-6 membered heteroaryl, or 9-10 membered heteroaryl; Ring moiety B is phenyl, naphthyl, 5-6 membered heteroaryl, 9-10 membered heteroaryl, or

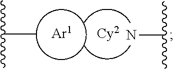

Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11} is independently selected from H, C₁₋₆ alkyl, and C₁₋₆ haloalkyl;
each R¹¹ is independently selected from C₁₋₆ alkyl and C₁₋₆ haloalkyl;
R² is selected from H, D, halo, CN, C₁₋₆ alkyl, C₂₋₆ alkenyl, C₂₋₆ alkynyl, C₁₋₆ haloalkyl, and C₃₋₅ cycloalkyl, wherein said C₁₋₆ alkyl, C₂₋₆ alkenyl, C₂₋₆ alkynyl, C₁₋₆ haloalkyl, and C₃₋₅ cycloalkyl are each optionally substituted with 1, 2, 3, or 4 independently selected R^{2A} substituents;
each R^{2A} is independently selected from D, halo, CN, C₁₋₆ alkyl, C₂₋₆ alkenyl, C₂₋₆ alkynyl, C₁₋₆ haloalkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21};
each R^{a21}, R^{c21}, and R^{d21} is independently selected from H, C₁₋₆ alkyl, and C₁₋₆ haloalkyl;
each R^{b21} is independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, C₁₋₆ haloalkyl, OR^{a3}, and NR^{c3}R^{d3};
each R^{a3}, R^{c3}, and R^{d3} is independently selected from H and C₁₋₆ alkyl;
R⁴ is selected from H, D, halo, CN, C₁₋₆ alkyl, C₂₋₆ alkenyl, C₂₋₆ alkynyl, and C₁₋₆ haloalkyl;
R⁵ is independently selected from D, halo, CN, C₁₋₆ alkyl, C₂₋₆ alkenyl, C₂₋₆ alkynyl, C₁₋₆ haloalkyl, C₃₋₄ cycloalkyl, OR^{a5} and NR^{c5}R^{d5}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{5A} substituents;
each R^{a5}, R^{c5}, and R^{d5} is independently selected from C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents;
each R^{5A} is independently selected from OH, CN, halo, C₁₋₃ alkyl, C₁₋₃ haloalkyl, amino, C₁₋₃ alkylamino, and di(C₁₋₃ alkyl)amino;
each R⁶ is independently selected from H, D, halo, CN, C₁₋₆ alkyl, C₂₋₆ alkenyl, C₂₋₆ alkynyl, C₁₋₆ haloalkyl, C₃₋₇ cycloalkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}S(O)₂R^{b6}, and S(O)₂NR^{c6}R^{d6}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents;
each R⁶⁰ is independently selected from H, C₁₋₆ alkyl, and C₁₋₆ haloalkyl;
or R⁶ and R⁶⁰, together with the carbon atom to which they are attached, form a C₃₋₇ cycloalkyl or a 4-7 membered heterocycloalkyl moiety, each of which are optionally substituted with 1, 2, 3, or 4 independently selected R^{7A} substituents;
each R^{a6}, R^{c6}, and R^{d6} is independently selected from H, C₁₋₆ alkyl, C₁₋₆ haloalkyl, C₂₋₆ alkenyl, and C₂₋₆ alkynyl, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, and C₂₋₆ alkynyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61};
each R^{a61}, R^{c61}, and R^{d61} is independently selected from H, C₁₋₆ alkyl, and C₁₋₆ haloalkyl;
each R^{b61} is independently selected from C₁₋₆ alkyl and C₁₋₆ haloalkyl;
each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl;
each R^{b71} is independently selected from C₁₋₆ alkyl and C₁₋₆ haloalkyl;
R^{W} is selected from:

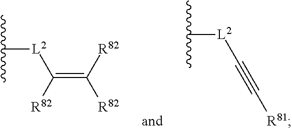

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, OR^{a8}, C(O)R^{b8}, C(O)NR^{c8}R^{d8}, and C(O)OR^{a5}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each R^{a8}, R^{c8}, and R^{d8} is independently selected from H and C₁₋₆ alkyl;
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R^{a10}, R^{b10}, and R^{d10} is independently selected from H, C₁₋₆ alkyl, and C₁₋₆ haloalkyl;
each R^{b10} is independently selected from C₁₋₆ alkyl and C₁₋₆ haloalkyl; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, N(CD₃)₂, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 122. The compound of embodiment 1, or a pharmaceutically acceptable salt thereof, wherein:

n is 0, 1, 2, 3, or 4;
p is 0, 1, 2, 3, or 4;
m is 1; s is 1; and Y¹ is a bond; or
m is 1; s is 1; and Y¹ is —CR⁶R⁶⁰—, —CR⁶R⁶⁰—C(O)—. —C(O)—CR⁶R⁶⁰—, or C(O); or
m is 0; s is 1; and Y¹ is —CR⁶R⁶⁰—, —CR⁶R⁶⁰—C(O)—. —C(O)—CR⁶R⁶⁰—, or C(O);
Cy¹ is 4-7 membered heterocycloalkyl having 1 or 2 nitrogen ring members, which is optionally substituted by 1, 2, 3, or 4 independently selected R¹⁰ substituents;
Ring moiety A is phenyl, 5-6 membered heteroaryl, or 9-10 membered heteroaryl;
Ring moiety B is phenyl, naphthyl, 5-6 membered heteroaryl, 9-10 membered

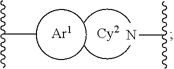

heteroaryl, or;
Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{al1}, SR^{al1}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}S(O)₂R^{b11}, and S(O)₂NR^{c11}R^{d11};
each R^{a11}, R^{c11}, and R^{d11} is independently selected from H, C₁₋₆ alkyl, and C₁₋₆ haloalkyl;
each R^{b11} is independently selected from C₁₋₆ alkyl and C₁₋₆ haloalkyl;
R² is selected from H, D, halo, CN, C₁₋₆ alkyl, C₂₋₆ alkenyl, C₂₋₆ alkynyl, C₁₋₆ haloalkyl, and C₃₋₅ cycloalkyl, wherein said C₁₋₆ alkyl, C₂₋₆ alkenyl, C₂₋₆ alkynyl, C₁₋₆ haloalkyl, and C₃₋₅ cycloalkyl are each optionally substituted with 1, 2, 3, or 4 independently selected R^{2A} substituents;
each R^{2A} is independently selected from D, halo, CN, C₁₋₆ alkyl, C₂₋₆ alkenyl, C₂₋₆ alkynyl, C₁₋₆ haloalkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a211}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21};
each R^{a21}, R^{c21}, and R^{d21} is independently selected from H, C₁₋₆ alkyl, and C₁₋₆ haloalkyl;
each R^{b21} is independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, and C₁₋₆ haloalkyl;
R⁴ is selected from H, D, halo, CN, C₁₋₆ alkyl, C₂₋₆ alkenyl, C₂₋₆ alkynyl, and C₁₋₆ haloalkyl;
R⁵ is independently selected from D, halo, CN, C₁₋₆ alkyl, C₂₋₆ alkenyl, C₂₋₆ alkynyl, C₁₋₆ haloalkyl, C₃₋₄ cycloalkyl, OR^{a5} and NR^{c5}R^{d5}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{5A} substituents;
each R^{a5}, R^{c}, and R^{d5} is independently selected from C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents;
each R^{5A} is independently selected from OH, CN, halo, C₁₋₃ alkyl, C₁₋₃ haloalkyl, amino, C₁₋₃ alkylamino, and di(C₁₋₃ alkyl)amino;
each R⁶ is independently selected from H, D, halo, CN, C₁₋₆ alkyl, C₂₋₆ alkenyl, C₂₋₆ alkynyl, C₁₋₆ haloalkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}S(O)₂R^{b6}, and S(O)₂NR^{c6}R^{d6}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents;
each R⁶⁰ is independently selected from H, C₁₋₆ alkyl, and C₁₋₆ haloalkyl;
or R⁶ and R⁶⁰, together with the carbon atom to which they are attached, form a 4-10 membered heterocycloalkyl moiety, which is optionally substituted with 1, 2, 3, or 4 independently selected R^{G} substituents;
each R^{a6}, R^{c6}, and R^{d6} is independently selected from H, C₁₋₆ alkyl, C₁₋₆ haloalkyl, C₂₋₆ alkenyl, and C₂₋₆ alkynyl, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, and C₂₋₆ alkynyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, OR^{a61}, SR^{a61}, C(O)R^{b61}, C(O)NR^{c61}R^{d61}, C(O)OR^{a11}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61};
each R^{a61}, R^{c61}, and R^{d61} is independently selected from H, C₁₋₆ alkyl, and C₁₋₆ haloalkyl;
each R^{b61} is independently selected from C₁₋₆ alkyl and C₁₋₆ haloalkyl;
R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d71};
each R^{a71}, R^{c7}, and R^{d71} is independently selected from H, C₁₋₆ alkyl, and C₁₋₆ haloalkyl;
each R^{b7} is independently selected from C₁₋₆ alkyl and C₁₋₆ haloalkyl;
R^{W} is selected from:

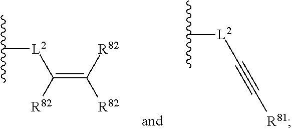

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, OR^{a8}, C(O)R^{b8}, C(O)NR^{c8}R^{d8}, and C(O)OR^{a8}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each R^{a8}, R^{c8}, and R^{d8} is independently selected from H and C₁₋₆ alkyl;
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl;
each R^{b10} is independently selected from C₁₋₆ alkyl and C₁₋₆ haloalkyl; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

Embodiment 123. The compound of embodiment 1, or a pharmaceutically acceptable salt thereof, wherein:
n is 0, 1, 2, 3, or 4;
p is 0, 1, 2, 3, or 4;
m is 1; s is 1; and Y¹ is a bond; or
m is 1; s is 1; and Y¹ is —CR⁶R⁶⁰—, —(CR⁶R⁶⁰)₂—, or C(O); or
m is 0; s is 1; and Y¹ is —CR⁶R⁶⁰—, —(CR⁶R⁶⁰)₂—, or C(O); Cy¹ is an azetidine ring, a pyrrolidine ring, or a piperidine ring, which are each optionally substituted by 1 or 2 independently selected R¹⁰ substituents;
Ring moiety A is phenyl, 5-6 membered heteroaryl, or 9-10 membered heteroaryl;
Ring moiety B is phenyl, naphthyl, 5-6 membered heteroaryl, 9-10 membered heteroaryl, or;

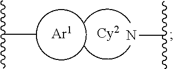

Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, OR^{a11}, SR^{a11}, C(O)R^{b11}, C(O)NR^{c11}R^{d1}, C(O)OR^{a11}, OC(O)R^{b11}, C(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11} is independently selected from H, C₁₋₆ alkyl, and C₁₋₆ haloalkyl;
each R^{b11} is independently selected from C₁₋₆ alkyl and C₁₋₆ haloalkyl;
R² is selected from H, D, halo, CN, C₁₋₆ alkyl, C₂₋₆ alkenyl, C₂₋₆ alkynyl, and C₁₋₆ haloalkyl;
R³ is selected from H, D, halo, CN, C₁₋₆ alkyl, C₂₋₆ alkenyl, C₂₋₆ alkynyl, and C₁₋₆ haloalkyl;
R⁴ is selected from H, D, halo, CN, C₁₋₆ alkyl, C₂₋₆ alkenyl, C₂₋₆ alkynyl, and C₁₋₆ haloalkyl;
each R⁵ is independently selected from halo, CN, C₁₋₃ alkyl, C₁₋₃ haloalkyl, OH, C₁₋₃ alkoxy, and C₁₋₃ haloalkoxy;
each R⁶ is independently selected from H, D, halo, CN, C₁₋₆ alkyl, and C₁₋₆ haloalkyl;
each R⁶⁰ is independently selected from H and C₁₋₃ alkyl;
or R⁶ and R⁶⁰, together with the carbon atom to which they are attached, form a C₃₋₇ cycloalkyl moiety optionally substituted with 1 or 2 independently selected R^{7A} substituents;
each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c7}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from:

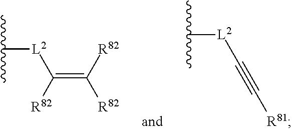

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, and C(O)OR^{a8}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each R^{a8} is independently selected from H and C₁₋₆ alkyl; and
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b11}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl; and
each R¹⁰ is independently selected from C₁₋₆ alkyl and C₁₋₆ haloalkyl.

Embodiment 124. The compound of embodiment 1, or a pharmaceutically acceptable salt thereof, wherein:
n is 0, 1, 2, 3, or 4;
p is 0, 1, 2, 3, or 4;
m is 1; s is 1; and Y¹ is a bond; or
m is 1; s is 1; and Y¹ is —CR⁶R⁶⁰— or C(O); or
m is 0; s is 1; and Y¹ is —CR⁶R⁶⁰— or C(O); Cy¹ is an azetidine ring, a pyrrolidine ring, or a piperidine ring, which are each optionally substituted by 1 or 2 independently selected R¹⁰ substituents;
Ring moiety A is phenyl, 5-6 membered heteroaryl, or 9-10 membered heteroaryl;
Ring moiety B is phenyl, naphthyl, 5-6 membered heteroaryl, 9-10 membered heteroaryl, or

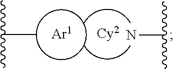

Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, OR^{a11}, SR^{a11}, C(O)R^{b11}, C(O)NR^{c11}R^{d1}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11} is independently selected from H, C₁₋₆ alkyl, and C₁₋₆ haloalkyl;
each R^{b11} is independently selected from C₁₋₆ alkyl and C₁₋₆ haloalkyl;
R² is selected from H, D, halo, CN, C₁₋₆ alkyl, C₂₋₆ alkenyl, C₂₋₆ alkynyl, and C₁₋₆ haloalkyl;
R³ is selected from H, D, halo, CN, C₁₋₆ alkyl, C₂₋₆ alkenyl, C₂₋₆ alkynyl, and C₁₋₆ haloalkyl;
R⁴ is selected from H, D, halo, CN, C₁₋₆ alkyl, C₂₋₆ alkenyl, C₂₋₆ alkynyl, and C₁₋₆ haloalkyl;
each R⁵ is independently selected from halo, CN, C₁₋₃ alkyl, C₁₋₃ haloalkyl, OH, C₁₋₃ alkoxy, and C₁₋₃ haloalkoxy;
each R⁶ is independently selected from H, D, halo, CN, C₁₋₆ alkyl, and C₁₋₆ haloalkyl;
each R⁶⁰ is independently selected from H and C₁₋₃ alkyl;
each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from:

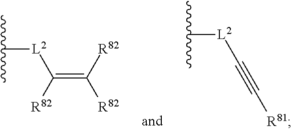

L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, and C(O)OR^{a8}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each R^{a}s is independently selected from H and C₁₋₆ alkyl; and
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b11}, NR^{c10}C(O)OR^{a10}, NR^{c10}C(O)NR^{c10}R^{d10}, NR^{c10}S(O)₂R^{b11}, NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl; and
each R^{b10} is independently selected from C₁₋₆ alkyl and C₁₋₆ haloalkyl.

Embodiment 125. The compound of any one of embodiments 119-124, or a pharmaceutically acceptable salt thereof, wherein:

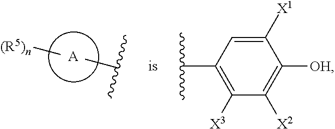

wherein X¹ and X² are each independently Cl or F, and X³ is H, Cl or F; or

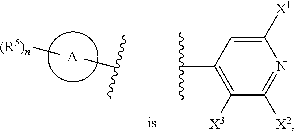

wherein X¹ and X² are each independently Cl, F, OH, and OCH₃; and X³ is H, Cl, or F.

Embodiment 126. The compound of any one of embodiments 119-124, or a pharmaceutically acceptable salt thereof, wherein:

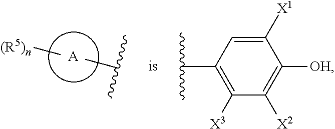

wherein X¹ and X² are each independently Cl or F, and X³ is H, Cl or F.

Embodiment 127. The compound of any one of embodiments 119-124, or a pharmaceutically acceptable salt thereof, wherein:

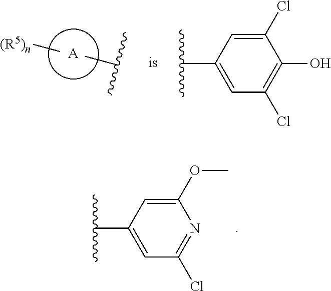

Embodiment 128. The compound of any one of embodiments 119-124, or a pharmaceutically acceptable salt thereof, wherein:

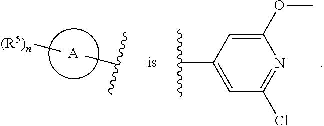

Embodiment 129. The compound of any one of embodiments 119-124, or a pharmaceutically acceptable salt thereof, wherein:

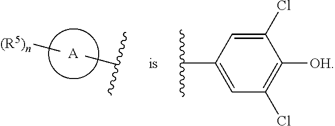

Embodiment 130. The compound of any one of embodiments 119-124, or a pharmaceutically acceptable salt thereof, wherein:

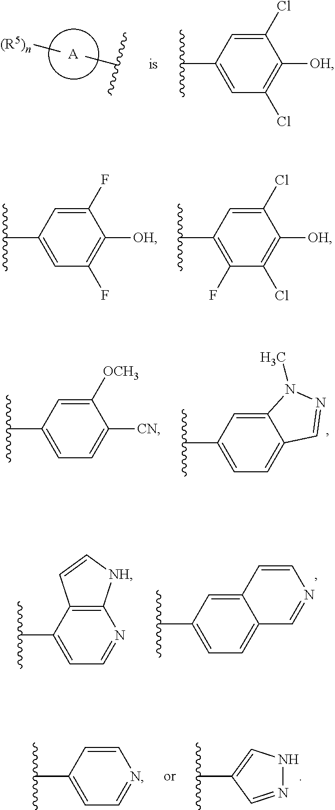

Embodiment 131. The compound of any one of embodiments 119-124, or a pharmaceutically acceptable salt thereof, wherein:

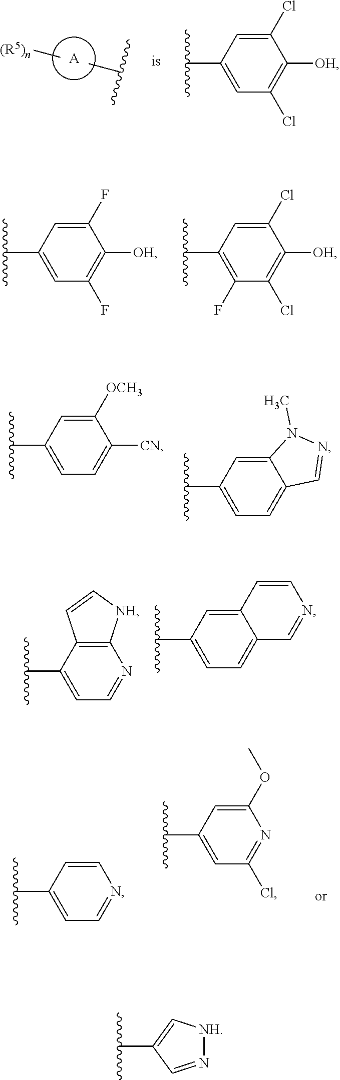

Embodiment 132. The compound of any one of embodiments 119-124, or a pharmaceutically acceptable salt thereof, wherein:

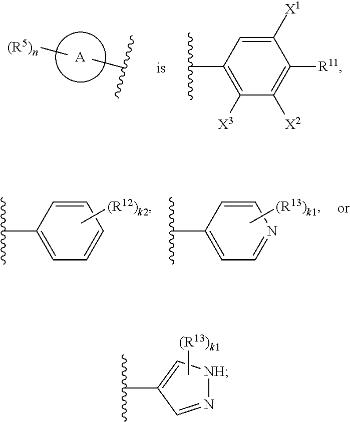

X¹ is Cl, F, or OCH₃;
X² is H, Cl, or F;
X³ is H, Cl or F;
R¹¹ is OH or CN;
k1 is 0, 1, or 2;
k2 is 2;
two R¹² substituents, together with the carbon atoms to which they are attached, form a pyrazole, pyrrole, or pyrimidine ring, which is optionally substituted by 1 or 2 independently selected R⁵ substituents selected from halo, CN, and C₁₋₃ alkyl; and
each R¹³ is independently selected from halo, CN, C₁₋₃ alkyl, and C₁₋₃ alkoxy.

Embodiment 133. The compound of any one of embodiments 119-124, or a pharmaceutically acceptable salt thereof, wherein:

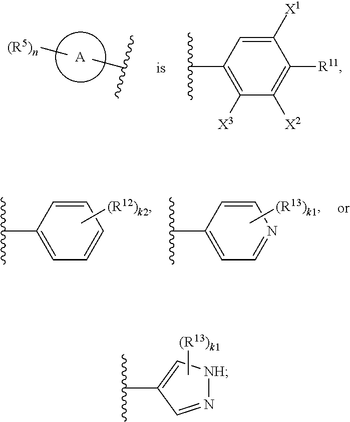

X¹ is Cl, F, or OCH₃;
X² is H, Cl, or F;
X³ is H, Cl or F;
R¹¹ is OH or CN;
k1 is 0, 1, or 2;
k2 is 2;
two R¹² substituents, together with the carbon atoms to which they are attached, form a pyrazole, pyrrole, or pyrimidine ring, which is optionally substituted by 1 or 2 independently selected R⁵ substituents selected from halo, CN, and C₁₋₃ alkyl; and
each R¹³ is independently selected from halo, CN, and C₁₋₃ alkyl.

Embodiment 134. The compound of any one of embodiments 1-21, 23-29, and 31-133, wherein the compound is a compound of Formula (II):

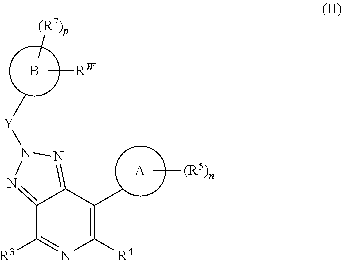

or a pharmaceutically acceptable salt thereof.

Embodiment 135. The compound of any one of embodiments 1-20, 22-29, and 31-133, wherein the compound is a compound of Formula (III):

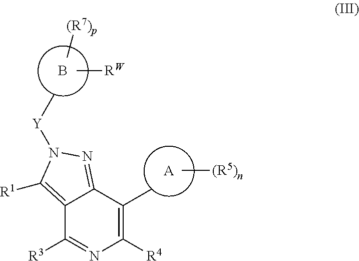

or a pharmaceutically acceptable salt thereof.

Embodiment 136. The compound of any one of embodiments 1-21, 23-28, and 30-133, wherein the compound is a compound of Formula (IV):

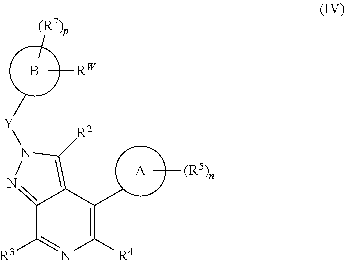

or a pharmaceutically acceptable salt thereof.

Embodiment 137. The compound of any one of embodiments 1-21, 23-29, 31-50, and 54-133, wherein the compound is a compound of Formula (V):

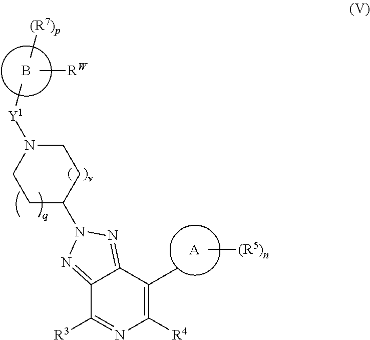

or a pharmaceutically acceptable salt thereof, wherein q is 0 or 1; and v is 0 or 1.

Embodiment 138. The compound of any one of embodiments 1-20, 22-28, 31-50, and 54-133, wherein the compound is a compound of Formula (VI):

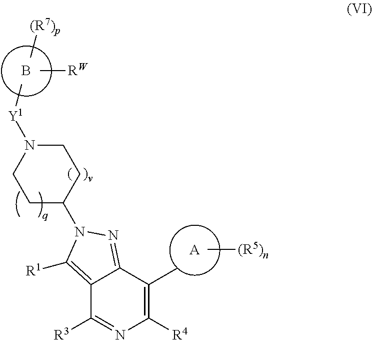

or a pharmaceutically acceptable salt thereof, wherein q is 0 or 1; and v is 0 or 1.

Embodiment 139. The compound of any one of embodiments 1-21, 23-28, 30-50, and 54-133, wherein the compound is a compound of Formula (VII):

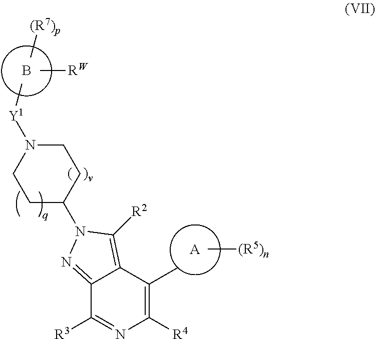

or a pharmaceutically acceptable salt thereof, wherein q is 0 or 1; and v is 0 or 1.

Embodiment 140. The compound of embodiment 1, or a pharmaceutically acceptable salt thereof, wherein the compound is selected from:
N-(4-(3-(7-(3,5-dichloro-4-hydroxyphenyl)-2H-[1,2,3]triazolo[4,5-c]pyridin-2-yl)azetidine-1-carbonyl)phenyl)acrylamide;
(S)—N-(4-(3-(7-(3,5-dichloro-4-hydroxyphenyl)-2H-[1,2,3]triazolo[4,5-c]pyridin-2-yl)pyrrolidine-1-carbonyl)phenyl)acrylamide;
rac-N-(4-(3-(7-(3,5-dichloro-4-hydroxyphenyl)-2H-[1,2,3]triazolo[4,5-c]pyridin-2-yl)piperidine-1-carbonyl)phenyl)acrylamide;
rac-N-(4-((2R,4S)-4-(7-(3,5-dichloro-4-hydroxyphenyl)-2H-[1,2,3]triazolo[4,5-c]pyridin-2-yl)-2-methylpyrrolidine-1-carbonyl)phenyl)acrylamide;
(S)—N-(4-(3-(7-(3,5-dichloro-4-hydroxyphenyl)-2H-[1,2,3]triazolo[4,5-c]pyridin-2-yl)pyrrolidine-1-carbonyl)-2-methoxyphenyl)acrylamide;
rac-N-(4-(3-(7-(3,5-dichloro-4-hydroxyphenyl)-3-(1-methyl-2-oxo-1,2-dihydropyridin-4-yl)-2H-pyrazolo[4,3-c]pyridin-2-yl)pyrrolidine-1-carbonyl)phenyl)acrylamide;
N-(4-((7-(3,5-dichloro-4-hydroxyphenyl)-2H-[1,2,3]triazolo[4,5-c]pyridin-2-yl)methyl)phenyl)acrylamide;
1-(5-((7-(3,5-dichloro-4-hydroxyphenyl)-2H-[1,2,3]triazolo[4,5-c]pyridin-2-yl)methyl)isoindolin-2-yl)prop-2-en-1-one;
rac-1-(5-(1-(7-(3,5-dichloro-4-hydroxyphenyl)-2H-[1,2,3]triazolo[4,5-c]pyridin-2-yl)ethyl)isoindolin-2-yl)prop-2-en-1-one;
1-(5-((4-(3,5-dichloro-4-hydroxyphenyl)-2H-pyrazolo[3,4-c]pyridin-2-yl)methyl)isoindolin-2-yl)prop-2-en-1-one;
1-(5-((7-(3,5-dichloro-4-hydroxyphenyl)-2H-pyrazolo[4,3-c]pyridin-2-yl)methyl)isoindolin-2-yl)prop-2-en-1-one;
(E)-2-(5-((7-(3,5-dichloro-4-hydroxyphenyl)-2H-[1,2,3]triazolo[4,5-c]pyridin-2-yl)methyl)isoindoline-2-carbonyl)-4-methylpent-2-enenitrile;
1-(5-((7-(3,5-dichloro-4-hydroxyphenyl)-2H-[1,2,3]triazolo[4,5-c]pyridin-2-yl)methyl)isoindolin-2-yl)but-2-yn-1-one;
1-(5-((7-(3,5-dichloro-4-hydroxyphenyl)-2H-[1,2,3]triazolo[4,5-c]pyridin-2-yl)methyl)isoindolin-2-yl)-2-fluoroprop-2-en-1-one;
(E)-1-(5-((7-(3,5-dichloro-4-hydroxyphenyl)-2H-[1,2,3]triazolo[4,5-c]pyridin-2-yl)methyl)isoindolin-2-yl)-4-(1H-1,2,4-triazol-1-yl)but-2-en-1-one;
(E)-1-(5-((7-(3,5-dichloro-4-hydroxyphenyl)-2H-[1,2,3]triazolo[4,5-c]pyridin-2-yl)methyl)isoindolin-2-yl)-3-(pyridin-4-yl)prop-2-en-1-one;
(E)-1-(5-((7-(3,5-dichloro-4-hydroxyphenyl)-2H-[1,2,3]triazolo[4,5-c]pyridin-2-yl)methyl)isoindolin-2-yl)-4,4,4-trifluorobut-2-en-1-one;
(E)-1-(5-((7-(3,5-dichloro-4-hydroxyphenyl)-2H-[1,2,3]triazolo[4,5-c]pyridin-2-yl)methyl)isoindolin-2-yl)-4-hydroxybut-2-en-1-one;
methyl (E)-4-(5-((7-(3,5-dichloro-4-hydroxyphenyl)-2H-[1,2,3]triazolo[4,5-c]pyridin-2-yl)methyl)isoindolin-2-yl)-4-oxobut-2-enoate;
(E)-1-(5-((7-(3,5-dichloro-4-hydroxyphenyl)-2H-pyrazolo[4,3-c]pyridin-2-yl)methyl)isoindolin-2-yl)-4,4-difluorobut-2-en-1-one;
(E)-4-(azetidin-1-yl)-1-(5-((7-(3,5-dichloro-4-hydroxyphenyl)-2H-[1,2,3]triazolo[4,5-c]pyridin-2-yl)methyl)isoindolin-2-yl)but-2-en-1-one;
1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-b]pyridin-6-yl)prop-2-en-1-one;
(E)-1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-b]pyridin-6-yl)-4-(diethylamino)but-2-en-1-one;
(E)-1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-b]pyridin-6-yl)-4-(piperidin-1-yl)but-2-en-1-one;
1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-d]pyrimidin-6-yl)prop-2-en-1-one; and
(E)-1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-d]pyrimidin-6-yl)-4-(piperidin-1-yl)but-2-en-1-one;
or a pharmaceutically acceptable salt of any of the aforementioned.

Embodiment 141. The compound of embodiment 1, or a pharmaceutically acceptable salt thereof, wherein the compound is selected from:
rac-(E)-1-(2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridin-6-yl)-4-(diethylamino)but-2-en-1-one;
(R,E)-1-(2-(1-(7-(3,5-dichloro-4-hydroxyphenyl)-4-methyl-2H-[1,2,3]triazolo[4,5-c]28lyridine-2-yl)ethyl)-5,7-dihydro-6H-pyrrolo[3,4-b]28lyridine-6-yl)-4-(diethylamino)but-2-en-1-one;
(S,E)-1-(2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridin-6-yl)-4-(diethylamino)but-2-en-1-one;
(E)-1-(2-(2-(7-(3,5-dichloro-4-hydroxyphenyl)-4-methyl-2H-[1,2,3]triazolo[4,5-c]pyridin-2-yl)propan-2-yl)-5,7-dihydro-6H-pyrrolo[3,4-b]pyridin-6-yl)-4-(dimethylamino)but-2-en-1-one;
(S,E)-1-(2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridin-6-yl)-4-(2,2-dimethylpyrrolidin-1-yl)but-2-en-1-one;
rac-1-(2-(1-(7-(3,5-dichloro-4-hydroxyphenyl)-4-methyl-2H-[1,2,3]triazolo[4,5-c]pyridin-2-yl)ethyl)-3-fluoro-5,7-dihydro-6H-pyrrolo[3,4-b]pyridin-6-yl)prop-2-en-1-one;
(E)-1-(2-(1-(7-(3,5-dichloro-4-hydroxyphenyl)-2H-[1,2,3]triazolo[4,5-c]pyridin-2-yl)cyclobutyl)-5,7-dihydro-6H-pyrrolo[3,4-b]pyridin-6-yl)-4-(diethylamino)but-2-en-1-one;
rac-(E)-1-(2-(cyclopropyl(7-(3,5-dichloro-4-hydroxyphenyl)-4-methyl-2H-[1,2,3]triazolo[4,5-c]pyridin-2-yl)methyl)-5,7-dihydro-6H-pyrrolo[3,4-b]pyridin-6-yl)-4-(dimethylamino)but-2-en-1-one;
rac-(E)-1-(2-(1-(7-(3,5-dichloro-4-hydroxyphenyl)-2H-[1,2,3]triazolo[4,5-c]pyridin-2-yl)propan-2-yl)-5,7-dihydro-6H-pyrrolo[3,4-b]pyridin-6-yl)-4-(diethylamino)but-2-en-1-one;
rac-(E)-1-(2-(1-(4-amino-7-(3,5-dichloro-4-hydroxyphenyl)-2H-[1,2,3]triazolo[4,5-c]pyridin-2-yl)ethyl)-5,7-dihydro-6H-pyrrolo[3,4-b]pyridin-6-yl)-4-(2,2-dimethylpyrrolidin-1-yl)but-2-en-1-one;
(E)-7-(3,5-dichloro-4-hydroxyphenyl)-2-((6-(4-(diethylamino)but-2-enoyl)-3-methoxy-6,7-dihydro-5H-pyrrolo[3,4-b]pyridin-2-yl)methyl)-2H-[1,2,3]triazolo[4,5-c]pyridine-4-carbonitrile;
rac-(E)-4-(3-azabicyclo[3.1.1]heptan-3-yl)-1-(3-(1-(7-(3,5-dichloro-4-hydroxyphenyl)-4-ethyl-2H-[1,2,3]triazolo[4,5-c]pyridin-2-yl)ethyl)-2-methoxy-5,7-dihydro-6H-pyrrolo[3,4-b]pyridin-6-yl)but-2-en-1-one;
rac-1-(2-(1-(7-(3,5-dichloro-4-hydroxyphenyl)-4-methoxy-2H-[1,2,3]triazolo[4,5-c]pyridin-2-yl)ethyl)-5,7-dihydro-6H-pyrrolo[3,4-b]pyridin-6-yl)prop-2-en-1-one;
rac-(E)-1-(2-(1-(6-chloro-7-(3,5-dichloro-4-hydroxyphenyl)-2H-[1,2,3]triazolo[4,5-c]pyridin-2-yl)ethyl)-5,7-dihydro-6H-pyrrolo[3,4-b]pyridin-6-yl)-4-(dimethylamino)but-2-en-1-one;
(E)-1-(5-(2-(7-(2-chloro-6-methoxypyridin-4-yl)-4-methyl-2H-[1,2,3]triazolo[4,5-c]pyridin-2-yl)propan-2-yl)indolin-1-yl)-4-(dimethylamino)but-2-en-1-one;
(E)-4-(bis(methyl-d3)amino)-1-(5-((7-(3,5-dichloro-4-hydroxyphenyl)-4-methyl-2H-[1,2,3]triazolo[4,5-c]pyridin-2-yl)methyl)-4-fluoroindolin-1-yl)but-2-en-1-one;
(E)-1-(2-((7-(3,5-dichloro-4-hydroxyphenyl)-4-methyl-2H-[1,2,3]triazolo[4,5-c]pyridin-2-yl)methyl)-3-ethyl-5,7-dihydro-6H-pyrrolo[3,4-b]pyridin-6-yl)-4-(piperidin-1-yl)but-2-en-1-one;
(E)-4-(3-azabicyclo[3.1.1]heptan-3-yl)-1-(2-(2-(7-(3,5-dichloro-4-hydroxyphenyl)-4-methyl-2H-[1,2,3]triazolo[4,5-c]pyridin-2-yl)propan-2-yl)-4-methoxy-5,7-dihydro-6H-pyrrolo[3,4-b]pyridin-6-yl)but-2-en-1-one;
(E)-4-(3-azabicyclo[3.1.1]heptan-3-yl)-1-(2-((7-(3,5-dichloro-4-hydroxyphenyl)-4-methyl-2H-[1,2,3]triazolo[4,5-c]pyridin-2-yl)methyl)-3-methoxy-5,7-dihydro-6H-pyrrolo[3,4-b]pyrazin-6-yl)but-2-en-1-one;
rac-(E)-1-(2-(1-(7-(3,5-dichloro-4-hydroxyphenyl)-4-methyl-2H-[1,2,3]triazolo[4,5-c]pyridin-2-yl)ethyl)-4-methoxy-5,7-dihydro-6H-pyrrolo[3,4-d]pyrimidin-6-yl)-4-(piperidin-1-yl)but-2-en-1-one;
rac-(E)-1-(5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[2,3-c]pyridin-1-yl)-4-(dimethylamino)but-2-en-1-one;
(S,E)-1-(5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[3,2-b]pyridin-1-yl)-4-(dimethylamino)but-2-en-1-one; and
(E)-1-(5-(2-(7-(2-chloro-6-methoxypyridin-4-yl)-4-methyl-2H-[1,2,3]triazolo[4,5-c]pyridin-2-yl)propan-2-yl)-2,3-dihydro-1H-pyrrolo[2,3-c]pyridin-1-yl)-4-(diethylamino)but-2-en-1-one;
   or a pharmaceutically acceptable salt of any of the aforementioned.

Embodiment 142. The compound of any one of embodiments 1-141, wherein the compound is deuterated.

Embodiment 143. A pharmaceutical composition comprising the compound of any one of embodiments 1-142, or a pharmaceutically acceptable salt thereof, and a pharmaceutically acceptable carrier.

Embodiment 144. A method of inhibiting VRK1, comprising contacting the VRK1 with the compound of any one of embodiments 1-142, or a pharmaceutically acceptable salt thereof.

Embodiment 145. A method of inhibiting VRK1 in a patient, comprising administering to the patient a compound of any one of embodiments 1-142, or a pharmaceutically acceptable salt thereof.

Embodiment 146. A method of selectively inhibiting VRK1 over VRK2, comprising contacting the VRK1 and VRK2 with a VRK1 inhibitor, or a pharmaceutically acceptable salt thereof.

Embodiment 147. A method of covalently binding a cysteine in VRK1 with a VRK1 inhibitor, or a pharmaceutically acceptable salt thereof.

Embodiment 148. The method of embodiment 147, further comprising binding with a water in the binding pocket of VRK1.

Embodiment 149. The method of embodiment 147 or 148, further comprising binding with Phe134 of VRK1.

Embodiment 150. The method of any one of embodiments 147-149, further comprising binding with Lys71 of VRK1.

Embodiment 151. The method of any one of embodiments 147-150, further comprising binding with Asp132 of VRK1.

Embodiment 152. The method of any one of embodiments 147-151, further comprising binding with the phosphate-binding loop of VRK1.

Embodiment 153. A method of covalently binding with a cysteine in VRK1, binding with a water in the binding pocket of VRK1, and binding with Phe134 of VRK1 with a VRK1 inhibitor, or a pharmaceutically acceptable salt thereof.

Embodiment 154. A method of covalently binding with a cysteine in VRK1, binding with a water in the binding pocket of VRK1, binding with Phe134 of VRK1, and binding with Lys71 of VRK1 with a VRK1 inhibitor, or a pharmaceutically acceptable salt thereof.

Embodiment 155. A method of covalently binding with a cysteine in VRK1, binding with a water in the binding pocket of VRK1, binding with Phe134 of VRK1, binding with Lys71 of VRK1, and binding with the phosphate-binding loop of VRK1 with a VRK1 inhibitor, or a pharmaceutically acceptable salt thereof.

Embodiment 156. A method of covalently binding with a cysteine in VRK1, binding with a water in the binding pocket of VRK1, binding with Phe134 of VRK1, binding with Asp132 of VRK1, and binding with the phosphate-binding loop of VRK1 with a VRK1 inhibitor, or a pharmaceutically acceptable salt thereof.

Embodiment 157. A method of covalently binding with a cysteine in VRK1, binding with a water in the binding pocket of VRK1, binding with Phe134 of VRK1, binding with Lys71 of VRK1, and binding with Asp132 of VRK1 with a VRK1 inhibitor, or a pharmaceutically acceptable salt thereof.

Embodiment 158. A method of covalently binding with a cysteine in VRK1, binding with a water in the binding pocket of VRK1, binding with Phe134 of VRK1, and binding with Asp132 of VRK1 with a VRK1 inhibitor, or a pharmaceutically acceptable salt thereof.

Embodiment 159. A method of covalently binding with a cysteine in VRK1, binding with a water in the binding pocket of VRK1, binding with Phe134 of VRK1, binding with Lys71 of VRK1, binding with Asp132 of VRK1, and binding with the phosphate-binding loop of VRK1 with a VRK1 inhibitor, or a pharmaceutically acceptable salt thereof.

Embodiment 160. The method of any one of embodiments 146-160, wherein the VRK1 inhibitor, or a pharmaceutically acceptable salt thereof, is a compound of any one of claims 1-142, or a pharmaceutically acceptable salt thereof.

Embodiment 161. A method of treating a disease or disorder associated with VRK1 in a patient, comprising administering to the patient a therapeutically effective amount of the compound of any one of embodiments 1-142, or pharmaceutically acceptable salt thereof.

Embodiment 162. The method of embodiment 161, wherein the disease or disorder is cancer.

Embodiment 163. The method of embodiment 162, wherein the cancer is sarcoma, colorectal adenocarcinoma, acute myeloid leukemia, glioblastoma, and neuroblastoma.

Embodiment 164. The method of embodiment 163, wherein the glioblastoma or neuroblastoma have overexpression of VRK1.

Embodiment 165. The method of embodiment 163, wherein the glioblastoma or neuroblastoma have underexpression of VRK2.

Embodiment 166. The method of embodiment 162, wherein the cancer is bone cancer, cancer of the neck and head, rectal cancer, esophageal cancer, sarcoma of soft tissue, neoplasm of the central nervous system (CNS), brain stem, glioma, colon cancer, Ewing's sarcoma, brain cancer, astrocytoma, neuroblastoma, intestinal cancer, nervous system cancers, osteosarcoma, cancer of the small bowel, adenocarcinoma of the small bowel, lymphoma of the small bowel, carcinoid tumors of the small bowel, Kaposi's sarcoma of the small bowel, leiomyoma of the small bowel, hemangioma of the small bowel, lipoma of the small bowel, neurofibroma of the small bowel, fibroma of the small bowel, cancer of the large bowel, adenocarcinoma of the large bowel, tubular adenoma of the large bowel, villous adenoma of the large bowel, hamartoma of the large bowel, or leiomyoma of the large bowel.

### EXAMPLES

Certain embodiments of the present disclosure will be described in greater detail by way of specific examples. The following examples are offered for illustrative purposes and are not intended to limit the present disclosure in any manner. Those of skill in the art will readily recognize a variety of non-critical parameters which can be changed or modified to yield essentially the same results. The compounds of the Examples have been found to inhibit the activity of VRK1 according to at least one assay described herein.

Experimental procedures for compounds of the present disclosure are provided below. Open Access Preparative LCMS Purification of some of the compounds prepared was performed on Waters mass directed fractionation systems. The basic equipment setup, protocols and control software for the operation of these systems have been described in detail in literature. See, e.g., Blom, “Two-Pump At Column Dilution Configuration for Preparative LC-MS”, K. Blom, J. Combi. Chem., 2002, 4, 295-301; Blom et al., “Optimizing Preparative LC-MS Configurations and Methods for Parallel Synthesis Purification”, J. Combi. Chem., 2003, 5, 670-83; and Blom et al., “Preparative LC-MS Purification: Improved Compound Specific Method Optimization”, J. Combi. Chem., 2004, 6, 874-883.

The compounds separated were typically subjected to analytical liquid chromatography mass spectrometry (LCMS) for purity analysis under the following conditions: Instrument=Agilent 1100 series, LC/MSD; Column: Waters Sunfire™ C₁₈ 5 μm, 2.1×50 mm, Buffers: mobile phase A: 0.025% TFA in water and mobile phase B: acetonitrile; gradient 2% to 80% B in 3 min with flow rate 2.0 mL/min.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:
pH=2 purifications: Waters Sunfire™ C₁₈ 5 μm, 30×100 mm or Waters XBridge™ C₁₈ 5 μm, 30×100 mm column, eluting with mobile phase A: 0.1% TFA (trifluoroacetic acid) in water and mobile phase B: acetonitrile; the flow rate was 60 mL/min, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).
pH=10 purifications: Waters XBridge™ C₁₈ 5 μm, 30×100 mm column, eluting with mobile phase A: 0.1% NH₄OH in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

The following abbreviations may be used herein: aq. (aqueous); br (broad); d (doublet); dd (doublet of doublets); DCM (dichloromethane); DMF (N, N-dimethylformamide); Et (ethyl); EtOAc (ethyl acetate); g (gram(s)); h (hrs)); HPLC (high performance liquid chromatography); Hz (hertz); J (coupling constant); LCMS (liquid chromatography-mass spectrometry); m (multiplet); M (molar); MS (Mass spectrometry); Me (methyl); MeCN (acetonitrile); MeOH (methanol); mg (milligram(s)); min. (minutes(s)); mL (milliliter(s)); mmol (millimole(s)); nM (nanomolar); NMR (nuclear magnetic resonance spectroscopy); Ph (phenyl); r.t. (room temperature), s (singlet); t (triplet or tertiary); TBS (tert-butyldimethylsilyl); tert (tertiary); tt (triplet of triplets); TFA (trifluoroacetic acid); THF (tetrahydrofuran); μg (microgram(s)); μL (microliter(s)); μM (micromolar); wt % (weight percent).

### Intermediate 1. 2,6-dichloro-4-(2-(isoindolin-5-ylmethyl)-2H-[1,2,3]triazolo[4,5-c]pyridin-7-yl)phenol

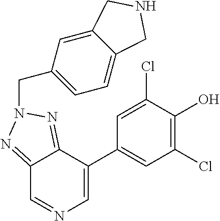 [SMILES: Oc1c(Cl)cc(-c2cncc3nn(Cc4ccc5c(c4)CNC5)nc23)cc1Cl]

### Step 1. tert-butyl 5-((7-bromo-2H-[1,2,3]triazolo[4,5-c]pyridin-2-yl)methyl)isoindoline-2-carboxylate

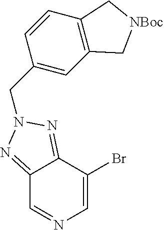 [SMILES: CC(C)(C)OC(=O)N1Cc2ccc(Cn3nc4cncc(Br)c4n3)cc2C1]

A mixture of 7-bromo-3H-[1,2,3]triazolo[4,5-c]pyridine hydrochloride (1 g, 4.25 mmol), tert-butyl 5-(bromomethyl)isoindoline-2-carboxylate (1.99 g, 6.37 mmol) and Cs₂CO₃ (4.15 g, 12.74 mmol) in DMF (20 mL) was heated to 70° C. for 1 h. Upon completion, the resulting mixture was cooled and diluted with water and extracted with ethyl acetate (50 mL). The organic layer was washed with brine, dried with Na₂SO₄, filtered, and concentrated in vacuo. The residue was purified by column chromatography (20-50% AcOEt in DCM). LCMS calculated for C₁₉H₂₁BrN₅O₂(M+H)⁺: m/z=430.1; found: 430.1.

### Step 2. 2,6-dichloro-4-(2-(isoindolin-5-ylmethyl)-2H-[1,2,3]triazolo[4,5-c]pyridin-7-yl)phenol

To a mixture of tert-butyl 5-((7-bromo-2H-[1,2,3]triazolo[4,5-c]pyridin-2-yl)methyl)isoindoline-2-carboxylate (338 mg, 0.785 mmol), (3,5-dichloro-4-hydroxyphenyl)boronic acid (244 mg, 1.178 mmol) and potassium phosphate (500 mg, 2.356 mmol) in 1,4-dioxane (6.5 mL) and water (1.3 mL) was added XPhos-Pd-G4 (67.6 mg, 0.079 mmol). The reaction mixture was then degassed for 10 min before heating to 95° C. After 1 h, the resulting mixture was cooled and diluted with water and extracted with ethyl acetate (50 mL). The organic layer was washed with brine, dried with Na₂SO₄, filtered, and concentrated in vacuo. The obtained crude product was dissolved in 2,2,2-trifluoroacetic acid (5 mL) and stirred for 20 mins. Upon completion, the reaction mixture was diluted with acetonitrile and purified by prep-LCMS (XBridge C18 column, eluting with a gradient of acetonitrile/water containing 0.1% TFA, at flow rate of 60 mL/min) to afford desired product. LCMS calculated for C₂₀H₁₆C₁₂N₅O (M+H)⁺: m/z=412.1; found: 412.1.

Intermediate 2. 2,6-dichloro-4-(2-((6,7-dihydro-5H-pyrrolo[3,4-b]pyridin-2-yl)methyl)-2H-[1,2,3]triazolo[4,5-c]pyridin-7-yl)phenol

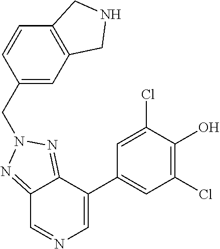 [SMILES: Oc1c(Cl)cc(-c2cncc3nn(Cc4ccc5c(c4)CNC5)nc23)cc1Cl]

### Step 1. tert-butyl 2-((7-bromo-2H-[1,2,3]triazolo[4,5-c]pyridin-2-yl)methyl)-5,7-dihydro-6H-pyrrolo[3,4-b]pyridine-6-carboxylate

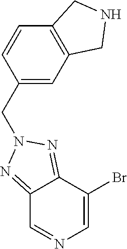 [SMILES: Brc1cncc2nn(Cc3ccc4c(c3)CNC4)nc12]

To a suspension of 7-bromo-3H-[1,2,3]triazolo[4,5-c]pyridine (360 mg, 1.809 mmol), tert-butyl 2-(hydroxymethyl)-5,7-dihydro-6H-pyrrolo[3,4-b]pyridine-6-carboxylate (498 mg, 1.990 mmol) and triphenylphosphine (712 mg, 2.71 mmol) in tetrahydrofuran (10 mL) at 0° C. was added diisopropyl azodicarboxylate (DIAD, 0.53 mL, 2.71 mmol). The suspension became a solution within 10 min. After stirring at 0° C. for 1 h, the resulting mixture was diluted with water and extracted with ethyl acetate (50 mL). The organic layer was washed with brine, dried with Na₂SO₄, filtered, and concentrated in vacuo. The residue was purified by column chromatography (20-50% AcOEt in DCM). LCMS calculated for C₁₈H₂₀BrN₆O₂ (M+H)⁺: m/z=431.1; found: 431.1.

### Step 2. 2,6-dichloro-4-(2-((6,7-dihydro-5H-pyrrolo[3,4-b]pyridin-2-yl)methyl)-2H-[1,2,3]triazolo[4,5-c]pyridin-7-yl)phenol

To a mixture of tert-butyl 2-((7-bromo-2H-[1,2,3]triazolo[4,5-c]pyridin-2-yl)methyl)-5,7-dihydro-6H-pyrrolo[3,4-b]pyridine-6-carboxylate (366 mg, 0.848 mmol), (3,5-dichloro-4-hydroxyphenyl)boronic acid (263 mg, 1.272 mmol) and potassium phosphate (540 mg, 2.54 mmol) was added XPhos-Pd-G4 (73.0 mg, 0.085 mmol). The reaction mixture was then degassed for 10 min before heating to 95° C. After 1 h, the resulting mixture was cooled and diluted with water and extracted with ethyl acetate (50 mL). The organic layer was washed with brine, dried with Na₂SO₄, filtered, and concentrated in vacuo. The obtained crude product was dissolved in 2,2,2-trifluoroacetic acid (5 mL) and stirred for 20 mins. Upon completion, the reaction mixture was diluted with acetonitrile and purified by prep-LCMS (XBridge C18 column, eluting with a gradient of acetonitrile/water containing 0.1% TFA, at flow rate of 60 mL/min) to afford desired product. LCMS calculated for C₁₉H₁₅C₁₂N₆O (M+H)⁺: m/z=413.1; found: 413.1.

Intermediate 3. 2,6-dichloro-4-(2-((6,7-dihydro-5H-pyrrolo[3,4-d]pyrimidin-2-yl)methyl)-2H-[1,2,3]triazolo[4,5-c]pyridin-7-yl)phenol

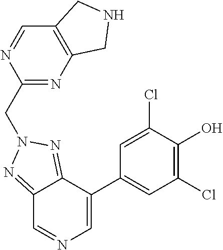 [SMILES: Oc1c(Cl)cc(-c2cncc3nn(Cc4ncc5c(n4)CNC5)nc23)cc1Cl]

### Step 1. tert-butyl 2-((7-bromo-2H-[1,2,3]triazolo[4,5-c]pyridin-2-yl)methyl)-5,7-dihydro-6H-pyrrolo[3,4-d]pyrimidine-6-carboxylate

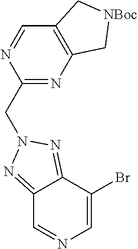 [SMILES: CC(C)(C)OC(=O)N1Cc2cnc(Cn3nc4cncc(Br)c4n3)nc2C1]

A solution of 7-bromo-3H-[1,2,3]triazolo[4,5-c]pyridine hydrochloride (150 mg, 0.637 mmol), tert-butyl 2-(chloromethyl)-5,7-dihydro-6H-pyrrolo[3,4-d]pyrimidine-6-carboxylate (258 mg, 0.956 mmol), tert-butyl 2-(chloromethyl)-5,7-dihydro-6H-pyrrolo[3,4-d]pyrimidine-6-carboxylate (258 mg, 0.956 mmol) and DIPEA (0.334 mL, 1.911 mmol) in DMF (6 mL) was heated to 70° C. and stirred for 2 h. Upon completion, the resulting mixture was cooled and diluted with water and extracted with ethyl acetate (50 mL). The organic layer was washed with brine, dried with Na₂SO₄, filtered, and concentrated in vacuo. The residue was purified by column chromatography (20-50% AcOEt in DCM). LCMS calculated for C₁₇H₁₉BrN₇O₂(M+H)⁺: m/z=432.1; found: 432.1.

### Step 2. 2,6-dichloro-4-(2-((6,7-dihydro-5H-pyrrolo[3,4-d]pyrimidin-2-yl)methyl)-2H-[1,2,3]triazolo[4,5-c]pyridin-7-yl)phenol

To a mixture of tert-butyl 2-((7-bromo-2H-[1,2,3]triazolo[4,5-c]pyridin-2-yl)methyl)-5,7-dihydro-6H-pyrrolo[3,4-d]pyrimidine-6-carboxylate (40.7 mg, 0.094 mmol), (3,5-dichloro-4-hydroxyphenyl)boronic acid (23.4 mg, 0.113 mmol) and potassium phosphate (60 mg, 0.283 mmol) in 1,4-dioxane (6.5 mL) and water (1.3 mL) was added XPhos-Pd-G4 (8.1 mg, 9.42 μmol). The reaction mixture was then degassed for 10 min before heating to 90° C. After 1 h, the resulting mixture was cooled and diluted with water and extracted with ethyl acetate (50 mL). The organic layer was washed with brine, dried with Na₂SO₄, filtered, and concentrated in vacuo. The obtained crude product was dissolved in 2,2,2-trifluoroacetic acid (5 mL) and stirred for 20 mins. Upon completion, the reaction mixture was diluted with acetonitrile and purified by prep-LCMS (XBridge C18 column, eluting with a gradient of acetonitrile/water containing 0.1% TFA, at flow rate of 60 mL/min) to afford desired product. LCMS calculated for C₁₈H₁₄C₁₂N₇O (M+H)⁺: m/z=414.1; found: 414.1.

### Intermediate 4. 2,6-dichloro-4-(2-(isoindolin-5-ylmethyl)-2H-pyrazolo[3,4-c]pyridin-4-yl)phenol

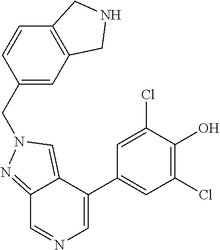 [SMILES: Oc1c(Cl)cc(-c2cncc3nn(Cc4ccc5c(c4)CNC5)cc23)cc1Cl]

### Step 1. tert-butyl 5-((4-bromo-2H-pyrazolo[3,4-c]pyridin-2-yl)methyl)isoindoline-2-carboxylate

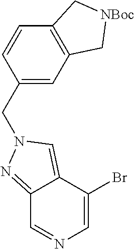 [SMILES: CC(C)(C)OC(=O)N1Cc2ccc(Cn3cc4c(Br)cncc4n3)cc2C1]

A mixture of 4-bromo-1H-pyrazolo[3,4-c]pyridine (200 mg, 1.010 mmol), tert-butyl 5-(bromomethyl)isoindoline-2-carboxylate (473 mg, 1.515 mmol) and Cs₂CO₃ (987 mg, 3.03 mmol) in DMF (6 mL) was heated to 70° C. and stirred for 2 h. Upon completion, the resulting mixture was cooled and diluted with water and extracted with ethyl acetate (50 mL). The organic layer was washed with brine, dried with Na₂SO₄, filtered, and concentrated in vacuo. The residue was purified by column chromatography (0-60% AcOEt in DCM). LCMS calculated for C₂₀H₂₂BrN₄O₂(M+H)⁺: m/z=429.1; found: 429.1.

### Step 2. 2,6-dichloro-4-(2-(isoindolin-5-ylmethyl)-2H-pyrazolo[3,4-c]pyridin-4-yl)phenol

To a mixture of tert-butyl 5-((4-bromo-2H-pyrazolo[3,4-c]pyridin-2-yl)methyl)isoindoline-2-carboxylate (22.9 mg, 0.053 mmol), (3,5-dichloro-4-hydroxyphenyl)boronic acid (13.2 mg, 0.064 mmol) and potassium phosphate (34 mg, 0.160 mmol) in 1,4-dioxane (0.5 mL) and water (0.1 mL) was added XPhos-Pd-G4 (4.6 mg, 5.34 μmol). The reaction mixture was then degassed for 10 min before heating to 90° C. After 1 h, the resulting mixture was cooled and diluted with water and extracted with ethyl acetate (10 mL). The organic layer was washed with brine, dried with Na₂SO₄, filtered, and concentrated in vacuo. The obtained crude product was dissolved in 2,2,2-trifluoroacetic acid (1 mL) and stirred for 20 mins. Upon completion, the reaction mixture was diluted with acetonitrile and purified by prep-LCMS (XBridge C18 column, eluting with a gradient of acetonitrile/water containing 0.1% TFA, at flow rate of 60 mL/min) to afford desired product. LCMS calculated for C₂₁H₁₇C₁₂N₄O (M+H)⁺: m/z=411.1; found: 411.1.

### Intermediate 5. 2,6-dichloro-4-(2-(isoindolin-5-ylmethyl)-2H-pyrazolo[4,3-c]pyridin-7-yl)phenol

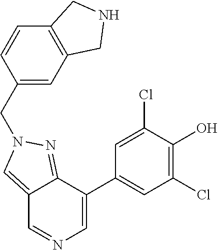 [SMILES: Oc1c(Cl)cc(-c2cncc3cn(Cc4ccc5c(c4)CNC5)nc23)cc1Cl]

### Step 1. tert-butyl 5-((7-bromo-2H-pyrazolo[4,3-c]pyridin-2-yl)methyl)isoindoline-2-carboxylate

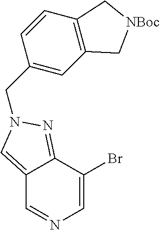 [SMILES: CC(C)(C)OC(=O)N1Cc2ccc(Cn3cc4cncc(Br)c4n3)cc2C1]

A mixture of 7-bromo-1H-pyrazolo[4,3-c]pyridine (200 mg, 1.010 mmol), tert-butyl 5-(bromomethyl)isoindoline-2-carboxylate (473 mg, 1.515 mmol) and Cs₂CO₃ (987 mg, 3.03 mmol) in DMF (6 mL) was heated to 70° C. and stirred for 2 h. Upon completion, the resulting mixture was cooled and diluted with water and extracted with ethyl acetate (50 mL). The organic layer was washed with brine, dried with Na₂SO₄, filtered, and concentrated in vacuo. The residue was purified by column chromatography (20-50% AcOEt in DCM). LCMS calculated for C₂₀H₂₂BrN₄O₂(M+H)⁺: m/z=429.1; found: 429.1.

### Step 2. 2,6-dichloro-4-(2-(isoindolin-5-ylmethyl)-2H-pyrazolo[4,3-c]pyridin-7-yl)phenol

To a mixture of tert-butyl 5-((7-bromo-2H-pyrazolo[4,3-c]pyridin-2-yl)methyl)isoindoline-2-carboxylate (22.9 mg, 0.053 mmol) in 1,4-dioxane (0.5 mL) and water (0.1 mL) was added (3,5-dichloro-4-hydroxyphenyl)boronic acid (13.2 mg, 0.064 mmol), potassium phosphate (34 mg, 0.160 mmol) and XPhos-Pd-G4 (4.6 mg, 5.34 μmol). The reaction mixture was then degassed for 10 min before heating to 90° C. After 1 h, the resulting mixture was cooled and diluted with water and extracted with ethyl acetate (10 mL). The organic layer was washed with brine, dried with Na₂SO₄, filtered, and concentrated in vacuo. The obtained crude product was dissolved in 2,2,2-trifluoroacetic acid (1 mL) and stirred for 20 mins. Upon completion, the reaction mixture was diluted with acetonitrile and purified by prep-LCMS (XBridge C18 column, eluting with a gradient of acetonitrile/water containing 0.1% TFA, at flow rate of 60 mL/min) to afford desired product. LCMS calculated for C₂₁H₁₇C₁₂N₄O (M+H)⁺: m/z=411.1; found: 411.1.

### Intermediate 6. Rac-2,6-dichloro-4-(2-(1-(isoindolin-5-yl)ethyl)-2H-[1,2,3]triazolo[4,5-c]pyridin-7-yl)phenol

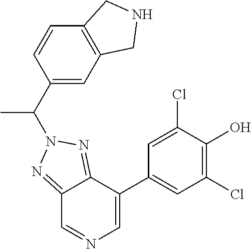 [SMILES: CC(c1ccc2c(c1)CNC2)n1nc2cncc(-c3cc(Cl)c(O)c(Cl)c3)c2n1]

### Step 1. Rac-tert-butyl 5-(1-hydroxyethyl)isoindoline-2-carboxylate

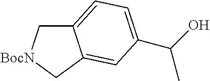 [SMILES: CC(O)c1ccc2c(c1)CN(C(=O)OC(C)(C)C)C2]

To a solution of tert-butyl 5-formylisoindoline-2-carboxylate (200 mg, 0.809 mmol) in THF (8 mL) at −78° C. was added methylmagnesium bromide (3M in THF, 0.809 mL, 2.426 mmol). The mixture was stirred at −78° C. for 1 h. Upon completion, the resulting mixture was quenched with aqueous NH₄Cl and extracted with ethyl acetate (50 mL). The organic layer was washed with brine, dried with Na₂SO₄, filtered, and concentrated in vacuo. The residue was purified by column chromatography (20-50% AcOEt in hexane). LCMS calculated for C₁₅H₂₂NO₃ (M+H)⁺: m/z=264.2; found: 264.2.

### Step 2. Rac-tert-butyl 5-(1-(7-bromo-2H-[1,2,3]triazolo[4,5-c]pyridin-2-yl)ethyl)isoindoline-2-carboxylate

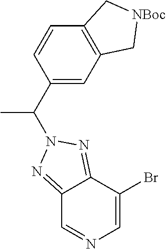 [SMILES: CC(c1ccc2c(c1)CN(C(=O)OC(C)(C)C)C2)n1nc2cncc(Br)c2n1]

To a suspension of 7-bromo-3H-[1,2,3]triazolo[4,5-c]pyridine hydrochloride (100 mg, 0.425 mmol), DIPEA (0.148 mL, 0.849 mmol), tert-butyl 5-(1-hydroxyethyl)isoindoline-2-carboxylate (112 mg, 0.425 mmol) and triphenylphosphine (223 mg, 0.849 mmol) in THF (2 mL) at 0° C. was added diisopropyl azodicarboxylate (DIAD, 0.165 mL, 0.849 mmol). The suspension became a solution within 10 min. After stirring at 0° C. for 1 h, the resulting mixture was diluted with water and extracted with ethyl acetate (50 mL). The organic layer was washed with brine, dried with Na₂SO₄, filtered, and concentrated in vacuo. The residue was purified by column chromatography (20-50% AcOEt in DCM). LCMS calculated for C₂₀H₂₃BrN₅O₂(M+H)⁺: m/z=444.1; found: 444.1.

### Step 3. Rac-2,6-dichloro-4-(2-(1-(isoindolin-5-yl)ethyl)-2H-[1,2,3]triazolo[4,5-c]pyridin-7-yl)phenol

To a mixture of tert-butyl 5-(1-(7-bromo-2H-[1,2,3]triazolo[4,5-c]pyridin-2-yl)ethyl)isoindoline-2-carboxylate (98 mg, 0.220 mmol) in 1,4-dioxane (5 mL) and water (1 mL) was added (3,5-dichloro-4-hydroxyphenyl)boronic acid (68.2 mg, 0.330 mmol), potassium phosphate (140 mg, 0.660 mmol) and XPhos-Pd-G4 (18.9 mg, 0.022 mmol). The reaction mixture was then degassed for 10 min before heating to 90° C. After 1 h, the resulting mixture was cooled and diluted with water and extracted with ethyl acetate (10 mL). The organic layer was washed with brine, dried with Na₂SO₄, filtered, and concentrated in vacuo. The obtained crude product was dissolved in 2,2,2-trifluoroacetic acid (3 mL) and stirred for 20 mins. Upon completion, the reaction mixture was diluted with acetonitrile and purified by prep-LCMS (XBridge C18 column, eluting with a gradient of acetonitrile/water containing 0.1% TFA, at flow rate of 60 mL/min) to afford desired product. LCMS calculated for C₂₁H₁₈C₁₂N₅O (M+H)⁺: m/z=426.1; found: 426.1.

### Intermediate 7. 4-(2-(4-aminobenzyl)-2H-[1,2,3]triazolo[4,5-c]pyridin-7-yl)-2,6-dichlorophenol

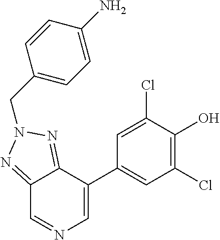 [SMILES: Nc1ccc(Cn2nc3cncc(-c4cc(Cl)c(O)c(Cl)c4)c3n2)cc1]

### Step 1. tert-butyl (4-((7-bromo-2H-[1,2,3]triazolo[4,5-c]pyridin-2-yl)methyl)phenyl)carbamate

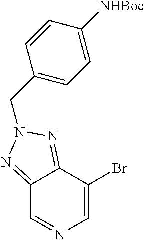 [SMILES: CC(C)(C)OC(=O)Nc1ccc(Cn2nc3cncc(Br)c3n2)cc1]

A mixture of 7-bromo-3H-[1,2,3]triazolo[4,5-c]pyridine hydrochloride (100 mg, 0.425 mmol), tert-butyl (4-(bromomethyl)phenyl)carbamate (182 mg, 0.637 mmol) and DIPEA (0.22 mL, 1.274 mmol) in DMF (20 mL) was heated to 70° C. for 1 h. Upon completion, the resulting mixture was cooled and diluted with water and extracted with ethyl acetate (50 mL). The organic layer was washed with brine, dried with Na₂SO₄, filtered, and concentrated in vacuo. The residue was purified by column chromatography (20-50% AcOEt in DCM). LCMS calculated for C₁₇H₁₉BrN₅O₂ (M+H)⁺: m/z=404.1; found: 404.1.

### Step 2. 4-(2-(4-aminobenzyl)-2H-[1,2,3]triazolo[4,5-c]pyridin-7-yl)-2,6-dichlorophenol

To a mixture of tert-butyl (4-((7-bromo-2H-[1,2,3]triazolo[4,5-c]pyridin-2-yl)methyl)phenyl)carbamate (21.6 mg, 0.053 mmol) in 1,4-dioxane (1 mL) and water (0.2 mL) was added (3,5-dichloro-4-hydroxyphenyl)boronic acid (16.6 mg, 0.080 mmol), potassium phosphate (34 mg, 0.160 mmol) and XPhos-Pd-G4 (4.6 mg, 5.34 μmol). The reaction mixture was then degassed for 10 min before heating to 95° C. After 1 h, the resulting mixture was cooled and diluted with water and extracted with ethyl acetate (50 mL). The organic layer was washed with brine, dried with Na₂SO₄, filtered, and concentrated in vacuo. The obtained crude product was dissolved in 2,2,2-trifluoroacetic acid (5 mL) and stirred for 20 mins. Upon completion, the reaction mixture was diluted with acetonitrile and purified by prep-LCMS (XBridge C18 column, eluting with a gradient of acetonitrile/water containing 0.1% TFA, at flow rate of 60 mL/min) to afford desired product. LCMS calculated for C₁₈H₁₄C₁₂N₅O (M+H)⁺: m/z=386.1; found: 386.1.

### Intermediate 8. (S)-2,6-dichloro-4-(2-(pyrrolidin-3-yl)-2H-[1,2,3]triazolo[4,5-c]pyridin-7-yl)phenol

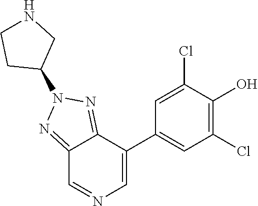 [SMILES: Oc1c(Cl)cc(-c2cncc3nn([C@H]4CCNC4)nc23)cc1Cl]

### Step 1. tert-butyl (S)-3-(7-bromo-2H-[1,2,3]triazolo[4,5-c]pyridin-2-yl)pyrrolidine-1-carboxylate

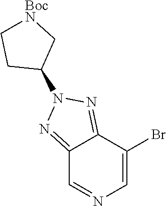 [SMILES: CC(C)(C)OC(=O)N1CC[C@H](n2nc3cncc(Br)c3n2)C1]

To a suspension of 7-bromo-3H-[1,2,3]triazolo[4,5-c]pyridine hydrochloride (50 mg, 0.212 mmol), DIPEA (0.074 mL, 0.425 mmol) and triphenylphosphine (111 mg, 0.425 mmol) in tetrahydrofuran (10 mL) at 0° C. was added diisopropyl azodicarboxylate (DIAD, 0.083 mL, 0.425 mmol). The suspension became a solution within 10 min. After stirring at 0° C. for 1 h, the resulting mixture was diluted with water and extracted with ethyl acetate (10 mL). The organic layer was washed with brine, dried with Na₂SO₄, filtered, and concentrated in vacuo. The residue was purified by column chromatography (20-50% AcOEt in DCM). LCMS calculated for C₁₄H₁₉BrN₅O₂(M+H)⁺: m/z=368.1; found: 368.1.

### Step 2. (S)-2,6-dichloro-4-(2-(pyrrolidin-3-yl)-2H-[1,2,3]triazolo[4,5-c]pyridin-7-yl)phenol

To a mixture of tert-butyl (S)-3-(7-bromo-2H-[1,2,3]triazolo[4,5-c]pyridin-2-yl)pyrrolidine-1-carboxylate (104 mg, 0.283 mmol) in 1,4-eioxane (2.5 mL) and water (0.5 mL) was added (3,5-dichloro-4-hydroxyphenyl)boronic acid (88 mg, 0.424 mmol), potassium phosphate (180 mg, 0.848 mmol) and XPhos-Pd-G4 (24.3 mg, 0.028 mmol). The reaction mixture was then degassed for 10 min before heating to 95° C. After 1 h, the resulting mixture was cooled and diluted with water and extracted with ethyl acetate (50 mL). The organic layer was washed with brine, dried with Na₂SO₄, filtered, and concentrated in vacuo. The obtained crude product was dissolved in 2,2,2-trifluoroacetic acid (5 mL) and stirred for 20 mins. Upon completion, the reaction mixture was diluted with acetonitrile and purified by prep-LCMS (XBridge C18 column, eluting with a gradient of acetonitrile/water containing 0.1% TFA, at flow rate of 60 mL/min) to afford desired product. LCMS calculated for C₁₅H₁₄C₁₂N₅O (M+H)⁺: m/z=350.0; found: 350.0.

### Intermediate 9. Rac-tert-butyl 3-(7-chloro-3-(1-methyl-2-oxo-1,2-dihydropyridin-4-yl)-2H-pyrazolo[4,3-c]pyridin-2-yl)pyrrolidine-1-carboxylate

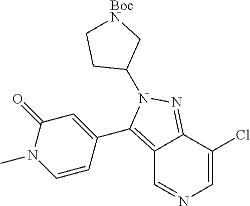 [SMILES: Cn1ccc(-c2c3cncc(Cl)c3nn2C2CCN(C(=O)OC(C)(C)C)C2)cc1=O]

### Step 1. Rac-tert-butyl 3-(7-chloro-3-iodo-2H-pyrazolo[4,3-c]pyridin-2-yl)pyrrolidine-1-carboxylate

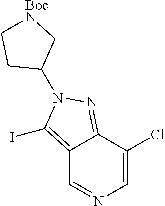 [SMILES: CC(C)(C)OC(=O)N1CCC(n2nc3c(Cl)cncc3c2I)C1]

A mixture of 7-chloro-3-iodo-1H-pyrazolo[4,3-c]pyridine (500 mg, 1.789 mmol), tert-butyl 3-bromopyrrolidine-1-carboxylate (895 mg, 3.58 mmol) and cesium carbonate (1749 mg, 5.37 mmol) in DMF (10 mL) was heated to 70° C. After 1 h, the mixture was cooled, diluted with water and extracted with ethyl acetate (50 mL). The organic layer was washed with brine, dried with Na₂SO₄, filtered, and concentrated in vacuo. The residue was purified by column chromatography (0-50% AcOEt in DCM). LCMS calculated for C₁₅H₁₉ClIN₄O₂(M+H)⁺: m/z=449.0; found: 449.0.

### Step 2. Rac-tert-butyl 3-(7-chloro-3-(1-methyl-2-oxo-1,2-dihydropyridin-4-yl)-2H-pyrazolo[4,3-c]pyridin-2-yl)pyrrolidine-1-carboxylate

To a mixture of tert-butyl 3-(7-chloro-3-iodo-2H-pyrazolo[4,3-c]pyridin-2-yl)pyrrolidine-1-carboxylate (106 mg, 0.236 mmol) in 1,4-dioxane (2 mL) and water (0.4 mL) was added 1-methyl-4-(4,4,5,5-tetramethyl-1,3,2-dioxaborolan-2-yl)pyridin-2(1H)-one (83 mg, 0.353 mmol), potassium phosphate (150 mg, 0.707 mmol) and tetrakis (27.2 mg, 0.024 mmol). The reaction mixture was then degassed for 10 min before heating to 110° C. After 2 h, the resulting mixture was cooled and diluted with water and extracted with ethyl acetate (50 mL). The organic layer was washed with brine, dried with Na₂SO₄, filtered, and concentrated in vacuo. The residue was purified by column chromatography (20-80% AcOEt in DCM). LCMS calculated for C₂₁H₂₅ClIN₅O₃(M+H)⁺: m/z=430.2; found: 430.2.

### Intermediate 10. Rac-tert-butyl 2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridine-6-carboxylate

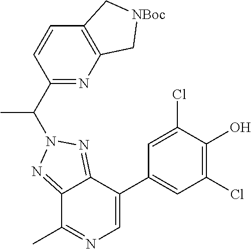 [SMILES: Cc1ncc(-c2cc(Cl)c(O)c(Cl)c2)c2nn(C(C)c3ccc4c(n3)CN(C(=O)OC(C)(C)C)C4)nc12]

### Step 1. Rac-tert-butyl 2-(1-hydroxyethyl)-5,7-dihydro-6H-pyrrolo[3,4-b]pyridine-6-carboxylate

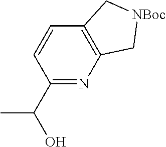 [SMILES: CC(O)c1ccc2c(n1)CN(C(=O)OC(C)(C)C)C2]

To a solution of tert-butyl 2-formyl-5,7-dihydro-6H-pyrrolo[3,4-b]pyridine-6-carboxylate (350 mg, 1.41 mmol) in THF (15 mL) at −78° C. was added methylmagnesium bromide (3M in THF, 1.41 mL, 4.23 mmol). The reaction mixture was stirred at −78° C. for 10 min. Upon completion, the mixture was quenched with aqueous NH₄Cl, warmed to room temperature and extracted with EtOAc (20 mL). The organic layer was washed with brine, dried with Na₂SO₄, filtered, and concentrated in vacuo. The residue was purified by column chromatography (20-50% EtOAc in hexanes). LCMS calculated for C₁₄H₂₁N₂O₃ (M+H)⁺: m/z=265.2; found: 265.2.

### Step 2. Rac-tert-butyl 2-(1-(4,7-dibromo-2H-[1,2,3]triazolo[4,5-c]pyridin-2-yl)ethyl)-5,7-dihydro-6H-pyrrolo[3,4-b]pyridine-6-carboxylate

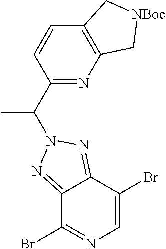 [SMILES: CC(c1ccc2c(n1)CN(C(=O)OC(C)(C)C)C2)n1nc2c(Br)cnc(Br)c2n1]

To a solution of 4,7-dibromo-2H-[1,2,3]triazolo[4,5-c]pyridine hydrochloride (951 mg, 3.03 mmol), rac-tert-butyl 2-(1-hydroxyethyl)-5,7-dihydro-6H-pyrrolo[3,4-b]pyridine-6-carboxylate (800 mg, 3.03 mmol), DIPEA (1.58 mL, 9.08 mmol) and triphenylphosphine (1.19 g, 4.54 mmol) in THF (10 mL) at 0° C. was added diisopropyl azodicarboxylate (0.883 mL, 4.54 mmol). After 15 min the bath was removed, and the mixture was stirred at room temperature overnight.

Upon completion, the mixture was diluted with water and extracted with EtOAc (10 mL). The organic layer was washed with brine, dried with Na₂SO₄, filtered, and concentrated in vacuo. The residue was purified by column chromatography (20-80% EtOAc in hexanes) to remove undesired regioisomers. LCMS calculated for C₁₉H₂₁Br₂N₆O₂ (M+H)⁺: m/z=523.0; found: 523.0.

### Step 3. Rac-tert-butyl 2-(1-(7-bromo-4-methyl-2H-[1,2,3]triazolo[4,5-c]pyridin-2-yl)ethyl)-5,7-dihydro-6H-pyrrolo[3,4-b]pyridine-6-carboxylate

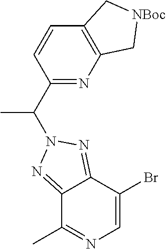 [SMILES: Cc1ncc(Br)c2nn(C(C)c3ccc4c(n3)CN(C(=O)OC(C)(C)C)C4)nc12]

Rac-tert-butyl 2-(1-(4,7-dibromo-2H-[1,2,3]triazolo[4,5-c]pyridin-2-yl)ethyl)-5,7-dihydro-6H-pyrrolo[3,4-b]pyridine-6-carboxylate (550 mg, 1.05 mmol) and tetrakis(triphenylphosphine)palladium(0) (121 mg, 0.105 mmol) were combined in THF (10 mL). The suspension was stirred for 5 min until it became a clear solution. Dimethylzinc (10% in hexane, 1.57 mL, 1.57 mmol) was added at room temperature. After 10 min, the mixture was quenched with aqueous NH₄OH and extracted with EtOAc (10 mL). The organic layer was washed with brine, dried with Na₂SO₄, filtered, and concentrated in vacuo. The residue was purified by column chromatography (20-50% EtOAc in DCM) to remove undesired regioisomers. LCMS calculated for C₂₀H₂₄BrN₆O₂(M+H)⁺: m/z=459.1; found: 459.1.

### Step 4. Rac-tert-butyl 2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridine-6-carboxylate

To a mixture of rac-tert-butyl 2-(1-(7-bromo-4-methyl-2H-[1,2,3]triazolo[4,5-c]pyridin-2-yl)ethyl)-5,7-dihydro-6H-pyrrolo[3,4-b]pyridine-6-carboxylate (600 mg, 1.31 mmol), (3,5-dichloro-4-hydroxyphenyl)boronic acid (405 mg, 1.96 mmol) and tripotassium phosphate (832 mg, 3.92 mmol) in 1,4-dioxane (10 mL) and water (2 mL) was added and XPhos-Pd-G4 (112 mg, 0.131 mmol). The reaction mixture was then degassed for 10 min before heating to 90° C. After 1 h, the resulting mixture was cooled and diluted with water and extracted with EtOAc (10 mL). The organic layer was washed with brine, dried with Na₂SO₄, filtered, and concentrated in vacuo. The residue was purified by column chromatography (20-80% EtOAc in DCM). LCMS calculated for C₂₆H₂₇C₁₂N₆O₃ (M+H)⁺: m/z=541.1; found: 541.1.

### Intermediate 11a. tert-Butyl (R)-2-(1-(7-bromo-4-methyl-2H-[1,2,3]triazolo[4,5-c]pyridin-2-yl)ethyl)-5,7-dihydro-6H-pyrrolo[3,4-b]pyridine-6-carboxylate

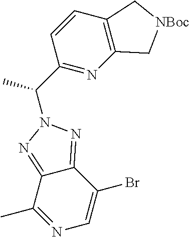 [SMILES: Cc1ncc(Br)c2nn([C@H](C)c3ccc4c(n3)CN(C(=O)OC(C)(C)C)C4)nc12]

Rac-tert-butyl 2-(1-(7-bromo-4-methyl-2H-[1,2,3]triazolo[4,5-c]pyridin-2-yl)ethyl)-5,7-dihydro-6H-pyrrolo[3,4-b]pyridine-6-carboxylate (Intermediate 10, step 3) was purified via chiral prep-SFC (Chiraltech_IC 5 um 30×250 mm column, eluting an isocratic mobile phase of 45% MeOH in CO₂ at a flow rate of 90 mL/min). The first eluted peak was collected and the volatiles removed in vacuo to afford the desired product. LCMS calculated for C₂₀H₂₄BrN₆O₂(M+H)⁺: m/z=459.1; found 459.1.

### Intermediate 11b. tert-Butyl (S)-2-(1-(7-bromo-4-methyl-2H-[1,2,3]triazolo[4,5-c]pyridin-2-yl)ethyl)-5,7-dihydro-6H-pyrrolo[3,4-b]pyridine-6-carboxylate

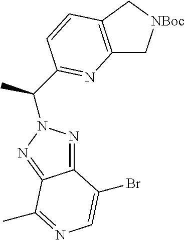 [SMILES: Cc1ncc(Br)c2nn([C@@H](C)c3ccc4c(n3)CN(C(=O)OC(C)(C)C)C4)nc12]

The second eluted peak from above chiral separation was collected and the volatiles removed in vacuo to afford the desired product. LCMS calculated for C₂₀H₂₄BrN₆O₂(M+H)⁺: m/z=459.1; found 459.1.

### Example 1. N-(4-(3-(7-(3,5-dichloro-4-hydroxyphenyl)-2H-[1,2,3]triazolo[4,5-c]pyridin-2-yl)azetidine-1-carbonyl)phenyl)acrylamide

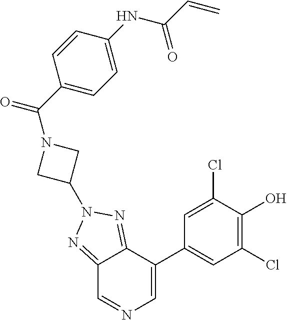 [SMILES: C=CC(=O)Nc1ccc(C(=O)N2CC(n3nc4cncc(-c5cc(Cl)c(O)c(Cl)c5)c4n3)C2)cc1]

### Step 1. tert-butyl 3-(7-bromo-2H-[1,2,3]triazolo[4,5-c]pyridin-2-yl)azetidine-1-carboxylate

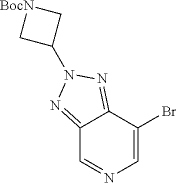 [SMILES: CC(C)(C)OC(=O)N1CC(n2nc3cncc(Br)c3n2)C1]

A mixture of 7-bromo-3H-[1,2,3]triazolo[4,5-c]pyridine hydrochloride (60 mg, 0.255 mmol), tert-butyl 3-iodoazetidine-1-carboxylate (108 mg, 0.382 mmol) and cesium carbonate (249 mg, 0.764 mmol) in DMF (2 mL) was heated to 70° C. for 1 h. Upon completion, the resulting mixture was cooled and diluted with water and extracted with ethyl acetate (10 mL). The organic layer was washed with brine, dried with Na₂SO₄, filtered, and concentrated in vacuo. The residue was purified by column chromatography (20-50% AcOEt in DCM). LCMS calculated for C₁₃H₁₇BrN₅O₂(M+H)⁺: m/z=354.1; found: 354.1.

### Step 2. 4-(2-(azetidin-3-yl)-2H-[1,2,3]triazolo[4,5-c]pyridin-7-yl)-2,6-dichlorophenol

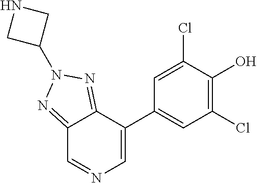 [SMILES: Oc1c(Cl)cc(-c2cncc3nn(C4CNC4)nc23)cc1Cl]

To a mixture of tert-butyl 3-(7-bromo-2H-[1,2,3]triazolo[4,5-c]pyridin-2-yl)azetidine-1-carboxylate (38.9 mg, 0.110 mmol) in 1,4-dioxane (1 mL) and water (0.2 mL) was added (3,5-dichloro-4-hydroxyphenyl)boronic acid (34.1 mg, 0.165 mmol), potassium phosphate (70 mg, 0.330 mmol) and XPhos-Pd-G4 (9.5 mg, 10.99 μmol). The reaction mixture was then degassed for 10 min before heating to 95° C. After 1 h, the resulting mixture was cooled and diluted with water and extracted with ethyl acetate (50 mL). The organic layer was washed with brine, dried with Na₂SO₄, filtered, and concentrated in vacuo. The obtained crude product was dissolved in 2,2,2-trifluoroacetic acid (1 mL) and stirred for 20 mins. Upon completion, the reaction mixture was diluted with acetonitrile and purified by prep-LCMS (XBridge C18 column, eluting with a gradient of acetonitrile/water containing 0.1% TFA, at flow rate of 60 mL/min) to afford desired product. LCMS calculated for C₁₄H₁₂C₁₂N₅O (M+H)⁺: m/z=336.0; found: 336.0.

### Step 3. N-(4-(3-(7-(3,5-dichloro-4-hydroxyphenyl)-2H-[1,2,3]triazolo[4,5-c]pyridin-2-yl)azetidine-1-carbonyl)phenyl)acrylamide

To a solution of 4-(2-(azetidin-3-yl)-2H-[1,2,3]triazolo[4,5-c]pyridin-7-yl)-2,6-dichlorophenol (11.7 mg, 0.035 mmol), 4-acrylamidobenzoic acid (10 mg, 0.052 mmol), DIPEA (30 μL, 0.174 mmol) in THF at 0° C. was added propylphosphonic anhydride (T3P, 31 μL, 50% in ethyl acetate, 0.052 mmol). The reaction mixture was stirred at 0° C. for 5 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₄H₁₉C₁₂N₆O₃ (M+H)⁺: m/z=509.1; found: 509.1.

### Example 2. (S)—N-(4-(3-(7-(3,5-dichloro-4-hydroxyphenyl)-2H-[1,2,3]triazolo[4,5-c]pyridin-2-yl)pyrrolidine-1-carbonyl)phenyl)acrylamide

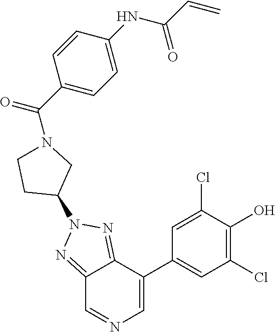 [SMILES: C=CC(=O)Nc1ccc(C(=O)N2CC[C@H](n3nc4cncc(-c5cc(Cl)c(O)c(Cl)c5)c4n3)C2)cc1]

To a solution of (S)-2,6-dichloro-4-(2-(pyrrolidin-3-yl)-2H-[1,2,3]triazolo[4,5-c]pyridin-7-yl)phenol (Intermediate 8, 12.2 mg, 0.035 mmol), 4-acrylamidobenzoic acid (10 mg, 0.052 mmol) and DIPEA (30 μL, 0.174 mmol) in THF (1 mL) was added propylphosphonic anhydride (T3P, 31 μL, 50% in ethyl acetate, 0.052 mmol) at 0° C. The reaction mixture was stirred at 0° C. for 5 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₅H₂₁Cl₂N₆O₃ (M+H)⁺: m/z=523.1; found: 523.1.

### Example 3. Rac-N-(4-(3-(7-(3,5-dichloro-4-hydroxyphenyl)-2H-[1,2,3]triazolo[4,5-c]pyridin-2-yl)piperidine-1-carbonyl)phenyl)acrylamide

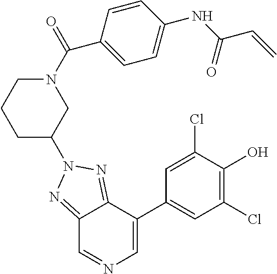 [SMILES: C=CC(=O)Nc1ccc(C(=O)N2CCCC(n3nc4cncc(-c5cc(Cl)c(O)c(Cl)c5)c4n3)C2)cc1]

### Step 1. Rac-tert-butyl 3-(7-bromo-2H-[1,2,3]triazolo[4,5-c]pyridin-2-yl)piperidine-1-carboxylate

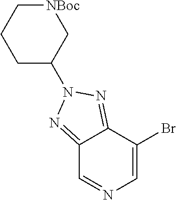 [SMILES: CC(C)(C)OC(=O)N1CCCC(n2nc3cncc(Br)c3n2)C1]

A mixture of 7-bromo-3H-[1,2,3]triazolo[4,5-c]pyridine hydrochloride (200 mg, 0.849 mmol), tert-butyl 3-iodopiperidine-1-carboxylate (396 mg, 1.274 mmol) and cesium carbonate (830 mg, 2.55 mmol) in DMF (2 mL) was heated to 70° C. for 1 h. Upon completion, the resulting mixture was cooled and diluted with water and extracted with ethyl acetate (20 mL). The organic layer was washed with brine, dried with Na₂SO₄, filtered, and concentrated in vacuo. The residue was purified by column chromatography (0-30% AcOEt in DCM). LCMS calculated for C₁₅H₂₁BrN₅O₂(M+H)⁺: m/z=382.1; found: 382.1.

### Step 2. Rac-2,6-dichloro-4-(2-(piperidin-3-yl)-2H-[1,2,3]triazolo[4,5-c]pyridin-7-yl)phenol

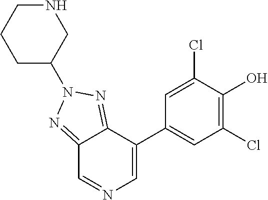 [SMILES: Oc1c(Cl)cc(-c2cncc3nn(C4CCCNC4)nc23)cc1Cl]

To a mixture of tert-butyl 3-(7-bromo-2H-[1,2,3]triazolo[4,5-c]pyridin-2-yl)piperidine-1-carboxylate (102 mg, 0.267 mmol), (3,5-dichloro-4-hydroxyphenyl)boronic acid (83 mg, 0.400 mmol) and potassium phosphate (170 mg, 0.801 mmol) in 1,4-dioxane (2.5 mL) and water (0.5 mL) was added XPhos-Pd-G4 (23 mg, 0.027 mmol). The reaction mixture was then degassed for 10 min before heating to 95° C. After 1 h, the resulting mixture was cooled and diluted with water and extracted with ethyl acetate (50 mL). The organic layer was washed with brine, dried with Na₂SO₄, filtered, and concentrated in vacuo.

The obtained crude product was dissolved in 2,2,2-trifluoroacetic acid (1 mL) and stirred for 20 mins. Upon completion, the reaction mixture was diluted with acetonitrile and purified by prep-LCMS (XBridge C18 column, eluting with a gradient of acetonitrile/water containing 0.1% TFA, at flow rate of 60 mL/min) to afford desired product. LCMS calculated for C₁₆H₁₆C₁₂N₅O (M+H)⁺: m/z=364.1; found: 364.1.

### Step 3. Rac-N-(4-(3-(7-(3,5-dichloro-4-hydroxyphenyl)-2H-[1,2,3]triazolo[4,5-c]pyridin-2-yl)piperidine-1-carbonyl)phenyl)acrylamide

To a solution of Rac-2,6-dichloro-4-(2-(piperidin-3-yl)-2H-[1,2,3]triazolo[4,5-c]pyridin-7-yl)phenol (12.7 mg, 0.035 mmol), 4-acrylamidobenzoic acid (10 mg, 0.052 mmol) and DIPEA (31 μL, 0.174 mmol) in THF (1 mL) was added propylphosphonic anhydride (T3P, 30 μL, 50% in ethyl acetate, 0.052 mmol) at 0° C. The reaction mixture was stirred at 0° C. for 5 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₆H₂₃C₁₂N₆O₃ (M+H)⁺: m/z=537.1; found: 537.1.

### Example 4. Rac-N-(4-((2R,4S)-4-(7-(3,5-dichloro-4-hydroxyphenyl)-2H-[1,2,3]triazolo[4,5-c]pyridin-2-yl)-2-methylpyrrolidine-1-carbonyl)phenyl)acrylamide

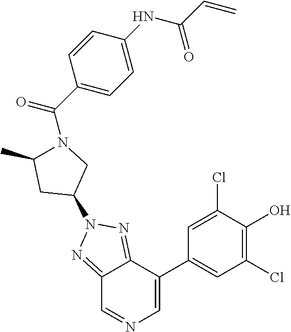 [SMILES: C=CC(=O)Nc1ccc(C(=O)N2C[C@@H](n3nc4cncc(-c5cc(Cl)c(O)c(Cl)c5)c4n3)C[C@H]2C)cc1]

This compound was synthesized based on a similar procedure as described in Example 3, using rac-trans-tert-butyl 4-bromo-2-methylpyrrolidine-1-carboxylate instead of tert-butyl 3-iodopiperidine-1-carboxylate in step 1. LCMS calculated for C₂₆H₂₃C₁₂N₆O₃ (M+H)⁺: m/z=537.1; found: 537.1.

### Example 5. (S)—N-(4-(3-(7-(3,5-dichloro-4-hydroxyphenyl)-2H-[1,2,3]triazolo[4,5-c]pyridin-2-yl)pyrrolidine-1-carbonyl)-2-methoxyphenyl)acrylamide

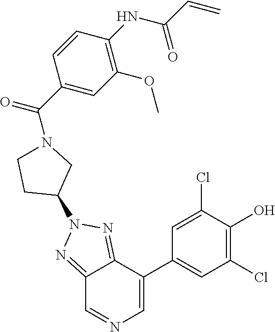 [SMILES: C=CC(=O)Nc1ccc(C(=O)N2CC[C@H](n3nc4cncc(-c5cc(Cl)c(O)c(Cl)c5)c4n3)C2)cc1OC]

### Step 1. 4-acrylamido-3-methoxybenzoic acid

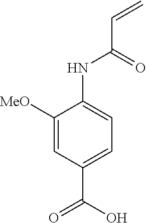 [SMILES: C=CC(=O)Nc1ccc(C(=O)O)cc1OC]

To a 40 mL scintillation vial equipped with a magnetic stir bar was added tert-butyl 4-amino-3-methoxybenzoate (95 mg, 0.425 mmol), triethylamine (0.12 mL, 0.851 mmol) and DCM (2 mL). At 0° C., acryloyl chloride (41 mg, 0.447 mmol) dissolved in DCM (5 mL) was added to the stirring mixture dropwise via an addition funnel over 15 min. The reaction mixture was stirred from 0° C. to room temperature for 20 min. After 1 h, the resulting mixture was cooled and diluted with water and extracted with ethyl acetate (50 mL). The organic layer was washed with brine, dried with Na₂SO₄, filtered, and concentrated in vacuo.

The obtained crude product was dissolved in 2,2,2-trifluoroacetic acid (1 mL) and stirred for 20 mins. Upon completion, the mixture was then concentrated in vacuo and carried forward to the next step without further purification. LCMS calculated for C₁₁H₁₂NO₄ (M+H)⁺: m/z=222.1; found: 222.1.

### Step 2. (S)—N-(4-(3-(7-(3,5-dichloro-4-hydroxyphenyl)-2H-[1,2,3]triazolo[4,5-c]pyridin-2-yl)pyrrolidine-1-carbonyl)-2-methoxyphenyl)acrylamide

To a solution of (S)-2,6-dichloro-4-(2-(pyrrolidin-3-yl)-2H-[1,2,3]triazolo[4,5-c]pyridin-7-yl)phenol (Intermediate 8, 10.5 mg, 0.030 mmol), 4-acrylamido-3-methoxybenzoic acid (10 mg, 0.045 mmol) and DIPEA (26 μL, 0.151 mmol) in THF (1 mL) at 0° C. was added propylphosphonic anhydride (T3P, 27 μL, 50% in ethyl acetate, 0.045 mmol). The reaction mixture was stirred at 0° C. for 5 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₆H₂₃C₁₂N₆O₄ (M+H)⁺: m/z=553.1; found: 553.1.

### Example 6. Rac-N-(4-(3-(7-(3,5-dichloro-4-hydroxyphenyl)-3-(1-methyl-2-oxo-1,2-dihydropyridin-4-yl)-2H-pyrazolo[4,3-c]pyridin-2-yl)pyrrolidine-1-carbonyl)phenyl)acrylamide

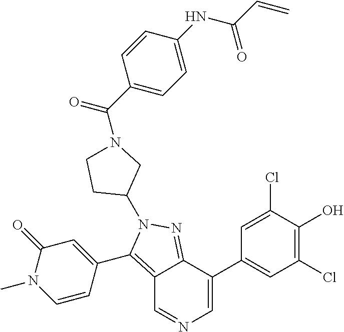 [SMILES: C=CC(=O)Nc1ccc(C(=O)N2CCC(n3nc4c(-c5cc(Cl)c(O)c(Cl)c5)cncc4c3-c3ccn(C)c(=O)c3)C2)cc1]

This compound was synthesized based on a similar procedure as described in Example 3, using Intermediate 9 instead of tert-butyl 3-(7-bromo-2H-[1,2,3]triazolo[4,5-c]pyridin-2-yl)piperidine-1-carboxylate in step 2. LCMS calculated for C₃₂H₂₇C₁₂N₆O₄ (M+H)⁺: m/z=629.1; found: 629.1.

### Example 7. N-(4-((7-(3,5-dichloro-4-hydroxyphenyl)-2H-[1,2,3]triazolo[4,5-c]pyridin-2-yl)methyl)phenyl)acrylamide

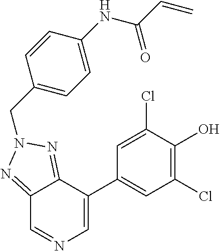 [SMILES: C=CC(=O)Nc1ccc(Cn2nc3cncc(-c4cc(Cl)c(O)c(Cl)c4)c3n2)cc1]

To a suspension of 4-(2-(4-aminobenzyl)-2H-[1,2,3]triazolo[4,5-c]pyridin-7-yl)-2,6-dichlorophenol (Intermediate 7, 5 mg, 0.013 mmol) and Et₃N (9 μL, 0.065 mmol) in THF (0.5 mL) at 0° C. was added acetyl chloride (2 μL, 0.026 mmol). The reaction mixture was stirred at −78° C. for 5 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₁H₁₆C₁₂N₅O₂ (M+H)⁺: m/z=440.1; found: 440.1.

### Example 8. 1-(5-((7-(3,5-dichloro-4-hydroxyphenyl)-2H-[1,2,3]triazolo[4,5-c]pyridin-2-yl)methyl)isoindolin-2-yl)prop-2-en-1-one

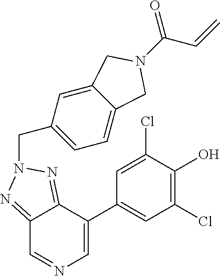 [SMILES: C=CC(=O)N1Cc2ccc(Cn3nc4cncc(-c5cc(Cl)c(O)c(Cl)c5)c4n3)cc2C1]

To a suspension of 2,6-dichloro-4-(2-(isoindolin-5-ylmethyl)-2H-[1,2,3]triazolo[4,5-c]pyridin-7-yl)phenol (Intermediate 1, 100 mg, 0.243 mmol) and Et₃N (0.17 mL, 1.213 mmol) in THF (2 mL) at −78° C. was added acryloyl chloride (0.039 mL, 0.485 mmol) solution (in 1 mL of THF) in small portions. The reaction mixture was stirred at −78° C. for 5 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₃H₁₈C₁₂N₅O₂ (M+H)⁺: m/z=466.1; found: 466.1. ¹H NMR (600 MHz, DMSO-d6) δ 10.6 (br. s, 1H), 9.49 (s, 1H), 8.83 (s, 1H), 8.21 (s, 2H), 7.47-7.35 (m, 3H), 6.65 (ddd, J=16.8, 10.2, 3.9 Hz, 1H), 6.23-6.17 (m, 2H), 5.74 (ddd, J=10.2, 2.4, 2.4 Hz, 1H), 4.94 (dd, J=3.0, 1.6 Hz, 2H), 4.70 (dd, J=3.0, 1.6 Hz, 2H).

### Example 9. Rac-1-(5-(1-(7-(3,5-dichloro-4-hydroxyphenyl)-2H-[1,2,3]triazolo[4,5-c]pyridin-2-yl)ethyl)isoindolin-2-yl)prop-2-en-1-one

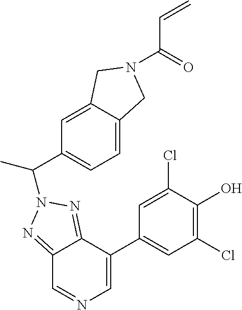 [SMILES: C=CC(=O)N1Cc2ccc(C(C)n3nc4cncc(-c5cc(Cl)c(O)c(Cl)c5)c4n3)cc2C1]

To a suspension of Rac-2,6-dichloro-4-(2-(1-(isoindolin-5-yl)ethyl)-2H-[1,2,3]triazolo[4,5-c]pyridin-7-yl)phenol (Intermediate 6, 10 mg, 0.023 mmol) and Et₃N (16 μL, 0.117 mmol) in THF (1 mL) at −78° C. was added acetyl chloride (3 μL, 0.047 mmol) solution (in 1 mL of THF) in small portions. The reaction mixture was stirred at −78° C. for 5 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₄H₂₀Cl₂N₅O₂ (M+H)⁺: m/z=480.1; found: 480.1).

### Example 10. 1-(5-((4-(3,5-dichloro-4-hydroxyphenyl)-2H-pyrazolo[3,4-c]pyridin-2-yl)methyl)isoindolin-2-yl)prop-2-en-1-one

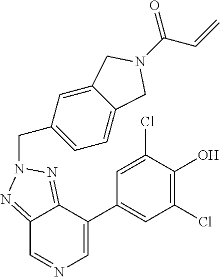 [SMILES: C=CC(=O)N1Cc2ccc(Cn3nc4cncc(-c5cc(Cl)c(O)c(Cl)c5)c4n3)cc2C1]

To a solution of 2,6-dichloro-4-(2-(isoindolin-5-ylmethyl)-2H-pyrazolo[3,4-c]pyridin-4-yl)phenol (Intermediate 4, 74.8 mg, 0.182 mmol), acrylic acid (25 μL, 0.364 mmol) and Et₃N (0.127 ml, 0.909 mmol) in THF (2 mL) at rt was added propylphosphonic anhydride (T3P, 0.216 ml, 0.364 mmol). The reaction mixture was stirred at 0° C. for 30 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₄H₁₉C₁₂N₄O₂ (M+H)⁺: m/z=465.1; found: 465.1). ¹H NMR (600 MHz, DMSO-d6) δ 10.67 (br. s, 1H), 9.49 (s, 1H), 9.25 (s, 1H), 8.38 (s, 1H), 7.86 (s, 2H), 7.47-7.35 (m, 3H), 6.64 (ddd, J=16.8, 10.8, 3.9 Hz, 1H), 6.21 (dd, J=16.8, 2.3 Hz, 1H), 5.88 (s, 2H), 5.73 (dd, J=10.8, 2.3 Hz, 1H), 4.99 (s, 2H), 4.69 (s, 2H).

### Example 11. 1-(5-((7-(3,5-dichloro-4-hydroxyphenyl)-2H-pyrazolo[4,3-c]pyridin-2-yl)methyl)isoindolin-2-yl)prop-2-en-1-one

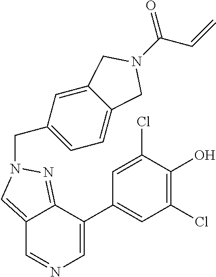 [SMILES: C=CC(=O)N1Cc2ccc(Cn3cc4cncc(-c5cc(Cl)c(O)c(Cl)c5)c4n3)cc2C1]

To a suspension of 2,6-dichloro-4-(2-(isoindolin-5-ylmethyl)-2H-pyrazolo[4,3-c]pyridin-7-yl)phenol (Intermediate 5, 15.4 mg, 0.038 mmol) and Et₃N (26 μL, 0.188 mmol) in THF (2 mL) at −78° C. was added acryloyl chloride (3 μL, 0.038 mmol). The reaction mixture was stirred at −78° C. for 5 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₄H₁₉C₁₂N₄O₂ (M+H)⁺: m/z=465.1; found: 465.1).

### Example 12. (E)-2-(5-((7-(3,5-dichloro-4-hydroxyphenyl)-2H-[1,2,3]triazolo[4,5-c]pyridin-2-yl)methyl)isoindoline-2-carbonyl)-4-methylpent-2-enenitrile

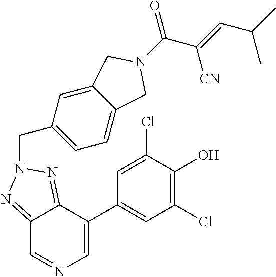 [SMILES: CC(C)/C=C(\C#N)C(=O)N1Cc2ccc(Cn3nc4cncc(-c5cc(Cl)c(O)c(Cl)c5)c4n3)cc2C1]

To a solution of (E)-2-cyano-4-methylpent-2-enoic acid (6.8 mg, 0.049 mmol) and 2,6-dichloro-4-(2-(isoindolin-5-ylmethyl)-2H-[1,2,3]triazolo[4,5-c]pyridin-7-yl)phenol (Intermediate 1, 10 mg, 0.024 mmol) in THF (0.5 mL) was added DIPEA (21 μL, 0.121 mmol) and propylphosphonic anhydride (T3P, 29 μL, 50% in ethyl acetate, 0.049 mmol). The reaction mixture was stirred at 0° C. for 30 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₇H₂₃C₁₂N₆O₂ (M+H)⁺: m/z=533.1; found: 533.1.

### Example 13. 1-(5-((7-(3,5-dichloro-4-hydroxyphenyl)-2H-[1,2,3]triazolo[4,5-c]pyridin-2-yl)methyl)isoindolin-2-yl)but-2-yn-1-one

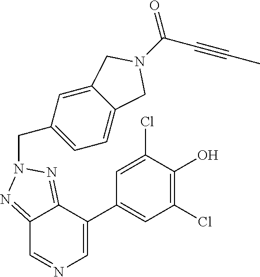 [SMILES: CC#CC(=O)N1Cc2ccc(Cn3nc4cncc(-c5cc(Cl)c(O)c(Cl)c5)c4n3)cc2C1]

To a solution of but-2-ynoic acid (4.1 mg, 0.049 mmol) and 2,6-dichloro-4-(2-(isoindolin-5-ylmethyl)-2H-[1,2,3]triazolo[4,5-c]pyridin-7-yl)phenol (Intermediate 1, 10 mg, 0.024 mmol) in THF (0.5 mL) was added DIPEA (21 μL, 0.121 mmol) and propylphosphonic anhydride (T3P, 29 μL, 50% in ethyl acetate, 0.049 mmol). The reaction mixture was stirred at 0° C. for 30 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₄H₁₈Cl₂N₅O₂ (M+H)⁺: m/z=478.1; found: 478.1.

### Example 14. 1-(5-((7-(3,5-dichloro-4-hydroxyphenyl)-2H-[1,2,3]triazolo[4,5-c]pyridin-2-yl)methyl)isoindolin-2-yl)-2-fluoroprop-2-en-1-one

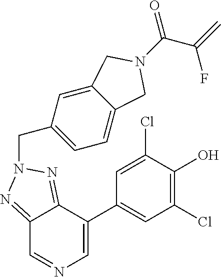 [SMILES: C=C(F)C(=O)N1Cc2ccc(Cn3nc4cncc(-c5cc(Cl)c(O)c(Cl)c5)c4n3)cc2C1]

To a solution of 2-fluoroacrylic acid (4.4 mg, 0.049 mmol) and 2,6-dichloro-4-(2-(isoindolin-5-ylmethyl)-2H-[1,2,3]triazolo[4,5-c]pyridin-7-yl)phenol (Intermediate 1, 10 mg, 0.024 mmol) in THF (0.5 mL) was added DIPEA (21 μL, 0.121 mmol) and propylphosphonic anhydride (T3P, 29 μL, 50% in ethyl acetate, 0.049 mmol). The reaction mixture was stirred at 0° C. for 30 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₃H₁₇C₁₂FN₅O₂(M+H)⁺: m/z=484.1; found: 484.1.

### Example 15. (E)-1-(5-((7-(3,5-dichloro-4-hydroxyphenyl)-2H-[1,2,3]triazolo[4,5-c]pyridin-2-yl)methyl)isoindolin-2-yl)-4-(1H-1,2,4-triazol-1-yl)but-2-en-lone

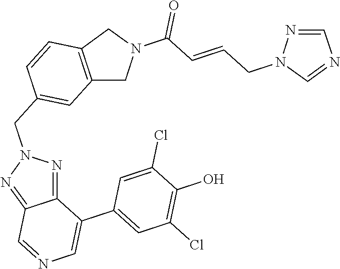 [SMILES: O=C(/C=C/Cn1cncn1)N1Cc2ccc(Cn3nc4cncc(-c5cc(Cl)c(O)c(Cl)c5)c4n3)cc2C1]

To a suspension of 2,6-dichloro-4-(2-(isoindolin-5-ylmethyl)-2H-[1,2,3]triazolo[4,5-c]pyridin-7-yl)phenol (10 mg, 0.024 mmol) and DIPEA (42 μL, 0.243 mmol), (E)-4-(1H-1,2,4-triazol-1-yl)but-2-enoic acid hydrochloride (13.8 mg, 0.073 mmol) and DIPEA (21 μL, 0.121 mmol) in THF (500 μL) was added propylphosphonic anhydride (T3P, 29 μL, 50% in ethyl acetate, 0.049 mmol). Stirred at room temperature for 30 minutes before the solution was diluted with MeOH and purified by prep-LCMS (XBridge C18 column, eluting with a gradient of acetonitrile/water containing 0.1% TFA, at flow rate of 60 mL/min) to afford desired product as a TFA salt. LCMS calculated for C₂₆H₂₁C₁₂N₈O₂ (M+H)⁺: 547.1; found 547.1.

### Example 16. (E)-1-(5-((7-(3,5-dichloro-4-hydroxyphenyl)-2H-[1,2,3]triazolo[4,5-c]pyridin-2-yl)methyl)isoindolin-2-yl)-3-(pyridin-4-yl)prop-2-en-1-one

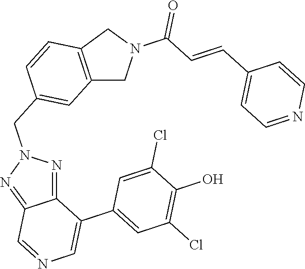 [SMILES: O=C(/C=C/c1ccncc1)N1Cc2ccc(Cn3nc4cncc(-c5cc(Cl)c(O)c(Cl)c5)c4n3)cc2C1]

To a suspension of 2,6-dichloro-4-(2-(isoindolin-5-ylmethyl)-2H-[1,2,3]triazolo[4,5-c]pyridin-7-yl)phenol (10 mg, 0.024 mmol), DIPEA (42.4 μL, 0.243 mmol) and (E)-3-(pyridin-4-yl)acrylic acid (10.85 mg; 0.073 mmol) in THF (500p) was added propylphosphonic anhydride (T3P, 29 μL, 50% in ethyl acetate, 0.049 mmol). Stirred at room temperature for 30 minutes before the solution was diluted with MeOH and purified by prep-LCMS (XBridge C18 column, eluting with a gradient of acetonitrile/water containing 0.1% TFA, at flow rate of 60 mL/min) to afford desired product as a TFA salt. LCMS calculated for C₂₈H₂₁C₁₂N₆O₂ (M+H)⁺: 543.1; found 543.1.

### Example 17. (E)-1-(5-((7-(3,5-dichloro-4-hydroxyphenyl)-2H-[1,2,3]triazolo[4,5-c]pyridin-2-yl)methyl)isoindolin-2-yl)-4,4,4-trifluorobut-2-en-1-one

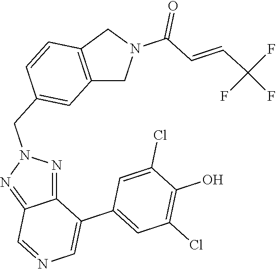 [SMILES: O=C(/C=C/C(F)(F)F)N1Cc2ccc(Cn3nc4cncc(-c5cc(Cl)c(O)c(Cl)c5)c4n3)cc2C1]

To a suspension of 2,6-dichloro-4-(2-(isoindolin-5-ylmethyl)-2H-[1,2,3]triazolo[4,5-c]pyridin-7-yl)phenol (Example 1, 20 mg, 49 μmol) and N—N-diisopropylethylamine (42 μL, 0.24 mmol) in THF (2 mL) at 0° C. was added (E)-4,4,4-trifluorobut-2-enoyl chloride (7 μg, 49 μmol) solution (in 1 ml of THF) in small portions. The reaction mixture was stirred at 0° C. for 15 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₄H₁₇C₁₂N₅F₃O₂ (M+H)⁺: m/z=534.1; found: 534.1.

### Example 18. (E)-1-(5-((7-(3,5-dichloro-4-hydroxyphenyl)-2H-[1,2,3]triazolo[4,5-c]pyridin-2-yl)methyl)isoindolin-2-yl)-4-hydroxybut-2-en-1-one

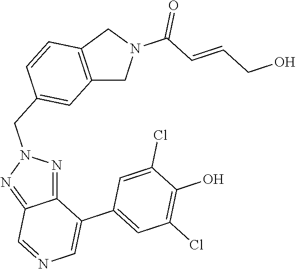 [SMILES: O=C(/C=C/CO)N1Cc2ccc(Cn3nc4cncc(-c5cc(Cl)c(O)c(Cl)c5)c4n3)cc2C1]

To a suspension 2,6-dichloro-4-(2-(isoindolin-5-ylmethyl)-2H-[1,2,3]triazolo[4,5-c]pyridin-7-yl)phenol (Example 1, 20 mg, 49 μmol), (E)-4-hydroxybut-2-enoic acid (5 mg, 49 μmol) and N—N-diisopropylethylamine (42 μL, 0.24 mmol) in THF (2 mL) at 23° C. was added propylphosphonic anhydride (T3P, 29 μL, 50% in ethyl acetate, 49 μmol). The reaction mixture was stirred at 23° C. for 10 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₄H₂₀Cl₂N₅O₃ (M+H)⁺: m/z=496.1; found: 496.1.

### Example 19. methyl (E)-4-(5-((7-(3,5-dichloro-4-hydroxyphenyl)-2H-[1,2,3]triazolo[4,5-c]pyridin-2-yl)methyl)isoindolin-2-yl)-4-oxobut-2-enoate

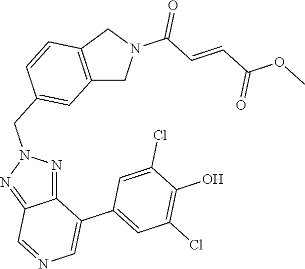 [SMILES: COC(=O)/C=C/C(=O)N1Cc2ccc(Cn3nc4cncc(-c5cc(Cl)c(O)c(Cl)c5)c4n3)cc2C1]

To a suspension of 2,6-dichloro-4-(2-(isoindolin-5-ylmethyl)-2H-[1,2,3]triazolo[4,5-c]pyridin-7-yl)phenol (Example 1, 20 mg, 49 μmol), (E)-4-methoxy-4-oxobut-2-enoic acid (6.3 mg, 49 μmol) and N—N-diisopropylethylamine (42 μL, 0.24 mmol) in THF (2 mL) at 23° C. was added propylphosphonic anhydride (T3P, 29 μl, 50% in ethyl acetate, 49 μmol). The reaction mixture was stirred at 23° C. for 10 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₅H₂₀C₁₂N₅O₄ (M+H)⁺: m/z=524.1; found: 524.1.

### Example 20. (E)-1-(5-((7-(3,5-dichloro-4-hydroxyphenyl)-2H-pyrazolo[4,3-c]pyridin-2-yl)methyl)isoindolin-2-yl)-4,4-difluorobut-2-en-1-one

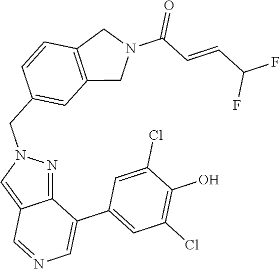 [SMILES: O=C(/C=C/C(F)F)N1Cc2ccc(Cn3cc4cncc(-c5cc(Cl)c(O)c(Cl)c5)c4n3)cc2C1]

To a suspension of 2,6-dichloro-4-(2-(isoindolin-5-ylmethyl)-2H-pyrazolo[4,3-c]pyridin-7-yl)phenol (Intermediate 5, 20 mg, 49 μmol), (E)-4,4-difluorobut-2-enoic acid (6 mg, 49 μmol) and N—N-diisopropylethylamine (42 μL, 0.24 mmol) in THF (2 ml) at 23° C. was added propylphosphonic anhydride (T3P, 29 μL, 50% in ethyl acetate, 49 μmol). The reaction mixture was stirred at 23° C. for 15 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₅H₁₉C₁₂F₂N₄O₂(M+H)⁺: m/z=515.1; found: 515.1. ¹H NMR (600 MHz, DMSO-d6) δ 9.39 (s, 1H), 9.17 (s, 1H), 8.60 (s, 1H), 8.22 (s, 2H), 7.38 (m, 2H), 6.93 (dd, J=15.3, 7.3 Hz, 1H), 6.66 (m, 1H), 5.88 (m, 2H), 4.97 (s, 2H), 4.72 (m, 2H).

### Example 21. (E)-4-(azetidin-1-yl)-1-(5-((7-(3,5-dichloro-4-hydroxyphenyl)-2H-[1,2,3]triazolo[4,5-c]pyridin-2-yl)methyl)isoindolin-2-yl)but-2-en-1-one

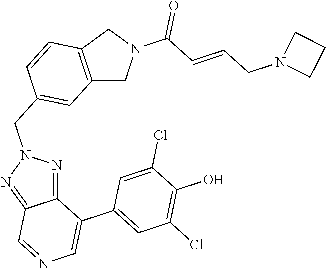 [SMILES: O=C(/C=C/CN1CCC1)N1Cc2ccc(Cn3nc4cncc(-c5cc(Cl)c(O)c(Cl)c5)c4n3)cc2C1]

To a suspension of 2,6-dichloro-4-(2-(isoindolin-5-ylmethyl)-2H-[1,2,3]triazolo[4,5-c]pyridin-7-yl)phenol (Intermediate 1, 10 mg, 0.024 mmol), DIPEA (42 μL, 0.243 mmol) and 4-(azetidin-1-yl)but-2-enoic acid (10.3 mg, 0.073 mmol) in THF (0.5 mL) was added propylphosphonic anhydride (T3P, 29 μL, 50% in ethyl acetate, 49 μmol). Stirred at room temperature for 30 minutes before the solution was diluted with MeOH and purified by prep-LCMS (XBridge C18 column, eluting with a gradient of acetonitrile/water containing 0.1% TFA, at flow rate of 60 mL/min) to afford desired product as a TFA salt. LCMS calculated for C₂₇H₂₅C₁₂N₆O₂ (M+H)⁺: 535.1; found 535.1. ¹H NMR (600 MHz, DMSO-d6) δ 9.87 (br. s, 1H), 9.45 (s, 1H), 8.82 (s, 1H), 8.20 (d, J=1.8 Hz, 2H), 7.49-7.42 (m, 1H), 7.18 (dd, J=2.6, 2.4 Hz, 1H), 6.70-6.62 (m, 1H), 6.59-6.51 (m, 1H), 6.19 (d, J=6.5 Hz, 2H), 4.94 (d, J=3.6 Hz, 2H), 4.70 (s, 2H), 4.16 (s, 2H), 3.99 (s, 4H), 2.33 (m, 2H).

### Example 22. 1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-b]pyridin-6-yl)prop-2-en-1-one

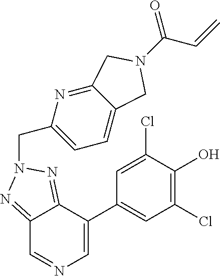 [SMILES: C=CC(=O)N1Cc2ccc(Cn3nc4cncc(-c5cc(Cl)c(O)c(Cl)c5)c4n3)nc2C1]

To a solution of 1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-b]pyridin-6-yl)prop-2-en-1-one (Intermediate 2, 80 mg, 0.194 mmol) acrylic acid (16 μL, 0.228 mmol) and DIPEA (132 μL, 0.759 mmol) in THF (2 mL) at 0° C. was added propylphosphonic anhydride (T3P, 90 μL, 50% in ethyl acetate, 0.303 mmol). The reaction mixture was stirred at 0° C. for 30 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₂H₁₇Cl₂N₆O₂ (M+H)⁺: m/z=467.1; found: 467.1. ¹H NMR (600 MHz, DMSO-d6) δ 10.61 (br. s, 1H), 9.51 (s, 1H), 8.85 (s, 1H), 8.22 (s, 2H), 7.86 (dd, J=22.8, 8.0 Hz, 1H), 7.44 (dd, J=23.8, 2.3 Hz, 1H), 6.66 (ddd, J=16.8, 10.8, 2.4 Hz, 1H), 6.31 (s, 2H), 6.22 (ddd, J=16.8, 8.8, 2.2 Hz, 1H), 5.74 (ddd, J=16.8, 10.0, 2.2 Hz, 1H), 5.00-4.63 (m, 4H).

### Example 23. (E)-1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-b]pyridin-6-yl)-4-(diethylamino)but-2-en-1-one

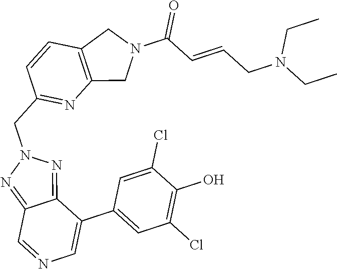 [SMILES: CCN(CC)C/C=C/C(=O)N1Cc2ccc(Cn3nc4cncc(-c5cc(Cl)c(O)c(Cl)c5)c4n3)nc2C1]

To a suspension of 2,6-dichloro-4-(2-((6,7-dihydro-5H-pyrrolo[3,4-b]pyridin-2-yl)methyl)-2H-[1,2,3]triazolo[4,5-c]pyridin-7-yl)phenol (Intermediate 2, 10 mg, 0.024 mmol), DIPEA (42 μL, 0.243 mmol) and (E)-4-(diethylamino)but-2-enoic acid hydrochloride (14.06 mg, 0.073 mmol) in THF (500 μL) was added propylphosphonic anhydride (T3P, 29 μl, 50% in ethyl acetate, 49 μmol). Stirred at room temperature for 30 minutes before the solution was diluted with MeOH and purified by prep-LCMS (XBridge C18 column, eluting with a gradient of acetonitrile/water containing 0.1% TFA, at flow rate of 60 mL/min) to afford desired product as a TFA salt. LCMS calculated for C₂₇H₂₈C₁₂N₇O₂ (M+H)⁺: 552.1; found 552.1. ¹H NMR (600 MHz, DMSO-d6) δ 8.83 (s, 1H), 8.20 (s, 1H), 7.87 (dd, J=14.3, 7.9 Hz, 1H), 7.45 (dd, J=17.6, 7.9 Hz, 1H), 6.86-6.77 (m, 1H), 6.70 (tt, J=15.0, 7.3 Hz, 1H), 6.31 (dd, J=10.4, 5.4 Hz, 1H), 5.00 (s, 1H), 4.87 (s, 1H), 4.74 (s, 1H), 4.64 (s, 1H), 4.15 (s, 1H), 3.99-3.94 (m, 1H), 3.94-3.90 (m, 2H), 3.22 (s, 1H), 3.19-3.08 (m, 4H), 1.28-1.14 (m, 6H).

### Example 24. (E)-1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-b]pyridin-6-yl)-4-(piperidin-1-yl)but-2-en-1-one

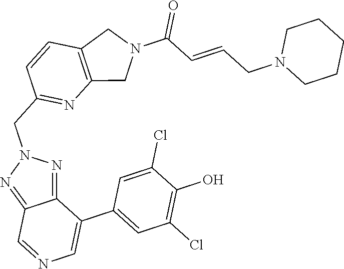 [SMILES: O=C(/C=C/CN1CCCCC1)N1Cc2ccc(Cn3nc4cncc(-c5cc(Cl)c(O)c(Cl)c5)c4n3)nc2C1]

To a suspension of 2,6-dichloro-4-(2-((6,7-dihydro-5H-pyrrolo[3,4-b]pyridin-2-yl)methyl)-2H-[1,2,3]triazolo[4,5-c]pyridin-7-yl)phenol (Intermediate 2, 10 mg, 0.024 mmol), (E)-4-(piperidin-1-yl)but-2-enoic acid hydrochloride (14.9 mg, 0.073 mmol) and DIPEA (42 μL, 0.243 mmol) in THF (500 μL) was added propylphosphonic anhydride (T3P, 29 μl, 50% in ethyl acetate, 49 μmol). Stirred at room temperature for 30 minutes before the solution was diluted with MeOH and purified by prep-LCMS (XBridge C18 column, eluting with a gradient of acetonitrile/water containing 0.1% TFA, at flow rate of 60 mL/min) to afford desired product as a TFA salt. LCMS calculated for C₂₈H₂₈C₁₂N₇O₂ (M+H)⁺: 564.1; found 564.1

### Example 25. 1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-d]pyrimidin-6-yl)prop-2-en-1-one

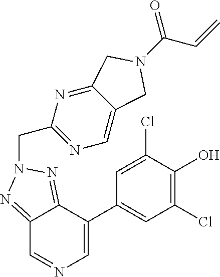 [SMILES: C=CC(=O)N1Cc2cnc(Cn3nc4cncc(-c5cc(Cl)c(O)c(Cl)c5)c4n3)nc2C1]

To a solution of 2,6-dichloro-4-(2-((6,7-dihydro-5H-pyrrolo[3,4-d]pyrimidin-2-yl)methyl)-2H-[1,2,3]triazolo[4,5-c]pyridin-7-yl)phenol (Intermediate 3, 15.6 mg, 0.038 mmol) and Et₃N (26 μL, 0.188 mmol) in THF (2 mL) at −78° C. was added acryloyl chloride (3 μL, 0.038 mmol) solution (in 1 mL of THF) in small portions. The reaction mixture was stirred at 0° C. for 30 min, then was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₁H₁₆C₁₂N₇O₂ (M+H)⁺: m/z=468.1; found: 468.1.

### Example 26. (E)-1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-d]pyrimidin-6-yl)-4-(piperidin-1-yl)but-2-en-1-one

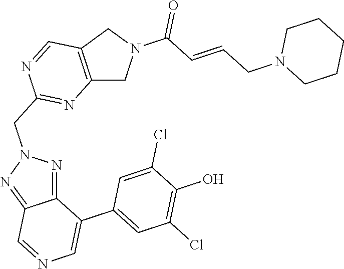 [SMILES: O=C(/C=C/CN1CCCCC1)N1Cc2cnc(Cn3nc4cncc(-c5cc(Cl)c(O)c(Cl)c5)c4n3)nc2C1]

To a suspension of 2,6-dichloro-4-(2-((6,7-dihydro-5H-pyrrolo[3,4-d]pyrimidin-2-yl)methyl)-2H-[1,2,3]triazolo[4,5-c]pyridin-7-yl)phenol (Intermediate 3, 0.024 mmol), (E)-4-(piperidin-1-yl)but-2-enoic acid hydrochloride (14.90 mg, 0.073 mmol) and DIPEA (42 μL, 0.243 mmol) in THF (500 μL) was added propylphosphonic anhydride (T3P, 29 μL, 50% in ethyl acetate, 49 μmol). Stirred at room temperature for 30 minutes before the solution was diluted with MeOH and purified by prep-LCMS (XBridge C18 column, eluting with a gradient of acetonitrile/water containing 0.1% TFA, at flow rate of 60 mL/min) to afford desired product as a TFA salt. LCMS calculated for C₂₇H₂₇C₁₂N₈O₂ (M+H)⁺: 565.1; found 565.1.

### Example 27. Rac-(E)-1-(2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridin-6-yl)-4-(diethylamino)but-2-en-1-one

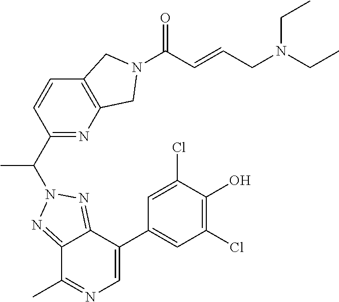 [SMILES: CCN(CC)C/C=C/C(=O)N1Cc2ccc(C(C)n3nc4c(-c5cc(Cl)c(O)c(Cl)c5)cnc(C)c4n3)nc2C1]

Rac-tert-butyl 2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridine-6-carboxylate (Intermediate 10, 100 mg, 0.185 mmol) was dissolved in DCM (1 mL). To this was then added 2,2,2 trifluoro acetic acid (1 mL). The mixture was stirred at room temperature for 1 h before the volatiles were removed in vacuo. The resulting oil was then dissolved in THF (1.8 mL). To this solution was then added Hunig's base (0.317 mL, 1.81 mmol), (E)-4-(diethylamino)but-2-enoic acid hydrochloride (52.7 mg, 0.272 mmol) and propylphosphonic anhydride (T3P, 162 μL, 50% in EtOAc, 0.272 mmol). The reaction mixture was stirred at room temperature for 45 min. Volatiles were removed in vacuo and the resulting oil was diluted with MeOH before being purified via prep-LCMS (XBridge C18 column, eluting with a gradient of acetonitrile/water containing 0.1% TFA, at flow rate of 60 mL/min) to afford desired product as a TFA salt. LCMS calculated for C₂₉H₃₂C₁₂N₇O₂ (M+H)⁺: m/z=580.2; found 580.2. ¹H NMR (600 MHz, DMSO_ht) δ 9.34 (s, 1H), 8.66 (s, 1H), 8.15 (s, 2H), 7.88-7.80 (m, 1H), 7.37 (d, J=8.0 Hz, 1H), 6.82 (t, J=15.1 Hz, 1H), 6.75-6.64 (m, 1H), 6.56 (p, J=7.0 Hz, 1H), 4.98 (s, 1H), 4.94-4.85 (m, 1H), 4.73 (s, 1H), 4.71-4.62 (m, 1H), 3.99-3.90 (m, 2H), 3.13 (tt, J=11.7, 5.6 Hz, 4H), 2.85 (d, J=1.8 Hz, 3H), 2.13 (d, J=7.0 Hz, 3H), 1.21 (q, J=7.1 Hz, 6H).

### Example 27a. (R,E)-1-(2-(1-(7-(3,5-dichloro-4-hydroxyphenyl)-4-methyl-2H-[1,2,3]triazolo[4,5-c]317yridine-2-yl)ethyl)-5,7-dihydro-6H-pyrrolo[3,4-b]317yridine-6-yl)-4-(diethylamino)but-2-en-1-one

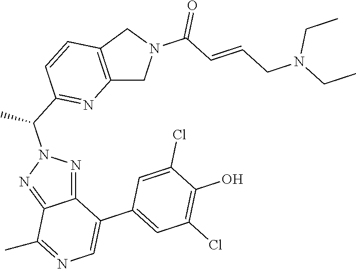 [SMILES: CCN(CC)C/C=C/C(=O)N1Cc2ccc([C@@H](C)n3nc4c(-c5cc(Cl)c(O)c(Cl)c5)cnc(C)c4n3)nc2C1]

This compound was synthesized based on a similar procedure as described in Example 27, using Intermediate 11a instead of Intermediate 10. LCMS calculated for C₂₉H₃₂C₁₂N₇O₂ (M+H)⁺: m/z=580.2; found 580.2.

### Example 27b. (S,E)-1-(2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridin-6-yl)-4-(diethylamino)but-2-en-1-one

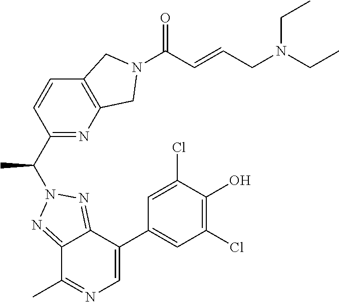 [SMILES: CCN(CC)C/C=C/C(=O)N1Cc2ccc([C@H](C)n3nc4c(-c5cc(Cl)c(O)c(Cl)c5)cnc(C)c4n3)nc2C1]

This compound was synthesized based on a similar procedure as described in Example 27, using Intermediate 11b instead of Intermediate 10. LCMS calculated for C₂₉H₃₂C₁₂N₇O₂ (M+H)⁺: m/z=580.2; found 580.2.

### Example 28. (E)-1-(2-(2-(7-(3,5-dichloro-4-hydroxyphenyl)-4-methyl-2H-[1,2,3]triazolo[4,5-c]pyridin-2-yl)propan-2-yl)-5,7-dihydro-6H-pyrrolo[3,4-b]pyridin-6-yl)-4-(dimethylamino)but-2-en-1-one

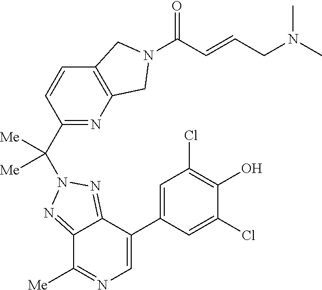 [SMILES: Cc1ncc(-c2cc(Cl)c(O)c(Cl)c2)c2nn(C(C)(C)c3ccc4c(n3)CN(C(=O)/C=C/CN(C)C)C4)nc12]

### Step 1. tert-Butyl 2-(2-(7-bromo-4-methyl-2H-[1,2,3]triazolo[4,5-c]pyridin-2-yl)propan-2-yl)-5,7-dihydro-6H-pyrrolo[3,4-b]pyridine-6-carboxylate

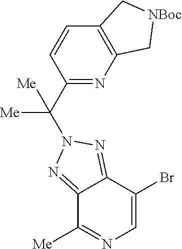 [SMILES: Cc1ncc(Br)c2nn(C(C)(C)c3ccc4c(n3)CN(C(=O)OC(C)(C)C)C4)nc12]

To a solution of rac-tert-butyl 2-(1-(7-bromo-4-methyl-2H-[1,2,3]triazolo[4,5-c]pyridin-2-yl)ethyl)-5,7-dihydro-6H-pyrrolo[3,4-b]pyridine-6-carboxylate (Intermediate 10, step 3, 220 mg, 0.479 mmol) in THF (3 mL) at −78° C. was added LiHMDS (1M in THF, 0.575 mL, 0.575 mmol). Reaction mixture stirred at −78° C. for 20 min, then added iodomethane (0.031 mL, 0.49 mmol). After stirring for additional 5 min at −78° C., the cooling bath was removed, and the reaction was continued to stir at room temperature for 5 min. Upon completion, the resulting mixture was quenched with saturated NH₄Cl solution (5 mL) and extracted with EtOAc (15 mL). The organic layer was washed with brine, dried with Na₂SO₄, filtered, and concentrated in vacuo. The residue was purified by column chromatography (0-40% EtOAc in hexanes) to give the desired product as a white solid. LCMS calculated for C₂₁H₂₆BrN₆O₂(M+H)⁺: m/z=473.1; found: 473.1.

### Step 2. (E)-1-(2-(2-(7-(3,5-dichloro-4-hydroxyphenyl)-4-methyl-2H-[1,2,3]triazolo[4,5-c]pyridin-2-yl)propan-2-yl)-5,7-dihydro-6H-pyrrolo[3,4-b]pyridin-6-yl)-4-(dimethylamino)but-2-en-1-one

To a solution of tert-butyl 2-(2-(7-bromo-4-methyl-2H-[1,2,3]triazolo[4,5-c]pyridin-2-yl)propan-2-yl)-5,7-dihydro-6H-pyrrolo[3,4-b]pyridine-6-carboxylate (25 mg, 0.053 mmol) in 1,4-dioxane (828 μL) and water (166 μL) was added 2,6-dichloro-4-(4,4,5,5-tetramethyl-1,3,2-dioxaborolan-2-yl)phenol (30.5 mg, 0.106 mmol), tripotassium phosphate (33.6 mg, 0.158 mmol) and XPhos-Pd-G4 (2.3 mg, 2.64 μmol). The reaction mixture was then degassed for 10 min before heating to 100° C. After 1 h, the resulting mixture was cooled and diluted with water. The pH of the solution was adjusted to 5 using 1 N HCl and extracted with EtOAc (5 mL). The organic layer was washed with brine, dried with Na₂SO₄, filtered, and concentrated in vacuo.

The obtained crude product was dissolved in DCM (0.5 mL) and 2,2,2-trifluoroacetic acid (0.5 mL) and stirred for 30 mins. Upon completion, the reaction mixture was concentrated in vacuo.

To the above crude product was added N-ethyl-N-isopropylpropan-2-amine (27 μL, 0.15 mmol), (E)-4-(diethylamino)but-2-enoic acid hydrochloride (7.2 mg, 0.037 mmol) and propylphosphonic anhydride (T3P, 50% in EtOAc, 18 μL, 0.031 mmol) in THF (1 mL). The reaction mixture stirred at room temperature for 10 mins before the solution was diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₈H₃₀Cl₂N₇O₂ (M+H)⁺: m/z=566.2; found: 566.1.

### Example 29. (S,E)-1-(2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridin-6-yl)-4-(2,2-dimethylpyrrolidin-1-yl)but-2-en-1-one

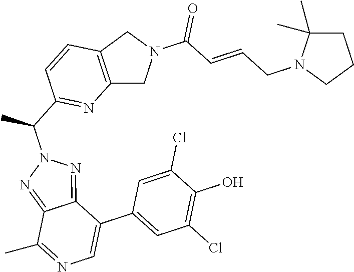 [SMILES: Cc1ncc(-c2cc(Cl)c(O)c(Cl)c2)c2nn([C@@H](C)c3ccc4c(n3)CN(C(=O)/C=C/CN3CCCC3(C)C)C4)nc12]

This compound was synthesized based on a similar procedure as described in Example 27, using Intermediate 11b instead of Intermediate 10 and using (E)-4-(2,2-dimethylpyrrolidin-1-yl)but-2-enoic acid hydrochloride instead of (E)-4-(diethylamino)but-2-enoic acid hydrochloride. LCMS calculated for C₃₁H₃₄C₁₂N₇O₂ (M+H)⁺: m/z=606.2; found: 606.2. ¹H NMR (600 MHz, DMSO_ht) δ 9.41 (s, 1H), 8.66 (s, 1H), 8.15 (d, J=2.5 Hz, 2H), 7.88-7.80 (m, 1H), 7.37 (d, J=8.0 Hz, 1H), 6.81 (t, J=15.9 Hz, 1H), 6.76-6.64 (m, 1H), 6.56 (p, J=7.0 Hz, 1H), 4.98 (s, 1H), 4.94-4.83 (m, 1H), 4.73 (s, 1H), 4.71-4.62 (m, 1H), 4.05 (t, J=14.3 Hz, 1H), 3.67 (s, 1H), 3.51 (s, 1H), 3.21 (s, 1H), 2.85 (s, 3H), 2.14 (d, J=6.9 Hz, 3H), 1.99 (q, J=7.6 Hz, 2H), 1.90-1.80 (m, 3H), 1.43 (d, J=5.7 Hz, 3H), 1.23 (d, J=5.2 Hz, 3H).

### Example 30. Rac-1-(2-(1-(7-(3,5-dichloro-4-hydroxyphenyl)-4-methyl-2H-[1,2,3]triazolo[4,5-c]pyridin-2-yl)ethyl)-3-fluoro-5,7-dihydro-6H-pyrrolo[3,4-b]pyridin-6-yl)prop-2-en-1-one

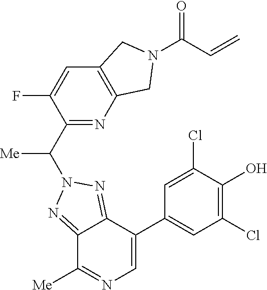 [SMILES: C=CC(=O)N1Cc2cc(F)c(C(C)n3nc4c(-c5cc(Cl)c(O)c(Cl)c5)cnc(C)c4n3)nc2C1]

### Step 1. Rac-tert-butyl 2-(1-(4,7-dibromo-2H-[1,2,3]triazolo[4,5-c]pyridin-2-yl)ethyl)-3-fluoro-5,7-dihydro-6H-pyrrolo[3,4-b]pyridine-6-carboxylate

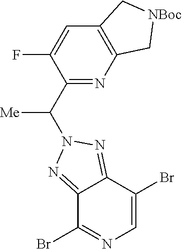 [SMILES: CC(c1nc2c(cc1F)CN(C(=O)OC(C)(C)C)C2)n1nc2c(Br)cnc(Br)c2n1]

To the magnetically stirred solution of 4,7-dibromo-2H-[1,2,3]triazolo[4,5-c]pyridine hydrochloride (167 mg, 0.531 mmol), Rac-tert-butyl 3-fluoro-2-(1-hydroxyethyl)-5,7-dihydro-6H-pyrrolo[3,4-b]pyridine-6-carboxylate (150 mg, 0.531 mmol) and triphenylphosphine (167 mg, 0.638 mmol) were combined in THF (5 mL) at 0° C. followed by addition of diisopropyl azodicarboxylate (124 mL, 0.638 mmol). The mixture was stirred at 0° C. for 45 min and then warmed to 23° C. and stirred for 15 min. Upon completion, the resulting mixture was quenched with saturated NaHCO₃ solution (10 mL) and extracted with EtOAc (30 mL). The organic layer was washed with brine, dried with Na₂SO₄, filtered, and concentrated in vacuo. The residue was purified by column chromatography (0-25% EtOAc in hexanes) to give the desired product as a white solid along with undesired regioisomer (removed chromatographically). LCMS calculated for C₁₉H₂₀Br₂FN₆O₂(M+H)⁺: m/z=541.0; found: 541.0.

### Step 2. Rac-tert-butyl 2-(1-(7-bromo-4-methyl-2H-[1,2,3]triazolo[4,5-c]pyridin-2-yl)ethyl)-3-fluoro-5,7-dihydro-6H-pyrrolo[3,4-b]pyridine-6-carboxylate

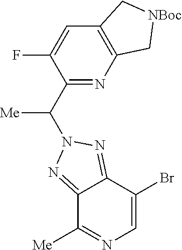 [SMILES: Cc1ncc(Br)c2nn(C(C)c3nc4c(cc3F)CN(C(=O)OC(C)(C)C)C4)nc12]

At a room temperature, Rac-tert-butyl 2-(1-(4,7-dibromo-2H-[1,2,3]triazolo[4,5-c]pyridin-2-yl)ethyl)-3-fluoro-5,7-dihydro-6H-pyrrolo[3,4-b]pyridine-6-carboxylate (150 mg, 0.277 mmol), tetrakis(triphenylphosphine)palladium(0) (32.0 mg, 0.028 mmol) were combined in THF (2 mL). dimethylzinc (1.2M in toluene) (0.346 mL, 0.415 mmol) was added at room temperature. The mixture was stirred at room temperature for 30 min. Upon completion, the resulting mixture was quenched with saturated NH₄Cl solution (5 mL) and extracted with EtOAc (15 mL). The organic layer was washed with brine, dried with Na₂SO₄, filtered, and concentrated in vacuo. The residue was purified by column chromatography (0-55% EtOAc in hexanes) to give the desired product the desired product as a white solid. LCMS calculated for C₂₀H₂₃BrFN₆O₂(M+H)⁺: m/z=477.1; found: 477.1.

### Step 3. Rac-1-(2-(1-(7-(3,5-dichloro-4-hydroxyphenyl)-4-methyl-2H-[1,2,3]triazolo[4,5-c]pyridin-2-yl)ethyl)-3-fluoro-5,7-dihydro-6H-pyrrolo[3,4-b]pyridin-6-yl)prop-2-en-1-one

To a magnetically stirred solution of Rac-tert-butyl 2-(1-(7-bromo-4-methyl-2H-[1,2,3]triazolo[4,5-c]pyridin-2-yl)ethyl)-3-fluoro-5,7-dihydro-6H-pyrrolo[3,4-b]pyridine-6-carboxylate (40 mg, 0.084 mmol) in 1,4-dioxane (828 μL) and water (166 μL) was added 2,6-dichloro-4-(4,4,5,5-tetramethyl-1,3,2-dioxaborolan-2-yl)phenol (48.4 mg, 0.168 mmol), potassium phosphate (53.4 mg, 0.251 mmol) and XPhos-Pd-G4 (3.61 mg, 4.19 μmol). The reaction mixture was then degassed for 10 min before heating to 100° C. After 1 h, the resulting mixture was cooled and diluted with water. The pH of the solution was adjusted to 5 using 1 N HCl and extracted with EtOAc (10 mL). The organic layer was washed with brine, dried with Na₂SO₄, filtered, and concentrated in vacuo.

The obtained crude product was dissolved in dichloromethane (0.5 mL) and 2,2,2-trifluoroacetic acid (0.5 mL) and stirred for 30 mins at 23° C. The obtained crude product was dissolved in dichloromethane (0.5 mL) and 2,2,2-trifluoroacetic acid (0.5 mL) and stirred for 30 mins at 23° C. Upon completion, the reaction mixture was concentrated in vacuo.

To the above crude product in THF (1 mL) was added N-ethyl-N-isopropylpropan-2-amine (16.9 mg, 0.131 mmol), acrylic acid (6.3 mg, 0.087 mmol) and propylphosphonic anhydride (T3P, 50% in EtOAc, 17 μL, 0.057 mmol). The reaction mixture stirred at room temperature for 10 minutes before the solution was diluted with acetonitrile and purified by prep-LCMS (XBridge C18 column, eluting with a gradient of acetonitrile/water containing 0.1% TFA, at flow rate of 60 mL/min) to afford desired product as a pale-yellow solid. LCMS calculated for C₂₄H₂₀C₁₂FN₆O₂(M+H)⁺: m/z=513.1; found: 513.1.

### Example 31. (E)-1-(2-(1-(7-(3,5-dichloro-4-hydroxyphenyl)-2H-[1,2,3]triazolo[4,5-c]pyridin-2-yl)cyclobutyl)-5,7-dihydro-6H-pyrrolo[3,4-b]pyridin-6-yl)-4-(diethylamino)but-2-en-1-one

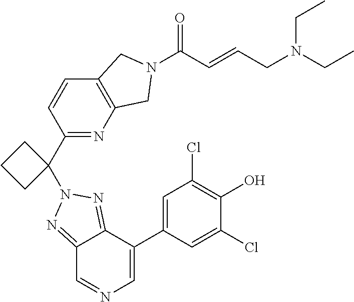 [SMILES: CCN(CC)C/C=C/C(=O)N1Cc2ccc(C3(n4nc5cncc(-c6cc(Cl)c(O)c(Cl)c6)c5n4)CCC3)nc2C1]

### Step 1. tert-Butyl 2-((7-bromo-2H-[1,2,3]triazolo[4,5-c]pyridin-2-yl)methyl)-5,7-dihydro-6H-pyrrolo[3,4-b]pyridine-6-carboxylate

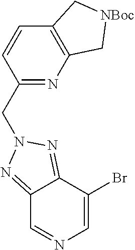 [SMILES: CC(C)(C)OC(=O)N1Cc2ccc(Cn3nc4cncc(Br)c4n3)nc2C1]

To a suspension of 7-bromo-3H-[1,2,3]triazolo[4,5-c]pyridine (360 mg, 1.809 mmol), tert-butyl 2-(hydroxymethyl)-5,7-dihydro-6H-pyrrolo[3,4-b]pyridine-6-carboxylate (498 mg, 1.990 mmol) and triphenylphosphine (712 mg, 2.71 mmol) in tetrahydrofuran (10 mL) at 0° C. was added diisopropyl azodicarboxylate (DIAD, 0.53 mL, 2.71 mmol). The suspension became a solution within 10 min. After stirring at 0° C. for 1 h, the resulting mixture was diluted with water and extracted with ethyl acetate (50 mL). The organic layer was washed with brine, dried with Na₂SO₄, filtered, and concentrated in vacuo. The residue was purified by column chromatography (20-50% AcOEt in DCM). LCMS calculated for C₁₈H₂₀BrN₆O₂ (M+H)⁺: m/z=431.1; found: 431.1.

### Step 2. tert-Butyl 2-(1-(7-bromo-2H-[1,2,3]triazolo[4,5-c]pyridin-2-yl)cyclobutyl)-5,7-dihydro-6H-pyrrolo[3,4-b]pyridine-6-carboxylate

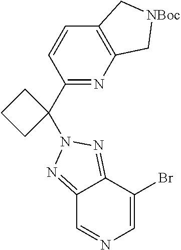 [SMILES: CC(C)(C)OC(=O)N1Cc2ccc(C3(n4nc5cncc(Br)c5n4)CCC3)nc2C1]

To a solution of tert-butyl 2-((7-bromo-2H-[1,2,3]triazolo[4,5-c]pyridin-2-yl)methyl)-5,7-dihydro-6H-pyrrolo[3,4-b]pyridine-6-carboxylate (50 mg, 0.116 mmol) in THF (1 mL) at −78° C. was added LiHMDS (0.23 mL, 0.232 mmol). Stirred at −78° C. for 20 min, then added 1,3-dibromopropane (23.4 mg, 0.116 mmol). The reaction was warmed to 23° C. and stirred at room temperature for 12 hr. Upon completion, the resulting mixture was quenched with saturated NH₄Cl solution (2 mL) and extracted with EtOAc (3 mL). The organic layer was washed with brine, dried with Na₂SO₄, filtered, and concentrated in vacuo. The residue was purified by column chromatography (0-50% EtOAc in hexanes) to give the desired product as a white solid. LCMS calculated for C₂₁H₂₄BrN₆O₂(M+H)⁺: m/z=471.1; found: 471.1.

### Step 3. (E)-1-(2-(1-(7-(3,5-dichloro-4-hydroxyphenyl)-2H-[1,2,3]triazolo[4,5-c]pyridin-2-yl)cyclobutyl)-5,7-dihydro-6H-pyrrolo[3,4-b]pyridin-6-yl)-4-(diethylamino)but-2-en-1-one

To a solution of tert-butyl 2-(1-(7-bromo-2H-[1,2,3]triazolo[4,5-c]pyridin-2-yl)cyclobutyl)-5,7-dihydro-6H-pyrrolo[3,4-b]pyridine-6-carboxylate (15 mg, 0.032 mmol) in 1,4-dioxane (828 μL) and water (166 μL) was added 2,6-dichloro-4-(4,4,5,5-tetramethyl-1,3,2-dioxaborolan-2-yl)phenol (18.4 mg, 0.064 mmol), potassium phosphate (20.2 mg, 0.095 mmol) and XPhos-Pd-G4 (1.369 mg, 1.591 μmol). The reaction mixture was then degassed for 10 min before heating to 100° C. After 1 h, the resulting mixture was cooled and diluted with water. The pH of the solution was adjusted to 5 using 1 N HCl and extracted with EtOAc (5 mL). The organic layer was washed with brine, dried with Na₂SO₄, filtered, and concentrated in vacuo.

The obtained crude product was dissolved in dichloromethane (0.5 mL) and 2,2,2-trifluoroacetic acid (0.5 mL) and stirred for 30 mins at 23° C. Upon completion, the reaction mixture was concentrated in vacuo.

To the above crude product was added N-ethyl-N-isopropylpropan-2-amine (27 μL, 0.154 mmol), (E)-4-(diethylamino)but-2-enoic acid hydrochloride (7.17 mg, 0.037 mmol) and propylphosphonic anhydride (T3P, 50% in EtOAc, 18 μL, 0.031 mmol) in THF (1 mL).

The reaction mixture stirred at room temperature for 10 minutes before the solution was diluted with acetonitrile and purified by prep-LCMS (XBridge C18 column, eluting with a gradient of acetonitrile/water containing 0.1% TFA, at flow rate of 60 mL/min) to afford desired product as an off white solid. LCMS calculated for C₃₀H₃₂C₁₂N₇O₂ (M+H)⁺: m/z=592.2; found: 592.2.

### Example 32. Rac-(E)-1-(2-(cyclopropyl(7-(3,5-dichloro-4-hydroxyphenyl)-4-methyl-2H-[1,2,3]triazolo[4,5-c]pyridin-2-yl)methyl)-5,7-dihydro-6H-pyrrolo[3,4-b]pyridin-6-yl)-4-(dimethylamino)but-2-en-1-one

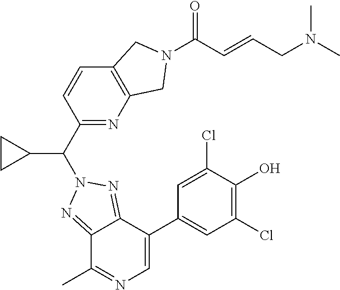 [SMILES: Cc1ncc(-c2cc(Cl)c(O)c(Cl)c2)c2nn(C(c3ccc4c(n3)CN(C(=O)/C=C/CN(C)C)C4)C3CC3)nc12]

### Step 1. Rac-tert-butyl 2-(cyclopropyl(hydroxy)methyl)-5,7-dihydro-6H-pyrrolo[3,4-b]pyridine-6-carboxylate

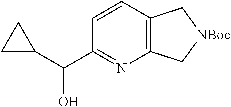 [SMILES: CC(C)(C)OC(=O)N1Cc2ccc(C(O)C3CC3)nc2C1]

To a solution of tert-butyl 2-formyl-5,7-dihydro-6H-pyrrolo[3,4-b]pyridine-6-carboxylate (500 mg, 2.014 mmol) in THF (10 mL) at −78° C. was added cyclopropylmagnesium bromide (0.5M in THF, 12.1 mL, 6.04 mmol). Stirred at −78° C. for 10 min. Upon completion, the mixture was quenched with aq. NH₄Cl and extracted with 10 EtOAc (50 mL) The organic layer was washed with brine, dried over Na₂SO₄, filtered and evaporated in vacuo. Purified via column chromatography (0-50% EtOAc in hexanes). LCMS calculated for C₁₆H₂₃N₂O₃ (M+H)⁺; m/z=291.2; found: 291.2.

### Step 2. Rac-tert-butyl 2-(cyclopropyl(4,7-dibromo-2H-[1,2,3]triazolo[4,5-c]pyridin-2-yl)methyl)-5,7-dihydro-6H-pyrrolo[3,4-b]pyridine-6-carboxylate

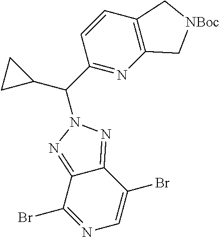 [SMILES: CC(C)(C)OC(=O)N1Cc2ccc(C(C3CC3)n3nc4c(Br)cnc(Br)c4n3)nc2C1]

4,7-dibromo-2H-[1,2,3]triazolo[4,5-c]pyridine (350 mg, 1.259 mmol), rac-tert-butyl 2-(cyclopropyl(hydroxy)methyl)-5,7-dihydro-6H-pyrrolo[3,4-b]pyridine-6-carboxylate (439 mg, 1.511 mmol) and triphenylphosphine (496 mg, 1.889 mmol) were combined in THF (12.6 mL). The mixture was cooled to 0° C. before diisopropyl azodicarboxylate (367 μL, 1.889 mmol) was added. After 15 minutes, the bath was removed and the reaction was stirred at room temperature overnight. Upon completion, the mixture was diluted with water and extracted with EtOAc (50 mL). The organic layer was washed with brine, dried with Na₂SO₄, filtered, and concentrated in vacuo. The residue was purified by column chromatography (20-80% EtOAc in hexanes) to remove undesired regioisomers. LCMS calculated for C₂₁H₂₃Br₂N₆O₂ (M+H)⁺: m/z=549.0; found: 549.0.

### Step 3. Rac-tert-butyl 2-((7-bromo-4-methyl-2H-[1,2,3]triazolo[4,5-c]pyridin-2-yl)(cyclopropyl)methyl)-5,7-dihydro-6H-pyrrolo[3,4-b]pyridine-6-carboxylate

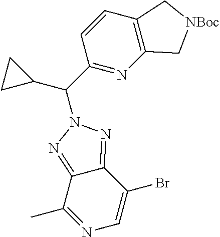 [SMILES: Cc1ncc(Br)c2nn(C(c3ccc4c(n3)CN(C(=O)OC(C)(C)C)C4)C3CC3)nc12]

In a room temperature water bath, Rac-tert-butyl 2-(cyclopropyl(4,7-dibromo-2H-[1,2,3]triazolo[4,5-c]pyridin-2-yl)methyl)-5,7-dihydro-6H-pyrrolo[3,4-b]pyridine-6-carboxylate (250 mg, 0.454 mmol), tetrakis(triphenylphosphine)palladium(0) (52.5 mg, 0.045 mmol) were combined in THF (4.5 mL). The suspension was stirred at room temperature for 10 minutes until it became a clear solution. Dimethylzinc (1.0M in heptane, 682 μL, 0.682 mmol) was added at room temperature. After 10 minutes, the reaction was quenched with a saturated solution of NH₄Cl and extracted with EtOAc (30 mL). The organic layer was then washed with brine, dried with Na₂SO₄, filtered and concentrated in vacuo. The residue was then purified via column chromatography (0-80% MTBE in hexanes to removed undesired regioisomers. LCMS calculated for C₂₂H₂₆BrN₆O₂(M+H)⁺: 485.1; found: 485.1.

### Step 4. Rac-tert-butyl 2-(cyclopropyl(7-(3,5-dichloro-4-hydroxyphenyl)-4-methyl-2H-[1,2,3]triazolo[4,5-c]pyridin-2-yl)methyl)-5,7-dihydro-6H-pyrrolo[3,4-b]pyridine-6-carboxylate

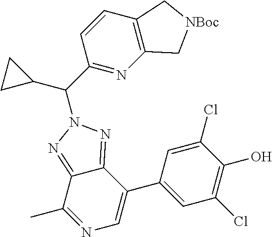 [SMILES: Cc1ncc(-c2cc(Cl)c(O)c(Cl)c2)c2nn(C(c3ccc4c(n3)CN(C(=O)OC(C)(C)C)C4)C3CC3)nc12]

To a solution of Rac-tert-butyl 2-((7-bromo-4-methyl-2H-[1,2,3]triazolo[4,5-c]pyridin-2-yl)(cyclopropyl)methyl)-5,7-dihydro-6H-pyrrolo[3,4-b]pyridine-6-carboxylate (160 mg, 0.330 mmol) in 1,4-dioxane (2747 μL) and water (549 μL) was added (3,5-dichloro-4-hydroxyphenyl)boronic acid (109 mg, 0.527 mmol), potassium phosphate (210 mg, 0.989 mmol) and XPhos-Pd-G4 (28.4 mg, 0.033 mmol). The reaction mixture was then degassed for 10 min before heating to 95° C. After 1 h, the mixture was cooled, diluted with water and extracted with EtOAc (50 mL). The organic layer was washed with brine, dried over Na₂SO₄, filtered and concentrated in vacuo. The residue was purified via column chromatography (0-80% EtOAc in hexanes). LCMS calculated for C₂₈H₂₉C₁₂N₆O₃ (M+H)⁺; m/z=567.1; found: 567.1.

### Step 5. Rac-(E)-1-(2-(cyclopropyl(7-(3,5-dichloro-4-hydroxyphenyl)-4-methyl-2H-[1,2,3]triazolo[4,5-c]pyridin-2-yl)methyl)-5,7-dihydro-6H-pyrrolo[3,4-b]pyridin-6-yl)-4-(dimethylamino)but-2-en-1-one

This compound was synthesized based on a similar procedure as described in Example 27, using Rac-tert-butyl 2-(cyclopropyl(7-(3,5-dichloro-4-hydroxyphenyl)-4-methyl-2H-[1,2,3]triazolo[4,5-c]pyridin-2-yl)methyl)-5,7-dihydro-6H-pyrrolo[3,4-b]pyridine-6-carboxylate instead of Intermediate 10 and using (E)-4-(dimethylamino)but-2-enoic acid hydrochloride instead of (E)-4-(diethylamino)but-2-enoic acid hydrochloride. LCMS calculated for C₂₉H₃₀Cl₂N₇O₂ (M+H)⁺: m/z=578.2; found: 578.2.

### Example 33. Rac-(E)-1-(2-(1-(7-(3,5-dichloro-4-hydroxyphenyl)-2H-[1,2,3]triazolo[4,5-c]pyridin-2-yl)propan-2-yl)-5,7-dihydro-6H-pyrrolo[3,4-b]pyridin-6-yl)-4-(diethylamino)but-2-en-1-one

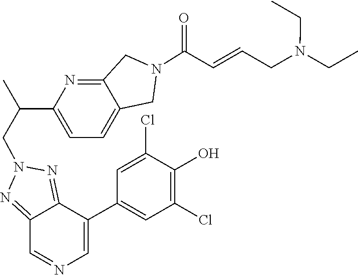 [SMILES: CCN(CC)C/C=C/C(=O)N1Cc2ccc(C(C)Cn3nc4cncc(-c5cc(Cl)c(O)c(Cl)c5)c4n3)nc2C1]

### Step 1. (9H-fluoren-9-yl)methyl 2-chloro-5,7-dihydro-6H-pyrrolo[3,4-b]pyridine-6-carboxylate

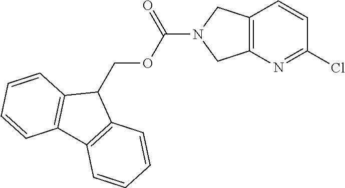 [SMILES: O=C(OCC1c2ccccc2-c2ccccc21)N1Cc2ccc(Cl)nc2C1]

To a solution of 2-chloro-6,7-dihydro-5H-pyrrolo[3,4-b]pyridine hydrochloride (1 g, 5.23 mmol) and DIPEA (2.28 mL, 13.09 mmol) in THF (20 mL) at room temperature was added (9H-fluoren-9-yl)methyl (2,5-dioxopyrrolidin-1-yl) carbonate (Fmoc-OSu, 2 g, 5.93 mmol). The mixture was stirred at room temperature overnight, then quenched with water, diluted with EtOAc. The layers were separated. The organic layer was washed with brine, dried over Na₂SO₄, filtered and evaporated. The residue was purified by column chromatography (20-80% EtOAc in hexanes). LCMS calculated for C₂₂H₁₈C₁N₂O₂(M+H)⁺: m/z=377.1; found: 377.1.

### Step 2. (9H-fluoren-9-yl)methyl 2-(prop-1-en-2-yl)-5,7-dihydro-6H-pyrrolo[3,4-b]pyridine-6-carboxylate

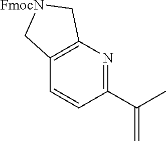

To a mixture of (9H-fluoren-9-yl)methyl 2-chloro-5,7-dihydro-6H-pyrrolo[3,4-b]pyridine-6-carboxylate (1.124 g, 2.98 mmol), trifluoro(prop-1-en-2-yl)-/4-borane, potassium salt (0.883 g, 5.97 mmol) and potassium phosphate (1.9 g, 8.95 mmol) in 1,4-dioxane (20 mL) and water (4 mL) was added XPhos-Pd-G4 (0.257 g, 0.298 mmol). The reaction mixture was then heated to 100° C. After stirring for 1 h, the mixture was cooled to room temperature, diluted with water and EtOAc. The layers were separated. The organic layer was washed with brine, dried over Na₂SO₄, filtered and evaporated. The residue was purified by column chromatography (20-60% EtOAc in hexanes). LCMS calculated for C₂₅H₂₃N₂O₂ (M+H)⁺: m/z=383.2; found: 383.2.

### Step 3. Rac-(9H-fluoren-9-yl)methyl 2-(1-(7-bromo-4-methyl-2H-[1,2,3]triazolo[4,5-c]pyridin-2-yl)propan-2-yl)-5,7-dihydro-6H-pyrrolo[3,4-b]pyridine-6-carboxylate

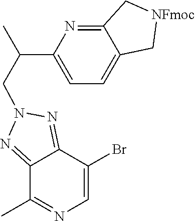

To a mixture of 7-bromo-4-methyl-3H-[1,2,3]triazolo[4,5-c]pyridine (250 mg, 1.173 mmol) and (9H-fluoren-9-yl)methyl 2-(prop-1-en-2-yl)-5,7-dihydro-6H-pyrrolo[3,4-b]pyridine-6-carboxylate (449 mg, 1.173 mmol) in toluene (6 mL) was added Ts-OH H₂O (670 mg, 3.52 mmol). The reaction mixture was then heated to 115° C., stirred at the same temperature overnight. The mixture was cooled to room temperature, quenched with aqueous sodium bicarbonate and diluted with EtOAc. The layers were separated. The organic layer was washed with brine, dried over Na₂SO₄, filtered and evaporated. The residue was purified by column chromatography (20-60% EtOAc in hexanes). LCMS calculated for C₃₁H₂₈BrN₆O₂(M+H)⁺: m/z=595.1; found: 595.1.

### Step 4. Rac-(9H-fluoren-9-yl)methyl 2-(1-(7-(3,5-dichloro-4-hydroxyphenyl)-2H-[1,2,3]triazolo[4,5-c]pyridin-2-yl)propan-2-yl)-5,7-dihydro-6H-pyrrolo[3,4-b]pyridine-6-carboxylate

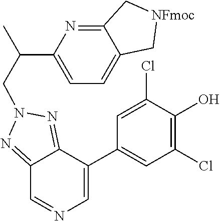

To a suspension of rac-(9H-fluoren-9-yl)methyl 2-(1-(7-bromo-2H-[1,2,3]triazolo[4,5-c]pyridin-2-yl)propan-2-yl)-5,7-dihydro-6H-pyrrolo[3,4-b]pyridine-6-carboxylate (196 mg, 0.338 mmol), (3,5-dichloro-4-hydroxyphenyl)boronic acid (105 mg, 0.506 mmol) and potassium phosphate (215 mg, 1.013 mmol) in 1,4-dioxane (2.5 mL) and water (0.5 mL) was added XPhos-Pd-G4 (29.1 mg, 0.034 mmol). The reaction mixture was then degassed for 10 min before heating to 100° C. Upon completion, the resulting mixture was cooled and diluted with water and extracted with EtOAc (20 mL). The organic layer was washed with brine, dried with Na₂SO₄, filtered, and concentrated in vacuo. The residue was purified by column chromatography (0-50% EtOAc in hex). LCMS calculated for C₃₆H₂₉C₁₂N₆O₃ (M+H)⁺: m/z=663.2; found: 663.2.

### Step 5. Rac-(E)-1-(2-(1-(7-(3,5-dichloro-4-hydroxyphenyl)-2H-[1,2,3]triazolo[4,5-c]pyridin-2-yl)propan-2-yl)-5,7-dihydro-6H-pyrrolo[3,4-b]pyridin-6-yl)-4-(diethylamino)but-2-en-1-one

To a solution of rac-(9H-fluoren-9-yl)methyl 2-(1-(7-(3,5-dichloro-4-hydroxyphenyl)-2H-[1,2,3]triazolo[4,5-c]pyridin-2-yl)propan-2-yl)-5,7-dihydro-6H-pyrrolo[3,4-b]pyridine-6-carboxylate in DCM (2.5 mL) was added diethylamine (0.35 mL, 3.38 mmol). The mixture was stirred at room temperature for 10 min, then evaporated and used for the next step without further purification.

To a suspension of above compound, (E)-4-(diethylamino)but-2-enoic acid (10.7 mg, 0.068 mmol) and DIPEA (0.040 mL, 0.227 mmol) in THF (2 mL) at 0° C. was added propylphosphonic anhydride (T3P, 50% in EtOAc, 0.013 mL, 0.045 mmol). The reaction mixture was stirred at 0° C. for 5 min, then diluted with acetonitrile and purified by prep-LCMS (XBridge C18 column, eluting with a gradient of acetonitrile/water containing 0.1% TFA, at flow rate of 60 mL/min) to afford desired product as a TFA salt. LCMS calculated for C₂₉H₃₂C₁₂N₇O₂ (M+H)⁺: m/z=580.2; found: 580.2.

### Example 34. Rac-(E)-1-(2-(1-(4-amino-7-(3,5-dichloro-4-hydroxyphenyl)-2H-[1,2,3]triazolo[4,5-c]pyridin-2-yl)ethyl)-5,7-dihydro-6H-pyrrolo[3,4-b]pyridin-6-yl)-4-(2,2-dimethylpyrrolidin-1-yl)but-2-en-1-one

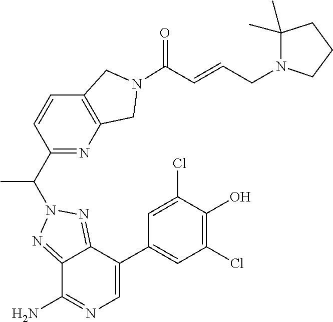 [SMILES: CC(c1ccc2c(n1)CN(C(=O)/C=C/CN1CCCC1(C)C)C2)n1nc2c(-c3cc(Cl)c(O)c(Cl)c3)cnc(N)c2n1]

### Step 1. Rac-tert-butyl 2-(1-(4-(bis(4-methoxybenzyl)amino)-7-bromo-2H-[1,2,3]triazolo[4,5-c]pyridin-2-yl)ethyl)-5,7-dihydro-6H-pyrrolo[3,4-b]pyridine-6-carboxylate

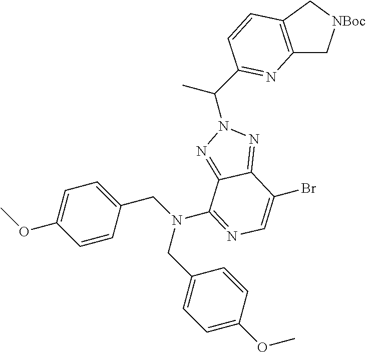 [SMILES: COc1ccc(CN(Cc2ccc(OC)cc2)c2ncc(Br)c3nn(C(C)c4ccc5c(n4)CN(C(=O)OC(C)(C)C)C5)nc23)cc1]

A vial was charged with Rac-tert-butyl 2-(1-(4,7-dibromo-2H-[1,2,3]triazolo[4,5-c]pyridin-2-yl)ethyl)-5,7-dihydro-6H-pyrrolo[3,4-b]pyridine-6-carboxylate (Intermediate 10, step 2, 250 mg, 0.477 mmol), bis(4-methoxybenzyl)amine (184 mg, 0.715 mmol) and DIPEA (167 μL, 0.954 mmol) and DMF (4.8 mL). The mixture was stirred at 120° C. for 2h. Upon completion, the mixture was diluted with water and extracted with EtOAc (50 mL). The organic phase was then washed with brine, dried over Na₂SO₄, filtered and concentrated in vacuo. The resulting residue was then purified via column chromatography (0-100% EtOAc in hexanes). LCMS calculated for C₃₅H₃₉BrN₇O₄(M+H)⁺; m/z=700.2; found: 700.2.

### Step 2. Rac-tert-butyl 2-(1-(4-(bis(4-methoxybenzyl)amino)-7-(3,5-dichloro-4-hydroxyphenyl)-2H-[1,2,3]triazolo[4,5-c]pyridin-2-yl)ethyl)-5,7-dihydro-6H-pyrrolo[3,4-b]pyridine-6-carboxylate

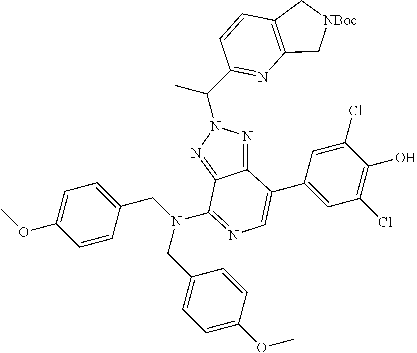 [SMILES: COc1ccc(CN(Cc2ccc(OC)cc2)c2ncc(-c3cc(Cl)c(O)c(Cl)c3)c3nn(C(C)c4ccc5c(n4)CN(C(=O)OC(C)(C)C)C5)nc23)cc1]

To a solution of rac-tert-butyl 2-(1-(4-(bis(4-methoxybenzyl)amino)-7-bromo-2H-[1,2,3]triazolo[4,5-c]pyridin-2-yl)ethyl)-5,7-dihydro-6H-pyrrolo[3,4-b]pyridine-6-carboxylate (200 mg, 0.285 mmol) in 1,4-dioxane (2.4 mL) and water (476 μL) was added 2,6-dichloro-4-(4,4,5,5-tetramethyl-1,3,2-dioxaborolan-2-yl)phenol (132 mg, 0.457 mmol), potassium phosphate (182 mg, 0.856 mmol) and XPhos-Pd-G4 (24.6 mg, 0.029 mmol). The reaction mixture was then degassed for 10 min before heating to 95° C. After 1 h, the mixture was cooled, diluted with water and extracted with EtOAc (50 mL). The organic layer was washed with brine, dried over Na₂SO₄, filtered and concentrated in vacuo. The residue was purified via column chromatography (0-80% EtOAc in hexanes). LCMS calculated for C₄₁H₄₂C₁₂N₇O₅ (M+H)+; m/z=782.2; found: 782.2.

### Step 3. Rac-(E)-1-(2-(1-(4-amino-7-(3,5-dichloro-4-hydroxyphenyl)-2H-[1,2,3]triazolo[4,5-c]pyridin-2-yl)ethyl)-5,7-dihydro-6H-pyrrolo[3,4-b]pyridin-6-yl)-4-(2,2-dimethylpyrrolidin-1-yl)but-2-en-1-one

Rac-tert-butyl 2-(1-(4-(bis(4-methoxybenzyl)amino)-7-(3,5-dichloro-4-hydroxyphenyl)-2H-[1,2,3]triazolo[4,5-c]pyridin-2-yl)ethyl)-5,7-dihydro-6H-pyrrolo[3,4-b]pyridine-6-carboxylate (10 mg, 0.15 mmol) was dissolved in DCM (0.5 mL) and 2,2,2 trifluoroacetic acid. Stirred at room temperature for 30 minutes before the volatiles were removed in vacuo. The resulting oil was then diluted with THF (0.5 mL). DIPEA (26 μL, 0.146 mmol), (E)-4-(2,2-dimethylpyrrolidin-1-yl)but-2-enoic acid hydrochloride (9.7 mg, 0.045 mmol) and HATU (6.7 mg, 0.018 mmol) were then added to the mixture. The suspension was stirred at room temperature for 15 minutes before the volatiles were removed in vacuo. The resulting oil was then diluted with 2,2,2 trifluoroacetic acid (1 mL) and heated to 100° C. Cooled to room temperature and diluted with MeOH. Purified via prep-LCMS (XBridge C18 column, eluting with a gradient of acetonitrile/water containing 0.1% TFA, at flow rate of 60 mL/min) to afford desired product as a TFA salt. LCMS calculated for C₃₀H₃₃C₁₂N₈O₂ (M+H)⁺; m/z=607.2; found: 607.2

### Example 35. (E)-7-(3,5-dichloro-4-hydroxyphenyl)-2-((6-(4-(diethylamino)but-2-enoyl)-3-methoxy-6,7-dihydro-5H-pyrrolo[3,4-b]pyridin-2-yl)methyl)-2H-[1,2,3]triazolo[4,5-c]pyridine-4-carbonitrile

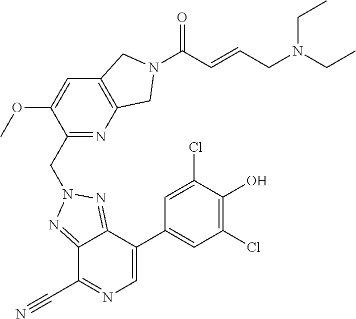 [SMILES: CCN(CC)C/C=C/C(=O)N1Cc2cc(OC)c(Cn3nc4c(-c5cc(Cl)c(O)c(Cl)c5)cnc(C#N)c4n3)nc2C1]

### Step 1. tert-Butyl 2-((4,7-dibromo-2H-[1,2,3]triazolo[4,5-c]pyridin-2-yl)methyl)-3-methoxy-5,7-dihydro-6H-pyrrolo[3,4-b]pyridine-6-carboxylate

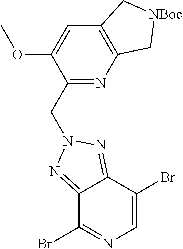 [SMILES: COc1cc2c(nc1Cn1nc3c(Br)cnc(Br)c3n1)CN(C(=O)OC(C)(C)C)C2]

To the magnetically stirred solution of 4,7-dibromo-2H-[1,2,3]triazolo[4,5-c]pyridine hydrochloride (561 mg, 1.784 mmol), tert-butyl 2-(hydroxymethyl)-3-methoxy-5,7-dihydro-6H-pyrrolo[3,4-b]pyridine-6-carboxylate (500 mg, 1.784 mmol) and triphenylphosphine (561 mg, 2.140 mmol) were combined in THF (5 mL) at 0° C. followed by addition of N-ethyl-N-isopropylpropan-2-amine (0.961 mL, 5.35 mmol) and diisopropyl azodicarboxylate (367 μL, 1.889 mmol). The mixture was stirred at 0° C. for 45 min and then warmed to 23° C. and stirred for 15 min. Upon completion, the resulting mixture was quenched with saturated NaHCO₃ solution (10 mL) and extracted with EtOAc (30 mL). The organic layer was washed with brine, dried with Na₂SO₄, filtered, and concentrated in vacuo. The residue was purified by column chromatography (0-55% EtOAc in hexanes) to give the desired product as a white solid along with undesired regioisomer (removed chromatographically). LCMS calculated for C₁₉H₂₁Br₂N₆O₃ (M+H)⁺: m/z=539.0; found: 539.0.

### Step 2. tert-Butyl 2-((7-bromo-4-cyano-2H-[1,2,3]triazolo[4,5-c]pyridin-2-yl)methyl)-3-methoxy-5,7-dihydro-6H-pyrrolo[3,4-b]pyridine-6-carboxylate

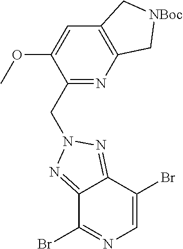 [SMILES: COc1cc2c(nc1Cn1nc3c(Br)cnc(Br)c3n1)CN(C(=O)OC(C)(C)C)C2]

To the magnetically stirred solution of tert-butyl 2-((4,7-dibromo-2H-[1,2,3]triazolo[4,5-c]pyridin-2-yl)methyl)-3-methoxy-5,7-dihydro-6H-pyrrolo[3,4-b]pyridine-6-carboxylate (50 mg, 0.093 mmol), tetrakis(triphenylphosphine)palladium (10.7 mg, 9.26 μmol) and zinc cyanide (16.3 mg, 0.139 mmol) were combined in DMF (1 mL). The mixture was stirred at 80° C. for 8 h. Upon completion, the resulting mixture was quenched with saturated NH₄Cl solution (5 mL) and extracted with EtOAc (5 mL). The organic layer was washed with brine, dried with Na₂SO₄, filtered, and concentrated in vacuo. The residue was purified by column chromatography (0-65% EtOAc in hexanes) to give the desired product the desired product as a white solid. LCMS calculated for C₂₀H₂₁BrN₇O₃ (M+H)⁺: m/z=486.1; found: 486.1.

### Step 3. (E)-7-(3,5-dichloro-4-hydroxyphenyl)-2-((6-(4-(diethylamino)but-2-enoyl)-3-methoxy-6,7-dihydro-5H-pyrrolo[3,4-b]pyridin-2-yl)methyl)-2H-[1,2,3]triazolo[4,5-c]pyridine-4-carbonitrile

To a magnetically stirred solution of tert-butyl 2-((7-bromo-4-cyano-2H-[1,2,3]triazolo[4,5-c]pyridin-2-yl)methyl)-3-methoxy-5,7-dihydro-6H-pyrrolo[3,4-b]pyridine-6-carboxylate (15 mg, 0.031 mmol) in 1,4-dioxane (828 μL) and water (166 μL) was added 2,6-dichloro-4-(4,4,5,5-tetramethyl-1,3,2-dioxaborolan-2-yl)phenol (10.7 mg, 0.037 mmol), potassium phosphate (19.6 mg, 0.093 mmol) and XPhos-Pd-G4 (1.3 mg, 1.542 μmol). The reaction mixture was then degassed for 10 min before heating to 100° C. After 1h, the resulting mixture was cooled and diluted with water. The pH of the solution was adjusted to 5 using 1 N HCl and extracted with EtOAc (5 mL). The organic layer was washed with brine, dried with Na₂SO₄, filtered, and concentrated in vacuo.

The obtained crude product was dissolved in dichloromethane (0.5 mL) and 2,2,2-trifluoroacetic acid (0.5 mL) and stirred for 30 mins at 23° C. Upon completion, the reaction mixture was concentrated in vacuo.

To the above crude product was added N-ethyl-N-isopropylpropan-2-amine (28 μL, 0.154 mmol), (E)-4-(diethylamino)but-2-enoic acid hydrochloride (7.2 mg, 0.037 mmol) and propylphosphonic anhydride (T3P, 50% in EtOAc, 18 μL, 0.031 mmol) in THF (1 mL). The reaction mixture stirred at room temperature for 10 minutes before the solution was diluted with acetonitrile and purified by prep-LCMS (XBridge C18 column, eluting with a gradient of acetonitrile/water containing 0.1% TFA, at flow rate of 60 mL/min) to afford desired product as a pale-yellow solid. LCMS calculated for C₂₉H₂₉C₁₂N₈O₃ (M+H)⁺: m/z=607.2; found: 607.2.

### Example 36. Rac-(E)-4-(3-azabicyclo[3.1.1]heptan-3-yl)-1-(3-(1-(7-(3,5-dichloro-4-hydroxyphenyl)-4-ethyl-2H-[1,2,3]triazolo[4,5-c]pyridin-2-yl)ethyl)-2-methoxy-5,7-dihydro-6H-pyrrolo[3,4-b]pyridin-6-yl)but-2-en-1-one

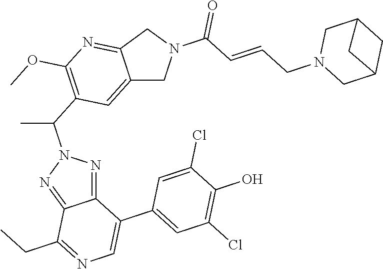 [SMILES: CCc1ncc(-c2cc(Cl)c(O)c(Cl)c2)c2nn(C(C)c3cc4c(nc3OC)CN(C(=O)/C=C/CN3CC5CC(C5)C3)C4)nc12]

### Step 1. Rac-tert-butyl 3-((4,7-dibromo-2H-[1,2,3]triazolo[4,5-c]pyridin-2-yl)methyl)-2-methoxy-5,7-dihydro-6H-pyrrolo[3,4-b]pyridine-6-carboxylate

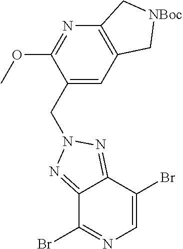 [SMILES: COc1nc2c(cc1Cn1nc3c(Br)cnc(Br)c3n1)CN(C(=O)OC(C)(C)C)C2]

To a solution of 4,7-dibromo-2H-[1,2,3]triazolo[4,5-c]pyridine (1.2 g, 4.3 mmol), tert-butyl 3-(hydroxymethyl)-2-methoxy-5,7-dihydro-6H-pyrrolo[3,4-b]pyridine-6-carboxylate (1.27 g, 4.5 mmol), DIPEA (1.5 mL, 8.6 mmol) and triphenylphosphine (1.7 g, 6.5 mmol) in THF (10 mL) at 0° C. was added diisopropyl azodicarboxylate (1.2 mL, 6.5 mmol). After 15 min the bath was removed, and the mixture was stirred at room temperature overnight. Upon completion, the mixture was diluted with water and extracted with EtOAc (20 mL). The organic layer was washed with brine, dried with Na₂SO₄, filtered, and concentrated in vacuo. The residue was purified by column chromatography (20-80% EtOAc in hexanes) to remove undesired regioisomers. LCMS calculated for C₁₉H₂₁Br₂N₆O₃ (M+H)⁺: m/z=539.0; found: 539.0.

### Step 2. tert-Butyl 3-((7-bromo-4-ethyl-2H-[1,2,3]triazolo[4,5-c]pyridin-2-yl)methyl)-2-methoxy-5,7-dihydro-6H-pyrrolo[3,4-b]pyridine-6-carboxylate

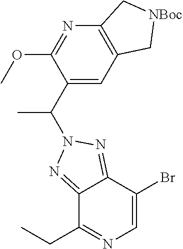 [SMILES: CCc1ncc(Br)c2nn(C(C)c3cc4c(nc3OC)CN(C(=O)OC(C)(C)C)C4)nc12]

In a room temperature water bath tert-butyl 3-((4,7-dibromo-2H-[1,2,3]triazolo[4,5-c]pyridin-2-yl)methyl)-2-methoxy-5,7-dihydro-6H-pyrrolo[3,4-b]pyridine-6-carboxylate (500 mg, 0.93 mmol) and tetrakis(triphenylphosphine)palladium(0) (107 mg, 0.09 mmol) were combined in THF (5 mL). The suspension was stirred for 5 min until it became a clear solution. Diethylzinc (1.0 M in hexane, 1.4 mL, 1.4 mmol) was added at room temperature. After 10 min, the mixture was quenched with aqueous NH₄OH and extracted with EtOAc (20 mL). The organic layer was washed with brine, dried with Na₂SO₄, filtered, and concentrated in vacuo. The residue was purified by column chromatography (20-50% EtOAc in hexanes) to get the product. LCMS calculated for C₂₁H₂₆BrN₆O₃(M+H)⁺: m/z=489.1; found: 489.1.

### Step 3. Rac-tert-butyl 3-(1-(7-bromo-4-ethyl-2H-[1,2,3]triazolo[4,5-c]pyridin-2-yl)ethyl)-2-methoxy-5,7-dihydro-6H-pyrrolo[3,4-b]pyridine-6-carboxylate

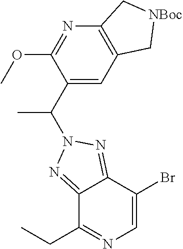 [SMILES: CCc1ncc(Br)c2nn(C(C)c3cc4c(nc3OC)CN(C(=O)OC(C)(C)C)C4)nc12]

To a solution of tert-butyl 3-((7-bromo-4-ethyl-2H-[1,2,3]triazolo[4,5-c]pyridin-2-yl)methyl)-2-methoxy-5,7-dihydro-6H-pyrrolo[3,4-b]pyridine-6-carboxylate (400 mg, 0.82 mmol) in THF (4 mL) was added lithium bis(trimethylsilyl)amide solution (1M in TIFF, 1.6 mL, 1.6 mmol) at −78° C. The reaction mixture was stirred at −78° C. for 20 min before methyl iodide (0.15 mL, 2.4 mmol) was added. The reaction mixture was then slowly allowed to warm up to room temperature over the period of 10 min. After 10 min, the mixture was quenched with aqueous NH₄Cl and extracted with EtOAc (20 mL). The organic layer was washed with brine, dried with Na₂SO₄, filtered, and concentrated in vacuo. The residue was purified by column chromatography (0-80% EtOAc in hexanes) to get the product. LCMS calculated for C₂₂H₂₈BrN₆O₃(M+H)⁺: m/z=503.1; found: 503.1.

### Step 4. Rac-2,6-dichloro-4-(4-ethyl-2-(1-(2-methoxy-6,7-dihydro-5H-pyrrolo[3,4-b]pyridin-3-yl)ethyl)-2H-[1,2,3]triazolo[4,5-c]pyridin-7-yl)phenol

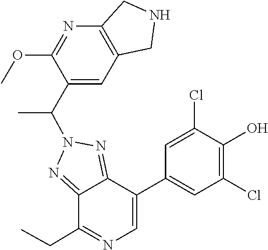 [SMILES: CCc1ncc(-c2cc(Cl)c(O)c(Cl)c2)c2nn(C(C)c3cc4c(nc3OC)CNC4)nc12]

To a mixture of rac-tert-butyl 3-(1-(7-bromo-4-ethyl-2H-[1,2,3]triazolo[4,5-c]pyridin-2-yl)ethyl)-2-methoxy-5,7-dihydro-6H-pyrrolo[3,4-b]pyridine-6-carboxylate (140 mg, 0.28 mmol), (3,5-dichloro-4-hydroxyphenyl)boronic acid (117 mg, 0.57 mmol) and potassium phosphate (180 mg, 0.85 mmol) was added XPhos-Pd-G4 (25.0 mg, 0.03 mmol) in 1,4-dioxane/water (5:1, 2.8 mL). The reaction mixture was then degassed for 10 min before heating to 95° C. for 1 h. After 1 h, the resulting mixture was cooled and diluted with water and extracted with EtOAc (50 mL). The organic layer was washed with brine, dried with Na₂SO₄, filtered, and concentrated in vacuo. The residue was purified by column chromatography (0-500 methanol in dichloromethane) to get the product, which was dissolved in dichloromethane/2,2,2-trifluoroacetic acid (1:1, 2 mL) and stirred for 20 mins. After completion, the reaction mixture was concentrated in vacuo. The crude material was used for the next step without further purification. LCMS calculated for C₂₃H₂₃Cl₂N₆O₂ (M+H)*: m/z 20=485.1; found: 485.1.

### Step 5. Rac-(E)-4-(3-azabicyclo[3.1.1]heptan-3-yl)-1-(3-(1-(7-(3,5-dichloro-4-hydroxyphenyl)-4-ethyl-2H-[1,2,3]triazolo[4,5-c]pyridin-2-yl)ethyl)-2-methoxy-5,7-dihydro-6H-pyrrolo[3,4-b]pyridin-6-yl)but-2-en-1-one

To a suspension of rac-2,6-dichloro-4-(4-ethyl-2-(1-(2-methoxy-6,7-dihydro-5H-pyrrolo[3,4-b]pyridin-3-yl)ethyl)-2H-[1,2,3]triazolo[4,5-c]pyridin-7-yl)phenol (14 mg, 0.028 mmol), (E)-4-(3-azabicyclo[3.1.1]heptan-3-yl)but-2-enoic acid (10 mg; 0.036 mmol) and DIPEA (30 μL, 0.17 mmol) in THF (1 mL) was added propylphosphonic anhydride (T3P, 5000 in EtOAc, 20 μL, 0.03 mmol). Stirred at room temperature for 30 minutes before the solution was diluted with MeOH and purified by prep-LCMS (XBridge C18 column, eluting with a gradient of acetonitrile/water containing 0.1% TFA, at flow rate of 60 mL/min) to afford desired product as a TFA salt. LCMS calculated for C₃₃H₃₆C₁₂N₇O₃ (M+H)⁺: 648.2; found: 648.2.

### Example 37. Rac-1-(2-(1-(7-(3,5-dichloro-4-hydroxyphenyl)-4-methoxy-2H-[1,2,3]triazolo[4,5-c]pyridin-2-yl)ethyl)-5,7-dihydro-6H-pyrrolo[3,4-b]pyridin-6-yl)prop-2-en-1-one

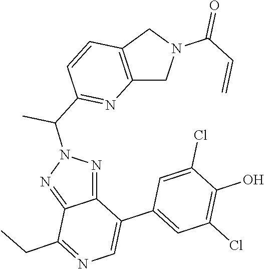 [SMILES: C=CC(=O)N1Cc2ccc(C(C)n3nc4c(-c5cc(Cl)c(O)c(Cl)c5)cnc(CC)c4n3)nc2C1]

### Step 1. Rac-tert-butyl 2-(1-(7-bromo-4-methoxy-2H-[1,2,3]triazolo[4,5-c]pyridin-2-yl)ethyl)-5,7-dihydro-6H-pyrrolo[3,4-b]pyridine-6-carboxylate

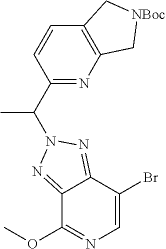 [SMILES: COc1ncc(Br)c2nn(C(C)c3ccc4c(n3)CN(C(=O)OC(C)(C)C)C4)nc12]

To a solution of Rac-tert-butyl 2-(1-(4,7-dibromo-2H-[1,2,3]triazolo[4,5-c]pyridin-2-yl)ethyl)-5,7-dihydro-6H-pyrrolo[3,4-b]pyridine-6-carboxylate (Intermediate 10, step 3, 58 mg, 0.111 mmol) in methanol (1.1 mL) was added sodium methoxide (5M in methanol, 0.11 mL, 0.55 mmol). The reaction mixture was stirred at 50° C. for 30 minutes. Upon full completion, the reaction mixture was cooled to room temperature, quenched with water and extracted with EtOAc. The combined organic extracts were dried over sodium sulfate and concentrated under reduced pressure to give light yellow residue, which was used directly without further purification in the next step. LCMS calculated for C₂₀H₂₄BrN₆O₃(M+H)⁺: m/z=475.1; found: 475.1.

### Step 2. tert-Butyl 2-(1-(7-(3,5-dichloro-4-hydroxyphenyl)-4-methoxy-2H-[1,2,3]triazolo[4,5-c]pyridin-2-yl)ethyl)-5,7-dihydro-6H-pyrrolo[3,4-b]pyridine-6-carboxylate

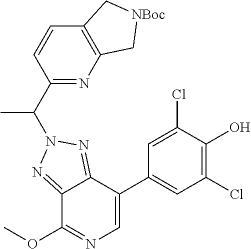 [SMILES: COc1ncc(-c2cc(Cl)c(O)c(Cl)c2)c2nn(C(C)c3ccc4c(n3)CN(C(=O)OC(C)(C)C)C4)nc12]

To a solution of tert-butyl 2-(1-(7-bromo-4-methoxy-2H-[1,2,3]triazolo[4,5-c]pyridin-2-yl)ethyl)-5,7-dihydro-6H-pyrrolo[3,4-b]pyridine-6-carboxylate (20 mg, 0.042 mmol) in 1,4-dioxane (350 μL) and water (70 μL) was added 2,6-dichloro-4-(4,4,5,5-tetramethyl-1,3,2-dioxaborolaN-2-yl)phenol (18.2 mg, 0.063 mmol), potassium phosphate (22.3 mg, 0.105 mmol) and XPhos-Pd-G4 (3.6 mg, 4.21 μmol). The reaction mixture was then degassed for 10 minutes before heating to 80° C. for 30 minutes. Upon full completion, the reaction mixture was concentrated and purified by flash column chromatography (0-80% EtOAc in hexanes) to give pure product. LCMS calculated for C₂₆H₂₇C₁₂N₆O₄ (M+H)⁺: m/z=557.1; found: 557.1.

### Step 3. 1-(2-(1-(7-(3,5-Dichloro-4-hydroxyphenyl)-4-methoxy-2H-[1,2,3]triazolo[4,5-c]pyridin-2-yl)ethyl)-5,7-dihydro-6H-pyrrolo[3,4-b]pyridin-6-yl)prop-2-en-1-one

To a solution of tert-butyl 2-(1-(7-(3,5-dichloro-4-hydroxyphenyl)-4-methoxy-2H-[1,2,3]triazolo[4,5-c]pyridin-2-yl)ethyl)-5,7-dihydro-6H-pyrrolo[3,4-b]pyridine-6-carboxylate in DCM (1.0 mL) was added TFA (1.0 mL, 3.38 mmol). The mixture was stirred at room temperature for 30 min, then evaporated and used for the next step without further purification.

To a solution of 2,6-dichloro-4-(2-(1-(6,7-dihydro-5H-pyrrolo[3,4-b]pyridin-2-yl)ethyl)-4-methoxy-2H-[1,2,3]triazolo[4,5-c]pyridin-7-yl)phenol (15 mg, 0.033 mmol) and Hunig's base (0.017 mL, 0.098 mmol) in THF (0.5 mL) at 0° C. was added acryloyl chloride (3 μL, 0.033 mmol). The reaction mixture was stirred at room temperature for a few minutes. Upon full completion, the reaction mixture was diluted with acetonitrile and purified via prep-LCMS (XBridge C18 column, eluting with a gradient of acetonitrile/water containing 0.1% TFA, at flow rate of 60 mL/min) to afford desired product as a TFA salt. LCMS calculated for C₂₄H₂₁Cl₂N₆O₃ (M+H)⁺: m/z=511.1; found: 511.1.

### Example 38. Rac-(E)-1-(2-(1-(6-chloro-7-(3,5-dichloro-4-hydroxyphenyl)-2H-[1,2,3]triazolo[4,5-c]pyridin-2-yl)ethyl)-5,7-dihydro-6H-pyrrolo[3,4-b]pyridin-6-yl)-4-(dimethylamino)but-2-en-1-one

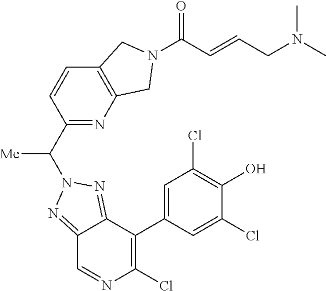 [SMILES: CC(c1ccc2c(n1)CN(C(=O)/C=C/CN(C)C)C2)n1nc2cnc(Cl)c(-c3cc(Cl)c(O)c(Cl)c3)c2n1]

### Step 1. tert-Butyl 2-(1-(7-bromo-6-chloro-2H-[1,2,3]triazolo[4,5-c]pyridin-2-yl)ethyl)-5,7-dihydro-6H-pyrrolo[3,4-b]pyridine-6-carboxylate

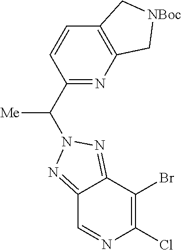 [SMILES: CC(c1ccc2c(n1)CN(C(=O)OC(C)(C)C)C2)n1nc2cnc(Cl)c(Br)c2n1]

7-bromo-6-chloro-2H-[1,2,3]triazolo[4,5-c]pyridine (400 mg, 1.713 mmol), Rac-tert-butyl 2-(1-hydroxyethyl)-5,7-dihydro-6H-pyrrolo[3,4-b]pyridine-6-carboxylate (Intermediate 10, step 1, 498 mg, 1.885 mmol) and triphenylphosphine (494 mg, 1.885 mmol) were combined in THF (6 mL). Diisopropyl azodicarboxylate (0.366 mL, 1.885 mmol) was added at 0° C. The mixture was stirred at 0° C. for 45 min and then warmed to 23° C. and stirred for 15 min. Upon completion, the resulting mixture was quenched with saturated NaHCO₃ solution (10 mL) and extracted with EtOAc (30 mL). The organic layer was washed with brine, dried with Na₂SO₄, filtered, and concentrated in vacuo. The residue was purified by column chromatography (0-55% EtOAc in hexanes) to give the desired product as a white solid along with undesired regioisomer (removed chromatographically). LCMS calculated for C₁₉H₂₁BrClN₆O₂(M+H)⁺: m/z=479.0; found: 479.1.

### Step 2. (E)-1-(2-(1-(6-chloro-7-(3,5-dichloro-4-hydroxyphenyl)-2H-[1,2,3]triazolo[4,5-c]pyridin-2-yl)ethyl)-5,7-dihydro-6H-pyrrolo[3,4-b]pyridin-6-yl)-4-(dimethylamino)but-2-en-1-one

To a solution of tert-butyl 2-(1-(7-bromo-6-chloro-2H-[1,2,3]triazolo[4,5-c]pyridin-2-yl)ethyl)-5,7-dihydro-6H-pyrrolo[3,4-b]pyridine-6-carboxylate (20 mg, 0.042 mmol) in 1,4-dioxane (828 μL) and water (166 μL) was added 2,6-dichloro-4-(4,4,5,5-tetramethyl-1,3,2-dioxaborolan-2-yl)phenol (24.1 mg, 0.083 mmol), potassium phosphate (26.5 mg, 0.125 mmol) and XPhos-Pd-G4 (1.8 mg, 2.084 μmol). The reaction mixture was then degassed for 10 min before heating to 100° C. After 1 h, the resulting mixture was cooled and diluted with water. The pH of the solution was adjusted to 5 using 1 N HCl and extracted with EtOAc (5 mL). The organic layer was washed with brine, dried with Na₂SO₄, filtered, and concentrated in vacuo.

The obtained crude product was dissolved in dichloromethane (0.5 mL) and 2,2,2-trifluoroacetic acid (0.5 mL) and stirred for 30 mins. Upon completion, the reaction mixture was concentrated in vacuo.

To the above crude product was added N-ethyl-N-isopropylpropan-2-amine (28 μL, 0.154 mmol), (E)-4-(diethylamino)but-2-enoic acid hydrochloride (7.2 mg, 0.037 mmol) and propylphosphonic anhydride (T3P, 50% in EtOAc, 18 μL, 0.031 mmol) in THF (1 mL). The reaction mixture stirred at room temperature for 10 minutes before the solution was diluted with acetonitrile and purified by prep-LCMS (XBridge C18 column, eluting with a gradient of acetonitrile/water containing 0.1% TFA, at flow rate of 60 mL/min) to afford desired product as a pale-yellow solid. LCMS calculated for C₂₆H₂₅C₁₃N₇O₂ (M+H)⁺: m/z=572.1; found: 572.1.

### Example 39. (E)-1-(5-(2-(7-(2-chloro-6-methoxypyridin-4-yl)-4-methyl-2H-[1,2,3]triazolo[4,5-c]pyridin-2-yl)propan-2-yl)indolin-1-yl)-4-(dimethylamino)but-2-en-1-one

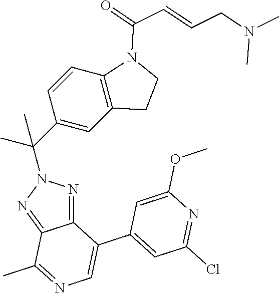 [SMILES: COc1cc(-c2cnc(C)c3nn(C(C)(C)c4ccc5c(c4)CCN5C(=O)/C=C/CN(C)C)nc23)cc(Cl)n1]

### Step 1. (9H-fluoren-9-yl)methyl 5-bromoindoline-1-carboxylate

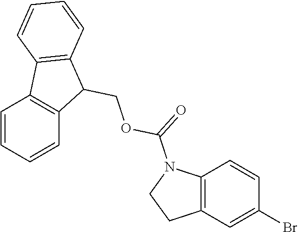 [SMILES: O=C(OCC1c2ccccc2-c2ccccc21)N1CCc2cc(Br)ccc21]

To a solution of 5-bromoindoline (1.7 g, 8.58 mmol) and DIPEA (3.0 ml, 17.17 mmol) in THF (20 ml) at room temperature was added (9H-fluoren-9-yl)methyl (2,5-dioxopyrrolidin-1-yl) carbonate (4.3 g, 12.87 mmol). The mixture was stirred at room temperature for 1 h. Upon completion, the resulting mixture was quenched with saturated NH₄Cl solution (10 mL) and extracted with EtOAc (30 mL). The organic layer was washed with brine, dried with Na₂SO₄, filtered, and concentrated in vacuo. The residue was purified by column chromatography (0-30% EtOAc in hexanes). LC-MS calculated for C₂₃H₁₉BrNO₂ (M+H)⁺: m/z=420.1; found: 420.1.

### Step 2. (9H-fluoren-9-yl)methyl 5-(prop-1-en-2-yl)indoline-1-carboxylate

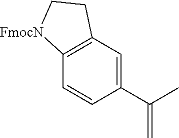

To a mixture of (9H-fluoren-9-yl)methyl 5-bromoindoline-1-carboxylate (700 mg, 1.665 mmol), trifluoro(prop-1-en-2-yl)-14-borane, potassium salt (493 mg, 3.33 mmol) and potassium phosphate (1061 mg, 5.00 mmol) in 1,4-dioxane (2 mL) and water (0.4 mL) was added XPhos-Pd-G4 (143 mg, 0.167 mmol). The reaction mixture was then degassed for 10 min before heating to 95° C., stirred for 1 h. Upon completion, the mixture was cooled to room temperature, diluted with water and extracted with EtOAc (30 mL). The organic layer was washed with brine, dried with Na₂SO₄, filtered, and concentrated in vacuo. The residue was purified by column chromatography (0-20% EtOAc in hexanes). LC-MS calculated for C₂₆H₂₄NO₂ (M+H)⁺: m/z=382.2; found: 382.2.

### Step 3. (9H-fluoren-9-yl)methyl 5-(2-(7-bromo-4-methyl-2H-[1,2,3]triazolo[4,5-c]pyridin-2-yl)propan-2-yl)indoline-1-carboxylate

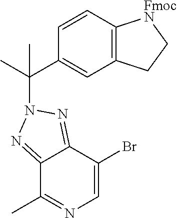

To a mixture of 7-bromo-4-methyl-3H-[1,2,3]triazolo[4,5-c]pyridine hydrochloride (400 mg, 1.603 mmol) and (9H-fluoren-9-yl)methyl 5-(prop-1-en-2-yl)indoline-1-carboxylate (917 mg, 2.405 mmol) in toluene (20 mL) was added Ts-OH H₂O (915 mg, 4.81 mmol). The reaction mixture was then heated to 115° C., stirred overnight. Upon completion, the mixture was cooled to room temperature, quenched with saturated NaHCO₃ solution (10 mL) and extracted with EtOAc (30 mL). The organic layer was washed with brine, dried with Na₂SO₄, filtered, and concentrated in vacuo. The residue was purified by column chromatography (0-50% EtOAc in hexanes). LC-MS calculated for C₃₂H₂₉BrN₅O₂(M+H)⁺: m/z=594.1; found: 594.1.

### Step 4. (9H-fluoren-9-yl)methyl 5-(2-(7-(2-chloro-6-methoxypyridin-4-yl)-4-methyl-2H-[1,2,3]triazolo[4,5-c]pyridin-2-yl)propan-2-yl)indoline-1-carboxylate

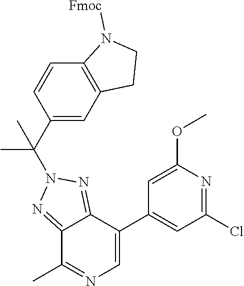

To a solution of (9H-fluoren-9-yl)methyl 5-(2-(7-bromo-4-methyl-2H-[1,2,3]triazolo[4,5-c]pyridin-2-yl)propan-2-yl)indoline-1-carboxylate (23 mg, 0.039 mmol) in 1,4-dioxane (269 μL) and water (53 μL) was added 2-chloro-6-methoxy-4-(4,4,5,5-tetramethyl-1,3,2-dioxaborolan-2-yl)pyridine (15.6 mg, 0.058 mmol), dipotassium monophosphate (16.7 mg, 0.097 mmol) and PdCl₂(dppf)-CH₂Cl₂ adduct (7.9 mg, 9.67 μmol). The reaction mixture was then degassed for 10 min before heating to 80° C. for 10 minutes. Upon full completion, the reaction mixture was concentrated and purified by flash column chromatography (0-80% EtOAc in hexanes) to give pure product. LCMS calculated for C₃₈H₃₄C₁N₆O₃(M+H)⁺: m/z=657.2; found: 657.2.

### Step 5. (E)-1-(5-(2-(7-(2-chloro-6-methoxypyridin-4-yl)-4-methyl-2H-[1,2,3]triazolo[4,5-c]pyridin-2-yl)propan-2-yl)indolin-1-yl)-4-(dimethylamino)but-2-en-1-one

To a solution of (9H-fluoren-9-yl)methyl 5-(2-(7-(2-chloro-6-methoxypyridin-4-yl)-4-methyl-2H-[1,2,3]triazolo[4,5-c]pyridin-2-yl)propan-2-yl)indoline-1-carboxylate (100 mg, 0.152 mmol) in DCM (2.5 mL) was added diethylamine (0.35 mL, 3.38 mmol). The mixture was stirred at room temperature for 10 minutes, then evaporated and used for the next step without further purification.

To a solution of 7-(2-chloro-6-methoxypyridin-4-yl)-2-(2-(indolin-5-yl)propan-2-yl)-4-methyl-2H-[1,2,3]triazolo[4,5-c]pyridine (3 mg, 6.90 μmol), (E)-4-(dimethylamino)but-2-enoic acid hydrochloride (1.7 mg, 10.35 μmol) and cesium carbonate (6.7 mg, 0.021 mmol) in acetonitrile (100 μL) and DMF (100 μL) was added HATU (3.9 mg, 10.35 μmol). The mixture was stirred at 50° C. for 15 minutes. Diluted with Methanol and purified via prep-LCMS (XBridge C18 column, eluting with a gradient of acetonitrile/water containing 0.1% TFA, at flow rate of 60 mL/min) to afford desired product as a TFA salt. LCMS calculated for C₂₉H₃₃C₁N₇O₂(M+H)⁺: m/z=546.3; found: 546.3.

### Example 40. (E)-4-(bis(methyl-d3)amino)-1-(5-((7-(3,5-dichloro-4-hydroxyphenyl)-4-methyl-2H-[1,2,3]triazolo[4,5-c]pyridin-2-yl)methyl)-4-fluoroindolin-1-yl)but-2-en-1-one

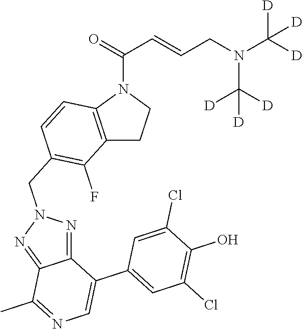 [SMILES: [2H]C([2H])([2H])N(C/C=C/C(=O)N1CCc2c1ccc(Cn1nc3c(-c4cc(Cl)c(O)c(Cl)c4)cnc(C)c3n1)c2F)C([2H])([2H])[2H]]

### Step 1. tert-Butyl 5-(chloromethyl)-4-fluoroindoline-1-carboxylate

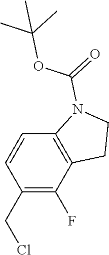 [SMILES: CC(C)(C)OC(=O)N1CCc2c1ccc(CCl)c2F]

To a solution of tert-butyl 4-fluoro-5-(hydroxymethyl)indoline-1-carboxylate (270 mg, 1.010 mmol) in DCM (5.0 mL) at 0° C. was added sulfurous dichloride (110 μL, 1.515 mmol). The reaction mixture was stirred at corresponding temperature for 30 minutes. Upon completion, the reaction mixture was concentrated and used directly for the next step without purification. LCMS calculated for C₁₄H₁₈ClFNO₂ (M+H)⁺: m/z=286.1; found: 286.1.

### Step 2. tert-Butyl 5-((7-bromo-4-methyl-2H-[1,2,3]triazolo[4,5-c]pyridin-2-yl)methyl)-4-fluoroindoline-1-carboxylate

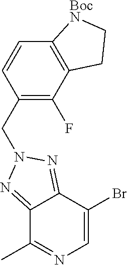 [SMILES: Cc1ncc(Br)c2nn(Cc3ccc4c(c3F)CCN4C(=O)OC(C)(C)C)nc12]

To a solution of 7-bromo-4-methyl-2H-[1,2,3]triazolo[4,5-c]pyridine (290 mg, 1.361 mmol), tert-butyl 5-(chloromethyl)-4-fluoroindoline-1-carboxylate (389 mg, 1.361 mmol) in DMF (6.8 mL) was added N-ethyl-N-isopropylpropan-2-amine (593 μL, 3.40 mmol). The reaction mixture was stirred at 70° C. for 2 hours. Upon completion, the reaction mixture was cooled to room temperature, quenched with water, extracted with EtOAc. The organic layers were concentrated, purified by flash column chromatography (0-40% EtOAc in hexanes) to give pure product. LCMS calculated for C₂₀H₂₂BrFN₅O₂(M+H)⁺: m/z=462.1; found: 462.1.

### Step 3. tert-Butyl 5-((7-(3,5-dichloro-4-hydroxyphenyl)-4-methyl-2H-[1,2,3]triazolo[4,5-c]pyridin-2-yl)methyl)-4-fluoroindoline-1-carboxylate

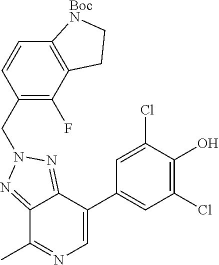 [SMILES: Cc1ncc(-c2cc(Cl)c(O)c(Cl)c2)c2nn(Cc3ccc4c(c3F)CCN4C(=O)OC(C)(C)C)nc12]

To a solution of tert-butyl 5-((7-bromo-4-methyl-2H-[1,2,3]triazolo[4,5-c]pyridin-2-yl)methyl)-4-fluoroindoline-1-carboxylate (100 mg, 0.216 mmol) in 1,4-dioxane (1.8 mL) and water (0.36 mL) was added 2,6-dichloro-4-(4,4,5,5-tetramethyl-1,3,2-dioxaborolaN-2-yl)phenol (94 mg, 0.324 mmol), potassium phosphate tribasic (115 mg, 0.541 mmol) and chloro(2-dicyclohexylphosphino-2′,4′,6′-tri-i-propyl-1,1′-biphenyl)(2′-amino-1,1′-biphenyl-2-yl) palladium(II) (34.0 mg, 0.043 mmol). The reaction mixture was then degassed for 10 minutes before heating to 100° C. for 30 minutes. Upon completion, the reaction mixture was cooled down to room temperature and dried over MgSO4. The filtrate was then concentrated and purified by flash column chromatography (0-80% EtOAc in hexanes) to give pure product. LCMS calculated for C₂₆H₂₅C₁₂FN₅O₃(M+H)⁺: m/z=544.1; found: 544.1.

### Step 4. (E)-4-(bis(methyl-d3)amino)-1-(5-((7-(3,5-dichloro-4-hydroxyphenyl)-4-methyl-2H-[1,2,3]triazolo[4,5-c]pyridin-2-yl)methyl)-4-fluoroindolin-1-yl)but-2-en-1-one

To a solution of tert-butyl 5-((7-(3,5-dichloro-4-hydroxyphenyl)-4-methyl-2H-[1,2,3]triazolo[4,5-c]pyridin-2-yl)methyl)-4-fluoroindoline-1-carboxylate in DCM (1.0 mL) was added TFA (1.0 mL, 3.38 mmol). The mixture was stirred at room temperature for 30 min, then evaporated and used for the next step without further purification.

To a solution of 2,6-dichloro-4-(2-((4-fluoroindolin-5-yl)methyl)-4-methyl-2H-[1,2,3]triazolo[4,5-c]pyridin-7-yl)phenol (10 mg, 0.023 mmol), N-ethyl-N-isopropylpropan-2-amine (19 μL, 0.113 mmol) in THF (225 μL) was added) was added (E)-4-(bis(methyl-d3)amino)but-2-enoyl chloride (4.1 mg, 0.027 mmol). The reaction mixture was stirred at room temperature for a few minutes. Upon completion, the reaction mixture was diluted with acetonitrile and purified via prep-LCMS (XBridge C18 column, eluting with a gradient of acetonitrile/water containing 0.1% TFA, at flow rate of 60 mL/min) to afford desired product as a TFA salt. LCMS calculated for C₂₇H₂₀D₆C₁₂FN₆O₂(M+H)⁺: m/z=561.2; found: 561.2.

### Example 41. (E)-1-(2-((7-(3,5-dichloro-4-hydroxyphenyl)-4-methyl-2H-[1,2,3]triazolo[4,5-c]pyridin-2-yl)methyl)-3-ethyl-5,7-dihydro-6H-pyrrolo[3,4-b]pyridin-6-yl)-4-(piperidin-1-yl)but-2-en-1-one

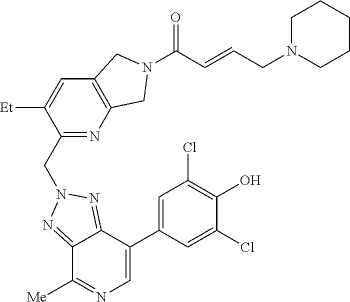 [SMILES: CCc1cc2c(nc1Cn1nc3c(-c4cc(Cl)c(O)c(Cl)c4)cnc(C)c3n1)CN(C(=O)/C=C/CN1CCCCC1)C2]

### Step 1. tert-Butyl 3-ethyl-2-(hydroxymethyl)-5,7-dihydro-6H-pyrrolo[3,4-b]pyridine-6-carboxylate

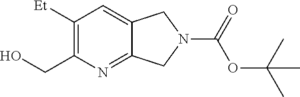 [SMILES: CCc1cc2c(nc1CO)CN(C(=O)OC(C)(C)C)C2]

To the magnetically stirred solution of tert-butyl 3-ethyl-2-formyl-5,7-dihydro-6H-pyrrolo[3,4-b]pyridine-6-carboxylate (500 mg, 1.809 mmol) in MeOH (8 mL) was added sodium borohydride (34.2 mg, 0.905 mmol) at 0° C. for 5 min and then warmed to room temperature and stirred for additional 15 min. Upon completion, the resulting mixture was quenched with saturated NH₄Cl solution (20 mL) and extracted with EtOAc (50 mL). The organic layer was washed with brine, dried with Na₂SO₄, filtered, and concentrated in vacuo to give the desired product as a colorless liquid. LC-MS calculated for C₁₅H₂₃N₂O₃ (M+H)⁺: m/z=279.2; found: 279.1.

### Step 2. tert-Butyl 2-((4,7-dibromo-2H-[1,2,3]triazolo[4,5-c]pyridin-2-yl)methyl)-3-ethyl-5,7-dihydro-6H-pyrrolo[3,4-b]pyridine-6-carboxylate

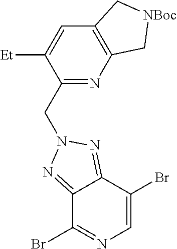 [SMILES: CCc1cc2c(nc1Cn1nc3c(Br)cnc(Br)c3n1)CN(C(=O)OC(C)(C)C)C2]

To the magnetically stirred solution of 4,7-dibromo-2H-[1,2,3]triazolo[4,5-c]pyridine hydrochloride (226 mg, 0.719 mmol), tert-butyl 3-ethyl-2-(hydroxymethyl)-5,7-dihydro-6H-pyrrolo[3,4-b]pyridine-6-carboxylate (200 mg, 0.719 mmol) and triphenylphosphine (226 mg, 0.862 mmol) were combined in THF (5 mL) at 0° C. followed by addition of diisopropyl azodicarboxylate (0.168 mL, 0.862 mmol). The mixture was stirred at 0° C. for 45 min and then warmed to 23° C. and stirred for 15 min. Upon completion, the resulting mixture was quenched with saturated NaHCO₃ solution (10 mL) and extracted with EtOAc (30 mL). The organic layer was washed with brine, dried with Na₂SO₄, filtered, and concentrated in vacuo. The residue was purified by column chromatography (0-40% EtOAc in hexanes) to give the desired product as a white solid along with undesired regioisomer (removed chromatographically). LC-MS calculated for C₂₀H₂₃Br₂N₆O₂ (M+H)⁺: m/z=537.0; found: 537.1.

### Step 3. tert-Butyl 2-((7-bromo-4-methyl-2H-[1,2,3]triazolo[4,5-c]pyridin-2-yl)methyl)-3-ethyl-5,7-dihydro-6H-pyrrolo[3,4-b]pyridine-6-carboxylate

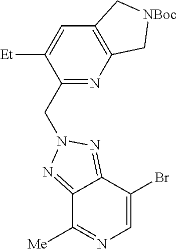 [SMILES: CCc1cc2c(nc1Cn1nc3c(Br)cnc(C)c3n1)CN(C(=O)OC(C)(C)C)C2]

At a room temperature, tert-butyl 2-((4,7-dibromo-2H-[1,2,3]triazolo[4,5-c]pyridin-2-yl)methyl)-3-ethyl-5,7-dihydro-6H-pyrrolo[3,4-b]pyridine-6-carboxylate (250 mg, 0.464 mmol), tetrakis(triphenylphosphine)palladium(0) (53.7 mg, 0.046 mmol) were combined in THF (5 mL). Dimethylzinc (1.2 M in toluene, 0.581 mL, 0.697 mmol) was added at room temperature and stirred at same temperature for 30 min. Upon completion, the resulting mixture was quenched with saturated NH₄Cl solution (5 mL) and extracted with EtOAc (35 mL). The organic layer was washed with brine, dried with Na₂SO₄, filtered, and concentrated in vacuo. The residue was purified by column chromatography (0-55% EtOAc in hexanes) to give the desired product the desired product as a white solid. LC-MS calculated for C₂₁H₂₆BrN₆O₂(M+H)⁺: m/z=473.1; found: 473.1.

### Step 4. (E)-1-(2-((7-(3,5-dichloro-4-hydroxyphenyl)-4-methyl-2H-[1,2,3]triazolo[4,5-c]pyridin-2-yl)methyl)-3-ethyl-5,7-dihydro-6H-pyrrolo[3,4-b]pyridin-6-yl)-4-(piperidin-1-yl)but-2-en-1-one

To a magnetically stirred solution of tert-butyl 2-((7-bromo-4-methyl-2H-[1,2,3]triazolo[4,5-c]pyridin-2-yl)methyl)-3-ethyl-5,7-dihydro-6H-pyrrolo[3,4-b]pyridine-6-carboxylate (30 mg, 0.063 mmol) in 1,4-dioxane (828 μL) and water (166 μL) was added 2,6-dichloro-4-(4,4,5,5-tetramethyl-1,3,2-dioxaborolan-2-yl)phenol (36.6 mg, 0.127 mmol), potassium phosphate (40.4 mg, 0.190 mmol) and XPhos-Pd-G4 (2.7 mg, 3.17 μmol). The reaction mixture was then degassed for 10 min before heating to 100° C. After 1 h, the resulting mixture was cooled and diluted with water. The pH of the solution was adjusted to 5 using 1 N HCl and extracted with EtOAc (10 mL). The organic layer was washed with brine, dried with Na₂SO₄, filtered, and concentrated in vacuo.

The obtained crude product was dissolved in dichloromethane (0.5 mL) and 2,2,2-trifluoroacetic acid (0.5 mL) and stirred for 30 mins at 23° C. Upon completion, the reaction mixture was concentrated in vacuo.

To a suspension of 2,6-dichloro-4-(2-((3-ethyl-6,7-dihydro-5H-pyrrolo[3,4-b]pyridin-2-yl)methyl)-4-methyl-2H-[1,2,3]triazolo[4,5-c]pyridin-7-yl)phenol (14 mg, 0.031 mmol), N-ethyl-N-isopropylpropan-2-amine (4 mg, 0.031 mmol)) and (E)-4-(piperidin-1-yl)but-2-enoic acid hydrochloride (6.3 mg, 0.031 mmol) in THF (1 mL) was added propylphosphonic anhydride (T3P, 50% in EtOAc, 9 μL, 0.031 mmol). The reaction mixture stirred at room temperature for 10 minutes before the solution was diluted with acetonitrile and purified by prep-LCMS (XBridge C18 column, eluting with a gradient of acetonitrile/water containing 0.1% TFA, at flow rate of 60 mL/min) to afford desired product as an off white solid. LCMS calculated for C₃₁H₃₄C₁₂N₇O₂ (M+H)⁺: m/z=606.2; found: 606.3.

### Example 42. (E)-4-(3-azabicyclo[3.1.1]heptan-3-yl)-1-(2-(2-(7-(3,5-dichloro-4-hydroxyphenyl)-4-methyl-2H-[1,2,3]triazolo[4,5-c]pyridin-2-yl)propan-2-yl)-4-methoxy-5,7-dihydro-6H-pyrrolo[3,4-b]pyridin-6-yl)but-2-en-1-one

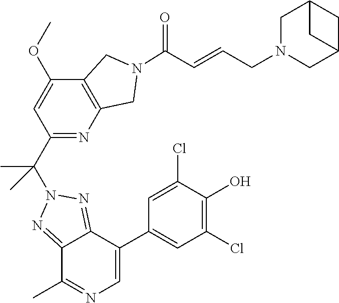 [SMILES: COc1cc(C(C)(C)n2nc3c(-c4cc(Cl)c(O)c(Cl)c4)cnc(C)c3n2)nc2c1CN(C(=O)/C=C/CN1CC3CC(C3)C1)C2]

### Step 1. Rac-tert-butyl 2-(1-hydroxyethyl)-4-methoxy-5,7-dihydro-6H-pyrrolo[3,4-b]pyridine-6-carboxylate

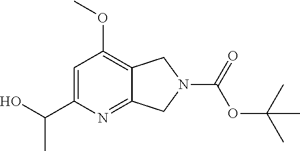 [SMILES: COc1cc(C(C)O)nc2c1CN(C(=O)OC(C)(C)C)C2]

To the magnetically stirred solution of tert-butyl 2-formyl-4-methoxy-5,7-dihydro-6H-pyrrolo[3,4-b]pyridine-6-carboxylate (0.9 g, 3.23 mmol) in Et₂O (10 mL) was added methylmagnesium bromide (1.4 mL, 4.85 mmol) at 0° C. for 5 min. The reaction was stirred at same temperature for 15 min and then warmed to room temperature and stirred for additional 15 min. Upon completion, the resulting mixture was quenched with saturated NH₄C₁ solution (10 mL) and extracted with EtOAc (50 mL). The organic layer was washed with brine, dried with Na₂SO₄, filtered, and concentrated in vacuo to give the desired product as a white solid. LCMS calculated for C₅H₂₃N₂O₄ (M+H)⁺: m/z=295.2; found: 295.1.

### Step 2. Rac-tert-butyl 2-(1-(4,7-dibromo-2H-[1,2,3]triazolo[4,5-c]pyridin-2-yl)ethyl)-4-methoxy-5,7-dihydro-6H-pyrrolo[3,4-b]pyridine-6-carboxylate

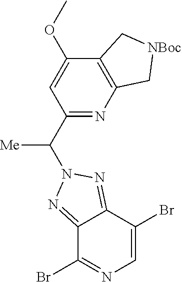 [SMILES: COc1cc(C(C)n2nc3c(Br)cnc(Br)c3n2)nc2c1CN(C(=O)OC(C)(C)C)C2]

To the magnetically stirred solution of 4,7-dibromo-2H-[1,2,3]triazolo[4,5-c]pyridine hydrochloride (1 g, 3.18 mmol), rac-tert-butyl 2-(1-hydroxyethyl)-4-methoxy-5,7-dihydro-6H-pyrrolo[3,4-b]pyridine-6-carboxylate (0.936 g, 3.18 mmol) and triphenylphosphine (1.0 g, 3.82 mmol) were combined in THF (10 mL) at 0° C. followed by addition of diisopropyl azodicarboxylate (0.742 mL, 3.82 mmol). The mixture was stirred at 0° C. for 45 min and then warmed to 23° C. and stirred for 15 min. Upon completion, the resulting mixture was quenched with saturated NaHCO₃ solution (10 mL) and extracted with EtOAc (50 mL). The organic layer was washed with brine, dried with Na₂SO₄, filtered, and concentrated in vacuo. The residue was purified by column chromatography (0-50% EtOAc in hexanes) to give the desired product as a white solid along with undesired regioisomer (removed chromatographically). LCMS calculated for C₂₀H₂₃Br₂N₆O₃ (M+H)⁺: m/z=553.0; found: 553.1.

### Step 3. Rac-tert-butyl 2-(1-(7-bromo-4-methyl-2H-[1,2,3]triazolo[4,5-c]pyridin-2-yl)ethyl)-4-methoxy-5,7-dihydro-6H-pyrrolo[3,4-b]pyridine-6-carboxylate

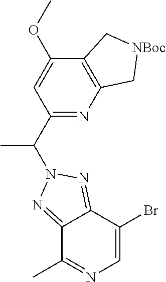 [SMILES: COc1cc(C(C)n2nc3c(Br)cnc(C)c3n2)nc2c1CN(C(=O)OC(C)(C)C)C2]

At a room temperature, rac-tert-butyl 2-(1-(4,7-dibromo-2H-[1,2,3]triazolo[4,5-c]pyridin-2-yl)ethyl)-4-methoxy-5,7-dihydro-6H-pyrrolo[3,4-b]pyridine-6-carboxylate (0.4 g, 0.722 mmol), tetrakis(triphenylphosphine)palladium(0) (0.083 g, 0.072 mmol) were combined in THF (5 mL). Dimethylzinc (1.2 M in toluene, 0.902 mL, 1.083 mmol) was added at room temperature. The mixture was stirred at room temperature for 30 min. Upon completion, the resulting mixture was quenched with saturated NH₄Cl solution (5 mL) and extracted with EtOAc (25 mL). The organic layer was washed with brine, dried with Na₂SO₄, filtered, and concentrated in vacuo. The residue was purified by column chromatography (0-50% EtOAc in hexanes) to give the desired product the desired product as a white solid. LCMS calculated for C₂₁H₂₆BrN₆O₃(M+H)⁺: m/z=489.1; found: 489.1.

### Step 4. tert-Butyl 2-(2-(7-bromo-4-methyl-2H-[1,2,3]triazolo[4,5-c]pyridin-2-yl)propan-2-yl)-4-methoxy-5,7-dihydro-6H-pyrrolo[3,4-b]pyridine-6-carboxylate

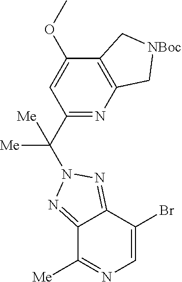 [SMILES: COc1cc(C(C)(C)n2nc3c(Br)cnc(C)c3n2)nc2c1CN(C(=O)OC(C)(C)C)C2]

To a solution of rac-tert-butyl 2-(1-(7-bromo-4-methyl-2H-[1,2,3]triazolo[4,5-c]pyridin-2-yl)ethyl)-4-methoxy-5,7-dihydro-6H-pyrrolo[3,4-b]pyridine-6-carboxylate (160 mg, 0.327 mmol) in THF (3 mL) at −78° C. was added LiHMDS (1 M in THF, 0.343 mL, 0.343 mmol). Reaction mixture stirred at −78° C. for 20 min, then added iodomethane (0.031 mL, 0.490 mmol). After stirring for additional 5 min at −78° C., cooling bath was removed and stirred at room temperature for 5 min. Upon completion, the resulting mixture was quenched with saturated NH₄Cl solution (10 mL) and extracted with EtOAc (15 mL). The organic layer was washed with brine, dried with Na₂SO₄, filtered, and concentrated in vacuo. The residue was purified by column chromatography (0-55% EtOAc in hexanes) to give the desired product as a white solid. along with undesired side product (removed chromatographically). LC-MS calculated for C₂₂H₂₈BrN₆O₃(M+H)⁺: m/z=503.1; found: 503.1.

### Step 5. (E)-4-(3-azabicyclo[3.1.1]heptan-3-yl)-1-(2-(2-(7-(3,5-dichloro-4-hydroxyphenyl)-4-methyl-2H-[1,2,3]triazolo[4,5-c]pyridin-2-yl)propan-2-yl)-4-methoxy-5,7-dihydro-6H-pyrrolo[3,4-b]pyridin-6-yl)but-2-en-1-one

To a magnetically stirred solution of tert-butyl 2-(2-(7-bromo-4-methyl-2H-[1,2,3]triazolo[4,5-c]pyridin-2-yl)propan-2-yl)-4-methoxy-5,7-dihydro-6H-pyrrolo[3,4-b]pyridine-6-carboxylate (50 mg, 0.099 mmol) in 1,4-dioxane (828 μL) and water (166 μL) was added 2,6-dichloro-4-(4,4,5,5-tetramethyl-1,3,2-dioxaborolan-2-yl)phenol (57.4 mg, 0.199 mmol), potassium phosphate (63.2 mg, 0.298 mmol) and XPhos-Pd-G4 (4.2 mg, 4.97 μmol). The reaction mixture was then degassed for 10 min before heating to 100° C. After 1h, the resulting mixture was cooled and diluted with water. The pH of the solution was adjusted to 5 using 1 N HCl and extracted with EtOAc (5 mL). The organic layer was washed with brine, dried with Na₂SO₄, filtered, and concentrated in vacuo.

The obtained crude product was dissolved in dichloromethane (0.5 mL) and 2,2,2-trifluoroacetic acid (0.5 mL) and stirred for 30 mins at 23° C. Upon completion, the reaction mixture was concentrated in vacuo.

To the above crude product in THF (1 mL) was added N-ethyl-N-isopropylpropan-2-amine (24 mg, 0.19 mmol), (E)-4-(3-azabicyclo[3.1.1]heptan-3-yl)but-2-enoic acid hydrochloride (16 mg, 0.074 mmol) and propylphosphonic anhydride (T3P, in 50% EtOAc, 0.027 mL, 0.093 mmol). The reaction mixture stirred at room temperature for 10 minutes before the solution was diluted with acetonitrile and purified by prep-LCMS (XBridge C18 column, eluting with a gradient of acetonitrile/water containing 0.1% TFA, at flow rate of 60 mL/min) to afford desired product as a pale-yellow solid. LCMS calculated for C₃₃H₃₆C₁₂N₇O₃ (M+H)⁺: m/z=648.2; found: 648.2. ¹H NMR (600 MHz, DMSO-d6) δ 10.31 (s, 1H), 8.73 (s, 1H), 8.19 (s, 2H), 6.99 and 6.91 (s, 1H), 6.77-6.66 (m, 2H), 4.98-4.77 (m, 2H), 4.73-4.48 (m, 2H), 4.18-3.95 (m, 2H), 3.88 and 3.87 (s, 3H), 3.72-3.62 (m, 2H), 3.34 (t, J=17.1 Hz, 2H), 2.92 and 2.91 (s, 3H), 2.44 (dt, J=13.4, 6.4 Hz, 2H), 2.29 (s, 3H), 2.28 (s, 3H), 2.26-2.19 (m, 1H), 2.13 (q, J=7.9 Hz, 1H), 1.80 (q, J=9.8 Hz, 1H), 1.47 (p, J=8.4 Hz, 1H).

### Example 43. (E)-4-(3-azabicyclo[3.1.1]heptan-3-yl)-1-(2-((7-(3,5-dichloro-4-hydroxyphenyl)-4-methyl-2H-[1,2,3]triazolo[4,5-c]pyridin-2-yl)methyl)-3-methoxy-5,7-dihydro-6H-pyrrolo[3,4-b]pyrazin-6-yl)but-2-en-1-one

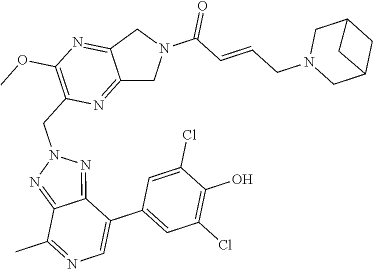 [SMILES: COc1nc2c(nc1Cn1nc3c(-c4cc(Cl)c(O)c(Cl)c4)cnc(C)c3n1)CN(C(=O)/C=C/CN1CC3CC(C3)C1)C2]

### Step 1. tert-Butyl 2-((4,7-dibromo-2H-[1,2,3]triazolo[4,5-c]pyridin-2-yl)methyl)-3-methoxy-5,7-dihydro-6H-pyrrolo[3,4-b]pyrazine-6-carboxylate

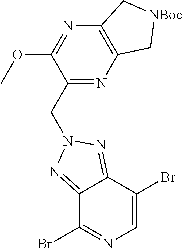 [SMILES: COc1nc2c(nc1Cn1nc3c(Br)cnc(Br)c3n1)CN(C(=O)OC(C)(C)C)C2]

This compound was prepared according to the procedures described in Example 36, step 1 with tert-butyl 2-(hydroxymethyl)-3-methoxy-5,7-dihydro-6H-pyrrolo[3,4-b]pyrazine-6-carboxylate replacing tert-butyl 3-(hydroxymethyl)-2-methoxy-5,7-dihydro-6H-pyrrolo[3,4-b]pyridine-6-carboxylate. LCMS calculated for C₁₈H₂₀Br₂N₇O₃ (M+H)⁺: m/z=540.0; found: 540.0.

### Step 2. 2,6-dichloro-4-(2-((3-methoxy-6,7-dihydro-5H-pyrrolo[3,4-b]pyrazin-2-yl)methyl)-4-methyl-2H-[1,2,3]triazolo[4,5-c]pyridin-7-yl)phenol

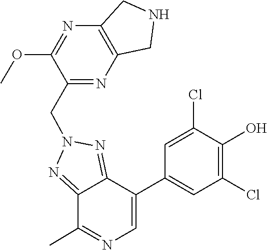 [SMILES: COc1nc2c(nc1Cn1nc3c(-c4cc(Cl)c(O)c(Cl)c4)cnc(C)c3n1)CNC2]

This compound was prepared according to the procedures described in Example 36, step 2 with dimethylzinc replacing diethylzinc and tert-butyl 2-((4,7-dibromo-2H-[1,2,3]triazolo[4,5-c]pyridin-2-yl)methyl)-3-methoxy-5,7-dihydro-6H-pyrrolo[3,4-b]pyrazine-6-carboxylate replacing tert-butyl 3-((4,7-dibromo-2H-[1,2,3]triazolo[4,5-c]pyridin-2-yl)methyl)-2-methoxy-5,7-dihydro-6H-pyrrolo[3,4-b]pyridine-6-carboxylate followed by the procedure described in step 4. LCMS calculated for C₂₀H₁₈C₁₂N₇O₂ (M+H)⁺: m/z=458.1; found: 458.1.

### Step 3. (E)-4-(3-azabicyclo[3.1.1]heptan-3-yl)-1-(2-((7-(3,5-dichloro-4-hydroxyphenyl)-4-methyl-2H-[1,2,3]triazolo[4,5-c]pyridin-2-yl)methyl)-3-methoxy-5,7-dihydro-6H-pyrrolo[3,4-b]pyrazin-6-yl)but-2-en-1-one

This compound was prepared according to the procedures described in Example 36, step 5 with 2,6-dichloro-4-(2-((3-methoxy-6,7-dihydro-5H-pyrrolo[3,4-b]pyrazin-2-yl)methyl)-4-methyl-2H-[1,2,3]triazolo[4,5-c]pyridin-7-yl)phenol replacing 2,6-dichloro-4-(4-ethyl-2-(1-(2-methoxy-6,7-dihydro-5H-pyrrolo[3,4-b]pyridin-3-yl)ethyl)-2H-[1,2,3]triazolo[4,5-c]pyridin-7-yl)phenol. LCMS calculated for C₃₀H₃₁Cl₂N₈O₃ (M+H)⁺: 621.2; found: 621.2. ¹H NMR (600 MHz, DMSO) δ 10.55 (bs, 1H), 10.06 (bs, 1H), 8.68 (s, 1H), 8.15 (s, 2H), 6.84-6.69 (m, 2H), 6.84-6.69 (m, 2H), 5.04-4.58 (m, 4H), 4.08-3.95 (m, 5H), 3.73-3.59 (m, 2H), 3.41-3.27 (m, 2H), 2.84 (s, 3H), 2.47-2.40 (m, 2H), 2.28-2.08 (m, 2H), 1.84-1.68 (m, 2H).

### Example 44. Rac-(E)-1-(2-(1-(7-(3,5-dichloro-4-hydroxyphenyl)-4-methyl-2H-[1,2,3]triazolo[4,5-c]pyridin-2-yl)ethyl)-4-methoxy-5,7-dihydro-6H-pyrrolo[3,4-d]pyrimidin-6-yl)-4-(piperidin-1-yl)but-2-en-1-one

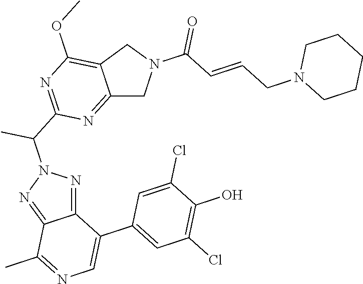 [SMILES: COc1nc(C(C)n2nc3c(-c4cc(Cl)c(O)c(Cl)c4)cnc(C)c3n2)nc2c1CN(C(=O)/C=C/CN1CCCCC1)C2]

### Step 1. Rac-2,6-dichloro-4-(2-(1-(4-methoxy-6,7-dihydro-5H-pyrrolo[3,4-d]pyrimidin-2-yl)ethyl)-4-methyl-2H-[1,2,3]triazolo[4,5-c]pyridin-7-yl)phenol

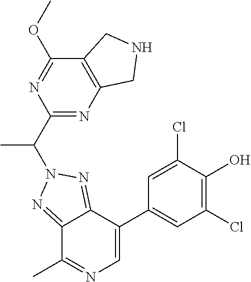 [SMILES: COc1nc(C(C)n2nc3c(-c4cc(Cl)c(O)c(Cl)c4)cnc(C)c3n2)nc2c1CNC2]

This compound was prepared according to the procedures described in Example 36, step 1 with tert-butyl-2-(hydroxymethyl)-4-methoxy-5,7-dihydro-6H-pyrrolo[3,4-d]pyrimidine-6-carboxylate replacing tert-butyl 3-(hydroxymethyl)-2-methoxy-5,7-dihydro-6H-pyrrolo[3,4-b]pyridine-6-carboxylate followed by the procedure described in Example 36, steps 2-4 (Step 2 with dimethylzinc replacing diethylzinc). LCMS calculated for C₂₁H₂₀Cl₂N₇O₂ (M+H)⁺: m/z=472.1; found: 472.1.

### Step 2. Rac-(E)-1-(2-(1-(7-(3,5-dichloro-4-hydroxyphenyl)-4-methyl-2H-[1,2,3]triazolo[4,5-c]pyridin-2-yl)ethyl)-4-methoxy-5,7-dihydro-6H-pyrrolo[3,4-d]pyrimidin-6-yl)-4-(piperidin-1-yl)but-2-en-1-one

This compound was prepared according to the procedures described in Example 10, step 5 with (E)-4-(piperidin-1-yl)but-2-enoic acid replacing (E)-4-(3-azabicyclo[3.1.1]heptan-3-yl)but-2-enoic acid and rac-2,6-dichloro-4-(2-(1-(4-methoxy-6,7-dihydro-5H-pyrrolo[3,4-d]pyrimidin-2-yl)ethyl)-4-methyl-2H-[1,2,3]triazolo[4,5-c]pyridin-7-yl)phenol replacing 2,6-dichloro-4-(4-ethyl-2-(1-(2-methoxy-6,7-dihydro-5H-pyrrolo[3,4-b]pyridin-3-yl)ethyl)-2H-[1,2,3]triazolo[4,5-c]pyridin-7-yl)phenol. LCMS calculated for C₃₀H₃₃C₁₂N₈O₃ (M+H)⁺: 623.2; found: 623.2.

### Example 45. Rac-(E)-1-(5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[2,3-c]pyridin-1-yl)-4-(dimethylamino)but-2-en-1-one

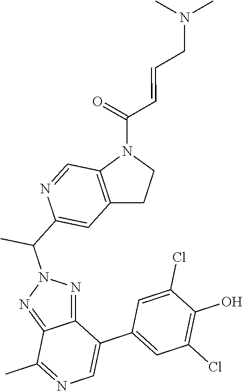 [SMILES: Cc1ncc(-c2cc(Cl)c(O)c(Cl)c2)c2nn(C(C)c3cc4c(cn3)N(C(=O)/C=C/CN(C)C)CC4)nc12]

### Step 1. Rac-tert-butyl 5-(1-(7-bromo-4-methyl-2H-[1,2,3]triazolo[4,5-c]pyridin-2-yl)ethyl)-2,3-dihydro-1H-pyrrolo[2,3-c]pyridine-1-carboxylate

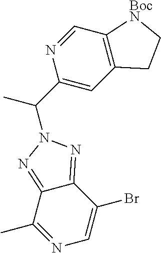 [SMILES: Cc1ncc(Br)c2nn(C(C)c3cc4c(cn3)N(C(=O)OC(C)(C)C)CC4)nc12]

This compound was synthesized based on a similar procedure to Intermediate 10, steps 1 to 3, using tert-butyl 5-formyl-2,3-dihydro-1H-pyrrolo[2,3-c]pyridine-1-carboxylate instead of tert-butyl 2-formyl-5,7-dihydro-6H-pyrrolo[3,4-b]pyridine-6-carboxylate in step 1. LCMS calculated for C₂₀H₂₄BrN₆O₂(M+H)⁺; m/z=459.1; found: 459.1.

### Step 2. Rac-tert-butyl 5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[2,3-c]pyridine-1-carboxylate

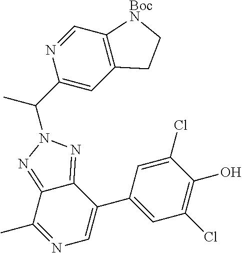 [SMILES: Cc1ncc(-c2cc(Cl)c(O)c(Cl)c2)c2nn(C(C)c3cc4c(cn3)N(C(=O)OC(C)(C)C)CC4)nc12]

To a solution of rac-tert-butyl 5-(1-(7-bromo-4-methyl-2H-[1,2,3]triazolo[4,5-c]pyridin-2-yl)ethyl)-2,3-dihydro-1H-pyrrolo[2,3-c]pyridine-1-carboxylate (150 mg, 0.327 mmol) in 1,4-dioxane (2.7 mL) and water (0.54 mL) was added 2,6-dichloro-4-(4,4,5,5-tetramethyl-1,3,2-dioxaborolan-2-yl)phenol (151 mg, 0.522 mmol), potassium phosphate (208 mg, 0.980 mmol) and XPhos-Pd-G4 (28.1 mg, 0.033 mmol). The reaction mixture was then degassed for 10 min before heating to 95° C. Cooled to room temperature before diluting with brine and extracting with EtOAc (50 mL). The organic phase was then further washed with brine, dried over Na₂SO₄, filtered and concentrated in vacuo. The resulting oil was then purified via column chromatography (0-20% DCM in MeOH). LCMS calculated for C₂₆H₂₇C₁₂N₆O₃ (M+H)⁺; m/z=541.1; found: 541.1.

### Step 3. Rac-(E)-1-(5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[2,3-c]pyridin-1-yl)-4-(dimethylamino)but-2-en-1-one

A vial was charged with rac-tert-butyl 5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[2,3-c]pyridine-1-carboxylate (20 mg, 0.037 mmol) and dry MeOH (1 mL). Cooled to 0° C. before acetyl chloride (0.039 mL, 0.554 mmol) was slowly added to the mixture. Stirred at room temperature for 1 hour. Upon completion, the volatiles were removed in vacuo and the resulting oil dissolved in acetonitrile (0.5 mL). To this was then added cesium carbonate (0.33 g, 0.102 mmol), (E)-4-(dimethylamino)but-2-enoic acid hydrochloride (0.015 g, 0.068 mmol) and HATU (0.019 g, 0.051 mmol). The suspension was then stirred at 50° C. for 1 hour before being diluted with MeOH, filtered and purified via prep-LCMS (XBridge C18 column, eluting with a gradient of acetonitrile/water containing 0.1% TFA, at flow rate of 60 mL/min) to afford the desired product as a TFA salt. LCMS calculated for C₂₇H₂₈C₁₂N₇O₂ (M+H)+; m/z=552.1; found: 552.1 ¹H NMR (600 MHz, DMSO_ht) δ 9.15 (s, 1H), 8.66 (s, 1H), 8.15 (s, 2H), 7.36 (s, 1H), 6.80 (s, 2H), 6.49 (q, J=7.0 Hz, 1H), 4.23 (t, J=8.7 Hz, 2H), 3.95 (s, 2H), 3.23 (t, J=8.8 Hz, 2H), 2.85 (s, 3H), 2.81-2.78 (m, 6H), 2.11 (d, J=6.9 Hz, 3H).

### Example 46. (S,E)-1-(5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[3,2-b]pyridin-1-yl)-4-(dimethylamino)but-2-en-1-one

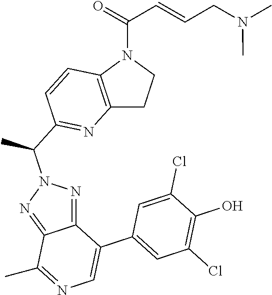 [SMILES: Cc1ncc(-c2cc(Cl)c(O)c(Cl)c2)c2nn([C@@H](C)c3ccc4c(n3)CCN4C(=O)/C=C/CN(C)C)nc12]

### Step 1. Rac-tert-butyl 5-(1-(7-bromo-4-methyl-2H-[1,2,3]triazolo[4,5-c]pyridin-2-yl)ethyl)-2,3-dihydro-1H-pyrrolo[3,2-b]pyridine-1-carboxylate

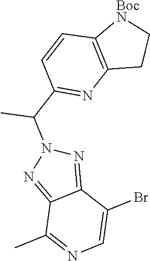 [SMILES: Cc1ncc(Br)c2nn(C(C)c3ccc4c(n3)CCN4C(=O)OC(C)(C)C)nc12]

This compound was synthesized based on a procedure similar to Intermediate 10, steps 1 to 3, using tert-butyl 5-formyl-2,3-dihydro-1H-pyrrolo[3,2-b]pyridine-1-carboxylate instead of tert-butyl 2-formyl-5,7-dihydro-6H-pyrrolo[3,4-b]pyridine-6-carboxylate in step 1. LCMS calculated for C₂₀H₂₄BrN₆O₂(M+H)⁺: m/z=459.1; found: 459.1.

### Step 2. tert-Butyl (S)-5-(1-(7-bromo-4-methyl-2H-[1,2,3]triazolo[4,5-c]pyridin-2-yl)ethyl)-2,3-dihydro-1H-pyrrolo[3,2-b]pyridine-1-carboxylate

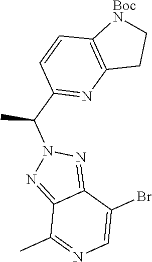 [SMILES: Cc1ncc(Br)c2nn([C@@H](C)c3ccc4c(n3)CCN4C(=O)OC(C)(C)C)nc12]

Rac-tert-butyl 5-(1-(7-bromo-4-methyl-2H-[1,2,3]triazolo[4,5-c]pyridin-2-yl)ethyl)-2,3-dihydro-1H-pyrrolo[3,2-b]pyridine-1-carboxylate was subjected to chiral prep-SFC purification (ChiralTech IC 5 um 30×250 mm column, eluting an isocratic mobile phase of 25% MeOH in CO₂, at a flow rate of 90 mL/min. The first eluted peak was collected and the volatiles removed in vacuo to afford the desired product. LCMS calculated for C₂₀H₂₄BrN₆O₂ (M+H)⁺: m/z=459.1; found: 459.1

### Step 3. tert-Butyl (S)-5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[3,2-b]pyridine-1-carboxylate

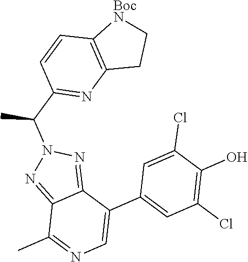 [SMILES: Cc1ncc(-c2cc(Cl)c(O)c(Cl)c2)c2nn([C@@H](C)c3ccc4c(n3)CCN4C(=O)OC(C)(C)C)nc12]

This compound was synthesized based on a procedure similar to Example 45, using tert-butyl (S)-5-(1-(7-bromo-4-methyl-2H-[1,2,3]triazolo[4,5-c]pyridin-2-yl)ethyl)-2,3-dihydro-1H-pyrrolo[3,2-b]pyridine-1-carboxylate instead of Rac-tert-butyl 5-(1-(7-bromo-4-methyl-2H-[1,2,3]triazolo[4,5-c]pyridin-2-yl)ethyl)-2,3-dihydro-1H-pyrrolo[2,3-c]pyridine-1-carboxylate in step 2. LCMS calculated for C₂₆H₂₇C₁₂N₆O₃(M+H)⁺; m/z=541.1, found: 541.1.

### Step 4. (S,E)-1-(5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[3,2-b]pyridin-1-yl)-4-(dimethylamino)but-2-en-1-one

To a cooled solution of tert-butyl (S)-5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[3,2-b]pyridine-1-carboxylate (110 mg, 0.203 mmol) in MeOH (2.5 mL) was slowly added acetyl chloride (0.217 mL, 3.05 mmol). Stirred at room temperature for 1 h before removing the volatiles in vacuo. The crude residue was then diluted with acetonitrile (1 mL). To the vial was then added cesium carbonate (0.199 g, 0.612 mmol), (E)-4-(dimethylamino)but-2-enoic acid hydrochloride (68 mg, 0.408 mmol) and HATU (116 mg, 0.306 mmol). The mixture was stirred at 50° C. for 1 h, then diluted with methanol and purified via prep-LCMS (XBridge C18 column, eluting with a gradient of acetonitrile/water containing 0.1% TFA, at flow rate of 60 mL/min) to afford the desired product as a TFA salt. LCMS calculated for C₂₇H₂₈C₁₂N₇O₂ (M+H)⁺; 552.1, found: 552.1.

### Example 47. (E)-1-(5-(2-(7-(2-chloro-6-methoxypyridin-4-yl)-4-methyl-2H-[1,2,3]triazolo[4,5-c]pyridin-2-yl)propan-2-yl)-2,3-dihydro-1H-pyrrolo[2,3-c]pyridin-1-yl)-4-(diethylamino)but-2-en-1-one

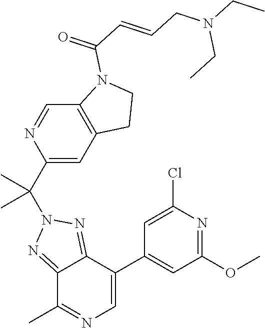 [SMILES: CCN(CC)C/C=C/C(=O)N1CCc2cc(C(C)(C)n3nc4c(-c5cc(Cl)nc(OC)c5)cnc(C)c4n3)ncc21]

### Step 1. tert-Butyl 5-(2-(7-bromo-4-methyl-2H-[1,2,3]triazolo[4,5-c]pyridin-2-yl)propan-2-yl)-2,3-dihydro-1H-pyrrolo[2,3-c]pyridine-1-carboxylate

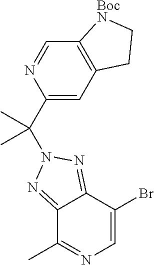 [SMILES: Cc1ncc(Br)c2nn(C(C)(C)c3cc4c(cn3)N(C(=O)OC(C)(C)C)CC4)nc12]

To a solution of Rac-tert-butyl 5-(1-(7-bromo-4-methyl-2H-[1,2,3]triazolo[4,5-c]pyridin-2-yl)ethyl)-2,3-dihydro-1H-pyrrolo[2,3-c]pyridine-1-carboxylate (Example 45, step 1, 175 mg, 0.381 mmol) and methyl iodide (25 μL, 0.40 mmol) in THF (4 mL) at −78° C. was added dropwise NaHMDS (1M in THF, 0.57 mL, 0.57 mmol). The bath was removed, and the reaction mixture continued to stir for 15 min. The mixture was quenched with satd. ammonium chloride and extracted with EtOAc, dried over anhydrous Na₂SO₄, and concentrated in vacuo. The residue was purified by FCC (eluting with 0~100% EtOAc in hexanes) to afford the title compound. LC-MS calc. for C₂₁H₂₆BrN₆O₂(M+H)⁺: m/z=473.1; found: 473.2.

### Step 2. 7-(2-Chloro-6-methoxypyridin-4-yl)-2-(2-(2,3-dihydro-1H-pyrrolo[2,3-c]pyridin-5-yl)propan-2-yl)-4-methyl-2H-[1,2,3]triazolo[4,5-c]pyridine

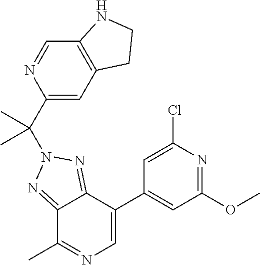 [SMILES: COc1cc(-c2cnc(C)c3nn(C(C)(C)c4cc5c(cn4)NCC5)nc23)cc(Cl)n1]

Under nitrogen, a solution of tert-butyl 5-(2-(7-bromo-4-methyl-2H-[1,2,3]triazolo[4,5-c]pyridin-2-yl)propan-2-yl)-2,3-dihydro-1H-pyrrolo[2,3-c]pyridine-1-carboxylate (60.0 mg, 0.127 mmol), 2-chloro-6-methoxy-4-(4,4,5,5-tetramethyl-1,3,2-dioxaborolan-2-yl)pyridine (41.0 mg, 0.152 mmol), bis(triphenylphosphine)palladium(II) chloride (8.9 mg, 0.013 mmol) and potassium phosphate (53.8 mg, 0.253 mmol) in dioxane (1.0 mL) and water (0.2 mL) was stirred at 100° C. for 5 min. Upon completion, the mixture was diluted with EtOAc, washed with water and brine, dried over anhydrous Na₂SO₄, and concentrated in vacuo. The residue was diluted with TFA (0.05 M) and stirred at room temp for 10 min. The mixture was concentrated and used in the next step without further purification. LC-MS calc. for C₂₂H₂₃C₁N₇O (M+H)⁺: m/z=436.2; found: 436.3.

### Step 3. (E)-1-(5-(2-(7-(2-Chloro-6-methoxypyridin-4-yl)-4-methyl-2H-[1,2,3]triazolo[4,5-c]pyridin-2-yl)propan-2-yl)-2,3-dihydro-1H-pyrrolo[2,3-c]pyridin-1-yl)-4-(diethylamino)but-2-en-1-one

To a suspension of (E)-4-(diethylamino)but-2-enoic acid hydrochloride (53.3 mg, 0.275 mmol) and 7-(2-chloro-6-methoxypyridin-4-yl)-2-(2-(2,3-dihydro-1H-pyrrolo[2,3-c]pyridin-5-yl)propan-2-yl)-4-methyl-2H-[1,2,3]triazolo[4,5-c]pyridine (120 mg, 0.275 mmol) in THF (1 mL) at room temp were added DIPEA (0.481 mL, 2.75 mmol) and propylphosphonic anhydride (T3P, 50% in EtOAc, 0.246 mL, 0.413 mmol). The reaction mixture was stirred at room temp for 1 h. Upon completion, the mixture was quenched with brine and extracted with EtOAc. The organic layers were dried and concentrated in vacuo. The residue was then diluted with MeOH (5 mL) and purified by prep.-LC-MS (XBRIDGE® C18 column, eluting with a gradient of acetonitrile/water containing 0.1% TFA, at flow rate of 60 mL/min.) to afford the product as a TFA salt. LC-MS calc. for C₃₀H₃₆C₁N₈O₂(M+H)⁺: m/z=575.3; found: 575.3. ¹H NMR (600 MHz, DMSO-d6) δ 9.67 (br. s, 1H), 9.12 (s, 1H), 8.91 (s, 1H), 7.87 (s, 1H), 7.65 (s, 1H), 7.14 (s, 1H), 6.93-6.77 (m, 2H), 4.22 (t, J 8.6 Hz, 2H), 3.04-3.97 (m, 2H), 3.91 (s, 3H), 3.23-3.05 (m, 6H), 2.90 (s, 3H), 2.25 (s, 6H), 1.23 (t, J=7.2 Hz, 6H).

### Example A. VRK1 and VRK2 Biochemical Activity Assay

Inhibitor potency was determined by luminescence-based ADP-Glo activity assay (Promega), which quantifies the ADP product of kinase reactions. Purified recombinant VRK proteins were custom-ordered from Proteos, Inc. (Kalamazoo, MI), with Baculovirus (Sf9) expression and containing 6×His tag and TEV-cleavage site at the N-terminus. Full-length construct was used for VRK1 (amino acids 1-396, Uniprot Q99986), while “Isoform 2” sequence was used for VRK2 (amino acids 2-394, Uniprot Q86Y07-2). ADP-Glo assay buffer contained 40 mM HEPES, pH 7.5, 0.02% CHAPS, 10 mM NaCl, 10 mM MgCl2, 1 mM EGTA, 0.05 mg/mL BSA, 0.5 mM TCEP, and 10% Glycerol. ATP substrate mixtures were prepared at 2× final concentrations containing ATP at 1×Km concentrations, determined experimentally to be 45 μM for VRK1 and 75 μM for VRK2. Enzyme was diluted in assay buffer to 2× final concentration immediately prior to initiating assay. Final enzyme concentrations were 20 nM VRK1 and 4 nM VRK2. DMSO or test compound in 100% DMSO at 100× concentration was added to white 384-well low-volume assay plates at 40 nL/well volume using an Echo® acoustic dispenser (Labcyte). A Tempest® liquid dispenser (Formulatrix) was used to add 2 L/well 2× enzyme mix, followed by 30 minute pre-incubation at ambient temperature. After pre-incubation, 2 L/well 2×ATP mix was added by MultiDrop Combi (ThermoFisher) to initiate reactions. After 180 min incubation, 2 l/well ADP-Glo reagent was added by Combi to quench the kinase reaction, and to deplete remaining ATP from the reactions. After 60 min incubation with ADP-Glo reagent, 2 L/well detection reagent was added by Combi to convert ADP product to luminescence signal. Luminescence was measured on a PHERAstar® FSX plate reader (BMG Labtech). Enzyme titrations were used to determine reaction rates and linearity, and to choose the concentration used in inhibitor studies for each enzyme. Chosen concentrations yielded robust assay statistics and reproducibility, with enzyme reactions and rate vs enzyme concentration relationships both in a linear range.

### Example B. VRK1 Competition Binding Assay

Inhibitor binding affinity was determined by competition Binding TR-FRET assay using a Fluorescein-labeled ATP-competitive ligand and LanthaScreen® Tb-Streptavidin detection reagent (ThermoFisher, Carlsbad, CA). Purified recombinant biotinylated VRK1 protein was custom ordered from Proteos, Inc. (Kalamazoo, MI), with Baculovirus (Sf9) expression and containing n-terminal avi-tag in addition to 6×His-tag, with in vitro biotinylation by co-expression with BirA. Full-length construct was used for VRK1 (amino acids 1-396, Uniprot Q99986). Binding assay buffer contained 40 mM HEPES, pH 7.5, 10 mM MgCl2, 10 mM NaCl, 1 mM EGTA, 0.02% CHAPS, 0.01% Brij-35, 0.1 mg/mL BSA, 2 mM DTT, and 5% Glycerol. DMSO or test compound in 100% DMSO at 200× concentration was added to white 384-well low-volume assay plates at 25 nL/well volume using an Echo® acoustic dispenser (Labcyte). Enzyme was diluted in assay buffer to 1.67× final concentration immediately prior to adding to assay. Final enzyme concentration was 0.15 nM VRK1. A Tempest® liquid dispenser (Formulatrix) was used to add 3 L/well 1.67× Enzyme mix, followed by 120 minute pre-incubation at ambient temperature. Ligand and detection mixture was prepared at 2.5× final concentrations containing custom Fluorescein-labeled ATP-competitive ligand synthesized internally at Incyte (final concentration 20 nM, 1×Kd concentration), and LanthaScreen® Tb-Streptavidin detection reagent (final concentration 0.4 nM). After pre-incubation, 2 l/well 2.5× ligand and detection mix was added by MultiDrop Combi (ThermoFisher). After 120 min incubation, LanthaScreen signal (337 nm emission, 520/490 nm dual emission) was measured on a PHERAstar® FSX plate reader (BMG Labtech). Positive signal is generated by TR-FRET between fluorescein ligand bound to enzyme in close proximity to Terbium-Streptavidin, which binds with high affinity to enzyme via Biotin tag. Displacement of ligand (competition) by test compound causes reduction of signal.

### Example C. IC₅₀ Data Analysis

The IC₅₀s for the assays in Example A and B are calculated by the following procedure. The data analysis tools integrated in Genedata Screener® software were used to analyze the inhibition data. For LanthaScreen® TR-FRET assay with dual emission, Ratiometric data were calculated and used for data fitting (“A” Channel, 520 nm emission counts, divided by “B” Channel, 490 nm emission counts, then multiplied by 10,000). Data were normalized to percent of vehicle control, after subtracting background (reference inhibitor control at 100×IC₅₀ to fully inhibit assay signal). The IC₅₀ values were derived by fitting the data to a 4-parameter logistic equation producing a sigmoidal dose-response curve using the following equation: Y=Bottom+(Top−Bottom)/(1+10\hat{ }([Log IC₅₀−X] Hill slope)), where X is the logarithm of inhibitor concentration and Y is the response.

### Potency and Selectivity Data

Tables 1 and 2 present the IC₅₀ data at VRK1 and the selectivity ratio of VRK2/VRK1 calculated by dividing the IC₅₀ value for VRK2 by the IC₅₀ value for VRK1.

In Table 1, the VRK1 IC₅₀ was measured as described in Example B, while the VRK2 IC₅₀ was measured as described in Example A.

In Table 2, the VRK1 and VRK2 IC₅₀ values were measured as described in Example A.

In Tables 1 and 2, + means an IC₅₀ of <10 nM; ++ means an IC₅₀ of 10 nM to <100 nM; +++ means an IC₅₀ of 100 nM to <1000 nM; and ++++ means an IC₅₀ of 1000 nM to 2000 nM.

TABLE 1

| Example No. | VRK1 IC₅₀ (nM) | Selectivity (VRK2/VRK1) |
|---|---|---|
| 1 | + | >100 |
| 2 | + | >100 |
| 4 | + | >100 |
| 6 | + | >100 |
| 8 | + | >100 |
| 9 | + | >100 |
| 10 | + | >100 |
| 11 | + | >100 |
| 13 | ++ | >100 |
| 14 | +++ | >100 |
| 16 | +++ | >10 |
| 17 | + | >100 |
| 18 | ++ | >100 |
| 19 | + | >100 |
| 20 | + | >100 |
| 21 | + | >100 |
| 22 | + | >100 |
| 23 | + | >100 |
| 24 | + | >100 |
| 25 | ++ | >100 |
| 26 | + | >100 |
| 27 | + | >100 |
| 27a | + | >100 |
| 27b | + | >100 |
| 28 | + | >100 |
| 29 | + | >100 |
| 30 | + | >100 |
| 31 | + | >100 |
| 32 | + | >100 |
| 33 | + | >100 |
| 34 | + | >100 |
| 35 | + | >100 |
| 36 | + | >100 |
| 37 | + | >100 |
| 38 | + | >10 |
| 39 | + | >100 |
| 40 | + | >100 |
| 41 | + | >100 |
| 42 | + | >100 |
| 43 | + | >100 |
| 44 | + | >100 |
| 45 | + | >100 |
| 46 | + | >100 |
| 47 | ++ | >10 |

TABLE 2

| Example No. | VRK1 IC₅₀ (nM) | Selectivity (VRK2/VRK1) |
|---|---|---|
| 3 | ++ | >100 |
| 5 | ++ | >100 |
| 7 | +++ | >10 |
| 12 | ++ | >10 |
| 15 | +++ | >10 |

### Example D. VRK1 Inhibitor Cellular Activity Assays

Human brain cancer cell lines were obtained from ATCC which included VRK1-dependent HS683 (HTB-138), U118MG (HTB-15) and VRK1-independent U87MG (HTB-14). A VRK1-independent line was engineered by transfecting HS683 cells to express VRK2 (termed HS683VRK2); HS683 cells were transduced with a lentiviral VRK2 expression construct from VectorBuilder, Inc and pooled transduced cells were selected for two weeks with 1 microgram per milliliter of puromycin (Gibco cat #A1113803) followed by confirmation of VRK2 expression via western blotting. All cells are maintained in growth medium DMEM (Gibco cat #1195065) supplemented with 10% FBS (Gibco cat #16140071) and are kept at 37° C. in a humidified 5% CO₂ incubator.

### Cellular Assay—VRK1 Inhibitor 6 day Viability Assays

Compounds are diluted at 1:3 ratios for dose-response determination in DMSO prior to dispensation to assay plates, with all wells receiving an equal amount of DMSO. DMSO alone serves as no treatment control (100% viability) and 20 micromolar Staurosporine (Selleck Chemicals LLC, Cat #S1421) serves as a positive control (0% viability). Compounds are dispensed to assay plates prior to addition of cells and growth medium at volumes indicated below. All cells are disassociated with TrypLE (Gibco cat #12604-013), counted, and resuspended in growth medium DMEM with 10% FBS. Cell viability assays are performed in either 96- or 384-well format. For 96-well assays, 100 microliters containing 800 cells and growth medium are seeded into black 96 well plates (Greiner cat #655090) containing 400 nanoliters per well of compounds diluted in DMSO. For 384-well assays, 50 microliters containing 400 cells and growth medium are seeded into white 384-well plates (Corning cat #3765) containing 200 nanoliters per well of compounds diluted in DMSO. Plates are incubated for six days at 37° C. in a humidified 5% CO₂ incubator. Following incubation, 50 microliters (96-well) or 25 microliters (384-well) of CellTiter Glo reagent (Promega cat #G7573) is added and plates are read on the Pherastar FSX (BMG Labtech) for luminescence. Data analyses are carried out using Genedata Screener SmartFit (Danaher Corporation), with CellTiter Glo raw data (luminescence) normalized to controls (DMSO 100% viability, Staurosporine, 0% viability). Fitting parameters can include dose response IC₅₀, Hill slope (n), maximum response (S_{∞}), and minimum response (S₀).

Various modifications of the contents of the present disclosure, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

What is claimed is:

1. A compound of Formula (I): 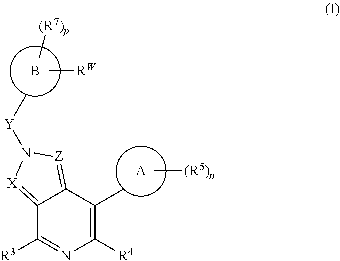
or a pharmaceutically acceptable salt thereof, wherein:
n is 0, 1, 2, 3, 4, 5, or 6;
p is 0, 1, 2, 3, 4, 5, or 6;
r is 1 or 2;
X is N or CR¹;
Z is N or CR²;
Y is -(Cy¹)ₘ-(Y¹)ₛ—, wherein Y¹ is attached to Ring moiety B;
m is 1; s is 1; and Y¹ is a bond; or
m is 1; s is 1; and Y¹ is —(CR⁶R⁶⁰)ᵣ—, —CR⁶R⁶⁰—C(O)—, —C(O)—CR⁶R⁶⁰—, or C(O); or
m is 0; s is 1; and Y¹ is —(CR⁶R⁶⁰)ᵣ—, —CR⁶R⁶⁰—C(O)—, —C(O)—CR⁶R⁶⁰—, or C(O);
Cy¹ is C₃₋₁₀ cycloalkyl, 6-10 membered aryl, 4-10 membered heterocycloalkyl, or 5-10 membered heteroaryl, which is optionally substituted by 1, 2, 3, 4, 5, or 6 independently selected R¹⁰ substituents;
Ring moiety A is C₃₋₁₂ cycloalkyl, 6-10 membered aryl, 4-12 membered heterocycloalkyl, or 5-10 membered heteroaryl;
Ring moiety B is C₃₋₁₂ cycloalkyl, 6-10 membered aryl, 4-12 membered heterocycloalkyl, or 5-10 membered heteroaryl;
R¹ is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{b1}, SR^{b1}, NHOR^{a1}, C(O)R^{b1}, C(O)NR^{c1}R^{d1}, C(O)NR^{c1}(OR^{a1}), C(O)OR^{a11}, OC(O)R^{b11}, OC(O)NR^{c1}R^{d1}, NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b11}, C(═NR^{e1})NR^{c1}R^{d1}, NR^{e1}C(═NR^{e1})NR^{c1}R^{d1}, NR^{c1}C(═NR^{e1})R^{b1}, NR^{c1}S(O)NR^{c1}R^{d1}, NR^{c1}S(O)R^{b1}, NR^{c1}S(O)₂R^{b1}, NR^{c1}S(O)(═NR^{e1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, OS(O)₂R^{b1}, S(O)(═NR^{e1})R^{b1}, SF₅, P(O)R^{f1}R^{g1}, OP(O)(OR^{h1})(ORⁱ¹), P(O)(OR^{h1})(ORⁱ¹), and BR^{j1}R^{k1}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h1} and Rⁱ¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j1} and R^{k1} is independently selected from OH, C₁₋₆ alkoxy, and C₁₋₆ haloalkoxy;
or any R^{j1} and R^{k1} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}NR^{d11}, NR^{c11}C(O)R^{b11}, NR^{c11}C(O)OR^{a11} NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h11} and Rⁱ¹¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j11} and R^{k11} is independently selected from OH, C₁₋₆ alkoxy, and C₁₋₆ haloalkoxy;
or any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12} N, R^{c12}C(═NR^{e12})R^{c12}R^{d12}, NR^{c12}C(═NR^{e12})R^{b12}, NR^{c12}S(O)NR^{c12}R^{d12}, NR^{c12}S(O)R^{b12}, NR^{c12}S(O)₂R^{b12}, NR^{c12}S(O)(═NR^{e12})R^{b12} NR^{c12}S(O)₂NR^{c12}R^{d12}S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c12} and R^{d12} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{b2}, SR^{b2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2}, NR^{c2}S(O)₂R^{b2}, NR^{c2}S(O)(═NR^{e2})R^{b2}, NR^{c2}S(O)₂NR^{c2}R^{d2}S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, S(O)(═NR^{e2})R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h2} and Rⁱ² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, NHOR^{a21}C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR²¹NR^{c21}R^{d21}, NR^{c2}C(O)R^{b21}, NR^{c21}C(O)OR^{a21}, NR^{c2}C(O)NR^{c21}R^{d21}, C(═NR^{e21})R^{b21}, C(═NR^{e21})NR^{c2}R^{d21}, NR^{c21}C(═NR^{e21})R^{c21}R^{d21}, NR^{c21}C(═NR^{e21})R^{b21}, NR^{c21}S(O)NR^{c21}R^{d21}, NR^{c2}S(O)R^{b21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
or, any R^{c21} and R^{d21} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2B} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f21} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a22}, SR^{a22}, NHOR^{a22}C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR²²NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22})R^{b22}, NR^{c22}S(O)NR^{c22}R^{d22}, NR^{c22}S(O)R^{b22}, NR^{c22}S(O)₂R^{b22}, NR^{c22}S(O)(═NR^{e22})R^{b22}, NR^{c22}S(O)₂NR^{c22}R^{d22}S(O)R^{b22}, S(O)NR^{c22}R^{d22}, S(O)₂R^{b22}, S(O)₂NR^{c22}R^{d22}, OS(O)(═NR^{e22})R^{b22}, OS(O)₂R^{b22}, S(O)(═NR^{e22})R^{b22}, SF₅, P(O)R^{f22}R^{g22}, OP(O)(OR^{h22})(ORⁱ²²), P(O)(OR^{h22})(ORⁱ²²), and BR^{j22}R^{k22}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c22} and R^{d22} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G}
each R^{b22} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f22} and R^{g22} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h22} and Rⁱ²² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j22} and R^{k22} is independently selected from OH, C₁₋₆ alkoxy, and C₁₋₆ haloalkoxy;
or any R^{j22} and R^{k22} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b3} is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4}, NR^{c4}C(═NR^{e4})R^{b4}, NR^{c4}S(O)NR^{c4}R^{d4}, NR^{c4}S(O)R^{b4}, NR^{c4}S(O)₂R^{b4}, NR^{c4}S(O)(═NR^{e4})R^{b4}, NR^{c4}S(O)₂NR^{c4}R^{d4}S(O)R^{b4}, S(O)NR^{c4}R^{d4}S(O)₂R^{b4}, and S(O)₂NR^{c4}R^{d4}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
each R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b4} is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e4} is independently selected from H, OH, CN, C₁₋₆ alkyl, C₁₋₆ alkoxy, C₁₋₆ haloalkyl, and C₁₋₆ haloalkoxy;
each R⁵ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}CC(O)R^{b5}, NR^{c5}CC(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
or, any R^{c5} and R^{d5} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)NR^{c51}R^{d51}, NR^{c51}S(O)R^{b51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, OS(O)₂R^{b51}, S(O)(═NR^{e51})R^{b51}, SF₅, P(O)R^{f51}R^{g51}, OP(O)(OR^{h51})(ORⁱ⁵¹), P(O)(OR^{h51})(ORⁱ⁵¹), and BR^{j51}R^{k51}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c51} and R^{d51} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b51} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f51} and R^{g51} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h51} and Rⁱ⁵¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j51} and R^{k51} is independently selected from OH, C₁₋₆ alkoxy, and C₁₋₆ haloalkoxy;
or any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶ is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c6}R^{d6}, C(═NR^{e6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)NR^{c6}R^{d6}, NR^{c6}S(O)R^{b6}, NR^{c6}S(O)₂R^{b6}, NR^{c6}S(O)(═NR^{e6})R^{b6}, NR^{c6}S(O)₂NR^{c6}R^{d6}, S(O)R^{b6}, S(O)NR^{c6}R^{d6}, S(O)₂R^{b6}, S(O)₂NR^{c6}R^{d6}, OS(O)(═NR^{e6})R^{b6}, OS(O)₂R^{b6}, S(O)(═NR^{e6})R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
or, any R^{c6} and R^{d6} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected RA substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f6} and R^{g6} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h6} and Rⁱ⁶ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j6} and R^{k6} is independently selected from OH, C₁₋₆ alkoxy, and C₁₋₆ haloalkoxy;
or any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, NHOR^{a61}, C(O)R^{b61}, C(O)NR^{c61}R^{d61}, C(O)NR^{c61}(OR^{a61}), C(O)OR^{a61}, OC(O)R^{b61}, OC(O)NR^{c6}R^{d61}, NR^{c61}R^{d61}, NR^{c61}NR^{c61}R^{d61}, NR^{c61}C(O)R^{b61}, NR^{c61}C(O)OR^{a61}, NR^{c61}C(O)NR^{c61}R^{d61}, C(═NR^{e61})R^{b61}, C(═NR^{e61})NR^{c61}R^{d61}, NR^{c61}C(═NR^{e61})NR^{c61}R^{d61}, NR^{c61}C(═NR^{e61})R^{b61}, NR^{c61}S(O)NR^{c61}R^{d61}, NR^{c6}S(O)R^{b61}, NR^{c61}S(O)₂R^{b61}, NR^{c61}S(O)(═NR^{e61})R^{b61}, NR^{c61}S(O)₂NR^{c61}R^{d61}, S(O)R^{b61}, S(O)NR^{c61}R^{d61}, S(O)₂R^{b61}, S(O)₂NR^{c6}R^{d61}, OS(O)(═NR^{e61})R^{b61}, OS(O)₂R^{b61}, S(O)(═NR^{e61})R^{b61}, SF₅, P(O)R^{f61}R^{g61}, OP(O)(OR^{h61})(ORⁱ⁶¹), P(O)(OR^{h61})(ORⁱ⁶¹), and BRⁱ⁶¹R^{k61}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{e61} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f61} and R^{g61} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h61} and Rⁱ⁶¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j61} and R^{k61} is independently selected from OH, C₁₋₆ alkoxy, and C₁₋₆ haloalkoxy;
or any R^{j61} and R^{k61} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, NHOR^{a62}C(O)R^{b62}, C(O)NR^{c62}R^{d62}, C(O)NR^{c62}(OR^{a62}), C(O)OR^{a62}, OC(O)R^{b62}, OC(O)NR^{c62}R^{d62}, NR^{c62}R^{d62}, NR^{c62}NR^{c62}R^{d62}, NR^{c62}C(O)R^{b62}, NR^{c62}C(O)OR^{a62}, NR^{c62}C(O)NR^{c62}R^{d62}, C(═NR^{e62})R^{b62}, C(═NR^{e62})NR^{c62}R^{d62}, NR^{c62}C(═NR^{e62}NR^{c62}R^{d62}, NR^{c62}C(═NR^{e62})R^{b62}, NR^{c62}S(O)NR^{c62}R^{d62}, NR^{c62}S(O)R^{b62}, NR^{c62}S(O)₂R^{b62}, NR^{c62}S(O)(═NR^{e62})R^{b62}, NR^{c62}S(O)₂NR^{c62}R^{d62}S(O)R^{b62}, S(O)NR^{c62}R^{d62}, S(O)₂R^{b62}, S(O)₂NR^{c62}R^{d62}, OS(O)(═NR^{e62})R^{b62}, OS(O)₂R^{b62}, S(O)(═NR^{e62})R^{b62}, SF₅, P(O)R^{f62}R^{g62}, OP(O)(OR^{h62})(ORⁱ⁶²), P(O)(OR^{h62})(ORⁱ⁶²), and BR^{j62}R^{k62}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a62}, R^{c62}, and R^{d62} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c62} and R^{d62} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b62} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e62} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f62} and R^{g62} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h62} and Rⁱ⁶² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j62} and R^{k62} is independently selected from OH, C₁₋₆ alkoxy, and C₁₋₆ haloalkoxy;
or any R^{j62} and R^{k62} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶⁰ is independently selected from H, D, C₁₋₆ alkyl, and C₁₋₆ haloalkyl;
or R⁶ and R⁶⁰, together with the carbon atom to which they are attached, form a C₃₋₁₀ cycloalkyl or a 4-10 membered heterocycloalkyl moiety, each of which is optionally substituted with 1, 2, 3, or 4 independently selected R^{7A} substituents;
each R⁷ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)NR^{c7}R^{d7}, NR^{c7}S(O)R^{b7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{d7}S(O)₂R^{b7}, S(O)₂NR^{c7}R^{d7}, OS(O)(═NR^{e7})R^{b7}, OS(O)₂R^{b7}, S(O)(═NR^{e7})R^{b7}, SF₅, P(O)R^{f7}R^{g7}, OP(O)(OR^{h7})(ORⁱ⁷), P(O)(OR^{h7})(ORⁱ⁷), and BR^{j7}R^{k7}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
or, any R^{c7} and R^{d7} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h7} and Rⁱ⁷ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j7} and R^{k7} is independently selected from OH, C₁₋₆ alkoxy, and C₁₋₆ haloalkoxy;
or any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7A} is independently selected from D, halo, CN, NO₂, C₁₋₆ alkyl, C₂₋₆ alkenyl, C₂₋₆ alkynyl, C₁₋₆ haloalkyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, NHOR^{a71}, C(O)R^{b71}, C(O)NR^{c71}R^{d71}, C(O)NR^{c71}(OR^{a71}), C(O)OR^{a71}, OC(O)R^{b71}, OC(O)NR^{c71}R^{d71}, NR^{c71}R^{d71}, NR^{c71}NR^{c71}R^{d71}, NR^{c71}C(O)R^{b71}, NR^{c71}C(O)OR^{a71}, NR^{c71}C(O)NR^{c71}R^{d71}, C(═NR^{e71})R^{b71}, C(═NR^{e71})NR^{c7}R^{d71}, NR^{c71}C(═NR^{e71})NR^{c71}R^{d71}, NR^{c71}C(═NR^{e71})R^{b71}, NR^{c71}S(O)NR^{c71}R^{c71}R^{d71}, S(O)R^{b71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, OS(O)₂R^{b71}, S(O)(═NR^{e71})R^{b71}, SF₅, P(O)R^{f71}R^{g71}, OP(O)(OR^{h71})(ORⁱ⁷¹), P(O)(OR^{h71})(ORⁱ⁷¹), and BR^{j71}R^{k71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B}
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{e71} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f71} and R^{g71} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h71} and Rⁱ⁷¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j71} and R^{k71} is independently selected from OH, C₁₋₆ alkoxy, and C₁₋₆ haloalkoxy;
or any R^{j71} and R^{k71} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a72}, SR^{a72}, NHOR^{a72}C(O)R^{b72}, C(O)NR^{c72}R^{d72}, C(O)NR^{c72}(OR^{a72}), C(O)OR^{a72}, OC(O)R^{b72}, OC(O)NR^{c72}R^{d72}, NR^{c72}R^{d72}, NR^{c72}NR^{c72}R^{d72}, NR^{c72}C(O)R^{b72}, NR^{c72}C(O)OR^{a72}, NR^{c72}C(O)NR^{c72}R^{d72}, C(═NR^{e72})R^{b72}, C(═NR^{e72})NR^{c72}R^{d72}, NR^{c72}C(═NR^{e72})NR^{c72}R^{d72}, NR^{c72}C(═NR^{e72})R^{b72}, NR^{c72}S(O)NR^{c72}R^{d72}, NR^{c72}S(O)R^{b72}, NR^{c72}S(O)₂R^{b72}, NR^{c72}S(O)(═NR^{e72})R^{b72}, NR^{c72}S(O)₂NR^{c72}R^{d72}S(O)R^{b72}, S(O)NR^{c72}R^{d72}, S(O)₂R^{b72}, S(O)₂NR^{c72}R^{d72}, OS(O)(═NR^{e72})R^{b72}, OS(O)₂R^{b72}, S(O)(═NR^{e72})R^{b72}, SFS, P(O)R^{f72}R^{g72}, OP(O)(OR^{h72})(ORⁱ⁷²), P(O)(OR^{h72})(ORⁱ⁷²), and BR^{j72}R^{k72}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a72}, R^{c72}, and R^{d72} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c72} and R^{d72} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b72} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e72} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f72} and R^{g72} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h72} and Rⁱ⁷² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j72} and R^{k72} is independently selected from OH, C₁₋₆ alkoxy, and C₁₋₆ haloalkoxy;
or any R^{j72} and R^{k72} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{W} is a covalent warhead moiety having a reactive functional group capable of covalently binding to one or more cysteine residues in a protein;
each R¹⁰ is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a10}, SR^{a10}, NHOR^{a10}, C(O)R^{b10}, C(O)NR^{c10}R^{d10}, C(O)NR^{c10}(OR^{a10}), C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)NR^{c10}R^{d10}, NR^{c10}S(O)R^{b10}, NR^{c10}S(O)₂R^{b11}, NR^{c10}S(O)₂NR^{c10}R^{d10}, S(O)R^{b10}, S(O)NR^{c10}R^{d10}, S(O)₂R^{b10}, and S(O)₂NR^{c10}R^{d10}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a10}, R^{c10}, and R^{d10} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c10} and R^{d10} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{3A} substituents;
each R^{b10} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{3A} substituents; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, N(CD₃)₂, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

2. The compound of claim 1, or a pharmaceutically acceptable salt thereof, wherein R^{W} is a means for covalently binding to cysteine residue in a protein.

3. The compound of claim 1, or a pharmaceutically acceptable salt thereof, wherein R^{W} is: 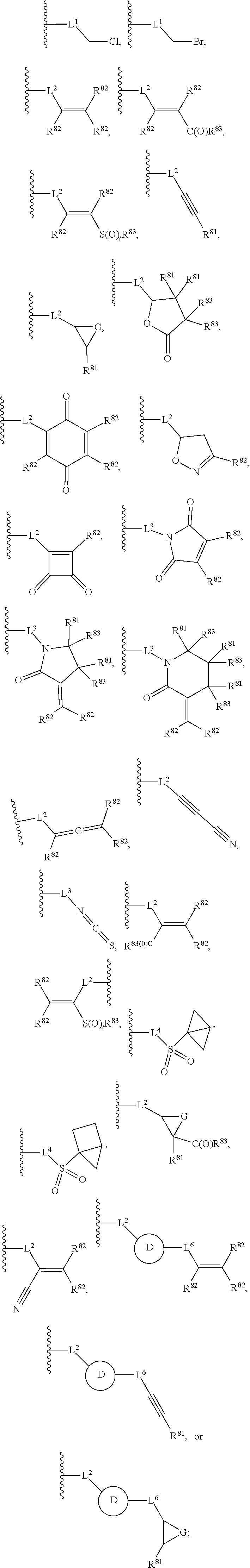
L¹ is -L-C(O)—, -L-NR⁹C(O)—, -L-OC(O)—, -L-S(O)—, -L-S(O)₂—, -L-NR⁹S(O)—, -L-OS(O)—, -L-NR⁹S(O)₂—, or -L-OS(O)₂—, wherein L¹ is attached to Ring moiety B through the L linking group;
L² is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NR⁹S(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—, wherein L² is attached to Ring moiety B through the L linking group;
L³ is -L-, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)₂—, or -L-OS(O)₂—, wherein L³ is attached to Ring moiety B through the L linking group;
L⁴ is -L-, -L-O—, L-S—, or -L-NR⁹—, wherein L⁴ is attached to Ring moiety B through the L linking group;
L⁶ is -L-, -L-O—, -L-NR⁹—, -L-S—, -L-C(O)—, -L-NR⁹C(O)—, -L-OC(O)—, -L-S(O)—, -L-S(O)₂—, -L-NR⁹S(O)—, -L-OS(O)—, -L-NR⁹S(O)NR⁹—, -L-NRS(O)O—, -L-OS(O)NR⁹—, -L-NR⁹S(O)₂—, -L-OS(O)₂—, -L-NR⁹S(O)₂NR⁹—, -L-NR⁹S(O)₂O—, -L-S(O)(NR⁹)—, -L-S(O)₂(NR⁹)—, or -L-OS(O)₂NR⁹—, wherein L⁶ is attached to Ring moiety D through the L linking group;
L is a bond or C₁₋₆ alkylene, wherein said C₁₋₆ alkylene is optionally substituted by 1, 2, 3, or 4 independently selected R^{G} substituents; or
L is —O—C₁₋₆ alkyl or —N(R^{N})—C₁₋₆ alkyl, wherein L is attached to Ring moiety A via the oxygen of the —O—C₁₋₆ alkyl or the nitrogen atom of the —N(R^{N})—C₁₋₆ alkyl group;
each R^{N} is independently H or C₁₋₆ alkyl;
Ring D is a 4-12 membered heterocycloalkyl, C₃₋₁₂ cycloalkyl, C₆₋₁₀ aryl, or a 5-10 membered heteroaryl, each of which is optionally substituted with 1, 2, 3, or 4 independently selected C₁₋₆ alkyl groups;
G is O or NR⁹;
each t is independently 0, 1, 2, or 3;
each R⁸¹ and R⁸³ are independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a8}, SR^{a5}, NHOR^{a8}, C(O)R^{b8}, C(O)NR^{c8}R^{d8}, C(O)NR^{c8}(OR^{a8}), C(O)OR^{a5}, OC(O)R^{h1}, OC(O)NR^{c8}R^{d8}, NR^{e8}R^{d8}, NR^{c8}NR^{c8}R^{d8}, NR^{c8}C(O)R^{b8}, NR^{c8}(O)OR^{a5}, NR^{c8}C(O)NR^{c8}R^{d8}, C(═NR^{e8})R^{b8}, C(═NR^{e8})NR^{c8}R^{d8}, NR^{c8}C(═NR^{e8})NR^{e8}R^{d8}, NR^{c8}C(═NR^{e8})R^{b8}, NR^{c8}S(O)NR^{c8}R^{d8}, NR^{c}'S(O)R^{b5}, NR^{c8}S(O)₂R^{b8}, NR^{c}'S(O)(═NR^{e8})R^{b5}, NR^{c8}S(O)₂NR^{c8}R^{d8}S(O)R^{b8}, S(O)NR^{e8}R^{d8}, S(O)₂R^{b8}, S(O)₂NR^{e8}R^{d8}, OS(O)(═NR^{e8})R^{b8}, OS(O)₂R^{b8}, S(O)(═NR^{e8})R^{b8}, SF₅, P(O)R^{f8}R^{g8}, OP(O)(OR^{h8})(ORⁱ⁸), P(O)(OR^{h8})(ORⁱ⁸), and BR^{j8}R^{k8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R⁸² is independently selected from H, D, halo, NO₂, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, C(O)R^{b8}, C(O)NR^{e8}R^{d8}, C(O)OR^{a8}, C(═NR^{e8})R^{b8}, C(═NR^{e8})NR^{e8}R^{d8}, S(O)R^{b8}, S(O)NR^{e8}R^{d8}S(O)₂R^{b8}, and S(O)₂NR^{c8}R^{d8}; wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a5}, R^{c8}, and R^{d8} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b8} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e8} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f8} and R^{g8} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h8} and Rⁱ⁸ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j8} and R^{k8} is independently selected from OH, C₁₋₆ alkoxy, and C₁₋₆ haloalkoxy;
or any R^{j8} and R^{k8} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
or any two R⁸¹ and R⁸² together with the atoms to which they are attached, form C₃₋₇ cycloalkyl, 4-7 membered heterocycloalkyl, phenyl, or 5-6-membered heteroaryl ring, each of which is optionally substituted with 1, 2, 3, or 4 independently selected R^{G} substituents;
each R⁹ is independently selected from H, halo, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, OR^{a9}, SR^{a9}, NHOR^{a9}, C(O)R^{b9}, C(O)NR^{c9}R^{d9}, C(O)NR^{c9}(OR^{a9}), C(O)OR^{a9}, OC(O)R^{b9}, OC(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, C(═NR^{e9})R^{b9}, C(═NR^{e9})NR^{c9}R^{d9}, NR^{c9}C(═NR^{e9})NR^{c9}R^{d9}, NR^{c9}C(═NR^{e9})R^{b9}, NR^{c9}S(O)NR^{c9}R^{d9}, NR^{c9}S(O)R^{b9}, NR^{c9}S(O)₂R^{b9}, NR^{c9}S(O)(═NR^{e9})R^{b9}, NR^{c9}S(O)₂NR^{c9}R^{d9}S(O)R^{b9}, S(O)NR^{c9}R^{d9}S(O)₂R^{b9}, S(O)₂NR^{c9}R^{d9}, OS(O)(═NR^{e9})R^{b9}, OS(O)₂R^{b9}, and S(O)(═NR^{e9})R^{b9}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{G} substituents;
each R^{a9}, R^{c9}, and R^{d9} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c9} and R^{d9} attached to the same N atom, together with the N atom to which they are attached, form a 4-7 membered heterocycloalkyl group, which is optionally substituted with 1, 2, 3, or 4 independently selected R^{G} substituents;
each R⁹ is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents; and
each R^{e9} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl.

4. The compound of claim 3, or a pharmaceutically acceptable salt thereof, wherein R^{W} is selected from.

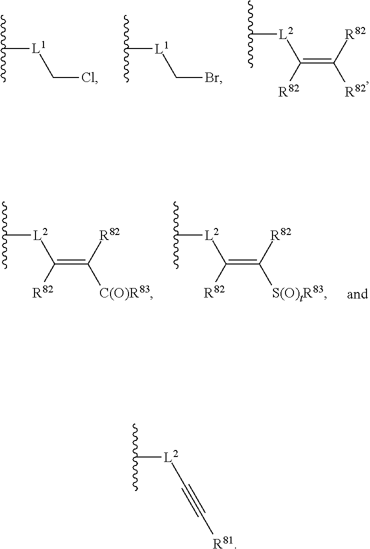

5. The compound of claim 3, or a pharmaceutically acceptable salt thereof, wherein R^{W} is selected from:

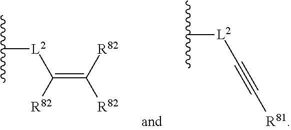

6. The compound of claim 3, or a pharmaceutically acceptable salt thereof, wherein L¹ is -L-C(O)—, -L-NR⁹C(O)—, -L-S(O)₂—, -L-NR⁹S(O)—, or -L-NR⁹S(O)₂—, or wherein L¹ is attached to Ring moiety B through the L linking group; L is a bond; and R⁹ is selected from H and C₁₋₆ alkyl.

7. The compound of claim 3, or a pharmaceutically acceptable salt thereof, wherein L² is —C(O)—, —NR⁹C(O)—, —S(O)₂—, or —NR⁹S(O)₂—; and R⁹ is selected from H and C₁₋₃ alkyl.

8. The compound of claim 3, or a pharmaceutically acceptable salt thereof, wherein R⁸¹, R⁸², and R⁸³ are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, OR^{a8}, C(O)R^{b8}, C(O)NR^{c8}R^{d8}, and C(O)OR^{a8}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino.

9. The compound of claim 1, or a pharmaceutically acceptable salt thereof, wherein n is 0, 1, 2, 3, or 4.

10. The compound of claim 1, or a pharmaceutically acceptable salt thereof, wherein p is 0, 1, or 2.

11. The compound of claim 1, or a pharmaceutically acceptable salt thereof, wherein R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1A} substituents.

12. The compound of claim 1, or a pharmaceutically acceptable salt thereof, wherein:
each R^{a1}, R^{c}, and R^{d1} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11} is independently selected from H, C₁₋₆ alkyl, and C₁₋₆ haloalkyl; and
each R^{b11} is independently selected from C₁₋₆ alkyl and C₁₋₆ haloalkyl.

13. The compound of claim 1, or a pharmaceutically acceptable salt thereof, wherein R² is selected from H, D, halo, CN, C₁₋₆ alkyl, C₂₋₆ alkenyl, C₂₋₆ alkynyl, and C₁₋₆ haloalkyl.

14. The compound of claim 1, or a pharmaceutically acceptable salt thereof, wherein m is 1; s is 1; and Y¹ is —CR⁶R⁶⁰— or C(O).

15. The compound of claim 1, or a pharmaceutically acceptable salt thereof, wherein Cy¹ is 4-10 membered heterocycloalkyl, which is optionally substituted by 1, 2, 3, or 4 independently selected R¹⁰ substituents.

16. The compound of claim 1, or a pharmaceutically acceptable salt thereof, wherein Cy¹ is an azetidine ring, a pyrrolidine ring, a piperidine ring, a piperazine ring, or an azepine ring, which are each optionally substituted by 1, 2, 3, or 4 independently selected R¹⁰ substituents.

17. The compound of claim 1, or a pharmaceutically acceptable salt thereof, wherein m is 0; s is 1; and Y¹ is —CR⁶R⁶⁰— or C(O).

18. The compound of claim 1, or a pharmaceutically acceptable salt thereof, wherein each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl; and
each R^{b10} is independently selected from C₁₋₆ alkyl and C₁₋₆ haloalkyl.

19. The compound of claim 1, or a pharmaceutically acceptable salt thereof, wherein each R⁶ is independently selected from H, D, halo, CN, C₁₋₆ alkyl, C₁₋₆ haloalkyl, and C₃₋₇ cycloalkyl.

20. The compound of claim 1, or a pharmaceutically acceptable salt thereof, wherein each R⁶⁰ is independently selected from H, D and C₁₋₃ alkyl.

21. The compound of claim 1, or a pharmaceutically acceptable salt thereof, wherein R⁶ and R⁶⁰, together with the carbon atom to which they are attached, form a C₃₋₁₀ cycloalkyl moiety, which is optionally substituted with 1, 2, 3, or 4 independently selected R^{7A} substituents.

22. The compound of claim 1, or a pharmaceutically acceptable salt thereof, wherein Ring moiety B is phenyl, naphthyl, 5-10 membered heteroaryl, or 9-12 membered heterocycloalkyl.

23. The compound of claim 1, or a pharmaceutically acceptable salt thereof, wherein Ring moiety B is phenyl, naphthyl, 5-6 membered heteroaryl, 9-10 membered heteroaryl, or 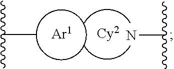
wherein:
Ar¹ is fused to Cy² and Ar¹ is bonded to Y;
Ar¹ is phenyl or 5-6 membered heteroaryl; and
Cy² is 4-8 membered heterocycloalkyl.

24. The compound of claim 1, or a pharmaceutically acceptable salt thereof, wherein R³ is selected from H, D, halo, CN, C₁₋₆ alkyl, C₂₋₆ alkenyl, C₂₋₆ alkynyl, C₁₋₆ haloalkyl, OR^{a3}, and NR^{c3}R^{d3}.

25. The compound of claim 1, or a pharmaceutically acceptable salt thereof, wherein each R^{a3}, R^{c3}, and R^{d3} is independently selected from H and C₁₋₆ alkyl.

26. The compound of claim 1, or a pharmaceutically acceptable salt thereof, wherein R⁴ is selected from H, D, halo, CN, C₁₋₆ alkyl, C₂₋₆ alkenyl, C₂₋₆ alkynyl, and C₁₋₆ haloalkyl.

27. The compound of claim 1, or a pharmaceutically acceptable salt thereof, wherein Ring moiety A is 6-10 membered aryl or 5-10 membered heteroaryl.

28. The compound of claim 1, or a pharmaceutically acceptable salt thereof, wherein each R⁵ is independently selected from halo, CN, C₁₋₃ alkyl, C₁₋₃ haloalkyl, OH, C₁₋₃ alkoxy, and C₁₋₃ haloalkoxy.

29. The compound of claim 1, or a pharmaceutically acceptable salt thereof, wherein: 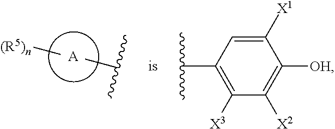
wherein X¹ and X² are each independently Cl or F, and X³ is H, Cl or F.

30. The compound of claim 1, or a pharmaceutically acceptable salt thereof, wherein: 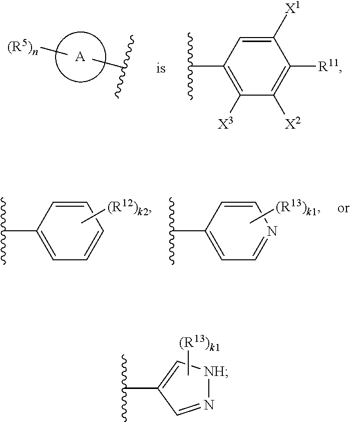
X¹ is Cl, F, or OCH₃;
X² is H, Cl, or F;
X³ is H, Cl or F;
R¹¹ is OH or CN;
k1 is 0, 1, or 2;
k2 is 2;
two R¹² substituents, together with the carbon atoms to which they are attached, form a pyrazole, pyrrole, or pyrimidine ring, which is optionally substituted by 1 or 2 substituents independently selected from halo, CN, and C₁₋₃ alkyl; and
each R¹³ is independently selected from halo, CN, C₁₋₃ alkyl, and methoxy.

31. The compound of claim 1, or a pharmaceutically acceptable salt thereof, wherein each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents.

32. The compound of claim 1, or a pharmaceutically acceptable salt thereof, wherein:
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents; and
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino.

33. The compound of claim 1, or a pharmaceutically acceptable salt thereof, wherein:
n is 0, 1, 2, 3, or 4;
p is 0, 1, 2, 3, or 4;
m is 1; s is 1; and Y¹ is a bond; or
m is 1; s is 1; and Y¹ is —CR⁶R⁶⁰—, —(CR⁶R⁶⁰)₂—, —CR⁶R⁶⁰—C(O)—, —C(O)—CR⁶R⁶⁰—, or C(O); or
m is 0; s is 1; and Y¹ is —CR⁶R⁶⁰—, —(CR⁶R⁶⁰)₂—, —CR⁶R⁶⁰—C(O)—, —C(O)—CR⁶R⁶⁰—, or C(O);
Cy¹ is 4-7 membered heterocycloalkyl having 1 or 2 nitrogen ring members, which is optionally substituted by 1, 2, 3, or 4 independently selected R¹⁰ substituents;
Ring moiety A is phenyl, 5-6 membered heteroaryl, or 9-10 membered heteroaryl;
Ring moiety B is phenyl, naphthyl, 5-6 membered heteroaryl, 9-10 membered heteroaryl, or 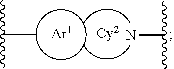
Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{al1}, SR^{al1}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11} is independently selected from H, C₁₋₆ alkyl, and C₁₋₆ haloalkyl;
each R^{b11} is independently selected from C₁₋₆ alkyl and C₁₋₆ haloalkyl;
R² is selected from H, D, halo, CN, C₁₋₆ alkyl, C₂₋₆ alkenyl, C₂₋₆ alkynyl, C₁₋₆ haloalkyl, and C₃₋₅ cycloalkyl, wherein said C₁₋₆ alkyl, C₂₋₆ alkenyl, C₂₋₆ alkynyl, C₁₋₆ haloalkyl, and C₃₋₅ cycloalkyl are each optionally substituted with 1, 2, 3, or 4 independently selected R^{2A} substituents;
each R^{2A} is independently selected from D, halo, CN, C₁₋₆ alkyl, C₂₋₆ alkenyl, C₂₋₆ alkynyl, C₁₋₆ haloalkyl, OR^{a21}, SR^{a21}, C(O)R^{b21}, C(O)NR^{c21}R^{d21}, C(O)OR^{a21}, OC(O)R^{b21}, OC(O)NR^{c21}R^{d21}, NR^{c21}R^{d21}, NR^{c21}C(O)R^{b21}, NR^{c21}C(O)OR^{a21}, NR^{c21}C(O)NR^{c21}R^{d21}, NR^{c21}S(O)₂R^{b21}, NR^{c21}S(O)₂NR^{c21}R^{d21}, S(O)₂R^{b21}, and S(O)₂NR^{c21}R^{d21};
each R^{a21}, R^{c21}, and R^{d21} is independently selected from H, C₁₋₆ alkyl, and C₁₋₆ haloalkyl;
each R^{b21} is independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, C₁₋₆ haloalkyl, OR^{a3}, and NR^{c3}R^{d3};
each R^{a3}, R^{c3}, and R^{d3} is independently selected from H and C₁₋₆ alkyl;
R⁴ is selected from H, D, halo, CN, C₁₋₆ alkyl, C₂₋₆ alkenyl, C₂₋₆ alkynyl, and C₁₋₆ haloalkyl;
R⁵ is independently selected from D, halo, CN, C₁₋₆ alkyl, C₂₋₆ alkenyl, C₂₋₆ alkynyl, C₁₋₆ haloalkyl, C₃₋₄ cycloalkyl, OR^{a5} and NR^{c5}R^{d5}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{5A} substituents;
each R^{a5}, R^{c5}, and R^{d5} is independently selected from C₁₋₆ alkyl, C₁₋₆ haloalkyl, C₂₋₆ alkenyl, C₂₋₆ alkynyl, C₃₋₄ cycloalkyl, and 4-5 membered heterocycloalkyl, which are each optionally substituted with 1, 2, 3, or 4 independently selected R^{5A} substituents;
each R^{5A} is independently selected from OH, CN, halo, C₁₋₃ alkyl, C₁₋₃ haloalkyl, amino, C₁₋₃ alkylamino, and di(C₁₋₃ alkyl)amino;
each R⁶ is independently selected from H, D, halo, CN, C₁₋₆ alkyl, C₂₋₆ alkenyl, C₂₋₆ alkynyl, C₁₋₆ haloalkyl, C₃₋₇ cycloalkyl, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, NR^{c6}S(O)₂NR^{c6}R^{d6}S(O)₂R^{b6}, and S(O)₂NR^{c6}R^{d6}, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents;
each R⁶⁰ is independently selected from H, C₁₋₆ alkyl, and C₁₋₆ haloalkyl;
or R⁶ and R⁶⁰, together with the carbon atom to which they are attached, form a C₃₋₇ cycloalkyl or a 4-7 membered heterocycloalkyl moiety, each of which are optionally substituted with 1, 2, 3, or 4 independently selected R^{7A} substituents;
each R^{a6}, R^{c6}, and R^{d6} is independently selected from H, C₁₋₆ alkyl, C₁₋₆ haloalkyl, C₂₋₆ alkenyl, and C₂₋₆ alkynyl, wherein said C₁₋₆ alkyl, C₂₋₆ alkenyl, C₂₋₆ alkynyl, and C₁₋₆ haloalkyl are each optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, and C₂₋₆ alkynyl, which are each optionally substituted with 1, 2, 3, or 4 independently selected R^{6A} substituents;
each R^{6A} is independently selected from D, halo, CN, C₁₋₆ alkyl, C₂₋₆ alkenyl, C₂₋₆ alkynyl, C₁₋₆ haloalkyl, OR^{a61}, SR^{a61}, C(O)R^{b61}, C(O)NR^{c61}R^{d61}, C(O)OR^{a61}, OC(O)R^{b61}, OC(O)NR^{c61}R^{d61}, NR^{c61}R^{d61}, NR^{c61}C(O)R^{b61}, NR^{c61}C(O)OR^{a61}, NR^{c61}C(O)NR^{c61}R^{d61}, NR^{c61}S(O)₂R^{b61}, NR^{c61}S(O)₂NR^{c61}R^{d61}, S(O)₂R^{b61}, and S(O)₂NR^{c61}R^{d61};
each R^{a61}, R^{c61}, and R^{d61} is independently selected from H, C₁₋₆ alkyl, and C₁₋₆ haloalkyl;
each R^{b61} is independently selected from C₁₋₆ alkyl and C₁₋₆ haloalkyl;
each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7A} substituents;
each R^{7A} is independently selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl;
each R^{b71} is independently selected from C₁₋₆ alkyl and C₁₋₆ haloalkyl;
R^{W} is selected from: 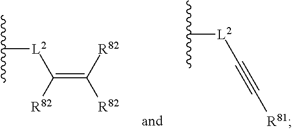
L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, OR^{a8}, C(O)R^{b8}, C(O)NR^{c8}R^{d8}, and C(O)OR^{a5}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each R^{a5}, R⁸, and R^{d8} is independently selected from H and C₁₋₆ alkyl;
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b11}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl;
each R¹⁰ is independently selected from C₁₋₆ alkyl and C₁₋₆ haloalkyl; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, N(CD₃)₂, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

34. The compound of claim 1, or a pharmaceutically acceptable salt thereof, wherein:
n is 0, 1, 2, 3, or 4;
p is 0, 1, 2, 3, or 4;
m is 1; s is 1; and Y¹ is a bond; or
m is 1; s is 1; and Y¹ is —CR⁶R⁶⁰—, —(CR⁶R⁶⁰)₂—, or C(O); or
m is 0; s is 1; and Y¹ is —CR⁶R⁶⁰—, —(CR⁶R⁶⁰)₂—, or C(O); Cy¹ is an azetidine ring, a pyrrolidine ring, or a piperidine ring, which are each optionally substituted by 1 or 2 independently selected R¹⁰ substituents;
Ring moiety A is phenyl, 5-6 membered heteroaryl, or 9-10 membered heteroaryl;
Ring moiety B is phenyl, naphthyl, 5-6 membered heteroaryl, 9-10 membered heteroaryl, or 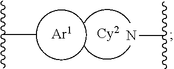
Ar¹ is fused to Cy² and Ar¹ is bonded to Y;
Ar¹ is phenyl or 5-6 membered heteroaryl;
Cy² is 4-8 membered heterocycloalkyl;
X is N or CR¹;
Z is N or CR²;
R¹ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{1A} substituents;
each R^{1A} is independently selected from D, halo, CN, C₁₋₆ alkyl, C₂₋₆ alkenyl, C₂₋₆ alkynyl, C₁₋₆ haloalkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11};
each R^{a11}, R^{c11}, and R^{d11} is independently selected from H, C₁₋₆ alkyl, and C₁₋₆ haloalkyl;
each R^{b11} is independently selected from C₁₋₆ alkyl and C₁₋₆ haloalkyl;
R² is selected from H, D, halo, CN, C₁₋₆ alkyl, C₂₋₆ alkenyl, C₂₋₆ alkynyl, and C₁₋₆ haloalkyl;
R³ is selected from H, D, halo, CN, C₁₋₆ alkyl, C₂₋₆ alkenyl, C₂₋₆ alkynyl, and C₁₋₆ haloalkyl;
R⁴ is selected from H, D, halo, CN, C₁₋₆ alkyl, C₂₋₆ alkenyl, C₂₋₆ alkynyl, and C₁₋₆ haloalkyl;
each R⁵ is independently selected from halo, CN, C₁₋₃ alkyl, C₁₋₃ haloalkyl, OH, C₁₋₃ alkoxy, and C₁₋₃ haloalkoxy;
each R⁶ is independently selected from H, D, halo, CN, C₁₋₆ alkyl, and C₁₋₆ haloalkyl;
each R⁶⁰ is independently selected from H and C₁₋₃ alkyl;
or R⁶ and R⁶⁰, together with the carbon atom to which they are attached, form a C₃₋₇ cycloalkyl moiety optionally substituted with 1 or 2 independently selected R^{7A} substituents;
each R⁷ is independently selected from halo, CN, C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, 5-6 membered heteroaryl, OR^{a7}, C(O)R^{b7}, C(O)NR^{c7}R^{d7}, C(O)OR^{a7}, and NR^{c7}R^{d7}, wherein said C₁₋₆ alkyl, C₂₋₆ alkenyl, C₂₋₆ alkynyl, C₁₋₆ haloalkyl, C₁₋₄ alkylidene-C₃₋₇ cycloalkyl, C₁₋₄ alkylidene-4-7-membered heterocycloalkyl, C₃₋₇ cycloalkyl, phenyl, 4-7 membered heterocycloalkyl, and 5-6 membered heteroaryl are each optionally substituted with 1, 2, 3, or 4 independently selected R^{7A} substituents;
each R^{a7}, R^{c7}, and R^{d7} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7A} substituents;
each R^{b7} is independently selected from C₁₋₆ alkyl and C₁₋₆ haloalkyl, which are each optionally substituted with 1, 2, 3, or 4 independently selected R^{7A} substituents;
each R^{7A} is independently selected from D, halo, CN, C₁₋₆ alkyl, C₂₋₆ alkenyl, C₂₋₆ alkynyl, C₁₋₆ haloalkyl, OR^{a71}, C(O)R^{b71}, C(O)NR^{c71}R^{d71}, C(O)OR^{a71}, and NR^{c71}R^{d71};
each R^{a71}, R^{c71}, and R^{d71} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B} substituents;
each R^{b71} is independently selected from C₁₋₆ alkyl and C₁₋₆ haloalkyl, which are each optionally substituted with 1, 2, 3, or 4 independently selected R^{7B} substituents; and
each R^{7B} is independently selected from D, OH, CN, halo, C₁₋₃ alkyl, C₂₋₃ alkenyl, C₂₋₃ alkynyl, C₁₋₃ haloalkyl, C₁₋₃ alkoxy, C₁₋₃ haloalkoxy, amino, C₁₋₃ alkylamino, and di(C₁₋₃ alkyl)amino;
R^{W} is selected from: 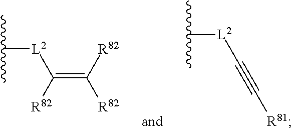
L² is —C(O)—, —NR⁹C(O)—, —S(O)₂—, or —NR⁹S(O)₂—;
provided that when L² is —C(O)— or —S(O)₂— and Z is Cy¹, then L² is attached to a nitrogen ring member of Cy¹;
R⁹ is selected from H and C₁₋₃ alkyl;
R⁸¹ and R⁸² are each independently selected from H, halo, CN, C₁₋₆ alkyl, C₁₋₆ haloalkyl, 5-6 membered heteroaryl, 4-7 membered heterocycloalkyl-C₁₋₄ alkyl, 5-6 membered heteroaryl-C₁₋₄ alkyl, and C(O)OR^{a8}, wherein said C₁₋₆ alkyl, C₁₋₆ haloalkyl, 5-6 membered heteroaryl, 4-7 membered heterocycloalkyl-C₁₋₄ alkyl, and 5-6 membered heteroaryl-C₁₋₄ alkyl are each optionally substituted by 1 or 2 substituents selected from halo, CN, C₁₋₃ alkyl, C₁₋₃ haloalkyl, OH, C₁₋₃ alkoxy, C₁₋₃ haloalkoxy, C₁₋₃ alkoxycarbonyl, amino, C₁₋₃ alkylamino, and di(C₁₋₃ alkyl)amino;
each R^{a8} is independently selected from H and C₁₋₆ alkyl; and
each R¹⁰ is independently selected from D, halo, CN, C₁₋₆ alkyl, C₂₋₆ alkenyl, C₂₋₆ alkynyl, C₁₋₆ haloalkyl, OR^{a10}, SR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b11}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10};
each R^{a10}, R^{c10}, and R^{d10} is independently selected from H, C₁₋₆ alkyl, and C₁₋₆ haloalkyl; and
each R^{b10} is independently selected from C₁₋₆ alkyl and C₁₋₆ haloalkyl.

35. The compound of claim 1, wherein the compound is a compound of Formula (II): 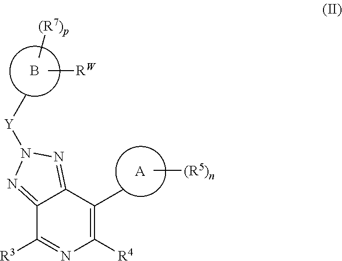
or a pharmaceutically acceptable salt thereof.

36. The compound of claim 1, wherein the compound is a compound of Formula (III): 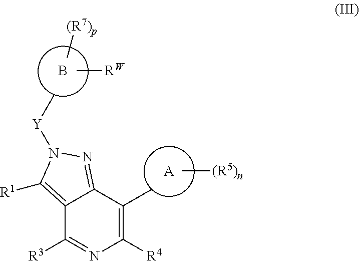
or a pharmaceutically acceptable salt thereof.

37. The compound of claim 1, wherein the compound is a compound of Formula (IV): 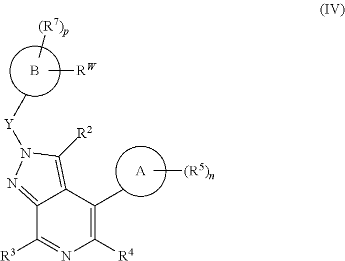
or a pharmaceutically acceptable salt thereof.

38. The compound of claim 1, wherein the compound is a compound of Formula (V): 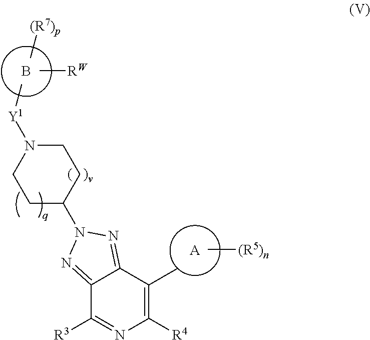
or a pharmaceutically acceptable salt thereof, wherein q is 0 or 1; and v is 0 or 1.

39. The compound of claim 1, wherein the compound is a compound of Formula (VI): 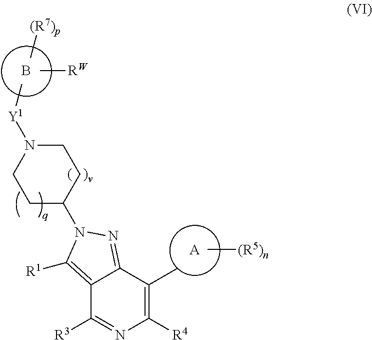
or a pharmaceutically acceptable salt thereof, wherein q is 0 or 1; and v is 0 or 1.

40. The compound of claim 1, wherein the compound is a compound of Formula (VII): 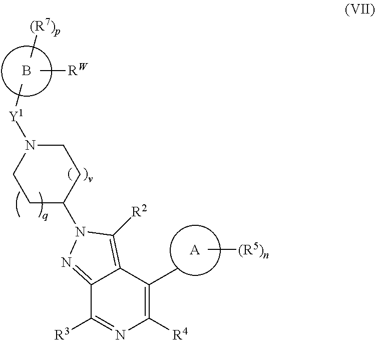
or a pharmaceutically acceptable salt thereof, wherein q is 0 or 1; and v is 0 or 1.

41. The compound of claim 1, or a pharmaceutically acceptable salt thereof, wherein the compound is selected from:
N-(4-(3-(7-(3,5-dichloro-4-hydroxyphenyl)-2H-[1,2,3]triazolo[4,5-c]pyridin-2-yl)azetidine-1-carbonyl)phenyl)acrylamide;
(S)—N-(4-(3-(7-(3,5-dichloro-4-hydroxyphenyl)-2H-[1,2,3]triazolo[4,5-c]pyridin-2-yl)pyrrolidine-1-carbonyl)phenyl)acrylamide;
rac-N-(4-(3-(7-(3,5-dichloro-4-hydroxyphenyl)-2H-[1,2,3]triazolo[4,5-c]pyridin-2-yl)piperidine-1-carbonyl)phenyl)acrylamide;
rac-N-(4-((2R,4S)-4-(7-(3,5-dichloro-4-hydroxyphenyl)-2H-[1,2,3]triazolo[4,5-c]pyridin-2-yl)-2-methylpyrrolidine-1-carbonyl)phenyl)acrylamide;
(S)—N-(4-(3-(7-(3,5-dichloro-4-hydroxyphenyl)-2H-[1,2,3]triazolo[4,5-c]pyridin-2-yl)pyrrolidine-1-carbonyl)-2-methoxyphenyl)acrylamide;
rac-N-(4-(3-(7-(3,5-dichloro-4-hydroxyphenyl)-3-(1-methyl-2-oxo-1,2-dihydropyridin-4-yl)-2H-pyrazolo[4,3-c]pyridin-2-yl)pyrrolidine-1-carbonyl)phenyl)acrylamide;
N-(4-((7-(3,5-dichloro-4-hydroxyphenyl)-2H-[1,2,3]triazolo[4,5-c]pyridin-2-yl)methyl)phenyl)acrylamide;
1-(5-((7-(3,5-dichloro-4-hydroxyphenyl)-2H-[1,2,3]triazolo[4,5-c]pyridin-2-yl)methyl)isoindolin-2-yl)prop-2-en-1-one;
rac-1-(5-(1-(7-(3,5-dichloro-4-hydroxyphenyl)-2H-[1,2,3]triazolo[4,5-c]pyridin-2-yl)ethyl)isoindolin-2-yl)prop-2-en-1-one;
1-(5-((4-(3,5-dichloro-4-hydroxyphenyl)-2H-pyrazolo[3,4-c]pyridin-2-yl)methyl)isoindolin-2-yl)prop-2-en-1-one;
1-(5-((7-(3,5-dichloro-4-hydroxyphenyl)-2H-pyrazolo[4,3-c]pyridin-2-yl)methyl)isoindolin-2-yl)prop-2-en-1-one;
(E)-2-(5-((7-(3,5-dichloro-4-hydroxyphenyl)-2H-[1,2,3]triazolo[4,5-c]pyridin-2-yl)methyl)isoindoline-2-carbonyl)-4-methylpent-2-enenitrile;
1-(5-((7-(3,5-dichloro-4-hydroxyphenyl)-2H-[1,2,3]triazolo[4,5-c]pyridin-2-yl)methyl)isoindolin-2-yl)but-2-yn-1-one;
1-(5-((7-(3,5-dichloro-4-hydroxyphenyl)-2H-[1,2,3]triazolo[4,5-c]pyridin-2-yl)methyl)isoindolin-2-yl)-2-fluoroprop-2-en-1-one;
(E)-1-(5-((7-(3,5-dichloro-4-hydroxyphenyl)-2H-[1,2,3]triazolo[4,5-c]pyridin-2-yl)methyl)isoindolin-2-yl)-4-(1H-1,2,4-triazol-1-yl)but-2-en-1-one;
(E)-1-(5-((7-(3,5-dichloro-4-hydroxyphenyl)-2H-[1,2,3]triazolo[4,5-c]pyridin-2-yl)methyl)isoindolin-2-yl)-3-(pyridin-4-yl)prop-2-en-1-one;
(E)-1-(5-((7-(3,5-dichloro-4-hydroxyphenyl)-2H-[1,2,3]triazolo[4,5-c]pyridin-2-yl)methyl)isoindolin-2-yl)-4,4,4-trifluorobut-2-en-1-one;
(E)-1-(5-((7-(3,5-dichloro-4-hydroxyphenyl)-2H-[1,2,3]triazolo[4,5-c]pyridin-2-yl)methyl)isoindolin-2-yl)-4-hydroxybut-2-en-1-one;
methyl (E)-4-(5-((7-(3,5-dichloro-4-hydroxyphenyl)-2H-[1,2,3]triazolo[4,5-c]pyridin-2-yl)methyl)isoindolin-2-yl)-4-oxobut-2-enoate;
(E)-1-(5-((7-(3,5-dichloro-4-hydroxyphenyl)-2H-pyrazolo[4,3-c]pyridin-2-yl)methyl)isoindolin-2-yl)-4,4-difluorobut-2-en-1-one;
(E)-4-(azetidin-1-yl)-1-(5-((7-(3,5-dichloro-4-hydroxyphenyl)-2H-[1,2,3]triazolo[4,5-c]pyridin-2-yl)methyl)isoindolin-2-yl)but-2-en-1-one;
1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-b]pyridin-6-yl)prop-2-en-1-one;
(E)-1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-b]pyridin-6-yl)-4-(diethylamino)but-2-en-1-one;
(E)-1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-b]pyridin-6-yl)-4-(piperidin-1-yl)but-2-en-1-one;
1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-d]pyrimidin-6-yl)prop-2-en-1-one; and
(E)-1-(2-((7-(3,5-dichloro-4-hydroxyphenyl)-2H-[1,2,3]triazolo[4,5-c]pyridin-2-yl)methyl)-5,7-dihydro-6H-pyrrolo[3,4-d]pyrimidin-6-yl)-4-(piperidin-1-yl)but-2-en-1-one;
or a pharmaceutically acceptable salt of any of the aforementioned.

42. The compound of claim 1, or a pharmaceutically acceptable salt thereof, wherein the compound is selected from:
rac-(E)-1-(2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridin-6-yl)-4-(diethylamino)but-2-en-1-one;
(R,E)-1-(2-(1-(7-(3,5-dichloro-4-hydroxyphenyl)-4-methyl-2H-[1,2,3]triazolo[4,5-c]47yridine-2-yl)ethyl)-5,7-dihydro-6H-pyrrolo[3,4-b]47yridine-6-yl)-4-(diethylamino)but-2-en-1-one;
(S,E)-1-(2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridin-6-yl)-4-(diethylamino)but-2-en-1-one;
(E)-1-(2-(2-(7-(3,5-dichloro-4-hydroxyphenyl)-4-methyl-2H-[1,2,3]triazolo[4,5-c]pyridin-2-yl)propan-2-yl)-5,7-dihydro-6H-pyrrolo[3,4-b]pyridin-6-yl)-4-(dimethylamino)but-2-en-1-one;
(S,E)-1-(2-(1-(7-(3,5-dichloro-4-hydroxyphenyl)-4-methyl-2H-[1,2,3]triazolo[4,5-c]pyridin-2-yl)ethyl)-5,7-dihydro-6H-pyrrolo[3,4-b]pyridin-6-yl)-4-(2,2-dimethylpyrrolidin-1-yl)but-2-en-1-one;
rac-1-(2-(1-(7-(3,5-dichloro-4-hydroxyphenyl)-4-methyl-2H-[1,2,3]triazolo[4,5-c]pyridin-2-yl)ethyl)-3-fluoro-5,7-dihydro-6H-pyrrolo[3,4-b]pyridin-6-yl)prop-2-en-1-one;
(E)-1-(2-(1-(7-(3,5-dichloro-4-hydroxyphenyl)-2H-[1,2,3]triazolo[4,5-c]pyridin-2-yl)cyclobutyl)-5,7-dihydro-6H-pyrrolo[3,4-b]pyridin-6-yl)-4-(diethylamino)but-2-en-1-one;
rac-(E)-1-(2-(cyclopropyl(7-(3,5-dichloro-4-hydroxyphenyl)-4-methyl-2H-[1,2,3]triazolo[4,5-c]pyridin-2-yl)methyl)-5,7-dihydro-6H-pyrrolo[3,4-b]pyridin-6-yl)-4-(dimethylamino)but-2-en-1-one;
rac-(E)-1-(2-(1-(7-(3,5-dichloro-4-hydroxyphenyl)-2H-[1,2,3]triazolo[4,5-c]pyridin-2-yl)propan-2-yl)-5,7-dihydro-6H-pyrrolo[3,4-b]pyridin-6-yl)-4-(diethylamino)but-2-en-1-one;
rac-(E)-1-(2-(1-(4-amino-7-(3,5-dichloro-4-hydroxyphenyl)-2H-[1,2,3]triazolo[4,5-c]pyridin-2-yl)ethyl)-5,7-dihydro-6H-pyrrolo[3,4-b]pyridin-6-yl)-4-(2,2-dimethylpyrrolidin-1-yl)but-2-en-1-one;
(E)-7-(3,5-dichloro-4-hydroxyphenyl)-2-((6-(4-(diethylamino)but-2-enoyl)-3-methoxy-6,7-dihydro-5H-pyrrolo[3,4-b]pyridin-2-yl)methyl)-2H-[1,2,3]triazolo[4,5-c]pyridine-4-carbonitrile;
rac-(E)-4-(3-azabicyclo[3.1.1]heptan-3-yl)-1-(3-(1-(7-(3,5-dichloro-4-hydroxyphenyl)-4-ethyl-2H-[1,2,3]triazolo[4,5-c]pyridin-2-yl)ethyl)-2-methoxy-5,7-dihydro-6H-pyrrolo[3,4-b]pyridin-6-yl)but-2-en-1-one;
rac-1-(2-(1-(7-(3,5-dichloro-4-hydroxyphenyl)-4-methoxy-2H-[1,2,3]triazolo[4,5-c]pyridin-2-yl)ethyl)-5,7-dihydro-6H-pyrrolo[3,4-b]pyridin-6-yl)prop-2-en-1-one;
rac-(E)-1-(2-(1-(6-chloro-7-(3,5-dichloro-4-hydroxyphenyl)-2H-[1,2,3]triazolo[4,5-c]pyridin-2-yl)ethyl)-5,7-dihydro-6H-pyrrolo[3,4-b]pyridin-6-yl)-4-(dimethylamino)but-2-en-1-one;
(E)-1-(5-(2-(7-(2-chloro-6-methoxypyridin-4-yl)-4-methyl-2H-[1,2,3]triazolo[4,5-c]pyridin-2-yl)propan-2-yl)indolin-1-yl)-4-(dimethylamino)but-2-en-1-one;
(E)-4-(bis(methyl-d3)amino)-1-(5-((7-(3,5-dichloro-4-hydroxyphenyl)-4-methyl-2H-[1,2,3]triazolo[4,5-c]pyridin-2-yl)methyl)-4-fluoroindolin-1-yl)but-2-en-1-one;
(E)-1-(2-((7-(3,5-dichloro-4-hydroxyphenyl)-4-methyl-2H-[1,2,3]triazolo[4,5-c]pyridin-2-yl)methyl)-3-ethyl-5,7-dihydro-6H-pyrrolo[3,4-b]pyridin-6-yl)-4-(piperidin-1-yl)but-2-en-1-one;
(E)-4-(3-azabicyclo[3.1.1]heptan-3-yl)-1-(2-(2-(7-(3,5-dichloro-4-hydroxyphenyl)-4-methyl-2H-[1,2,3]triazolo[4,5-c]pyridin-2-yl)propan-2-yl)-4-methoxy-5,7-dihydro-6H-pyrrolo[3,4-b]pyridin-6-yl)but-2-en-1-one;
(E)-4-(3-azabicyclo[3.1.1]heptan-3-yl)-1-(2-((7-(3,5-dichloro-4-hydroxyphenyl)-4-methyl-2H-[1,2,3]triazolo[4,5-c]pyridin-2-yl)methyl)-3-methoxy-5,7-dihydro-6H-pyrrolo[3,4-b]pyrazin-6-yl)but-2-en-1-one;
rac-(E)-1-(2-(1-(7-(3,5-dichloro-4-hydroxyphenyl)-4-methyl-2H-[1,2,3]triazolo[4,5-c]pyridin-2-yl)ethyl)-4-methoxy-5,7-dihydro-6H-pyrrolo[3,4-d]pyrimidin-6-yl)-4-(piperidin-1-yl)but-2-en-1-one;
rac-(E)-1-(5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[2,3-c]pyridin-1-yl)-4-(dimethylamino)but-2-en-1-one;
(S,E)-1-(5-(1-(7-(3,5-dichloro-4-hydroxyphenyl)-4-methyl-2H-[1,2,3]triazolo[4,5-c]pyridin-2-yl)ethyl)-2,3-dihydro-1H-pyrrolo[3,2-b]pyridin-1-yl)-4-(dimethylamino)but-2-en-1-one; and
(E)-1-(5-(2-(7-(2-chloro-6-methoxypyridin-4-yl)-4-methyl-2H-[1,2,3]triazolo[4,5-c]pyridin-2-yl)propan-2-yl)-2,3-dihydro-1H-pyrrolo[2,3-c]pyridin-1-yl)-4-(diethylamino)but-2-en-1-one;
or a pharmaceutically acceptable salt of any of the aforementioned.

43. The compound of claim 1, or a pharmaceutically acceptable salt thereof, wherein the compound is deuterated.

44. A pharmaceutical composition comprising the compound of claim 1, or a pharmaceutically acceptable salt thereof, and a pharmaceutically acceptable carrier.

45. A method of inhibiting VRK1, comprising contacting the VRK1 with the compound of claim 1, or a pharmaceutically acceptable salt thereof.

46. A method of inhibiting VRK1 in a patient, comprising administering to the patient a compound of claim 1, or a pharmaceutically acceptable salt thereof.

47. A method of selectively inhibiting VRK1 over VRK2, comprising contacting the VRK1 and VRK2 with a VRK1 inhibitor, or a pharmaceutically acceptable salt thereof.

48. A method of covalently binding a cysteine in VRK1 with a VRK1 inhibitor, or a pharmaceutically acceptable salt thereof.

49. The method of claim 48, further comprising binding with a water in the binding pocket of VRK1.

50. The method of claim 48, further comprising binding with Phe134 of VRK1.

51. The method of claim 48, further comprising binding with Lys71 of VRK1.

52. The method of claim 48, further comprising binding with Asp132 of VRK1.

53. The method of claim 48, further comprising binding with the phosphate-binding loop of VRK1.

54. The method of claim 47, wherein the VRK1 inhibitor is a compound of Formula (I): 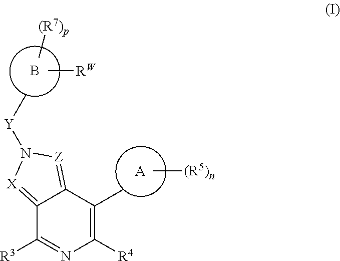
or a pharmaceutically acceptable salt thereof, wherein:
n is 0, 1, 2, 3, 4, 5, or 6;
p is 0, 1, 2, 3, 4, 5, or 6;
r is 1 or 2;
X is N or CR¹;
Z is N or CR²;
Y is -(Cy¹)ₘ-(Y¹)ₛ—, wherein Y¹ is attached to Ring moiety B;
m is 1; s is 1; and Y¹ is a bond; or
m is 1; s is 1; and Y¹ is —(CR⁶R⁶⁰)ᵣ—, —CR⁶R⁶⁰—C(O)—, —C(O)—CR⁶R⁶⁰—, or C(O); or
m is 0; s is 1; and Y¹ is —(CR⁶R⁶⁰)ᵣ—, —CR⁶R⁶⁰—C(O)—, —C(O)—CR⁶R⁶⁰—, or C(O);
Cy¹ is C₃₋₁₀ cycloalkyl, 6-10 membered aryl, 4-10 membered heterocycloalkyl, or 5-10 membered heteroaryl, which is optionally substituted by 1, 2, 3, 4, 5, or 6 independently selected R¹⁰ substituents;
Ring moiety A is C₃₋₁₂ cycloalkyl, 6-10 membered aryl, 4-12 membered heterocycloalkyl, or 5-10 membered heteroaryl;
Ring moiety B is C₃₋₁₂ cycloalkyl, 6-10 membered aryl, 4-12 membered heterocycloalkyl, or 5-10 membered heteroaryl;
R¹ is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{b1}, SR^{b1}, NHOR^{a11}, C(O)R^{b11}, C(O)NR^{c11}R^{d1}, C(O)NR^{c1}(OR^{a1}), C(O)OR^{a11}, OC(O)R^{b11}, OC(O)NR^{c1}R^{d1}, NR^{c1}R^{d1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}C(═NR^{e1})R^{b1}, NR^{c1}S(O)NR^{c1}R^{d1}, NR^{c1}S(O)R^{b1}, NR^{c1}S(O)₂R^{b1}, NR^{c1}S(O)(═NR^{e1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, OS(O)₂R^{b1}, S(O)(═NR^{e1})R^{b1}, SF₅, P(O)R^{f1}R^{g1}, OP(O)(OR^{h1})(ORⁱ¹), P(O)(OR^{h1})(ORⁱ¹), and BR^{j1}R^{k1}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f1} and R^{g1} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h1} and Rⁱ¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j1} and R^{k1} is independently selected from OH, C₁₋₆ alkoxy, and C₁₋₆ haloalkoxy;
or any R^{j1} and R^{k1} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}NR^{d11}, NR^{c11}C(O)R^{b11}, NR^{c11}C(O)OR^{a11} NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹), and BR^{j11}R^{k11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h11} and Rⁱ¹¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j11} and R^{k11} is independently selected from OH, C₁₋₆ alkoxy, and C₁₋₆ haloalkoxy;
or any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12} N, R^{c12}C(═NR^{e12})R^{c12}R^{d12}, NR^{c12}C(═NR^{e12})R^{b12}, NR^{c12}S(O)NR^{c12}R^{d12}, NR^{c12}S(O)R^{b12}, NR^{c12}S(O)₂R^{b12}, NR^{c12}S(O)(═NR^{e12})R^{b12}, NR^{c12}S(O)₂NR^{c12}R^{d12}S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c12} and R^{d12} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{b2}, SR^{b2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2}, NR^{c2}S(O)₂R^{b2}, NR^{c2}S(O)(═NR^{e2})R^{b2}, NR^{c2}S(O)₂NR^{c2}R^{d2}S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, S(O)(═NR^{e2})R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{2A} substituents;
or, any R^{c2} and R^{d2} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{2A} substituents;
each R^{b2} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f2} and R^{g2} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h2} and Rⁱ² is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a21}, SR^{a21}, NHOR^{a21}C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR²¹NR^{c21}R^{d21}, NR^{c2}C(O)R^{b21}, NR^{c21}C(O)OR^{a21}, NR^{c2}C(O)NR^{c21}R^{d21}, C(═NR^{e21})R^{b21}, C(═NR^{e21})NR^{c2}R^{d21}, NR^{c21}C(═NR^{e21})R^{c21}R^{d21}, NR^{c21}C(═NR^{e21})R^{b21}, NR^{c21}S(O)NR^{c21}R^{d21}, NR^{c2}S(O)R^{b21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, S(O)(═NR^{e21})R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{2B} substituents;
or, any R^{c21} and R^{d21} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{2B} substituents;
each R^{b21} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f21} and R^{g21} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h21} and Rⁱ²¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a22}, SR^{a22}, NHOR^{a22}, C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR²²NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22})R^{b22}, NR^{c22}S(O)NR^{c22}R^{d22}, NR^{c22}S(O)R^{b22}, NR^{c22}S(O)₂R^{b22}, NR^{c22}S(O)(═NR^{e22})R^{b22}, NR^{c22}S(O)₂NR^{c22}R^{d22}S(O)R^{b22}, S(O)NR^{c22}R^{d22}, S(O)₂R^{b22}, S(O)₂NR^{c22}R^{d22}, OS(O)(═NR^{e22})R^{b22}, OS(O)₂R^{b22}, S(O)(═NR^{e22})R^{b22}, SF₅, P(O)R^{f22}R^{g22}, OP(O)(OR^{h22})(ORⁱ²²), P(O)(OR^{h22})(ORⁱ²²), and BR^{j22}R^{k22}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c22} and R^{d22} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G}each R^{b22} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f22} and R^{g22} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h22} and Rⁱ²² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j22} and R^{k22} is independently selected from OH, C₁₋₆ alkoxy, and C₁₋₆ haloalkoxy;
or any R^{j22} and R^{k22} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R³ is selected from H, D, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b3} is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4}, NR^{c4}C(═NR^{e4})R^{b4}, NR^{c4}S(O)NR^{c4}R^{d4}, NR^{c4}S(O)R^{b4}, NR^{c4}S(O)₂R^{b4}, NR^{c4}S(O)(═NR^{e4})R^{b4}, NR^{c4}S(O)₂NR^{c4}R^{d4}S(O)R^{b4}, S(O)NR^{c4}R^{d4}S(O)₂R^{b4}, and S(O)₂NR^{c4}R^{d4}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
each R^{a4}, R^{c4}, and R^{d4} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b4} is independently selected from C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e4} is independently selected from H, OH, CN, C₁₋₆ alkyl, C₁₋₆ alkoxy, C₁₋₆ haloalkyl, and C₁₋₆ haloalkoxy;
each R⁵ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}CC(O)R^{b5}, NR^{c5}CC(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
or, any R^{c5} and R^{d5} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)NR^{c51}R^{d51}, NR^{c51}S(O)R^{b51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, OS(O)₂R^{b51}, S(O)(═NR^{e51})R^{b51}, SF₅, P(O)R^{f51}R^{g51}, OP(O)(OR^{h51})(ORⁱ⁵¹), P(O)(OR^{h51})(ORⁱ⁵¹), and BR^{j51}R^{k51}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c51} and R^{d51} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b51} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f51} and R^{g51} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h51} and Rⁱ⁵¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j51} and R^{k51} is independently selected from OH, C₁₋₆ alkoxy, and C₁₋₆ haloalkoxy;
or any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶ is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c6}R^{d6}, C(═NR^{e6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)NR^{c6}R^{d6}, NR^{c6}S(O)R^{b6}, NR^{c6}S(O)₂R^{b6}, NR^{c6}S(O)(═NR^{e6})R^{b6}, NR^{c6}S(O)₂NR^{c6}R^{d6}, S(O)R^{b6}, S(O)NR^{c6}R^{d6}, S(O)₂R^{b6}, S(O)₂NR^{c6}R^{d6}, OS(O)(═NR^{e6})R^{b6}, OS(O)₂R^{b6}, S(O)(═NR^{e6})R^{b6}, SF₅, P(O)R^{f6}R^{g6}, OP(O)(OR^{h6})(OR¹⁶), P(O)(OR^{h6})(OR¹⁶), and BR^{j6}R^{k6}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{6A} substituents;
or, any R^{c6} and R^{d6} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{6A} substituents;
each R^{b6} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected RA substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f6} and R^{g6} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h6} and Rⁱ⁶ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j6} and R^{k6} is independently selected from OH, C₁₋₆ alkoxy, and C₁₋₆ haloalkoxy;
or any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6A} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a61}, SR^{a61}, NHOR^{a61}, C(O)R^{b61}, C(O)NR^{c61}R^{d61}, C(O)NR^{c61}(OR^{a61}), C(O)OR^{a61}, OC(O)R^{b61}, OC(O)NR^{c6}R^{d61}, NR^{c61}R^{d61}, NR^{c61}NR^{c61}R^{d61}, NR^{c61}C(O)R^{b61}, NR^{c61}C(O)OR^{a61}, NR^{c61}C(O)NR^{c61}R^{d61}, C(═NR^{e61})R^{b61}, C(═NR^{e61})NR^{c61}R^{d61}, NR^{c61}C(═NR^{e61})NR^{c61}R^{d61}, NR^{c61}C(═NR^{e61})R^{b61}, NR^{c61}S(O)NR^{c61}R^{d61}, NR^{c6}S(O)R^{b61}, NR^{c61}S(O)₂R^{b61}, NR^{c61}S(O)(═NR^{e61})R^{b61}, NR^{c61}S(O)₂NR^{c61}R^{d61}, S(O)R^{b61}, S(O)NR^{c61}R^{d61}, S(O)₂R^{b61}, S(O)₂NR^{c6}R^{d61}, OS(O)(═NR^{e61})R^{b61}, OS(O)₂R^{b61}, S(O)(═NR^{e61})R^{b61}, SF₅, P(O)R^{f61}R^{g61}, OP(O)(OR^{h61})(ORⁱ⁶¹), P(O)(OR^{h61})(ORⁱ¹⁶), and BRⁱ⁶¹R^{k61}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
each R^{a61}, R^{c61}, and R^{d61} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{6B} substituents;
or, any R^{c61} and R^{d61} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{6B} substituents;
each R^{b61} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{6B} substituents;
each R^{e61} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f61} and R^{g61} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h61} and Rⁱ⁶¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j61} and R^{k61} is independently selected from OH, C₁₋₆ alkoxy, and C₁₋₆ haloalkoxy;
or any R^{j61} and R^{k61} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{6B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a62}, SR^{a62}, NHOR^{a62}C(O)R^{b62}, C(O)NR^{c62}R^{d62}, C(O)NR^{c62}(OR^{a62}), C(O)OR^{a62}, OC(O)R^{b62}, OC(O)NR^{c62}R^{d62}, NR^{c62}R^{d62}, NR^{c62}NR^{c62}R^{d62}, NR^{c62}C(O)R^{b62}, NR^{c62}C(O)OR^{a62}, NR^{c62}C(O)NR^{c62}R^{d62}, C(═NR^{e62})R^{b62}, C(═NR^{e62})NR^{c62}R^{d62}, NR^{c62}C(═NR^{e62}NR^{c62}R^{d62}, NR^{c62}C(═NR^{e62})R^{b62}, NR^{c62}S(O)NR^{c62}R^{d62}, NR^{c62}S(O)R^{b62}, NR^{c62}S(O)₂R^{b62}, NR^{c62}S(O)(═NR^{e62})R^{b62}, NR^{c62}S(O)₂NR^{c62}R^{d62}S(O)R^{b62}, S(O)NR^{c62}R^{d62}, S(O)₂R^{b62}, S(O)₂NR^{c62}R^{d62}, OS(O)(═NR^{e62})R^{b62}, OS(O)₂R^{b62}, S(O)(═NR^{e62})R^{b62}, SF₅, P(O)R^{f62}R^{g62}, OP(O)(OR^{h62})(ORⁱ⁶²), P(O)(OR^{h62})(ORⁱ⁶²), and BR^{j62}R^{k62}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a62}, R^{c62}, and R^{d62} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c62} and R^{d62} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b62} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e62} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f62} and R^{g62} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇cycloalkyl, phenyl, 4-7 membered heterocycloalkyl, 5-6 membered heteroaryl, C₃₋₇cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h62} and Rⁱ⁶² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j62} and R^{k62} is independently selected from OH, C₁₋₆ alkoxy, and C₁₋₆ haloalkoxy;
or any R^{j62} and R^{k62} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R⁶⁰ is independently selected from H, D, C₁₋₆ alkyl, and C₁₋₆ haloalkyl;
or R⁶ and R⁶⁰, together with the carbon atom to which they are attached, form a C₃₋₁₀ cycloalkyl or a 4-10 membered heterocycloalkyl moiety, each of which is optionally substituted with 1, 2, 3, or 4 independently selected R^{7A} substituents;
each R⁷ is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)NR^{c7}R^{d7}, NR^{c7}S(O)R^{b7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{d7}S(O)₂R^{b7}, S(O)₂NR^{c7}R^{d7}, OS(O)(═NR^{e7})R^{b7}, OS(O)₂R^{b7}, S(O)(═NR^{e7})R^{b7}, SF₅, P(O)R^{f7}R^{g7}, OP(O)(OR^{h7})(OR¹⁷), P(O)(OR^{h7})(OR¹⁷), and BR^{j7}R^{k7}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{7A} substituents;
or, any R^{c7} and R^{d7} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{7A} substituents;
each R^{b7} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f7} and R^{g7} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h7} and Rⁱ⁷ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j7} and R^{k7} is independently selected from OH, C₁₋₆ alkoxy, and C₁₋₆ haloalkoxy;
or any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7A} is independently selected from D, halo, CN, NO₂, C₁₋₆ alkyl, C₂₋₆ alkenyl, C₂₋₆ alkynyl, C₁₋₆ haloalkyl, C₃₋₇cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a71}, SR^{a71}, NHOR^{a71}, C(O)R^{b71}, C(O)NR^{c71}R^{d71}, C(O)NR^{c71}(OR^{a71}), C(O)OR^{a71}, OC(O)R^{b71}, OC(O)NR^{c71}R^{d71}, NR^{c71}R^{d71}, NR^{c71}NR^{c71}R^{d71}, NR^{c71}C(O)R^{b71}, NR^{c71}C(O)OR^{a71}, NR^{c71}C(O)NR^{c71}R^{d71}, C(═NR^{e71})R^{b71}, C(═NR^{e71})NR^{c7}R^{d71}, NR^{c71}C(═NR^{e71})NR^{c71}R^{d71}, NR^{c71}C(═NR^{e71})R^{b71}, NR^{c71}S(O)NR^{c71}R^{d71}, R^{c71}S(O)R^{b71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, OS(O)₂R^{b71}, S(O)(═NR^{e71})R^{b71}, SF₅, P(O)R^{f71}R^{g71}, OP(O)(OR^{h71})(ORⁱ⁷¹), P(O)(OR^{h71})(ORⁱ⁷¹), and BR^{j71}R^{k71}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{7B} substituents;
or, any R^{c71} and R^{d71} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{7B}
each R^{b71} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{7B} substituents;
each R^{e71} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f71} and R^{g71} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h71} and Rⁱ⁷¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j71} and R^{k71} is independently selected from OH, C₁₋₆ alkoxy, and C₁₋₆ haloalkoxy;
or any R^{j71} and R^{k71} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{7B} is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a72}, SR^{a72}, NHOR^{a72}C(O)R^{b72}, C(O)NR^{c72}R^{d72}, C(O)NR^{c72}(OR^{a72}), C(O)OR^{a72}, OC(O)R^{b72}, OC(O)NR^{c72}R^{d72}, NR^{c72}R^{d72}, NR^{c72}NR^{c72}R^{d72}, NR^{c72}C(O)R^{b72}, NR^{c72}C(O)OR^{a72}, NR^{c72}C(O)NR^{c72}R^{d72}, C(═NR^{e72})R^{b72}, C(═NR^{e72})NR^{c72}R^{d72}, NR^{c72}C(═NR^{e72})NR^{c72}R^{d72}, NR^{c72}C(═NR^{e72})R^{b72}, NR^{c72}S(O)NR^{c72}R^{d72}, NR^{c72}S(O)R^{b72}, NR^{c72}S(O)₂R^{b72}, NR^{c72}S(O)(═NR^{e72})R^{b72}, NR^{c72}S(O)₂NR^{c72}R^{d72}S(O)R^{b72}, S(O)NR^{c72}R^{d72}, S(O)₂R^{b72}, S(O)₂NR^{c72}R^{d72}, OS(O)(═NR^{e72})R^{b72}, OS(O)₂R^{b72}, S(O)(═NR^{e72})R^{b72}, SFS, P(O)R^{f72}R^{g72}, OP(O)(OR^{h72})(ORⁱ⁷²), P(O)(OR^{h72})(ORⁱ⁷²), and BR^{j72}R^{k72}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a72}, R^{c72}, and R^{d72} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c72} and R^{d72} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b72} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e72} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f72} and R^{g72} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h72} and Rⁱ⁷² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j72} and R^{k72} is independently selected from OH, C₁₋₆ alkoxy, and C₁₋₆ haloalkoxy;
or any R^{j72} and R^{k72} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R^{W} is a covalent warhead moiety having a reactive functional group capable of covalently binding to one or more cysteine residues in a protein;
each R¹⁰ is independently selected from D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a10}, SR^{a10}, NHOR^{a10}, C(O)R^{b10}, C(O)NR^{c10}R^{d10}, C(O)OR^{a10}, OC(O)R^{b10}, OC(O)NR^{c10}R^{d10}, NR^{c10}R^{d10}, NR^{c10}C(O)R^{b10}, NR^{c10}C(O)OR^{a10}, NR^{c10}C(O)NR^{c10}R^{d10}, NR^{c10}S(O)₂R^{b10}, NR^{c10}S(O)₂NR^{c10}R^{d10}, S(O)₂R^{b10}, and S(O)₂NR^{c10}R^{d10}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a10}, R^{c10}, and R^{d10} is independently selected from H, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c10} and R^{d10} attached to the same N atom, together with the N atom to which they are attached, form a 4-7 membered heterocycloalkyl group, wherein the 4-10 membered heterocycloalkyl group is optionally substituted with 1, 2, 3, or 4 independently selected R^{3A} substituents;
each R^{b10} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{3A} substituents; and
each R^{G} is independently selected from D, OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, N(CD₃)₂, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

55. A method of treating a disease or disorder associated with VRK1 in a patient, comprising administering to the patient a therapeutically effective amount of the compound of claim 1, or pharmaceutically acceptable salt thereof.

56. The method of claim 55, wherein the disease or disorder is cancer.

57. The method of claim 56, wherein the cancer is sarcoma, colorectal adenocarcinoma, acute myeloid leukemia, glioblastoma, and neuroblastoma.

58. The method of claim 57, wherein the glioblastoma or neuroblastoma have overepression of VRK1.

59. The method of claim 57, wherein the glioblastoma or neuroblastoma have underexpression of VRK2.

60. The method of claim 56, wherein the cancer is bone cancer, cancer of the neck and head, rectal cancer, esophageal cancer, sarcoma of soft tissue, neoplasm of the central nervous system (CNS), brain stem, glioma, colon cancer, Ewing's sarcoma, brain cancer, astrocytoma, neuroblastoma, intestinal cancer, nervous system cancers, osteosarcoma, cancer of the small bowel, adenocarcinoma of the small bowel, lymphoma of the small bowel, carcinoid tumors of the small bowel, Kaposi's sarcoma of the small bowel, leiomyoma of the small bowel, hemangioma of the small bowel, lipoma of the small bowel, neurofibroma of the small bowel, fibroma of the small bowel, cancer of the large bowel, adenocarcinoma of the large bowel, tubular adenoma of the large bowel, villous adenoma of the large bowel, hamartoma of the large bowel, or leiomyoma of the large bowel.
